(19) United States
(12) Patent Application Publication
(10) Pub. No.: US 2024/0043400 A1
(43) Pub. Date: Feb. 8, 2024

(54) Octahydroisoquinolinyl Derivatives

(71) Applicant: UCB Biopharma SRL, Brussels (BE)

(72) Inventors: Ali Ates, Brussels (BE); Pierre Burssens, Brussels (BE); Adrian Hall, Brussels (BE); Laurent Provins, Brussels (BE); David Skolc, Brussels (BE); Anne Valade, Brussels (BE)

(21) Appl. No.: 18/265,156

(22) PCT Filed: Dec. 1, 2021
(86) PCT No.: PCT/EP2021/083833

§ 371 Date: Jun. 2, 2023

(30) Foreign Application Priority Data
Dec. 3, 2020 (EP) 20211398.1

(51) Int. Cl. C07D 401/06; C07D 217/04; C07D 471/04; C07D 401/10

(52) U.S. Cl. CPC C07D 401/06; C07D 217/04; C07D 471/04; C07D 401/10

(57) ABSTRACT

The present invention relates to octahydroisoquinolinyl derivatives according to formula (I), which are Positive Allosteric Modulators of D1 and accordingly of benefit as pharmaceutical agents for the treatment of diseases in which D1 receptors play a role.

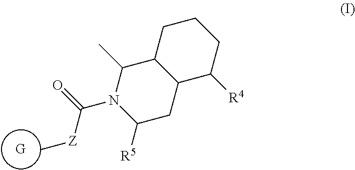

The invention relates to octahydroisoquinolinyl derivatives and their use in therapy. In particular the present invention relates to pharmacologically active fused ocathydroisoquinolinyl derivatives and analogs thereof. More particularly, the present invention relates to substituted 3, 4, 4a, 5, 6, 7, 8, 8a-octahydro-1H-isoquinol-2-yl derivatives and analogs thereof.

The compounds according to the present invention are D1 Positive Allosteric Modulators and accordingly of benefit as pharmaceutical agents for the treatment of diseases in which D1 receptors play a role.

The monoamine dopamine acts via two families of GPCRs to modulate motor function, reward mechanisms, cognitive processes and other physiological functions. Specifically, dopamine is acting upon neurons via D1-like, comprising dopamine D1 and D5, receptors which couple mainly to the Gs G-protein and thereby stimulate cAMP production, and D2-like, which comprise D2, D3 and D4, receptors which couple to Gi/qG-proteins and which attenuate cAMP production. These receptors are widely expressed in different brain regions. In particular, D1 receptors are involved in numerous physiological functions and behavioural processes. D1 receptors are, for instance, involved in synaptic plasticity, cognitive function and goal-directed motor functions, but also in reward processes. Due to their role in several physiological/neurological processes, D1 receptors have been implicated in a variety of disorders including cognitive and negative symptoms in schizophrenia, cognitive impairment related to neuroleptic therapy, Mild Cognitive Impairment (MCI), impulsivity, Attention-Deficit Hyperactivity Disorder (ADHD), Parkinson's disease and other movement disorders, dystonia, Parkinson's dementia, Huntington's disease, dementia with Lewy Body, Alzheimer's disease, drug addiction sleep disorders, apathy, traumatical spinal cord injury or neuropathic pain.

It has proven difficult to develop orally-bioavailable small molecules targeting D1 receptors. D1 agonists developed so far are generally characterized by a catechol moiety and their clinical use has therefore been limited to invasive therapies. Achieving sufficient selectivity has also been challenging due to the high degree of homology in the ligand binding site between dopamine receptors subtypes (e.g. dopamine D1 and D5). Also, D1 agonists are associated with potentially limiting side effects including but not limited to dyskinesia and hypotension.

There is therefore a need to design new agents that could modulate D1 receptors.

There has been much interest in the identification of allosteric modulators of GPCRs, both as tools to understand receptor mechanisms and as potential therapeutic agents. GPCRs represent the largest family of cell-surface receptors and a large number of marketed drugs directly activate or block signaling pathways mediated by these receptors. However, for some GPCRs (e.g. peptide receptors), it has proven challenging to develop small molecules or to achieve sufficient selectivity due to the high degree of homology in the ligand binding site between subtypes (e.g. dopamine D1 and D5 or D2 and D3). Accordingly, much drug research has shifted to the identification of small molecules which target sites distinct from the orthosteric natural agonist. Ligands which bind to these sites induce a conformational change in the GPCR thereby allosterically modulating the receptor function. Allosteric ligands have a diverse range of activities including the ability to potentiate (positive allosteric modulator, PAM) or attenuate (negative allosteric modulator, NAM) the effects of the endogenous ligand, by affecting affinity and/or efficacy. As well as subtype selectivity, allosteric modulators may present other potential advantages from a drug discovery perspective such as a lack of direct effect or intrinsic efficacy; only potentiating the effect of the native transmitter where and when it is released; reduced propensity for inducing desensitization arising from constant exposure to an agonist as well as reduced propensity to induce target-related side-effects.

The compounds according to the present invention potentiates the effect of D1 agonists or of the endogenous ligand on D1 receptors through an allosteric mechanism and is therefore a D1 Positive Allosteric Modulator (D1 PAM).

The compounds in accordance with the present invention, being D1 PAMs, are therefore beneficial in the treatment and/or prevention of diseases and disorders in which D1 receptors play a role. Such diseases include cognitive and negative symptoms in schizophrenia, cognitive impairment related to neuroleptic therapy, Mild cognitive impairment (MCI), impulsivity, Attention-Deficit Hyperactivity Disorder (ADHD), Parkinson's disease and other movement disorders, dystonia, Parkinson's dementia, Huntington's disease, dementia with Lewy Body, Alzheimer's disease, drug addiction, sleep disorders, apathy, traumatic spinal cord injury or neuropathic pain.

International patent application WO2014/193781 A1 discloses certain 3,4-dihydroisoquinolin-2 (1H)-yl derivatives useful for the treatment of cognitive impairment associated with Parkinson's disease or Schizophrenia.

International patent application WO2016/055479 discloses substituted 3,4-dihydroisoquinolin-2 (1H)-yl derivatives and analogs thereof which may be useful for the treatment of diseases in which D1 receptors play a role.

However, there remains a need to develop potent D1 positive allosteric modulators combining advantageous pharmacokinetic and pharmacodynamic properties while reducing side effects traditionally associated with treatments involving selective D1 agonists, such as for example hypotension or dyskinesia.

The present invention provides a compound of formula (I), or a pharmaceutically acceptable salt thereof,

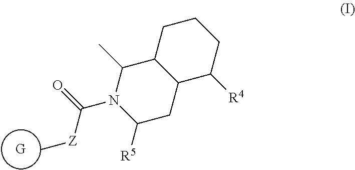

wherein

Z represents CH₂ or NH;

R⁴ represents C₁₋₆ alkyl optionally substituted by one or more substituents selected from hydroxy, halogen and C₁₋₆ alkyl; or C₁₋₆ alkyne optionally substituted by one or more substituents selected from hydroxy and C₁₋₆ alkyl; or C₅₋₈ heteroaryl, optionally substituted by one or more substituents selected from halogen, cyano, C₁₋₆ alkyl and C₁₋₆ alkoxy;

R⁵ represents hydrogen or a C₁₋₆ alkyl optionally substituted by one or more substituents selected from hydroxy and halogen; and

G represents an aromatic group selected from the group consisting of (G^{a}), (G^{b}) and (G^{c});

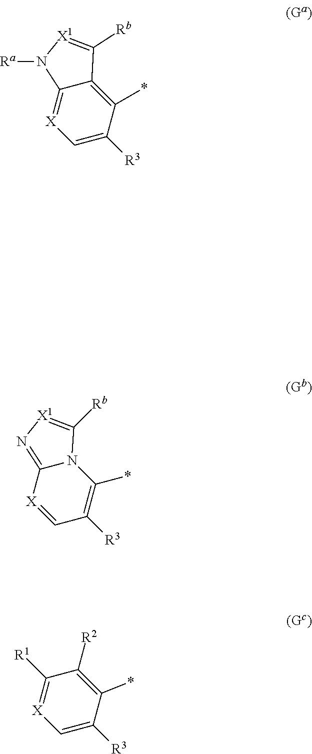

wherein

the asterisk (*) represents the point of attachment of G to the remainder of the molecule;

X represents CH, C—F or N;

R¹ represents hydrogen; or C₁₋₆ alkyl or C₁₋₆ alkoxy optionally substituted by one or more substituents selected from hydroxy and halogen;

R² and R³ represent independently halogen or cyano;

X¹ represents CH or N;

R^{a} represents hydrogen or C₁₋₆ alkyl; and

R^{b} represents C₁₋₆ alkyl or halogen.

None of the prior art available to date discloses or suggests the precise structural class of substituted octahydrohydroisoquinolinyl derivatives as provided by the present invention.

The term “C₁₋₆alkyl” as used herein refers to aliphatic hydrocarbon groups which may be straight or branched and may comprise 1 to 6 carbon atoms in the chain. Suitable alkyl groups which may be present on the compounds of use in the invention include straight-chained and branched C₁₋₄ alkyl groups. Illustrative C₁₋₆ alkyl groups include methyl, ethyl, propyl and butyl.

The term “C₁₋₆ alkoxy” refers to a group of formula —O—R where R is an optionally substituted “C₁₋₆ alkyl”. Suitable alkoxy groups according to the present invention include methoxy.

The term “heteroaryl” as used herein represents aromatic carbocyclic groups of from 5 to 14 carbon atoms, having a single ring or multiple condensed rings, wherein one or more of the said carbon atoms have been replaced by one or more heteroatoms selected from oxygen, sulphur and nitrogen.

Where any of the groups in the compounds of formula (I) above is stated to be optionally substituted, this group may be unsubstituted, or substituted by one or more substituents. Typically, such groups will be unsubstituted, or substituted by one or two substituents. Suitable substitutents for each particular groups of compounds formula (I) are further described here after in the present specification.

Formula (I) and the formulae depicted hereinafter are intended to represent all individual stereoisomers and all possible mixtures thereof, unless stated or shown otherwise.

Stereoisomers of compounds of formula (I) include cis and trans isomers, optical isomers such as R and S enantiomers, diastereomers, geometric isomers, rotational isomers, atropisomers, and conformational isomers of the compounds of formula (I), including compounds exhibiting more than one type of isomerism; and mixtures thereof (such as racemates and diastereomeric pairs).

Compounds of formula (I) include asymmetric carbon atoms. The carbon-carbon bonds of the compounds of formula (I) are depicted herein using a solid line ( [symbol] ), a solid wedge ( [symbol] ) or a dotted wedge ( [symbol] ). The use of a solid line to depict bonds to asymmetric carbon atoms is meant to indicate that all possible stereoisomers (e.g., specific enantiomers, racemic mixtures, etc.) at that carbon atom are included. The use of either a solid or dotted wedge to depict bonds to asymmetric carbon atoms is meant to indicate that only the stereoisomer shown is meant to be included. It is possible that compounds of formula (I) may contain more than one asymmetric carbon atom. In those compounds, the use of a solid line to depict bonds to asymmetric carbon atoms is meant to indicate that all possible stereoisomers are meant to be included.

Examples of stereoisomers according to the present invention include compounds represented by formula (IA) and (IA-a) as depicted here below.

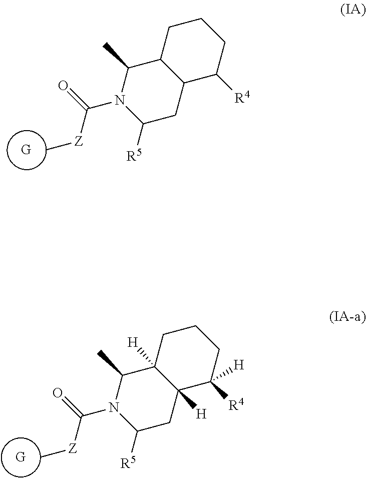

wherein G, R⁴, R⁵ and Z are as defined here above for compounds of formula (I).

Some of the compounds of formula (I) may exist in tautomeric forms. Such forms although not explicity indicated in the above formula are intended to be included within the scope of the present invention. Examples of tautomers include keto (CH₂C═O)↔enol (CH═CHOH) tautomers or amide (NHC═O)↔hydroxyimine (N═COH) tautomers. Formula (I) and the formulae depicted hereinafter are intended to represent all individual tautomers and all possible mixtures thereof, unless stated or shown otherwise.

It is also to be understood that each individual atom present in formula (I), or in the formula depicted hereinafter, may in fact be present in the form of any of its naturally occurring isotopes, with the most abundant isotope(s) being preferred. Thus, by way of example, each individual hydrogen atom present in formula (I), or in the formula depicted hereinafter, may be present as a ¹H, ²H (deuterium) or ³H (tritium) atom, preferably ¹H or ²H. Similarly, by way of example, each individual carbon atom present in formula (I), or in the formulae depicted hereinafter, may be present as a ¹²C, ¹³C or ¹⁴C atom, preferably ¹²C.

Specific embodiments of compounds of formula (I) according to the present invention are described hereafter.

In one embodiment Z represents CH₂. In another embodiment, Z represents N.

In one embodiment G represents (G^{a}). In a second embodiment, G represents (G^{b}). In a third embodiment G represents (G^{c}).

In a first embodiment X represents CH. In a second embodiment, X represents N. In a third embodiment, X represents C—F.

In a first embodiment, R¹ represents hydrogen.

In a second embodiment, R¹ represents C₁₋₆ alkyl optionally substituted by one or more substituents selected from hydroxy and halogen. In a first aspect according to this embodiment, R¹ represents a C₁₋₆ alkyl substituted by one or more hydroxy. Examples of R¹ according to this aspect are hydroxymethyl, hydroxyethyl, and hydroxypropyl. In a second aspect according to this embodiment, R¹ represents a C₁₋₆ alkyl substituted by one or more hydroxy and by one or more halogen. Examples of R¹ according to this aspect are (difluoro)(hydroxy)ethyl and (difluoro)(hydroxy)propyl. In a third aspect, R¹ represents C₁₋₆ alkyl optionally substituted by one or more substituents selected halogen.

In a third embodiment, R¹ represents C₁₋₆ alkoxy optionally substituted by one or more substituents selected from hydroxy and halogen. In a first aspect according to this embodiment, R¹ represents C₁₋₆alkoxy. Examples of R¹ according to this aspect are methoxy and deuteriated methoxy (CD₃O—). In a second aspect according to this embodiment, R¹ represents C₁₋₆ alkoxy substituted by one or more halogen. An example of R¹ according to this aspect is difluoromethoxy.

Generally, R¹ represents hydrogen, C₁₋₆ alkyl substituted by one or more hydroxy, C₁₋₆ alkyl substituted by one or more hydroxy and by one or more halogen, C₁₋₆ alkoxy, or C₁₋₆ alkoxy substituted by one or more halogen.

Suitably, R¹ represents C₁₋₆ alkyl substituted by one or more hydroxy, C₁₋₆ alkyl substituted by one or more hydroxy and by one or more halogen, C₁₋₆ alkoxy, or C₁₋₆ alkoxy substituted by one or more halogen.

Typically, R¹ represents hydrogen, hydroxymethyl, hydroxyethyl, (hydroxy)propyl, (hydroxy)(difluoro)ethyl, (hydroxy)(difluoro)propyl, methoxy, deuteriated methoxy, or difluoromethoxy.

Ideally, R¹ represents hydroxymethyl, 1-hydroxyethyl, 2-hydroxypropan-2-yl, 2,2-difluoro-1-hydroxyethyl, 1,1-difluoro-2-hydroxypropan-2-yl, methoxy, deuteriated methoxy, or difluoromethoxy.

Illustratively, R¹ represents hydrogen, hydroxymethyl, 1-hydroxyethyl, 2-hydroxypropan-2-yl, 2,2-difluoro-1-hydroxyethyl, 1,1-difluoro-2-hydroxypropan-2-yl, methoxy, deuteriated methoxy, or difluoromethoxy.

Selectively, R¹ represents hydroxymethyl, 1-hydroxyethyl, 2-hydroxypropan-2-yl, 2,2-difluoro-1-hydroxyethyl, 1,1-difluoro-2-hydroxypropan-2-yl, methoxy, deuteriated methoxy, or difluoromethoxy.

In a first embodiment, R² represents halogen. In a first aspect of this embodiment, R² represents chloro. In a second aspect of this embodiment, R² represents bromo. In a third aspect of this embodiment, R² represents fluoro. In a second embodiment, R² represents cyano.

Illustratively, R² represents chloro or cyano.

In a first embodiment, R³ represents halogen. In a first aspect of this embodiment, R³ represents chloro. In a second aspect of this embodiment, R³ represents bromo. In a third aspect of this embodiment, R³ represents fluoro. In a second embodiment, R³ represents cyano.

Illustratively, R³ represents chloro or cyano.

In one embodiment, X¹ represents CH. In another embodiment, X¹ represents N.

In one embodiment, R^{a} represents hydrogen. In a second embodiment, R^{a} represents C₁₋₆ alkyl. An example of R^{a} according to this aspect is methyl.

In one embodiment, R^{b} represents C₁₋₆ alkyl. An example of R^{b} according to this embodiment is methyl. In a second embodiment, R^{b} represents halogen, particularly chloro.

In a first embodiment, R⁴ represents C₁₋₆ alkyl optionally substituted by one or more substituents selected from hydroxy, halogen and C₁₋₆ alkyl. In a first aspect of this embodiment, R⁴ represents C₁₋₆ alkyl. In a second aspect of this embodiment, R⁴ represents C₁₋₆ alkyl substituted by one or more hydroxy and by one or more halogen. Examples of R⁴ according to this aspect are (trifluoro)(hydroxy)ethyl, (difluoro)(hydroxy)ethyl, (difluoro)(hydroxy)propyl and (trifluoro)(hydroxy)propyl. In a third aspect of this embodiment, R⁴ represents C₁₋₆ alkyl substituted by one or more C₁₋₆ alkyl and by one or more hydroxy. An example of R⁴ according to this aspect is (hydroxy)(methyl)butyl.

In a second embodiment, R⁴ represents C₁₋₆ alkyne optionally substituted by one or more substituent selected from hydroxy and C₁₋₄ alkyl. In a first aspect of this embodiment, R⁴ represents C₁₋₆ alkyne. In a second aspect of this embodiment, R⁴ represents C₁₋₆ alkyne substituted by one or more hydroxy and by one or more C₁₋₆ alkyl. An example of R⁴ according to this aspect is (hydroxy)(methyl)butynyl.
In a third embodiment, R⁴ represents C₅₋₈ heteroaryl optionally substituted by trifluoromethyl, halogen, cyano, C₁₋₆ alkyl or C₁₋₆ alkoxy. In one aspect of this embodiment, R⁴ represents C₅₋₈ heteroaryl. An example of R⁴ according to this aspect is 2H-triazol-4-yl.

Generally, R⁴ represents C₁₋₆ alkyl substituted by one or more hydroxy and by one or more halogen, C₁₋₆ alkyl substituted by one or more C₁₋₆ alkyl and by one or more hydroxy, C₁₋₆ alkyne substituted by one or more hydroxy and one or more C₁-6 alkyl, or C₅₋₈ heteroaryl.

Suitably, R⁴ represents C₁₋₆ alkyl substituted by one or more hydroxy and by one or more halogen, C₁₋₆ alkyl substituted by one or more C₁₋₆ alkyl and by one or more hydroxy, or C₁₋₆ alkyne substituted by one or more hydroxy and one or more C₁₋₆ alkyl.

Typically, R⁴ represents (trifluoro)(hydroxy)ethyl, (difluoro)(hydroxy)ethyl, (difluoro)(hydroxy)propyl, (trifluoro)(hydroxy)propyl, (hydroxy)(methyl)butyl, (hydroxy)(methyl)butynyl or 2H-triazol-4-yl.

In a particular embodiment, R⁴ represents (trifluoro)(hydroxy)ethyl, (difluoro)(hydroxy)ethyl, (difluoro)(hydroxy)propyl, (trifluoro)(hydroxy)propyl, (hydroxy)(methyl)butyl or (hydroxy)(methyl)butynyl.

Illustratively, R⁴ represents 2,2,2-trifluoro-1-hydroxyethyl, 2,2-difluoro-1-hydroxyethyl, 1,1-difluoro-2-hydroxypropan-2-yl, 1,1,1-trifluoro-2-hydroxypropan-2-yl, 3-hydroxy-3-methylbutyl, hydroxy-3-methylbut-1-ynyl or 2H-triazol-4-yl.

In a further particular embodiment, R⁴ represents 2,2,2-trifluoro-1-hydroxyethyl, 2,2-difluoro-1-hydroxyethyl, 1,1-difluoro-2-hydroxypropan-2-yl, 1,1,1-trifluoro-2-hydroxypropan-2-yl, 3-hydroxy-3-methylbutyl, or hydroxy-3-methylbut-1-ynyl.

In a first embodiment, R⁵ represents hydrogen. In a second embodiment, R⁵ represents a C₁₋₆ alkyl optionally substituted by one or more substituents selected from hydroxy and halogen. In a first aspect of this embodiment, R⁵ represents a C₁₋₆ alkyl. In a second aspect of this embodiment, R⁵ represents a C₁₋₆ alkyl substituted by hydroxy. An example of R⁵ according to this aspect is (hydroxy)methyl.

Generally, R⁵ represents hydrogen or a C₁₋₆ alkyl substituted by hydroxy.

Typically, R⁵ represents hydrogen or (hydroxy)methyl.

Ideally, R⁵ represents hydrogen.

In a particular embodiment, the present invention relates to a particular subclass of compounds of formula (I) represented by formula (IB),

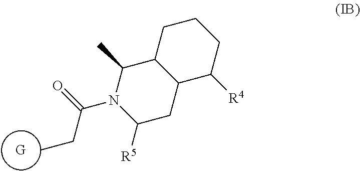

wherein G, R⁴, and R⁵ are as defined above.

A particular subgroup of compounds of formula (IB) according to the present invention is represented by formula (IB-a),

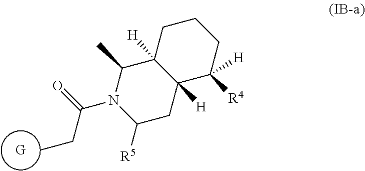

wherein G, R⁴ and R⁵ are as defined above.

In a particular aspect, the present invention relates to a particular sub-group of compounds of formula (IB-a) represented by formula (IB-aa),

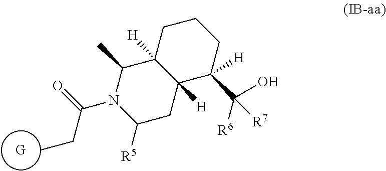

wherein

R⁶ and R⁷ represent independently hydrogen or a C₁₋₆ alkyl, which group may be optionally substituted by one or more halogen; and

G and R⁵ are as defined here above.

In a particular embodiment R⁶ represents hydrogen or C₁₋₆ alkyl and R⁷ represents C₁₋₆ alkyl, which group may be optionally substituted by one or more halogen.

In a first embodiment, R⁶ represents hydrogen. In a second embodiment, R⁶ represents C₁₋₆ alkyl. In one aspect of this embodiment R⁶ represents methyl. In a third embodiment, R⁶ represents C₁₋₆ alkyl substituted by one or more halogen. In a first aspect of this embodiment, R⁶ represents fluoromethyl. In a second aspect of this embodiment, R⁶ represents difluoromethyl. In a third aspect of this embodiment, R⁶ represents trifluoromethyl.

Generally, R⁶ represents hydrogen, C₁₋₆ alkyl or C₁₋₆ alkyl substituted by one or more halogen.

Suitably, R⁶ represents hydrogen or C₁₋₆ alkyl.

Illustratively, R⁶ represents hydrogen or methyl.

In a first embodiment, R⁷ represents hydrogen. In a second embodiment, R⁷ represents C₁₋₆ alkyl. In one aspect of this embodiment R⁶ represents methyl. In a third embodiment, R⁷ represents C₁₋₆ alkyl substituted by one or more halogen. In a first aspect of this embodiment, R⁷ represents fluoromethyl. In a second aspect of this embodiment, R⁷ represents difluoromethyl. In a third aspect of this embodiment, R⁷ represents trifluoromethyl.

Generally, R⁷ represents hydrogen, C₁₋₆ alkyl or C₁₋₆ alkyl substituted by one or more halogen.

Suitably, R⁷ represents C₁₋₆ alkyl substituted by one or more halogen.

Illustratively, R⁷ represents trifluoromethyl or difluoromethyl.

In a specific embodiment, the present invention relates to compounds represented by formula (IB-aa) as shown above, wherein,

G represents (G^{c});

X represents C—H or N;

R¹ represents C₁₋₆ alkyl substituted by one or more hydroxy or C₁₋₆ alkoxy;

R² and R³ represent independently halogen or cyano;

R⁵ represents hydrogen;

R⁶ represents hydrogen or C₁₋₆ alkyl; and

R⁷ represents C₁₋₆ alkyl substituted by one or more halogen.

Illustratively, the present invention relates to compounds represented by formula (IB-aa) as shown above, wherein,

G represents (G^{c});

X represents C—H or N;

R¹ represents 1-hydroxyethyl, methoxy or deuteriated methoxy;

R² and R³ represent independently chloro or cyano;

R⁵ represents hydrogen;

R⁶ represents hydrogen or methyl; and

R⁷ represents trifluoromethyl or difluoromethyl.

It will be apparent for the person skilled in the art that compounds represented by formula (IB-aa) wherein R⁶ and R⁷ are different may exist in the form of two different stereoisomers wherein the carbon bearing the hydroxy, R⁶ and R⁷ groups has an absolute stereochemical configuration of (R) or (S).

In a particular aspect, the carbon bearing the hydroxy, R⁶ and R⁷ in compounds of formula (IB-aa) has an absolute stereochemical configuration (S).

Specific novel compounds in accordance with the present invention include each of the compounds whose preparation is described in the accompanying Examples, their individual stereoisomers, and pharmaceutically acceptable salts and solvates thereof.

Therefore, in a particular aspect, the present invention relates to compounds of formula (I) which are selected from the group consisting of:
2-[2-[(1S,4aR,5R,8aS)-1-methyl-5-[(1 S)-2,2,2-trifluoro-1-hydroxy-ethyl]-3,4,4a,5,6,7,8,8a-octahydro-1H-isoquinolin-2-yl]-2-oxo-ethyl]-3-chloro-4-methoxybenzonitrile;
2-[2-[(1S,4aR,5R,8aS)-1-methyl-5-[(1R)-2,2,2-trifluoro-1-hydroxy-ethyl]-3,4,4a,5,6,7,8,8a-octahydro-1H-isoquinolin-2-yl]-2-oxo-ethyl]-3-chloro-4-methoxybenzonitrile;
2-[2-[(1S,4aR,5R,8aS)-1-methyl-5-[(1 S)-2,2,2-trifluoro-1-hydroxy-ethyl]-3,4,4a,5,6,7,8,8a-octahydro-1H-isoquinolin-2-yl]-2-oxo-ethyl]-3-chloro-6-methoxybenzonitrile;
1-[(1 S,4aR,5R,8aS)-1-methyl-5-[(1 S)-2,2,2-trifluoro-1-hydroxy-ethyl]-3,4,4a,5,6,7,8,8a-octahydro-1H-isoquinolin-2-yl]-2-(3,5-dichloro-2-methoxypyridin-4-yl)ethanone;
2-[2-[(1 S,4aR,5R,8aS)-1-methyl-5-[(1 S)-2,2,2-trifluoro-1-hydroxy-ethyl]-3,4,4a,5,6,7,8,8a-octahydro-1H-isoquinolin-2-yl]-2-oxo-ethyl]-3-chloro-4-(trideuteriomethoxy)benzonitrile;
1-[(1 S,4aR,5R,8aS)-1-methyl-5-[(1 S)-2,2,2-trifluoro-1-hydroxy-ethyl]-3,4,4a,5,6,7,8,8a-octahydro-1H-isoquinolin-2-yl]-2-(3,6-dichloro-[1,2,4]triazolo[4,3-a]pyridin-5-yl)ethanone;
1-[(1 S,4aR,5R,8aS)-1-methyl-5-[(1 S)-2,2,2-trifluoro-1-hydroxy-ethyl]-3,4,4a,5,6,7,8,8a-octahydro-1H-isoquinolin-2-yl]-2-[3,5-dichloro-2-(hydroxymethyl)-4-pyridyl]ethanone;
1-[(1 S,4aR,5R,8aS)-1-methyl-5-[(1 S)-2,2,2-trifluoro-1-hydroxy-ethyl]-3,4,4a,5,6,7,8,8a-octahydro-1H-isoquinolin-2-yl]-2-(3,5-dichloro-7-fluoro-1H-indazol-4-yl)ethanone;
1-[(1 S,4aR,5R,8aS)-1-methyl-5-[(1 S)-2,2,2-trifluoro-1-hydroxy-ethyl]-3,4,4a,5,6,7,8,8a-octahydro-1H-isoquinolin-2-yl]-2-(3,5-dichloro-1-methyl-indol-4-yl)ethenone;
1-[(1 S,4aR,5R,8aS)-1-methyl-5-[(1 S)-2,2,2-trifluoro-1-hydroxy-ethyl]-3,4,4a,5,6,7,8,8a-octahydro-1H-isoquinolin-2-yl]-2-[2,6-dichloro-3-(difluoromethoxy)phenyl]ethanone;
1-[(1 S,4aR,5R,8aS)-1-methyl-5-[(1 S)-2,2,2-trifluoro-1-hydroxy-ethyl]-3,4,4a,5,6,7,8,8a-octahydro-1H-isoquinolin-2-yl]-2-[3,5-dichloro-2-[(1 S)-1-hydroxyethyl]-4-pyridyl]ethanone;
1-[(1 S,4aR,5R,8aS)-1-methyl-5-[(1 S)-2,2,2-trifluoro-1-hydroxy-ethyl]-3,4,4a,5,6,7,8,8a-octahydro-1H-isoquinolin-2-yl]-2-[3,5-dichloro-2-[(1R)-1-hydroxyethyl]-4-pyridyl]ethanone;
2-[2-[(1S,4aR,5R,8aS)-5-[(1R)-2,2-difluoro-1-hydroxy-ethyl]-1-methyl-3,4,4a,5,6,7,8,8a-octahydro-1H-isoquinolin-2-yl]-2-oxo-ethyl]-3-chloro-4-methoxy-benzonitrile;
2-[2-[(1S,4aR,5R,8aS)-5-[(1 S)-2,2-difluoro-1-hydroxy-ethyl]-1-methyl-3,4,4a,5,6,7,8,8a-octahydro-1H-isoquinolin-2-yl]-2-oxo-ethyl]-3-chloro-4-methoxy-benzonitrile;
2-[2-[(1 S,4aR,5R,8aS)-1-methyl-5-[(1R)-2,2,2-trifluoro-1-hydroxy-1-methyl-ethyl]-3,4,4a,5,6,7,8,8a-octahydro-1H-isoquinolin-2-yl]-2-oxo-ethyl]-3-chloro-4-methoxy-benzonitrile;
2-[2-[(1 S,4aR,5R,8aS)-1-methyl-5-[(1 S)-2,2,2-trifluoro-1-hydroxy-1-methyl-ethyl]-3,4,4a,5,6,7,8,8a-octahydro-1H-isoquinolin-2-yl]-2-oxo-ethyl]-3-chloro-4-methoxy-benzonitrile;
1-[(1 S,4aR,8aS)-1-methyl-5-[(1R)-2,2,2-trifluoro-1-hydroxy-ethyl]-3,4,4a,5,6,7,8,8a-octahydro-1H-isoquinolin-2-yl]-2-(3,5-dichloro-1-methyl-indazol-4-yl)ethanone;
1-[(1 S,4aR,8aS)-1-methyl-5-[(1 S)-2,2,2-trifluoro-1-hydroxy-ethyl]-3,4,4a,5,6,7,8,8a-octahydro-1H-isoquinolin-2-yl]-2-(3,5-dichloro-1-methyl-indazol-4-yl)ethanone;
(1S,4aR,5R,8aS)—N-(2,6-dichlorophenyl)-1-methyl-5-[(1 S)-2,2,2-trifluoro-1-hydroxy-ethyl]-3,4,4a,5,6,7,8,8a-octahydro-1H-isoquinoline-2-carboxamide;
1-[(1 S,4aR,5R,8aS)-1-methyl-5-[(1 S)-2,2,2-trifluoro-1-hydroxy-ethyl]-3,4,4a,5,6,7,8,8a-octahydro-1H-isoquinolin-2-yl]-2-[3,5-dichloro-2-(1-hydroxy-1-methyl-ethyl)-4-pyridyl]ethanone;
1-[(1 S,4aR,5R,8aS)-1-methyl-5-[(1 S)-2,2,2-trifluoro-1-hydroxy-ethyl]-3,4,4a,5,6,7,8,8a-octahydro-1H-isoquinolin-2-yl]-2-[3,5-dichloro-2-[(1R)-2,2-difluoro-1-hydroxy-ethyl]-4-pyridyl]ethanone;
1-[(1 S,4aR,5R,8aS)-1-methyl-5-[(1 S)-2,2,2-trifluoro-1-hydroxy-ethyl]-3,4,4a,5,6,7,8,8a-octahydro-1H-isoquinolin-2-yl]-2-[3,5-dichloro-2-[(1 S)-2,2-difluoro-1-hydroxy-ethyl]-4-pyridyl]ethanone;
1-[(1 S,4aR,5R,8aS)-1-methyl-5-[(1R)-2,2,2-trifluoro-1-hydroxy-ethyl]-3,4,4a,5,6,7,8,8a-octahydro-1H-isoquinolin-2-yl]-2-[3,5-dichloro-2-[(1R)-2,2-difluoro-1-hydroxy-1-methyl-ethyl]-4-pyridyl]ethanone;
1-[(1 S,4aR,5R,8aS)-1-methyl-5-[(1R)-2,2,2-trifluoro-1-hydroxy-ethyl]-3,4,4a,5,6,7,8,8a-octahydro-1H-isoquinolin-2-yl]-2-[3,5-dichloro-2-[(1R)-2,2-difluoro-1-hydroxy-1-methyl-ethyl]-4-pyridyl]ethanone;
1-[(1 S,4aR,5R,8aS)-5-[(1S)-2,2-difluoro-1-hydroxy-ethyl]-1-methyl-3,4,4a,5,6,7,8,8a-octahydro-1H-isoquinolin-2-yl]-2-[3,5-dichloro-2-[(1 S)-1-hydroxyethyl]-4-pyridyl]ethanone;
1-[(1 S,4aR,5R,8aS)-5-[(1S)-2,2-difluoro-1-hydroxy-ethyl]-1-methyl-3,4,4a,5,6,7,8,8a-octahydro-1H-isoquinolin-2-yl]-2-[3,5-dichloro-2-[(1R)-1-hydroxyethyl]-4-pyridyl]ethanone;
1-[(1 S,4aR,5R,8aS)-5-[(1S)-2,2-difluoro-1-hydroxy-1-methyl-ethyl]-1-methyl-3,4,4a,5,6,7,8,8a-octahydro-1H-isoquinolin-2-yl]-2-[3,5-dichloro-2-[(1 S)-1-hydroxyethyl]-4-pyridyl]ethanone;
1-[(1 S,4aR,5R,8aS)-5-[(1S)-2,2-difluoro-1-hydroxy-1-methyl-ethyl]-1-methyl-3,4,4a,5,6,7,8,8a-octahydro-1H-isoquinolin-2-yl]-2-[3,5-dichloro-2-[(1R)-1-hydroxyethyl]-4-pyridyl]ethanone;
2-[2-[(1S,4aS,8aS)-5-(3-hydroxy-3-methyl-but-1-ynyl)-1-methyl-3,4,4a,5,6,7,8,8a-octahydro-1H-isoquinolin-2-yl]-2-oxo-ethyl]-3-chloro-4-methoxy-benzonitrile;
2-[2-[(1S,4aS,5S,8aS)-5-(3-hydroxy-3-methyl-butyl)-1-methyl-3,4,4a,5,6,7,8,8a-octahydro-1H-isoquinolin-2-yl]-2-oxo-ethyl]-3-chloro-4-methoxy-benzonitrile;
1-[(1 S,4aS,5S,8aS)-5-(3-hydroxy-3-methyl-butyl)-1-methyl-3,4,4a,5,6,7,8,8a-octahydro-1H-isoquinolin-2-yl]-2-(3,5-dichloro-1-methyl-indazol-4-yl)ethanone;
1-[(1 S,4aS,5S,8aS)-5-(3-hydroxy-3-methyl-butyl)-1-methyl-3,4,4a,5,6,7,8,8a-octahydro-1H-isoquinolin-2-yl]-2-[3,5-dichloro-2-(hydroxymethyl)-4-pyridyl]ethanone;
1-[(1 S,4aS,5S,8aS)-5-(3-hydroxy-3-methyl-butyl)-1-methyl-3,4,4a,5,6,7,8,8a-octahydro-1H-isoquinolin-2-yl]-2-[3,5-dichloro-2-[(1S)-1-hydroxyethyl]-4-pyridyl]ethanone;
1-[(1 S,4aS,5S,8aS)-5-(3-hydroxy-3-methyl-butyl)-1-methyl-3,4,4a,5,6,7,8,8a-octahydro-1H-isoquinolin-2-yl]-2-[3,5-dichloro-2-[(1R)-1-hydroxyethyl]-4-pyridyl]ethanone;
2-[2-[(1 S,4aR,5R,8aS)-1-methyl-5-(2H-triazol-4-yl)-3,4,4a,5,6,7,8,8a-octahydro-1H-isoquinolin-2-yl]-2-oxoethyl]-3-chloro-4-methoxybenzonitrile;
1-[(1 S,3R,4aR,5R,8aS)-3-(hydroxymethyl)-1-methyl-5-[(1 S)-2,2,2-trifluoro-1-hydroxy-ethyl]-3,4,4a,5,6,7,8,8a-octahydro-1H-isoquinolin-2-yl]-2-(3,5-dichloro-1-methyl-indazol-4-yl)ethanone; and
1-[(1 S,3R,4aS,5S,8aR)-3-(hydroxymethyl)-1-methyl-5-[(1S)-2,2,2-trifluoro-1-hydroxy-ethyl]-3,4,4a,5,6,7,8,8a-octahydro-1H-isoquinolin-2-yl]-2-(3,5-dichloro-1-methyl-indazol-4-yl)ethanone.

The present invention also provides a compound of formula (I) as defined above or a pharmaceutically acceptable salt thereof, for use in therapy.

In another aspect, the present invention also provides a compound of formula (I) as defined above, or a pharmaceutically acceptable salt thereof, for use in the treatment of diseases and/or disorders in which D1 receptors play a role.

In another aspect, the present invention provides a compound of formula (I) as defined above, or a pharmaceutically acceptable salt thereof, for use in the treatment and/or prevention of cognitive and negative symptoms in schizophrenia, cognitive impairment related to neuroleptic therapy, Mild Cognitive impairment (MCI), impulsivity, Attention-Defficit Hyperactivity Disorder (ADHD), Parkinson's disease and other movement disorders, dystonia, Parkinson's dementia, Huntington's disease, dementia with Lewy Body, Alzheimer's disease drug addiction, sleep disorders, apathy, traumatic spinal cord injury or neuropathic pain.

In a particular embodiment of this aspect, the present invention provides a compound of formula (I) as defined above, or a pharmaceutically acceptable salt thereof for use in the treatment of Parkinson's disease and other movement disorders, Alzheimer's disease, or cognitive and negative symptoms in schizophrenia.

Therefore, in one particular aspect, the present invention provides a compound of formula (I), as defined above, or a pharmaceutically acceptable salt thereof, for use in the treatment of Parkinson's disease and other movement disorders.

In a further aspect, the present invention provides for the use of a compound of formula (I) as defined above, or a pharmaceutically acceptable salt thereof, for the manufacture of a medicament useful for the treatment and/or prevention of diseases and/or disorders in which D1 receptors play a role.

In another further aspect, the present invention provides for the use of a compound of formula (I) as defined above, or a pharmaceutically acceptable salt thereof, for the manufacture of a medicament useful for the treatment and/or prevention of cognitive and negative symptoms in schizophrenia, cognitive impairment related to neuroleptic therapy, Mild Cognitive Impairment (MCI), impulsivity, Attention-Deficit Hyperactivity Disorder (ADHD), Parkinson's disease and other movement disorders, dystonia, Parkinson's dementia, Huntington's disease, dementia with Lewy Body, Alzheimer's disease, drug addiction, sleep disorders, apathy, traumatic spinal cord injury or neuropathic pain.

In a particular embodiment of this aspect, the present invention provides for the use of a compound of formula (I) as defined above, or a pharmaceutically acceptable salt thereof for the manufacture of a medicament useful for the treatment of Parkinson's disease and other movement disorders, Alzheimer's disease, or cognitive and negative symptoms in schizophrenia.

In one particular aspect, the present invention provides for the use of a compound of formula (I), as defined above, or a pharmaceutically acceptable salt thereof, for the manufacture of a medicament useful for the treatment of Parkinson's disease and other movement disorders.

The present invention also provides a method for the treatment and/or prevention of disorders for which the administration of D1 positive allosteric modulator is indicated, which comprises administering to a patient in need of such treatment an effective amount of a compound of formula (I) as defined above, or a pharmaceutically acceptable salt thereof.

In another aspect, the present invention provides a method for the treatment and/or prevention of cognitive and negative symptoms in schizophrenia, cognitive impairment related to neuroleptic therapy, Mild Cognitive Impairment (MCI), impulsivity, Attention-Deficit Hyperactivity Disorder (ADHD), Parkinson's disease and other movement disorders, dystonia, Parkinson's dementia, Huntington's disease, dementia with Lewy Body, Alzheimer's disease, drug addiction, sleep disorders, apathy, traumatic spinal cord injury or neuropathic pain, which comprises administering to a patient in need of such treatment an effective amount of a compound of formula (I) as defined above, or a pharmaceutically acceptable salt thereof.

In a particular embodiment of this aspect, the present invention provides a method for the treatment of Parkinson's disease and other movement disorders, Alzheimer's disease, or cognitive and negative symptoms in schizophrenia, which comprises administering to a patient in need of such treatment of an effective amount of a compound of formula (I) as defined above, or a pharmaceutically acceptable salt thereof.

In one particular aspect, the present invention provides a method for the treatment of Parkinson's disease and other movement disorders, which comprises administering to a patient in need of such treatment of an effective amount of a compound of formula (I) as defined above, or a pharmaceutically acceptable salt thereof.

Activity in any of the above-mentioned therapeutic indications or disorders can of course be determined by carrying out suitable clinical trials in a manner known to a person skilled in the relevant art for the particular indication and/or in the design of clinical trials in general.

For use in medicine, the salts of the compounds of formula (I) will be pharmaceutically acceptable salts. Other salts may, however, be useful in the preparation of the compounds of use in the invention or of their pharmaceutically acceptable salts. Standard principles underlying the selection and preparation of pharmaceutically acceptable salts are described, for example, in Handbook of Pharmaceutical Salts: Properties, Selection and Use, ed. P. H. Stahl & C. G. Wermuth, Wiley-VCH, 2002. Suitable pharmaceutically acceptable salts of the compound of formula (I) include acid addition salts which may, for example, be formed by mixing a solution of the compound of formula (I) with a solution of a pharmaceutically acceptable acid.

The present invention includes within its scope solvates of the compounds of formula (I) above. Such solvates may be formed with common organic solvents or water.

The present invention also includes within its scope co-crystals of the compounds of formula (I) above. The technical term “co-crystal” is used to describe the situation where neutral molecular components are present within a crystalline compound in a definite stoichiometric ratio. The preparation of pharmaceutical co-crystals enables modifications to be made to the crystalline form of an active pharmaceutical ingredient, which in turn can alter its physicochemical properties without compromising its intended biological activity (see Pharmaceutical Salts and Co-crystals, ed. J. Wouters & L. Quere, RSC Publishing, 2012).

Compounds according to the present invention may exist in different polymorphic forms. Although not explicitly indicated in the above formula, such forms are intended to be included within the scope of the present invention.

The invention also includes within its scope pro-drug forms of the compounds of formula (I) and its various sub-scopes and sub-groups.

For treating diseases, compounds of formula (I) or their pharmaceutically acceptable salts may be employed at an effective daily dosage and administered in the form of a pharmaceutical composition.

Therefore, another embodiment of the present invention concerns a pharmaceutical composition comprising an effective amount of a compound of formula (I) or a pharmaceutically acceptable salt thereof in combination with a pharmaceutically acceptable diluent or carrier.

To prepare a pharmaceutical composition according to the invention, one or more of the compounds of formula (I) or a pharmaceutically acceptable salt thereof is intimately admixed with a pharmaceutical diluent or carrier according to conventional pharmaceutical compounding techniques known to the skilled practitioner.

Suitable diluents and carriers may take a wide variety of forms depending on the desired route of administration, e.g., oral, rectal, parenteral or intranasal.

Pharmaceutical compositions comprising compounds according to the invention can, for example, be administered orally, parenterally, i.e. intravenously, intramuscularly or subcutaneously, intrathecally, by inhalation or intranasally.

Pharmaceutical compositions suitable for oral administration can be solids or liquids and can, for example, be in the form of tablets, pills, dragees, gelatin capsules, solutions, syrups, chewing-gums and the like.

To this end the active ingredient may be mixed with an inert diluent or a non-toxic pharmaceutically acceptable carrier such as starch or lactose. Optionally, these pharmaceutical compositions can also contain a binder such as microcrystalline cellulose, gum tragacanth or gelatine, a disintegrant such as alginic acid, a lubricant such as magnesium stearate, a glidant such as colloidal silicon dioxide, a sweetener such as sucrose or saccharin, or colouring agents or a flavouring agent such as peppermint or methyl salicylate.

The invention also contemplates compositions which can release the active substance in a controlled manner. Pharmaceutical compositions which can be used for parenteral administration are in conventional form such as aqueous or oily solutions or suspensions generally contained in ampoules, disposable syringes, glass or plastics vials or infusion containers.

In addition to the active ingredient, these solutions or suspensions can optionally also contain a sterile diluent such as water for injection, a physiological saline solution, oils, polyethylene glycols, glycerine, propylene glycol or other synthetic solvents, antibacterial agents such as benzyl alcohol, antioxidants such as ascorbic acid or sodium bisulphite, chelating agents such as ethylene diamine-tetra-acetic acid, buffers such as acetates, citrates or phosphates and agents for adjusting the osmolarity, such as sodium chloride or dextrose.

These pharmaceutical forms are prepared using methods which are routinely used by pharmacists.

The amount of active ingredient in the pharmaceutical compositions can fall within a wide range of concentrations and depends on a variety of factors such as the patient's sex, age, weight and medical condition, as well as on the method of administration. Thus the quantity of compound of formula (I) in compositions for oral administration is at least 0.5% by weight and can be up to 80% by weight with respect to the total weight of the composition.

In accordance with the invention it has also been found that the compounds of formula (I) or the pharmaceutically acceptable salts thereof can be administered alone or in combination with other pharmaceutically active ingredients.

In compositions for parenteral administration, the quantity of compound of formula (I) present is at least 0.5% by weight and can be up to 33% by weight with respect to the total weight of the composition. For the preferred parenteral compositions, the dosage unit is in the range 0.5 mg to 3000 mg of compounds of formula (I).

The daily dose can fall within a wide range of dosage units of compound of formula (I) and is generally in the range 0.5 to 3000 mg. However, it should be understood that the specific doses can be adapted to particular cases depending on the individual requirements, at the physician's discretion.

It will be apparent to the person skilled in the art that there are various synthetic pathways that can lead to the compounds according to the invention. The following processes are aimed at illustrating some of these synthetic pathways but should not be construed in any way as a limitation on how the compounds according to the invention should be made.

Compounds of formula (I) wherein Z═NH may be prepared by a process involving the reaction of an intermediate of formula (II-U) with an intermediate of formula (III)

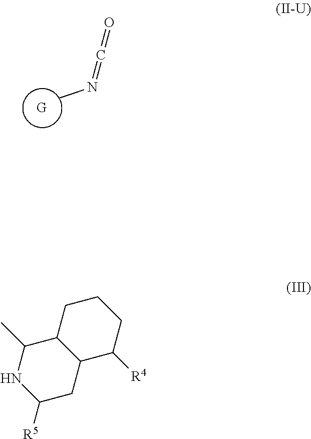

wherein G, R⁴ and R⁵ are as defined here above.

The reaction is conveniently performed in the presence of a base e.g. triethylamine, in a suitable solvent e.g. dichloromethane at room temperature.

Compounds of formula (I) wherein Z═CH₂ may be prepared by a process involving the reaction of an intermediate of formula (II) with an intermediate of formula (III),

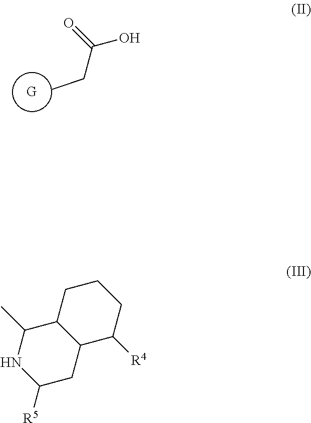

wherein G, R⁴ and R⁵ are as defined here above.

The reaction is conveniently performed in the presence of 1-(3-dimethylaminopropyl)-3-ethylcarbodiimide hydrochloride and 1-hydroxybenzotriazole hydrate, in a suitable solvent e.g. dimethylformamide, with a catalytic amount of 4-methylmorpholine.

Alternatively, the reaction may be performed in the presence of classical coupling agents such as benzotriazolyl derivatives (BOP and the like) or uronium derivatives (HBTU, COMU® and the like) or other reagents known by the person skilled in the art, in the presence of a base such as triethylamine or diisopropylethylamine in a solvent such as N,N-dimethylformamide or dichloromethane.

Compounds of formula (I), wherein R⁵ represents hydrogen and wherein R⁴ represents a C₁₋₆ alkyl substituted by a hydroxy group, i.e. wherein R⁴═—C(OH)R⁶R⁷, may be prepared by a process involving reaction of an intermediate (Ia),

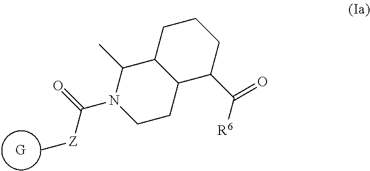

wherein G and Z are as defined here above and R⁶ is as defined here below.

When R⁶ represents hydrogen and R⁷ represents difluoromethyl or trifluoromethyl, the reaction is conveniently performed in the presence of difluoro- or trifluromethyl-trimethylsilane, in the presence of cesium fluoride, in a suitable solvent, e.g. DMF.

When R⁶ represents difluoromethyl or trifluoromethyl and R⁷ represents methyl, the reaction may be performed using methylmagnesium halide, e.g. methylmagnesium chloride, in a suitable solvent, e.g. THF, according to methods known to the person skilled in the art.

Intermediates of formula (Ia) wherein R⁶ represents hydrogen may be prepared by functional groups transformations of an intermediate of formula (Ib),

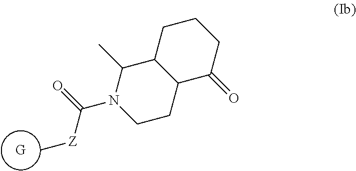

wherein G and Z have the same definition as above.

This reaction may be performed according to a two-steps sequence involving (i) a Wittig reaction with a phosphorus ylide prepared from a phosphonium salt, preferably (methoxymethyl)triphenylphosphonium chloride, and a base such as n-butyllithium or sodium tert-butoxide in tetrahydrofuran at −78° C. followed by (ii) acidic hydrolysis of the enol ether intermediate with a solution of an acid such as hydrochloric acid at room temperature.

Intermediates of formula (Ia) wherein R⁶ represents difluoromethyl or trifluoromethyl may be prepared by oxidation of a compound of formula (I) wherein R⁴ represents —C(OH)R⁶R⁷ and wherein R⁷ represents hydrogen. This reaction may be performed using an oxidizing agent such as Dess-Martin periodinane or any other reagent known to the person skilled in the art.

Compounds of formula (I), wherein R⁵ represents hydrogen and wherein R⁴ represents a C₁₋₆ alkyl substituted by a hydroxy group of formula —(CH₂)₂C(R^{t}R^{u})OH, may be prepared by a process involving the reduction of an intermediate (Ic),

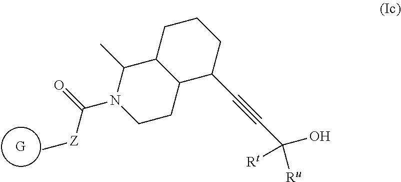

wherein G and Z have the same definition as above and wherein R^{t} and R^{u}═C₁-C₃ alkyl.

This reaction may be conveniently performed under hydrogen pressure in the presence of a catalytic amount of Pd/C or any other catalyst known to the person skilled in the art in a suitable solvent such as ethanol at room temperature.

Intermediates (Ic) may be prepared by reaction of an intermediate (Id) with a ketone of formula R^{t}R^{u}C═O,

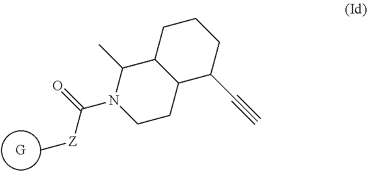

wherein G, Z, R^{t} and R^{u} have the same definition as above. This reaction may be performed by deprotonation with a strong base e.g. n-butyllithium in a suitable solvent such as THF at −78° C. followed by hydroxyalkylation with a suitable ketone R^{t}R^{u}C═O.

Intermediates of formula (Id) may be conveniently prepared by reaction of an intermediate of formula (Ia) wherein R⁶═H. This reaction may be performed using 1-diazo-1-dimethoxyphosphoryl-propan-2-one in a suitable solvent such as methanol in presence of a base such as potassium carbonate at room temperature (Seyferth-Gilbert homologation with Ohira-Bestmann reagent) or by any method known to the person skilled in the art.

Some compounds of formula (I), wherein R⁵ represents hydrogen and R⁴ represents C₅₋₈ heteroaryl, i.e. wherein R⁴=2,3,4-triazolyl, may be prepared by reaction of intermediate (Id) with an azido reagent such as sodium azide or trimethylsilylazide or according to any method known to the person skilled in the art.

Compounds of formula (I) wherein G represents (G), X represents N, and R¹ represents a C₁₋₆ alkyl substituted by a hydroxy group of formula C(OH)R^{w}R^{z} may be prepared by a process involving reaction of an intermediate (Ie),

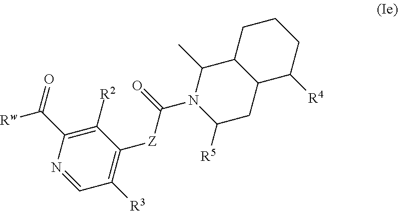

wherein Z, R², R³, R⁴ and R⁵ have the same definition as above for compound of formula (I), and R^{w} is as defined here below.

When R^{w} represents methyl and R^{z} represents hydrogen, the reaction is conveniently performed using a reducing agent such as sodium borohydride in a suitable solvent such as methanol at 0° C. or according to any method known to the person skilled in the art.

When R^{w} represents methyl and R^{z} represents methyl, the reaction is conveniently performed using methyllithium in a suitable solvent such as THF at 0° C. or according to any method known to the person skilled in the art.

When R^{w} represents hydrogen or methyl and R^{z} represents difluoromethyl or trifuoromethyl, the reaction is conveniently performed in the presence of difluoro- or trifluromethyl-trimethylsilane, in the presence of cesium fluoride, in a suitable solvent, e.g. DMF.

Intermediates of formula (Ie) wherein R^{w} represents hydrogen may be prepared by oxidation of a compound of formula (I) wherein R¹ represents CH₂OH and Z, R², R³, R⁴ and R⁵ have the same definition as above. This reaction may be conveniently performed using an oxidizing agent such as manganese dioxide in a suitable solvent such as 1-4-dioxane at 70° C. or by any other method known to the person skilled in the art.

Intermediates of formula (Ie) wherein R^{w} represents methyl may be conveniently prepared by acidic hydrolysis of an intermediate of formula (If),

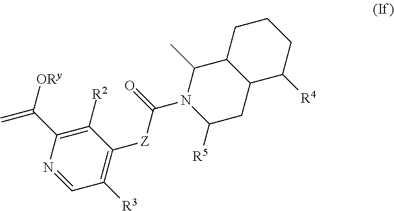

wherein Z, R², R³, R⁴ and R⁵ have the same definition as above and R^{y} represents a C₁₋₃ alkyl. This reaction may be conveniently performed using an acid such as hydrochloric acid in a suitable solvent such as THF at room temperature.

Intermediates of formula (If) wherein Z represents CH₂ may be prepared by a process involving the reaction of an intermediate of formula (IIf) with an intermediate of formula (III),

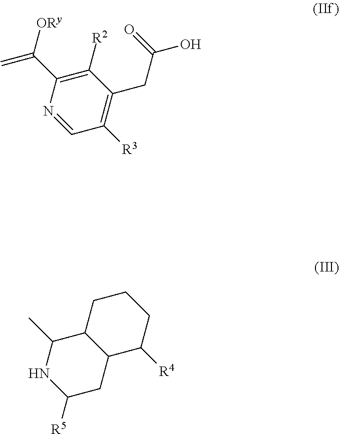

wherein R^{y}, R², R³, R⁴ and R⁵ are as defined here above.

The reaction is conveniently performed in the presence of 1-(3-dimethylaminopropyl)-3-ethylcarbodiimide hydrochloride and 1-hydroxybenzotriazole hydrate, in a suitable solvent e.g. dimethylformamide, with a catalytic amount of 4-methylmorpholine.

Alternatively, the reaction may be performed in the presence of classical coupling agents such as benzotriazolyl derivatives (BOP and the like) or uronium derivatives (HBTU, COMU® and the like) or other reagents known by the person skilled in the art, in the presence of a base such as triethylamine or diisopropylethylamine in a solvent such as N,N-dimethylformamide or dichloromethane.

Compounds of formula (I), wherein R⁵ represents a C₁-C₆ alkyl substituted by a hydroxy group, in particular CH₂—OH, may be prepared by a process involving the reaction of an intermediate of formula (II) when Z represents CH₂ or an intermediate of formula (II-U) when Z represents NH as defined above with an intermediate of formula (III-S),

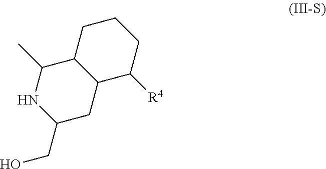

wherein X, R², R³ and R⁴ are as defined here above.

When Z represents CH₂, the reaction is conveniently performed in the presence of 1-(3-dimethylaminopropyl)-3-ethylcarbodiimide hydrochloride and 1-hydroxybenzotriazole hydrate, in a suitable solvent e.g. dimethylformamide, with a catalytic amount of 4-methylmorpholine.

Alternatively, the reaction may be performed in the presence of classical coupling agents such as benzotriazolyl derivatives (BOP and the like) or uronium derivatives (HBTU, COMU® and the like) or other reagents known by the person skilled in the art, in the presence of a base such as triethylamine or diisopropylethylamine in a solvent such as N,N-dimethylformamide or dichloromethane.

When Z represents NH, the reaction is conveniently performed in the presence of a base e.g. triethylamine, in a suitable solvent e.g. dichloromethane at room temperature. The hydroxyl group may first be protected with a suitable protecting group such as a tert-butyldimethylsilyl group or any other group known to the person skilled in the art and deprotected after the coupling reaction by any method known to the person skilled in the art.

Intermediates of formula (III-S) may be prepared by ring-opening of an intermediate of formula XII,

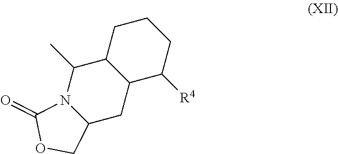

wherein R⁴ has the same definition as above. This reaction is conveniently performed using a base such as sodium hydroxide in a suitable solvent such as ethanol at 80° C.

Intermediates of formula (Ib) may be prepared according to a process involving reacting an intermediate of formula (II) when Z represents CH₂ or an intermediate of formula (II-U) when Z represents NH as defined above with an intermediate of formula (IV),

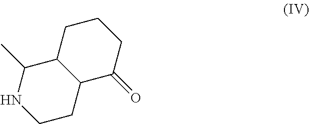

under conditions similar to those described for the coupling of intermediates of formula (II) with intermediates of formula (III).

Intermediates of formula (IV) may be prepared by deprotection of an intermediate of formula (V),

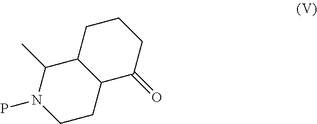

wherein P is a protecting group e.g. tert-butoxy carbonyl (Boc) group or a benzyloxycarbonyl (Cbz). This reaction is conveniently performed in the presence of an acid, e.g. trifluoroacetic acid or hydrochloric acid or according to any method known to the person skilled in the art.

Intermediates of formula (V) may be prepared by oxidation of an intermediate of formula (VI),

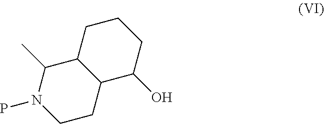

This reaction may be performed using an oxidizing agent, e.g. sodium hypochlorite, in acidic medium at low temperature, or any other oxidizing agent known to the person skilled in the art.

Intermediates of formula (VI) may be prepared by reduction of a phenolic intermediate of formula (VII)

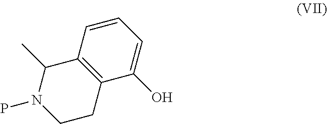

This reaction may be performed by hydrogenation in the presence of a metal catalyst, e.g. rhodium on activated charcoal, in a polar solvent, e.g. isopropanol, at a temperature ranging from 80 to 110° C., or according to any conditions known to the person skilled in the art.

Intermediates of formula (VI) may be prepared by hydroxylation of an intermediate of formula (VIII),

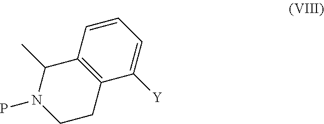

wherein Y is an halogen such as a bromine.

This reaction may be performed using a metal hydroxide, e.g. potassium hydroxide, in the presence of a palladium catalyst, e.g. t-BuXPhos-palladium, in a polar solvent such as 1,4-dioxane/water, at a temperature ranging from 75 to 90° C., or according to conditions known to the person skilled in the art.

Intermediates of formula (VIII) may be prepared by a process involving reaction of an intermediate of formula (IX),

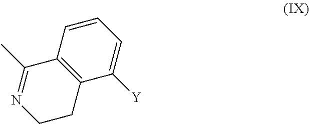

wherein Y is as defined here above.

The reaction is conveniently performed in the presence of a suitable reducing agent, e.g. sodium borohydride, in a suitable solvent, e.g. ethanol, at low temperature, according to methods known to the skilled person in the art.

Intermediates of formula (VIII) may be prepared by a process involving reaction of an intermediate of formula (X),

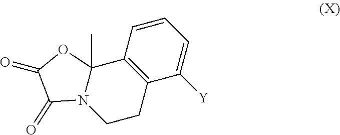

wherein Y is as defined here above.

The reaction is conveniently performed in the presence of oxalyl chloride in a suitable solvent, e.g. dichloromethane, in the presence of a transition metal salt, e.g. iron chloride, at low temperature.

Intermediate of formula (X) may be prepared by a process involving reaction of commercially available intermediate (XI),

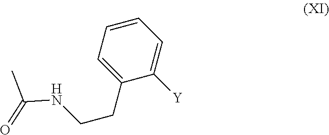

wherein Y is as defined here above.

Intermediates of formula (XII) wherein R⁴ represents C₁₋₆ alkyl substituted by a hydroxy group i.e. C(OH)R⁶R⁷, may be prepared by reduction of an intermediate of formula (XIII)

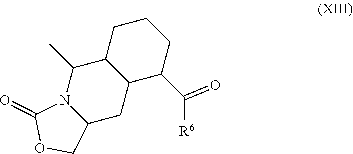

When R⁶ represents hydrogen and R⁷ represents difluoromethyl or trifluoromethyl, the reaction is conveniently performed in the presence of difluoro- or trifluoromethyl-trimethylsilane, in the presence of cesium fluoride, in a suitable solvent, e.g. DMF.

When R⁶ represents difluoromethyl or trifluoromethyl and R⁷ represents methyl, the reaction may be performed using methylmagnesium halide, e.g. methylmagnesium chloride, in a suitable solvent, e.g. THF, according to methods known to the person skilled in the art.

Intermediates of formula (XIII) wherein R⁶ represents hydrogen may be prepared by functional groups transformations of an intermediate of formula XIV,

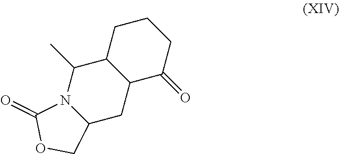 [SMILES: CC1C2CCCC(=O)C2CC2COC(=O)N21]

This reaction may be performed according to a two-steps sequence involving (i) a Wittig reaction with a phosphorus ylide prepared from a phosphonium salt, preferably (methoxymethyl)triphenylphosphonium chloride, and sodium tert-butoxide in tetrahydrofuran at −78° C. followed by (ii) acidic hydrolysis of the enol ether intermediate with a solution of an acid such as hydrochloric acid at room temperature.

Intermediates of formula (XIII) wherein R⁶ represents difluoromethyl or trifluoromethyl may be prepared by oxidation of a compound of formula (XII) wherein R⁴ represents —C(OH)R⁶R⁷ and wherein R⁷ represents hydrogen. This reaction may be conveniently performed using Dess-Martin periodinane or by any oxidizing agent known to the person skilled in the art.

Intermediates of formula (XIV) may be prepared by oxidation of an intermediate of formula (XV),

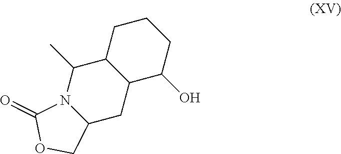 [SMILES: CC1C2CCCC(O)C2CC2COC(=O)N21]

This reaction may be performed using an oxidizing agent, e.g. Dess-Martin periodinane, at room temperature, or any other oxidizing agent known to the person skilled in the art.

Intermediates of formula (XV) may be prepared by reduction of a phenolic intermediate of formula (XVI),

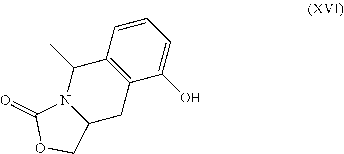 [SMILES: CC1c2cccc(O)c2CC2COC(=O)N21]

This reaction may be performed by hydrogenation in the presence of a metal catalyst, e.g. rhodium on activated charcoal, in a polar solvent, e.g. isopropanol, at a temperature ranging from 80 to 110° C., or according to any conditions known to the person skilled in the art.

Intermediates of formula (XVI) may be prepared by hydroxylation of an intermediate of formula (XVII),

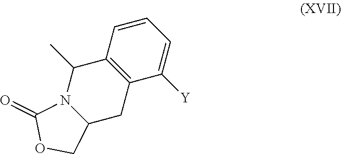

wherein Y is an halogen such as a bromine.

This reaction may be performed using a metal hydroxide, e.g. potassium hydroxide, in the presence of a palladium catalyst, e.g. t-BuXPhos-palladium, in a polar solvent such as 1,4-dioxane/water, at a temperature ranging from 80 to 100° C., or according to conditions known to the person skilled in the art.

Intermediate of formula (XVII) may be prepared by a process involving reaction of intermediates of formula (XVIII)

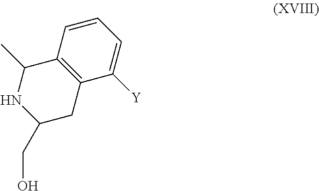

wherein Y represents halogen, i.e. bromine.

This reaction may be prepared using a coupling agent such as carbonyldiimidazole (CDI) in a suitable solvent such as DCM or DMF in the presence of a base such as diisopropylethylamine at room temperature or according to any method known to the person skilled in the art.

Intermediate of formula (XVIII) may be prepared by deprotection of an intermediate of formula (XIX),

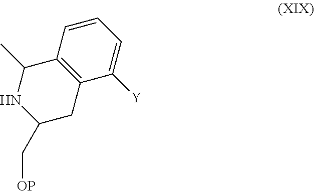

Wherein Y represents halogen i.e. bromine and P represents a protecting group such as tert-butyldimethylsilyl. This reaction may be performed in the presence of an acid such as hydrochloric acid in a polar solvent such as 2-propanol at room temperature or according to any method known to the person skilled in the art.

Intermediate of formula (XIX) may be prepared by a process involving reaction of an intermediate of formula (XX), wherein Y and P are as defined above.

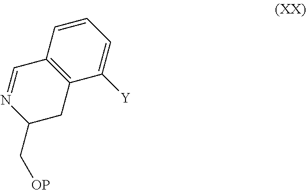

The reaction is conveniently performed in the presence of methyl magnesium chloride, in a suitable solvent e.g. tetrahydrofuran, at low temperature.

Intermediate (XX) may be prepared by a two-steps process involving reaction of intermediate of formula (XXI),

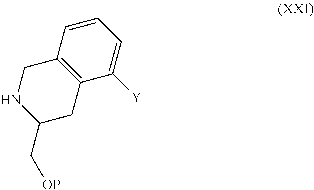

wherein Y is as defined above and P represents hydrogen or tert-butyl-dimethylsilyl.

In a first step, intermediate (XXII) wherein P represents hydrogen is reacted with tert-butyldimethylsilyl chloride in the presence of a suitable base e.g. 4-dimethylamino-pyridine at room temperature, to afford intermediate (XXI) wherein P represents tert-butyl-dimethylsilyl.

In a second step, intermediate (XXI) wherein P represents tert-butyl-dimethylsilyl is reacted with N-chlorosuccinimide (NCS), in a suitable solvent, e.g. THF to afford intermediate (XX).

Intermediate (XXII) wherein P represents hydrogen may be prepared by a process involving intermediate of formula (XXIII), wherein Y is as defined above.

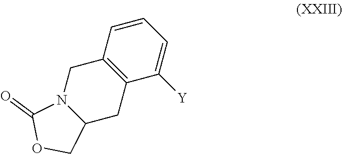

The reaction is conveniently performed in the presence of a strong base, e.g. sodium hydroxide, in a suitable solvent, e.g. mixture of ethanol and water, at high temperature.

Intermediate of formula (XXIII) may be prepared by a process involving reaction of intermediate (XXIV),

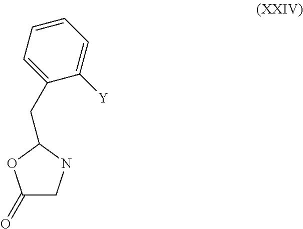

wherein Y is as defined here above.

The reaction is conveniently performed in the presence of trimethylsilyltriflate and paraformaldehyde, in a suitable solvent e.g. dichloromethane.

Intermediate (XXIV) may be prepared by a 2 steps process involving commercially available intermediate (XXV),

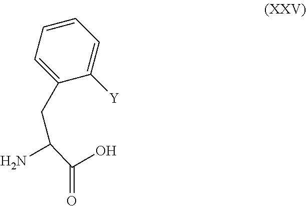

wherein Y is as defined above.

The reaction is conveniently performed according to the methods described in the accompanying examples or according to methods known to the person skilled in the art.

Intermediates of formula (III) may alternatively be prepared by a process involving reaction of an intermediate of formula (IIIa),

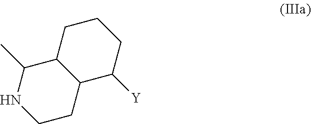

wherein Y represents halogen, e.g. bromo.

Some intermediates of formula (III) may be prepared by a process involving coupling of an intermediate of formula (IIIa) with a compound of formula R⁴—Y¹, wherein Y¹ represents hydrogen, halogen, or boronic acid derivative, in the presence of a transition metal complex, generally a palladium complex, and a base, according to methods known to the person skilled in the art. The reaction is conveniently performed at elevated temperature in a suitable solvent.

Some of these conditions for particular groups are described hereafter:
(i) When R⁴ represents C₁₋₆ alkyl the reaction maybe performed by reacting intermediate (IIIa) first with a vinyl boronic acid/boronate ester in the presence of a transition metal catalyst, e.g. tetrakis(triphenylphosphine)palladium (0) and a base, followed by a reduction under pressure of hydrogen, in the presence of a transition metal catalyst, e.g. Pd/C, in a suitable solvent, e.g. ethanol, under conditions known to the person skilled in the art.
(ii) When R⁴ represents C₅₋₈ heteroaryl, the reaction maybe performed by reacting intermediate (IIIa) with heteroaryl boronic acid/boronate ester in the presence of a transition metal catalyst, e.g. tetrakis(triphenylphosphine)palladium (0) and a base under conditions known to the person skilled in the art.

Intermediates of formula (IIIa) may be prepared by hydrogenation of an intermediate of formula (VIII) in the presence of a catalyst such rhodium on charcoal in a suitable solvent such as methanol or by any method known to the person skilled in the art. The person skilled in the art may consider to first protect the amine with a protecting group such as tert-butoxycarbonyl (Boc) before the hydrogenation step and subsequently deprotect it according to any method (s)he would know.

Intermediates of formula (III) wherein R⁴ represents C₁₋₆ alkyl substituted by a hydroxy group i.e. C(OH)R⁶R⁷, may be prepared by deprotection of an intermediate (IIIb),

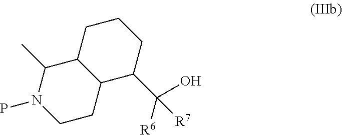

wherein P is a protecting group e.g. tert-butoxy carbonyl (Boc) group or a benzyloxycarbonyl (Cbz). This reaction is conveniently performed in the presence of an acid, e.g. trifluoroacetic acid or hydrochloric acid or according to any method known to the person skilled in the art.

Intermediates of formula (IIIb) may be prepared by reduction of an intermediate of formula (IIIc),

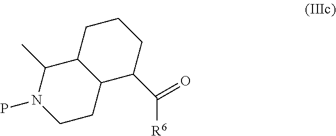

wherein P is as defined here above and R⁶ is as defined here below.

When R⁶ represents hydrogen and R⁷ represents difluoromethyl or trifluoromethyl, the reaction is conveniently performed in the presence of difluoro- or trifluromethyl-trimethylsilane, in the presence of cesium fluoride, in a suitable solvent, e.g. DMF.

When R⁶ represents difluoromethyl or trifluoromethyl and R⁷ represents methyl, the reaction may be performed using methylmagnesium halide, e.g. methylmagnesium chloride, in a suitable solvent, e.g. THF, according to methods known to the person skilled in the art.

Intermediates of formula (IIIc) wherein R⁶ represents hydrogen may be prepared by functional groups transformations of an intermediate of formula V wherein P has the same definition as above. This reaction may be performed according to a two-steps sequence involving (i) a Wittig reaction with a phosphorus ylide prepared from a phosphonium salt, preferably (methoxymethyl)triphenylphosphonium chloride, and n-butyllithium in tetrahydrofuran at −78° C. followed by (ii) acidic hydrolysis of the enol ether intermediate with a solution of an acid such as hydrochloric acid at room temperature.

Intermediates of formula (IIIc) wherein R⁶ represents difluoromethyl or trifluoromethyl may be prepared by oxidation of a compound of formula (IIIb) wherein R⁴ represents —C(OH)R⁶R⁷ and wherein R⁷ represents hydrogen. This reaction may be performed using any oxidizing agent known to the person skilled in the art.

Alternatively, some intermediates of formula (III) may be prepared by hydrolysis of compounds of formula (I) wherein X, R¹, R², R³ and R⁴ are as defined here above. This reaction may be performed by hydrolysis in basic conditions using metal hydroxides such as lithium hydroxide in aqueous medium at high temperature or according to any conditions known to the person skilled in the art.

Intermediates of formula (II), wherein G represents (G^{c}), may be prepared by a process involving reaction of an intermediate of formula (IIa),

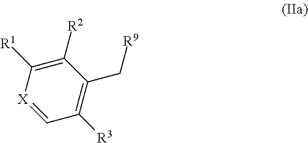

wherein

R⁹ represents cyano or —COOR^{c};

R^{c} represents C₁₋₆ alkyl; and

X, R¹, R² and R³ are as defined above.

When R⁹ represents —COOR^{c}, the reaction is conveniently performed in the presence of a suitable base, e.g. lithium hydroxide, in a suitable solvent, e.g. water, according to methods known to the person skilled in the art.

When R⁹ represents cyano, the reaction is conveniently be performed in the presence of a strong acid, e.g. sulphuric acid, or a strong base, e.g. sodium hydroxide, in a suitable solvent, e.g. polar solvent such as water or ethanol, at elevated temperature.

Intermediates of formula (IIa) may be prepared by a process involving decarboxylation of an intermediate of formula (IIb),

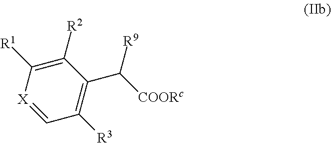

wherein X, R¹, R², R³, R^{c} and R⁹ as defined here above.

When R⁹ represents —COOR^{c}, and R^{c} is as defined here above, decarboxylation is conveniently performed in the presence of lithium chloride, in a suitable solvent e.g. mixture of water and dimethylsulphoxide, at elevated temperature.

When R⁹ represents cyano, decarboxylation is conveniently performed in the presence of a suitable acid, e.g. trifluoroacetic acid, in a suitable solvent e.g. dichloromethane, at elevated temperature.

Alternatively, intermediates of formula (IIa) and (IIb), may be prepared by a process involving reaction of an intermediate of formula (IIc),

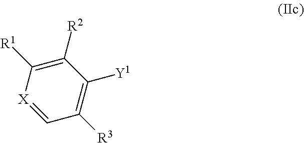

wherein Y¹ represents halogen, e.g. fluoro, bromo or iodo and X, R¹, R², R³ are as defined above;

with a compound of formula CHR^{d}R⁹;

wherein

R^{d} represents respectively hydrogen or M-Y; or —COOR^{c};

M is a metal, e.g. zinc; and

R^{c}, R⁹ and Y are as defined here above.

When R^{d} represents hydrogen, the reaction is conveniently performed in the presence of a suitable base, e.g. lithium hydroxide, in a suitable solvent, e.g. water, according to methods known to the person skilled in the art.

When R^{d} represents —COOR^{c}, the reaction is conveniently performed in the presence of an inorganic base, e.g. cesium carbonate, in a suitable solvent, e.g. dimethylformamide, at elevated temperature.

When R^{d} represents M-Y, the reaction is conveniently performed in the presence of a transition metal catalyst complex, e.g. tri[(tert-butyl)phosphine]Pd(II), in a suitable solvent, e.g. THF, at elevated temperature.

Alternatively, intermediates of formula (II) wherein G represents (G^{c}), may be prepared by a process involving carboxylation of an intermediate of formula (IId)

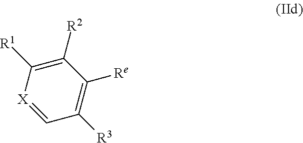

wherein R^{e} represents methyl and X, R¹, R² and R³ are as defined above. This reaction is conveniently performed using a base such as potassium tert-butoxide and dimethylcarbonate at room temperature in a suitable solvent such as DMF.

Intermediates of formula (IIf) wherein G represents (G^{c}), may be prepared by a process involving the reaction of an intermediate of formula (IIg),

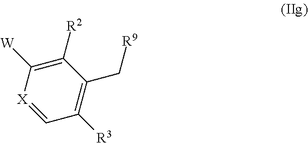

wherein W represents 1-ethoxyvinyl, R⁹ represents —COOR^{c}, R^{c} represents C₁₋₆ alkyl and X, R² and R³ are as defined above. The reaction is conveniently performed in the presence of a suitable base, e.g. lithium hydroxide, in a suitable solvent, e.g. water, according to methods known to the person skilled in the art.

Intermediates of formula (IIg), may be prepared by a coupling reaction from an intermediate of formula (IIh),

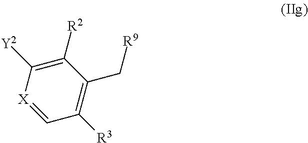

wherein Y² represents halogen, X, R⁹, R^{c}, R² and R³ are as defined above. The reaction may be performed by Stille-type coupling of a stannyl reagent such as tributyl(1-ethoxyvinyl)tin in the presence of a palladium catalyst such as tetrakis(triphenylphosphine)palladium(0) in a suitable solvent such as toluene at high temperature or by any alternative method known to the person skilled in the art.

Intermediates of formula (II), wherein G represents (G^{a}) or (G^{b}), respectively represented by formula II-(G^{a}) or II-(G^{b})

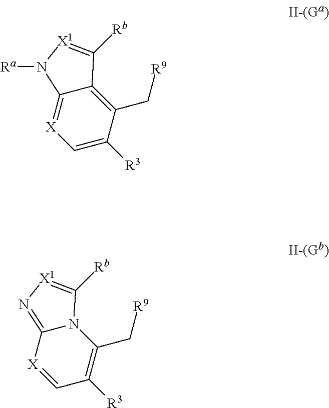

wherein R^{a} represents hydrogen or C₁₋₆ alkyl, i.e. methyl, R^{b} represents C₁₋₆ alkyl or halogen, i.e. chlorine, i.e. fluorine, X¹ represents N or CH and X, R³ and R⁹ are as defined above, may be prepared according to methods described above for intermediates of formula (II), wherein G represents (G^{c}).

Alternatively, intermediates of formula II-Ga) wherein R^{b} represents halogen, i.e. chlorine may be prepared by halogenation of intermediates of formula II-(Ga) wherein R^{b} represents hydrogen. This reaction may conveniently be performed using a chlorinating agent such as N-chlorosuccinimide in a suitable solvent such as dichloromethane at room temperature or by any method known to the person skilled in the art.

Alternatively, intermediates of formula II-(Ga) wherein X¹ represents N, R^{b} represents hydrogen and R³ represents halogen, i.e. chlorine, may be prepared by reaction of an intermediate of formula II-(G^{aa}),

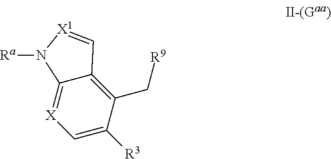

wherein R³ represents amino and R^{a}, X, X¹ and R⁹ are as defined above.

This reaction is conveniently performed by adding concentrated hydrochloric acid and sodium nitrite, followed by further addition of hydrochloric acid and copper chloride (II). The reaction is conveniently performed at low temperature.

Intermediates of formula II-(G^{aa}) may be prepared by reduction of an intermediate of formula II-(G^{aa}) wherein R³ represents nitro. This reaction is conveniently performed by Pd/C catalyzed hydrogenation under high pressure, in a suitable solvent e.g. methanol.

Intermediates II-(G^{aa}) wherein R³ represents nitro may be prepared from intermediates of formula (II-G^{ab})

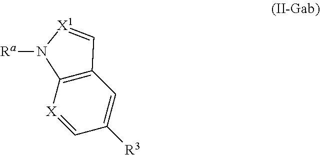

wherein R^{a}, X, X¹ are as defined above and R³ is nitro.

This reaction is conveniently performed using a reagent of formula X³—CH₂—R⁹ wherein X³ represents halogen, i.e chlorine and R⁹ is as defined above, in the presence of a base such as potassium tert-butoxide in a suitable solvent, such as THF, at low temperature.

Alternatively, intermediates of formula II-(G^{b}) wherein R^{b} represents halogen, i.e. chlorine may be prepared from an intermediate of formula II-(G^{d}),

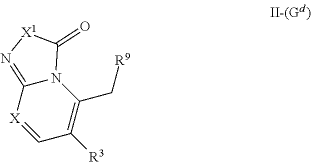

wherein X¹ represents N and X, R³ and R⁹ are as defined above. This reaction may be performed using phosphorus oxychloride in the presence of N,N-dimethylaniline at a temperature ranging from 90 to 120° C. or by any alternative method known to the person skilled in the art.

Intermediates of formula II-(G^{d}) may be prepared from an intermediate of formula (II-Ge),

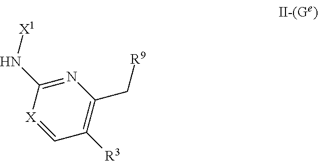

wherein X¹ represents NH₂, and X, R³ and R⁹ are as defined above. This reaction may be performed using a coupling agent such as carbonyldiimidazole in a suitable solvent such as THF at room temperature.

Intermediates of formula (IId), (lie), II-(G^{a}), II-(G^{ab}), and II-(G^{e}) are either commercially available or may be prepared by processes involving sequences of reactions known to the person skilled in the art

Where a mixture of products is obtained from any of the processes described above for the preparation of compounds or intermediates according to the invention, the desired product can be separated therefrom at an appropriate stage by conventional methods such as preparative HPLC; or normal phase column chromatography utilising, for example, silica and/or alumina in conjunction with an appropriate solvent system.

Where the above-described processes for the preparation of the compounds according to the invention give rise to mixtures of stereoisomers, these isomers may be separated by conventional techniques. In particular, where it is desired to obtain a particular enantiomer of a compound of formula (I) or of intermediates (II) or (Ill) this may be produced from a corresponding mixture of enantiomers using any suitable conventional procedure for resolving enantiomers. Thus, for example, diastereomeric derivatives, e.g. salts, may be produced by reaction of a mixture of enantiomers of formula (I), e.g. a racemate, and an appropriate chiral compound, e.g. a chiral base. The diastereomers may then be separated by any convenient means, for example by crystallisation, and the desired enantiomer recovered, e.g. by treatment with an acid in the instance where the diastereomer is a salt. In another resolution process a racemate of formula (I) may be separated using chiral HPLC or chiral SFC.

Moreover, if desired, a particular enantiomer may be obtained by using an appropriate chiral intermediate in one of the processes described above. Alternatively, a particular enantiomer may be obtained by performing an enantiomer-specific enzymatic biotransformation, e.g. an ester hydrolysis using an esterase, and then purifying only the enantiomerically pure hydrolysed acid from the unreacted ester antipode. Chromatography, recrystallisation and other conventional separation procedures may also be used with intermediates or final products where it is desired to obtain a particular geometric isomer of the invention. Alternatively, the non-desired enantiomer may be racemized into the desired enantiomer, in the presence of an acid or a base, according to methods known to the person skilled in the art, or according to methods described in the accompanying Examples.

During any of the above synthetic sequences it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 3^{rd} edition, 1999. The protecting groups may be removed at any convenient subsequent stage utilising methods known from the art.

The compounds of formula (I) according to the present invention does not directly activate the dopamine D1 receptor, but potentiates the effect of D1 agonists or the endogenous ligand on D1 receptors, dopamine, through an allosteric mechanism, and is therefore D1 positive allosteric modulator (D1 PAM).

Dopamine and other D1 agonists directly activate the dopamine D1 receptor by themselves.

Assays have been designed to measure the effects of compounds in accordance with the present invention in the absence of dopamine (“activation assay”) and in the presence of dopamine (“potentiation assay”).

The activation assay measures the stimulation of the production of cyclic adenosinemonophosphate (cAMP) in the Homogeneous Time Resolved Fluorescent (HTRF) assay, with the maximum increase in cAMP by increasing concentrations of the endogenous agonist, dopamine, defined as 100% activation.

When tested, compounds of formula (I) according to the present invention lacks significant direct agonist-like effects in that it produces less than 20% of activation (compared to dopamine maximal response) when present in a concentration of 10 μM.

The potentiation assay measures the ability of compounds to increase the levels of cAMP produced by a low-threshold concentration of dopamine. The concentration of dopamine used ([EC₂₀]) is designed to produce 20% stimulation compared to the maximal response (100%) seen with increasing the concentration of dopamine. To measure this potentiation increasing concentrations of the compound with the [EC₂₀] of dopamine are incubated and the potentiation is measured as increases in cAMP production and concentration of compound which produces 50% of the potentiation of the cAMP levels is measured.

When tested in the cAMP HTRF assay, compounds of formula (I) according to the the present invention have generally exhibited a value of pEC₅₀ of greater than about 5.5, ideally greater than about 6.5, appositely greater than about 7.0, which shows that they are D1 Positive Allosteric Modulators. Specific values are reported in Table A of the Examples.

GABA_{A} receptor inhibition is known to be intimately linked to seizures and epilepsy. It is therefore desirable to develop compounds which are D1 Positive Allosteric Modulators and which at the same time minimize such effects.

When tested in a GABA_{A} receptor inhibition assay as described herein, it is therefore desirable that compounds of formula (I) according to the present invention display a percentage of inhibition of the GABA_{A} receptor of less than or equal to about 20%, ideally less than about 10%, appositely less than about 5%, when measured at a concentration of 10 μM of a compound of formula (I), as further indicated in Table B of the Examples.

A problem which can be faced when developing compounds for use in therapy is the capacity for certain compounds to inhibit CYP450 enzymes. The inhibition of such enzymes may impact the exposure of such compounds or of other compounds which could be co-administered therewith to a patient, thereby potentially altering their respective safety or efficacy. It is therefore desirable to develop compounds which minimize such potential for inhibition.

The CYP450 inhibition potential of compound of formula (I) according to the present invention has been tested by measuring the potential decrease of CYP450 activities in human hepatocytes incubated with increasing concentrations of compounds according to the present invention.

When tested in the CYP3A4 inhibition assay at 20 μM concentration according to the protocol described in the present patent application, compounds of formula (I) according to the present invention exhibit generally a percentage of inhibition lower than about 80%, suitably lower than or equal to about 70%, ideally lower than or equal to about 60%, ideally lower than or equal to about 40%, and appositely lower than or equal to about 20%, as further indicated in Table C of the Examples.

### EXPERIMENTAL SECTION

### Abbreviations/Recurrent Reagents

Ac: Acetyl
ACN: Acetonitrile
Brine: Saturated aqueous sodium chloride solution
nBu: n-butyl
tBu: tert-butyl
tBuXPhos palladacycle: [2-(Di-tert-butylphosphino)-2′,4′,6′-triisopropyl-1,1′-biphenyl][2-(2-aminoethyl)phenyl)]palladium(II) chloride
CDI: Carbonyldiimidazole
dba: dibenzylideneacetonate
DCM: Dichloromethane
DEA: Diethylamine
DHP: 3,4-Dihydropyran
DIPEA: N,N-Diisopropylethyamine
DMAP: 4-Dimethylaminopyridine
DMF: N,N-Dimethylformamide
DMSO: Dimethylsulfoxide
EC_{20/50}: concentration which produces 20%/50% of the maximum response
Erel: relative efficacy
ES⁺: Electrospray Positive lonisation
Et: Ethyl
EtOH: Ethanol
Et₂O: Diethyl ether
EtOAc: Ethyl acetate
h: Hour
HBTU: [Benzotriazol-1-yloxy(dimethylamino)methylene]-dimethyl-ammonium hexafluorophosphate
HEPES: 4-(2-hydroxyethyl)-1-piperazineethanesulfonic acid
HPLC: High Performance Liquid Chromatography
HTRF: Homogenous time-resolved fluorescence
IPAC: Isopropyl acetate
LC: Liquid Chromatography
LCMS: Liquid Chromatography Mass Spectrometry
LDA: Lithium diisopropylamide
Me: Methyl
MeOH: Methanol
min.: minutes
NCS: N-Chlorosuccinimide
NMR: Nuclear magnetic resonance
iPr: isopropyl
iPrOH: isopropanol
p-TSA: p-toluenesulfonic acid
rt: room temperature
RT: Retention Time
SFC: Supercritical Fluid Chromatography
SPE: Solid phase extraction
TEA: Triethylamine
TFA: Trifluoroacetic acid
THF: Tetrahydrofuran
TLC: Thin Layer Chromatography
TMS: Trimethylsilyl
UPLC: Ultra High Performance Liquid Chromatography
Xantphos: 4,5-Bis(diphenylphosphino)-9,9-dimethylxanthene
cAMP: cyclic adenosinemonophosphate
IUPAC names have been generated using Biovia Draw Version 19.1 (2019) or version 20.1 (2020).

Analytical Methods

All reactions involving air or moisture-sensitive reagents were performed under a nitrogen or argon atmosphere using dried solvents and glassware. Experiments requiring microwave irradiation are performed on a Biotage Initiator Sixty microwave oven upgraded with version 2.0 of the operating software. Experiments are run to reach the required temperature as quickly as possible (maximum irradiation power: 400 W, no external cooling). Commercial solvents and reagents were generally used without further purification, including anhydrous solvents when appropriate (generally Sure-Seal™ products from Aldrich Chemical Company or AcroSeal™ from ACROS Organics). In general reactions were followed by thin layer chromatography, HPLC or mass spectrometry analyses.

HPLC analyses are performed with Shimadzu HPLC system equipped with LC-2010 CHT module, SPD-M20A photodiode array detector (210-400 nm), by using column YMC Triart C-18 (150×4.6) mm 3p. Gradient elution is done with 5 mM ammonium formate in water+0.1% Ammonia (Phase A), and Acetonitrile+5% solvent A+0.1% Ammonia (Phase B), with gradient 5-95% in 8.0 min hold till 13.0 min, 5% B at 15.0 min hold till 18.0 min. HPLC flow rate.

It will be apparent to the one skilled in the art that different retention times (RT) may be obtained for LC data if different analytical conditions are used.

Mass spectrometric measurements in LCMS mode are performed using different methods and instrument as follows:

Basic LCMS Method 1:

A Shimadzu 2010EV single quadrupole mass spectrometer is used for LC-MS analysis. This spectrometer is equipped with an ESI source and LC-20AD binary gradient pump, SPD-M20A photodiode array detector (210-400 nm). Data is acquired in a full MS scan from m/z 70 to 1200 in positive and negative mode. The reverse phase analysis is carried out by using Waters XBridge C 18 (30×2.1) mm 2.5μ column Gradient elution is done with 5 mM ammonium formate in H₂O+0.1% NH₄OH (solvent A), or ACN+5% solvent A+0.1% NH₄OH (solvent B), with gradient 5-95% B in 4.0 min hold till 5.0 min, 5% B at 5.1 min hold till 6.5 min. HPLC flow rate: 1.0 mL/min, injection volume: 5 μL.

Basic LCMS Method 2:

A QDA Waters simple quadrupole mass spectrometer is used for LCMS analysis. This spectrometer is equipped with an ESI source and an UPLC Acquity Classic with diode array detector (210 to 400 nm). Data is acquired in a full MS scan from m/z 70 to 800 in positive/negative modes with a basic elution. The reverse phase separation is carried out at 45° C. on a Waters Acquity UPLC BEH C18 1.7 μm (2.1×50 mm) column for basic elution. Gradient elution is done with H₂O/ACN/ammonium formate (95/5/63 mg/L)+100 μL/L NH₄OH (solvent A) and ACN/H₂O/ammonium formate (95/5/63 mg/L)+100 μL/L NH₄OH (solvent B). Injection volume: 1 μL. Full flow in MS.

| Time (min) | A (%) | B (%) | Flow (mL/min) |
|---|---|---|---|
| 0 | 99 | 1 | 0.4 |
| 0.3 | 99 | 1 | 0.4 |
| 3.2 | 0 | 100 | 0.4 |
| 3.25 | 0 | 100 | 0.5 |
| 4 | 0 | 100 | 0.5 |

Acid LCMS Method 1:

A QDA Waters simple quadrupole mass spectrometer is used for LCMS analysis. This spectrometer is equipped with an ESI source and an UPLC Acquity with diode array detector (200 to 400 nm). Data is acquired in a full MS scan from m/z 70 to 800 in positive/negative modes with an acidic elution. The reverse phase separation is carried out at 45° C. on a Waters Acquity UPLC HSS T3 1.8 μm (2.1×50 mm) column for acidic elution. Gradient elution is done with H₂O/ACN/TFA (95/5/0.05%) (solvent A) and ACN (solvent B).

| Time (min) | A (%) | B (%) | Flow (mL/min) |
|---|---|---|---|
| 0 | 99 | 1 | 0.4 |
| 0.3 | 99 | 1 | 0.4 |
| 3.2 | 5 | 95 | 0.4 |
| 3.25 | 5 | 95 | 0.5 |
| 4 | 5 | 95 | 0.5 |

Some reaction mixtures could be treated using Isolute® separator phase cartridges (from Biotage), acidic columns or catch and release SPE (Solid Phase Extraction) cartridges. Crude materials could be purified by normal phase chromatography, preparative TLC, (acidic or basic) reverse phase chromatography, chiral separation trituration or recrystallization.

Normal phase chromatography was performed using silica gel columns (100:200 mesh silica gel or cartridges for normal phase column chromatography systems such as Isolera™ Four from Biotage® or Teledyne Isco CombiNormal phase Column®).

Preparative reverse phase chromatographies are performed as follows:

Basic LCMS Prep:

LCMS purification (Basic mode, LCMS prep) using SQD Waters single quadrupole mass spectrometer is used for LCMS purification. This spectrometer is equipped with an ESI source, Waters 2525 binary pump coupled with 2767 sample Manager and with diode array detector (210 to 400 nm.) Data are acquired in a full MS scan from m/z 100 to 850 in positive and negative modes with a basic elution.

LCparameters: The reverse phase separation is carried out at room temperature on a Waters XBridge OBD MS C18 column (5 μm, 30×50 mm). Gradient elution is performed with solvent A1 (H₂O+NH₄HCO₃ 10 mM+50 μl/L NH₄OH) and solvent B1 (100% ACN) (pH ˜8.5). HPLC flow rate: 35 mL/min to 45 mL/min, injection volume: 990 μL. The splitting ratio is set at +/−1/6000 to MS.

| Time (min) | A1 (%) | B1(%) | Flow (mL/min) |
|---|---|---|---|
| 0 | 95 | 5 | 35 |
| 1 | 95 | 5 | 35 |
| 7 | 10 | 90 | 35 |
| 7.5 | 5 | 95 | 35 |
| 9 | 5 | 95 | 35 |
| 9.1 | 5 | 95 | 45 |
| 12 | 5 | 95 | 45 |

Acidic LCMS Prep:

LCMS purification (acidic mode, LCMS prep) using SQD Waters single quadrupole mass spectrometer is used for LCMS purification. This spectrometer is equipped with an ESI source, Waters 2525 binary pump coupled with 2767 sample Manager and with diode array detector (210 to 400 nm.) Data are acquired in a full MS scan from m/z 100 to 850 in positive mode with an acidic elution.

LC parameters: The reverse phase separation is carried out at room temperature on a Waters Sunfire ODB MS C18 column (5 μm, 30×50 mm). Gradient elution is performed with solvent A2 (Water/TFA: 99.5%+0.5% TFA) and solvent B2 (ACN/TFA: 99.5%+0.5%) (pH ˜2). HPLC flow rate: 35 mL/min to 45 mL/min, injection volume: 990 μL. The splitting ratio is set at +/−1/6000 to MS.

| Time (min) | A2 (%) | B2(%) | Flow (mL/min) |
|---|---|---|---|
| 0 | 95 | 5 | 35 |
| 1 | 95 | 5 | 35 |
| 7 | 10 | 90 | 35 |
| 7.5 | 5 | 95 | 35 |
| 9.1 | 5 | 95 | 45 |
| 12 | 5 | 95 | 45 |

Products were generally dried under vacuum before final analyses and submission to biological testing.

NMR spectra were recorded on different instruments:
a BRUKER AVANCEIII 400 MHz-Ultrashield NMR Spectrometer fitted with a Windows 7 Professional workstation running Topspin 3.2 software and a 5 mm Double Resonance Broadband Probe (PABBI ¹H/¹⁹F—BB Z-GRD Z82021/0075) or a 1 mm Triple Resonance Probe (PATXI ¹H/D-¹³C/¹⁵N Z-GRD Z868301/004).
a Varian MR 400 MHz NMR Spectrometer fitted with a Linux 3.2 software with operating system Redhat enterprise Linux 5.1. and 5 mm inverse ¹H/¹³C probe head, or Varian VNMR 400 MHz NMR fitted with Linux 3.2 software with operating system Redhat enterprise Linux 6.3 and 5 mm inverse ¹H/¹³C/¹⁹F triple probe head.

Chemical shifts are referenced to signals deriving from residual protons of the deuterated solvents (DMSO-d₆, MeOH-d₄ or CDCl₃). Chemical shifts are given in parts per million (ppm) and coupling constants (J) in Hertz (Hz). Spin multiplicities are given as broad (br), singlet (s), doublet (d), triplet (t), quartet (q) and multiplet (m).

All final products were analysed by LCMS in both basic and acid modes, as follows:

Basic LCMS Method 3:

A QDA Waters simple quadrupole mass spectrometer is used for LCMS analysis. This spectrometer is equipped with an ESI source and an UPLC Acquity Classic with diode array detector (210 to 400 nm). Data is acquired in a full MS scan from m/z 70 to 800 in positive/negative modes with a basic elution. The reverse phase separation is carried out at 45° C. on a Waters Acquity UPLC BEH C18 1.7 μm (2.1×100 mm) column for basic elution. Gradient elution is done with H₂O/ACN/ammonium formate (95/5/63 mg/L)+100 μL/L NH₄OH (solvent A) and ACN/H₂O/ammonium formate (95/5/63 mg/L)+100 μL/L NH₄OH (solvent B). Injection volume: 1 μL. Full flow in MS.

| Time (min) | A (%) | B (%) | Flow (mL/min) |
|---|---|---|---|
| 0 | 99 | 1 | 0.4 |
| 0.8 | 99 | 1 | 0.4 |
| 5.30 | 0 | 100 | 0.4 |
| 5.35 | 0 | 100 | 0.5 |
| 7.30 | 0 | 100 | 0.5 |

Acid LCMS Method 2:

A QDA Waters simple quadrupole mass spectrometer is used for LCMS analysis. This spectrometer is equipped with an ESI source and an UPLC Acquity Hclass with diode array detector (210 to 400 nm). Data are acquired in a full MS scan from m/z 70 to 800 in positive/negative modes with an acidic elution. The reverse phase separation is carried out at 45° C. on a Waters Acquity UPLC HSS T3 1.8 μm (2.1×100 mm) column for acidic elution. Gradient elution is done with H₂O/ACN/TFA (95/5/0.05%) (solvent A and ACN (solvent B).

| Time (min) | A (%) | B (%) | Flow (mL/min) |
|---|---|---|---|
| 0 | 99 | 1 | 0.4 |
| 0.8 | 99 | 1 | 0.4 |
| 5.3 | 5 | 95 | 0.4 |
| 5.35 | 5 | 95 | 0.5 |
| 7.3 | 5 | 95 | 0.5 |

### SYNTHETIC INTERMEDIATES

### A. Synthesis of Intermediates of Formula (II)

### A.1. Synthesis of 2-(2-chloro-6-cyano-3-methoxyphenyl)acetic acid a3

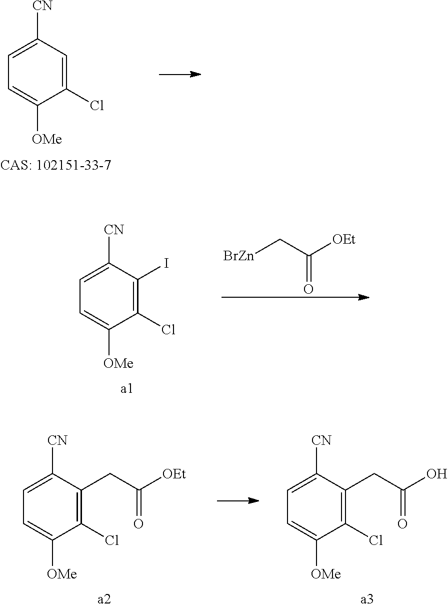

### A.1.1. Synthesis of 3-chloro-2-iodo-4-methoxybenzonitrile a1

To a solution of 3-chloro-4-methoxy-benzonitrile (commercial, 12.0 g, 71.8 mmol) in THF (150 mL) was added LDA (51.0 mL, 165 mmol) at −78° C. and reaction mixture was stirred at same temperature for 45 min. I₂ (27.0 g, 108 mmol) was added at −78° C. and reaction mixture was stirred at same temperature for 3 h. Progress of the reaction was monitored by TLC and LCMS. After completion, the reaction mixture was quenched with a saturated aqueous solution of NH₄Cl (200 mL), extracted with EtOAc (2×200 mL) and washed with H₂O (100 mL). The organic layer was dried over anhydrous Na₂SO₄ and concentrated under vacuum. The crude residue was purified by normal phase column chromatography (elution: 10% EtOAc in hexanes) to afford 10.5 g of 3-chloro-2-iodo-4-methoxybenzonitrile a1 as a pink solid.

Yield: 50%.

¹H NMR (400 MHz, DMSO-d₆): δ 7.84 (d, J=8.58 Hz, 1H), 7.33 (d, J=8.11 Hz, 1H), 3.94 (s, 3H).

### A.1.2. Synthesis of ethyl 2-(2-chloro-6-cyano-3-methoxyphenyl)acetate a2

To a solution of 3-chloro-2-iodo-4-methoxybenzonitrile a1 (10.0 g, 34.1 mmol) in THF (150 mL) was added Pd(OAc)₂ (0.76 g, 3.41 mmol) and (tBu)₃P·HBF₄ (1.97 g, 6.82 mmol), followed by addition of ethoxycarbonyl methylzinc bromide (11.8 g, 51.1 mmol). The reaction mixture was heated at 60° C. for 8 h. Progress of the reaction was monitored by TLC and LCMS. After completion, the reaction mixture was filtered through a pad of Celite® and filtrate was quenched with a saturated aqueous solution of NH₄Cl (50 mL) and extracted with EtOAc (3×150 mL). The organic layer was dried over anhydrous Na₂SO₄ and concentrated under vacuum. The crude residue obtained was purified by normal phase column chromatography (elution: from 0 to 12% EtOAc in hexanes) to afford 6.86 g of ethyl 2-(2-chloro-6-cyano-3-methoxyphenyl)acetate a2 as a light yellow solid.

Yield: 79%.

¹H NMR (400 MHz, DMSO-d₆): δ 7.88 (d, J=8.80 Hz, 1H), 7.29 (d, J=8.80 Hz, 1H), 4.13 (q, J=7.34 Hz, 2H), 4.00 (s, 2H), 3.96 (s, 3H), 1.19 (t, J=7.09 Hz, 3H).

### A.1.3. Synthesis of 2-(2-chloro-6-cyano-3-methoxyphenyl)acetic acid a3

To a solution of ethyl 2-(2-chloro-6-cyano-3-methoxyphenyl)acetate a2 (2.20 g, 8.69 mmol) in THF (5 mL) and H₂O (5 mL) was added LiOH (0.62 g, 26.0 mmol). The reaction mixture was stirred at rt for 16 h. Progress of reaction was monitored by TLC and LCMS. After completion, the reaction mixture was concentrated under vacuum. The crude residue was acidified to pH 2 with a 2N aqueous solution of HCl and extracted with EtOAc (50 mL). The organic layer was washed with H₂O (2×50 mL), dried over anhydrous Na₂SO₄ and concentrated under vacuum to afford 1.60 g of 2-(2-chloro-6-cyano-3-methoxyphenyl)acetic acid a3 as an off-white solid, which was used in the next steps without further purification.

Yield (crude): 84%.

HPLC (Basic mode): 99% purity.

¹H NMR (400 MHz, DMSO-d₆): δ 12.86 (brs, 1H), 7.86 (d, J=8.80 Hz, 1H), 7.27 (d, J=8.80 Hz, 1H), 3.96 (s, 3H), 3.91 (s, 2H).

### A.2. Synthesis of 2-(6-chloro-2-cyano-3-methoxyphenyl)acetic acid a9

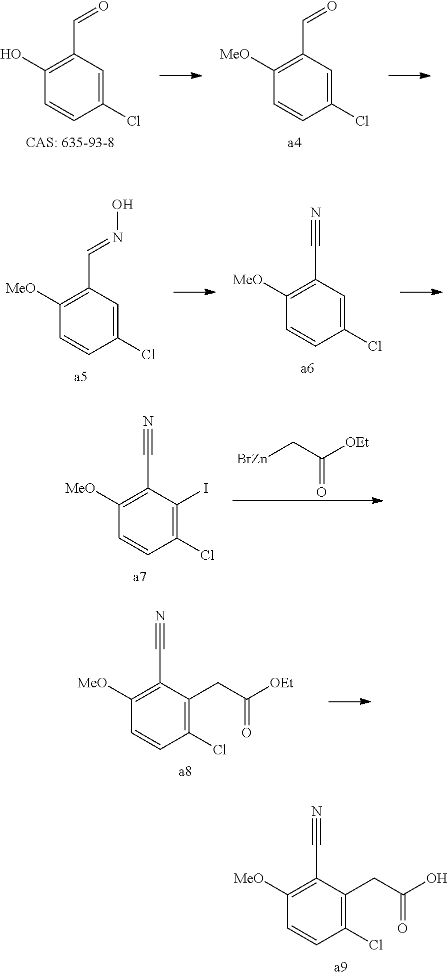

### A.2.1. Synthesis of 5-chloro-2-methoxybenzaldehyde a4

To a solution of 5-chloro-2-hydroxy-benzaldehyde (commercial, 15.0 g, 96.1 mmol) in acetone (150 mL) was added K₂CO₃ (16.4 g, 119 mmol) followed by dropwise addition of Mel (14.7 mL, 240 mmol) and reaction mixture was heated to reflux for 5 h. Progress of the reaction was monitored by TLC and LCMS. After completion, the reaction mixture was concentrated under vacuum. The crude residue was extracted with EtOAc (3×300 mL). The organic layer was dried over anhydrous Na₂SO₄ and concentrated under vacuum. The crude residue obtained was purified by normal phase column chromatography (elution: 8% EtOAc in hexanes) to afford 15.0 g of 5-chloro-2-methoxybenzaldehyde a4 as a white solid.

Yield: 92%.

¹H NMR (400 MHz, DMSO-d₆): δ 10.29 (s, 1H), 7.71 (dd, J=8.8, 2.45 Hz, 1H), 7.62 (d, J=2.45 Hz, 1H), 7.29 (d, J=8.8 Hz, 1H), 3.93 (s, 3H).

A.2.2. Synthesis of (NE)-N-[(5-chloro-2-methoxyphenyl)methylidene]hydroxylamine a5 To a solution of 5-chloro-2-methoxybenzaldehyde a4 (15.0 g, 88.2 mmol) in EtOH (150 mL) was added NH₂OH·HCl (9.13 g, 132 mmol). The reaction mixture was stirred at rt for 16 h. Progress of the reaction was monitored by TLC and LCMS. After completion, the reaction mixture was concentrated under vacuum. The crude residue was diluted with H₂O (200 mL) and extracted with EtOAc (3×250 mL). The organic layer was dried over anhydrous Na₂SO₄ and concentrated under vacuum to afford 15.1 g of (NE)-N-[(5-chloro-2-methoxyphenyl)methylidene]hydroxylamine a5 as a brown liquid, which was used in the next steps without further purification.

Yield (crude): 92%.

¹H NMR (400 MHz, DMSO-d₆): δ 11.45 (d, J=1.96 Hz, 1H), 8.23 (d, J=1.96 Hz, 1H), 7.59 (d, J=2.45 Hz, 1H), 7.35-7.46 (m, 1H), 7.04-7.17 (m, 1H), 3.83 (s, 3H).

### A.2.3. Synthesis of 5-chloro-2-methoxybenzonitrile a6

A stirred solution (NE)-N-[(5-chloro-2-methoxyphenyl)methylidene]hydroxylamine a5 (15.0 g, 81.0 mmol) in Ac₂O (100 mL) was heated at 100° C. for 16 h. Progress of the reaction was monitored by TLC. After completion, the reaction mixture was diluted with H₂O (200 mL) and extracted with EtOAc (3×300 mL). The organic layer was dried over anhydrous Na₂SO₄ and concentrated under vacuum. The crude residue obtained was purified by normal phase column chromatography (elution: 15% EtOAc in hexanes) to afford 7.68 g of 5-chloro-2-methoxybenzonitrile a6 as an off-white solid.

Yield: 57%.

¹H NMR (400 MHz, DMSO-d₆): δ 7.86-7.98 (m, 1H), 7.67-7.77 (m, 1H), 7.27 (dd, J=8.56, 4.16 Hz, 1H), 3.91 (s, 3H).

### A.2.4. Synthesis of 3-chloro-2-iodo-6-methoxybenzonitrile a7

To a solution of 5-chloro-2-methoxybenzonitrile a6 (4.00 g, 23.9 mmol) in THF (50 mL) was added LDA (26.3 mL, 52.6 mmol) at −78° C. The reaction mixture was stirred at same temperature for 45 min followed by addition of I₂ (7.29 g, 28.7 mmol). The reaction mixture was stirred at −78° C. for 45 min. Progress of the reaction was monitored by TLC and LCMS.

After completion, the reaction mixture was quenched with a saturated aqueous solution of NH₄Cl (40 mL) and extracted with EtOAc (3×200 mL). The organic layer was washed with H₂O (100 mL), dried over anhydrous Na₂SO₄ and concentrated under vacuum. The crude residue obtained was purified by normal phase column chromatography (elution: 20% EtOAc in hexanes) to afford 2.6 g of 3-chloro-2-iodo-6-methoxybenzonitrile a7 as an off-white solid.

Yield: 37%.

¹H NMR (400 MHz, DMSO-d₆): δ 7.84 (d, J=9.29 Hz, 1H), 7.31 (d, J=9.29 Hz, 1H), 3.87-3.95 (s, 3H).

### A.2.5. Synthesis of ethyl 2-(6-chloro-2-cyano-3-methoxyphenyl) acetate a8

To a solution of 3-chloro-2-iodo-6-methoxybenzonitrile a7 (5.00 g, 17.0 mmol) in THF (120 mL) was added ethoxycarbonyl methylzinc bromide (51.0 mL, 25.5 mmol) followed by addition of Pd(tBu₃P)₂ (0.43 g, 0.85 mmol). The reaction mixture was heated at 50° C. for 6 h. Progress of the reaction was monitored by TLC and LCMS. After completion, the reaction mixture was diluted with H₂O (100 mL) and extracted with EtOAc (2×250 mL). The organic layer was dried over anhydrous Na₂SO₄ and concentrated under vacuum. The crude residue obtained was purified by normal phase column chromatography (elution: 20% EtOAc in hexanes) to afford 2.10 g of ethyl 2-(6-chloro-2-cyano-3-methoxyphenyl) acetate a8 as a brown solid.

Yield: 9%.

¹H NMR (400 MHz, DMSO-d₆): δ 7.78 (d, J=8.80 Hz, 1H), 7.24 (d, J=8.80 Hz, 1H), 4.12 (q, J=6.85 Hz, 2H), 3.93 (brs, 3H), 3.91 (brs, 2H), 1.18 (t, J=7.09 Hz, 3H).

### A.2.6. Synthesis of 2-(6-chloro-2-cyano-3-methoxyphenyl)acetic acid a9

To a solution of ethyl 2-(6-chloro-2-cyano-3-methoxyphenyl) acetate a8 (2.00 g, 7.90 mmol) in THF (15 mL) and H₂O (15 mL) was added LiOH (0.57 g, 23.7 mmol). The reaction mixture was stirred at rt for 16 h. Progress of reaction was monitored by TLC and LCMS. After completion, the reaction mixture was concentrated under vacuum. The crude residue was diluted with H₂O (25 mL) and acidified with a 6N aqueous solution of HCl to pH 2 and extracted with EtOAc (200 mL). The organic layer was washed with H₂O (200 mL), dried over anhydrous Na₂SO₄ and concentrated under vacuum. The crude residue obtained was purified by normal phase column chromatography (elution: 4% MeOH in DCM) to afford 1.10 g of 2-(6-chloro-2-cyano-3-methoxyphenyl)acetic acid a9 as an off-white solid.

Yield: 62%.

HPLC (Basic mode): 98% purity.

¹H NMR (400 MHz, DMSO-d₆): δ 12.91 (s, 1H), 7.77 (d, J=8.80 Hz, 1H), 7.23 (d, J=9.29 Hz, 1H), 3.93 (s, 3H), 3.86 (s, 2H).

### A.3. Synthesis of 2-(3,5-dichloro-2-methoxy-4-pyridyl)acetic acid a15

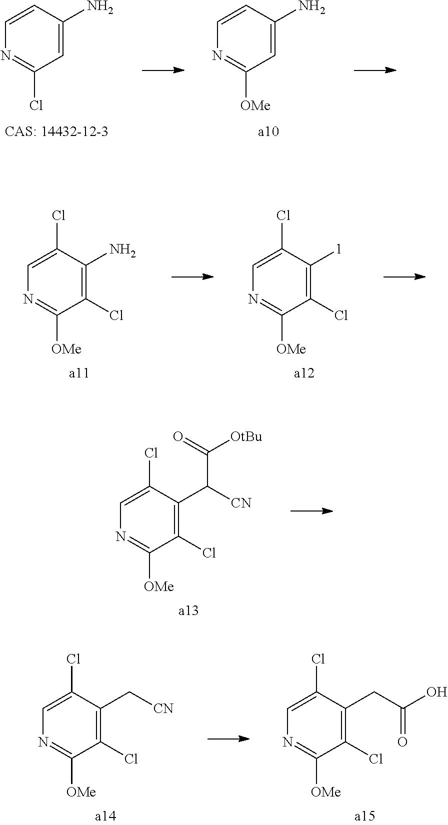

### A.3.1. Synthesis of 2-methoxypyridin-4-amine a10

To a solution of NaOMe (672 mL, 3.11 mol) at rt, 2-chloropyridin-4-amine (commercial, 50.0 g, 389 mmol) was added and the reaction mixture was heated at 160° C. for 8 h in an autoclave. Progress of the reaction was monitored by TLC. After completion, the reaction mixture was concentrated under vacuum, then the obtained residue was diluted with ice cold H₂O (1 L). The compound was extracted with a solution of 5% MeOH in DCM. The organic layer was dried over Na₂SO₄ and concentrated under vacuum. The crude residue was diluted with EtOAc (1 L), then the organic layer was washed with brine, dried over anhydrous Na₂SO₄ and concentrated under vacuum to afford 15.0 g of 2-methoxypyridin-4-amine a10 as a pale yellow sticky mass, which was used in the next steps without further purification.

Yield (crude): 31%.

Basic LCMS Method 1 (ES+): 125 (M+H)+.

¹H NMR (400 MHz, DMSO-d₆): δ 7.60-7.64 (m, 1H), 6.16 (dd, J=5.61, 2.02 Hz, 1H), 5.88 (brs, 2H), 5.80 (d, J=1.80 Hz, 1H), 3.68-3.73 (s, 3H).

### A.3.2. Synthesis of 3,5-dichloro-2-methoxy-pyridin-4-amine all

To a solution of 2-methoxypyridin-4-amine a10 (30.0 g, 242 mmol) in ACN (1 L) at rt, NCS (129 g, 967 mmol) was added portion wise and the reaction mixture was stirred at rt for 16 h. Progress of the reaction was monitored by TLC. After completion, the reaction mixture was concentrated under vacuum. The crude residue was diluted with a 20% aqueous solution of potassium carbonate (500 mL). The compound was extracted with EtOAc. The organic layer was washed with brine, dried over anhydrous Na₂SO₄ and concentrated under vacuum. The crude residue was purified by normal phase column chromatography (elution: 50% EtOAc in hexanes) to afford 35.1 g of 3,5-dichloro-2-methoxy-pyridin-4-amine all.

Yield: 75%.

Basic LCMS Method 1 (ES+): 194/196/198 (M+H)+.

¹H NMR (400 MHz, DMSO-d₆): δ 7.70-7.91 (s, 1H), 6.50 (s, 2H), 3.80-3.97 (s, 3H).

### A.3.3. Synthesis of 3,5-dichloro-4-iodo-2-methoxy-pyridine a12

To a solution of Cul (59.0 g, 311 mmol) in ACN (1 L) was added dropwise tBuONO (93.0 mL, 777 mmol) at 50° C. The reaction mixture was heated at 80° C. for 30 min. A solution of 3,5-dichloro-2-methoxy-pyridin-4-amine all (30.0 g, 155 mmol) in ACN (500 mL) was added in portions (evolution of nitrogen gas was observed) and the reaction mixture was stirred at 80° C. for 2 h. Progress of the reaction was monitored by TLC. After completion, the reaction mixture was concentrated under vacuum and the crude residue was diluted EtOAc (100 mL) and hexane (2 L). The resulting suspension was passed through a short silica pad and the filtrate was concentrated under vacuum to afford 34.9 g of 3,5-dichloro-4-iodo-2-methoxy-pyridine a12 as a pale yellow solid.

Yield: 74%.

Basic LCMS Method 1 (ES⁺): 305 (M+2)⁺.

¹H NMR (400 MHz, DMSO-d₆): δ 8.19-8.34 (s, 1H), 3.87-4.00 (s, 3H).

### A.3.4. Synthesis of tert-butyl 2-cyano-2-(3,5-dichloro-2-methoxypyridin-4-yl)acetate a13

To a solution of 3,5-dichloro-4-iodo-2-methoxy-pyridine a12 (10.0 g, 32.9 mmol), tbutyl 2-cyanoacetate (9.40 mL, 65.8 mmol) and cesium carbonate (42.9 g, 132 mmol) in DMF (160 mL) was added Cul (0.63 g, 3.29 mmol) and the reaction mixture was stirred at 100° C. for 3 h. Progress of the reaction was monitored by TLC. After completion, the reaction mixture was poured onto ice cold water and neutralized with a 6N aqueous solution of HCl. The compound was extracted in EtOAc. The organic layer was washed with brine, dried over anhydrous Na₂SO₄, concentrated under vacuum and the crude residue was purified by normal phase column chromatography (elution: 20% EtOAc in hexanes) to afford 6.70 g of tert-butyl 2-cyano-2-(3,5-dichloro-2-methoxypyridin-4-yl)acetate a13.

Yield: 64%.

¹H NMR (400 MHz, DMSO-d₆): δ 8.39-8.53 (s, 1H), 6.32 (s, 1H), 3.92-4.07 (s, 3H), 1.42 (s, 9H).

### A.3.5. Synthesis of 2-(3,5-dichloro-2-methoxy-4-pyridyl)acetonitrile a14

To a solution of tert-butyl 2-cyano-2-(3,5-dichloro-2-methoxypyridin-4-yl)acetate a13 (20.0 g, 63.0 mmol) in DCM (500 mL) was added TFA (80 mL) at rt and the reaction mixture was refluxed for 2 h. Progress of the reaction was monitored by TLC. After completion, the reaction mixture was concentrated under vacuum and the crude residue was neutralized with a saturated aqueous solution of sodium bicarbonate. The compound was extracted in EtOAc. The organic layer was dried over anhydrous Na₂SO₄ and concentrated under vacuum to afford 13.5 g of 2-(3,5-dichloro-2-methoxy-4-pyridyl)acetonitrile a14 as yellow solid, which was used in the next steps without further purification.

Yield (crude): 98%.

¹H NMR (400 MHz, DMSO-d₆): δ 8.31-8.47 (s, 1H), 4.19-4.30 (m, 2H), 3.86-4.06 (s, 3H).

### A.3.6. Synthesis of 2-(3,5-dichloro-2-methoxy-4-pyridyl)acetic acid a15

To a solution of 2-(3,5-dichloro-2-methoxy-4-pyridyl)acetonitrile a14 (13.5 g, 62.0 mmol) in EtOH (300 mL) was added a 10N aqueous solution of NaOH (93.5 mL, 933 mmol) and the reaction mixture was refluxed for 12 h. Progress of the reaction was monitored by TLC. After completion, the reaction mixture was diluted with H₂O, then NH₄Cl (60 g) was added. Solvent was removed under vacuum and the aqueous layer was acidified to pH 5 with a 6N aqueous solution of HCl. The compound was extracted with a 5% solution of MeOH in DCM. The organic layer was dried over anhydrous Na₂SO₄ and concentrated under vacuum. The crude residue was purified by normal phase column chromatography (elution: 5% MeOH in DCM). The crude residue was further washed with a solution of 50% DCM in hexanes, filtered and dried to afford 5 g of 2-(3,5-dichloro-2-methoxy-4-pyridyl)acetic acid a15 as an off-white solid.

Yield: 34%.

Basic LCMS Method 1 (ES⁺): 237/239/241 (M+H)⁺.

¹H NMR (400 MHz, CD₃OD): δ 8.03-8.18 (s, 1H), 3.99 (d, J=3.02 Hz, 3H), 3.26-3.42 (s, 2H).

### A.4. Synthesis of 2-[2-chloro-6-cyano-3-(trideuteriomethoxy)phenyl]acetic acid a21

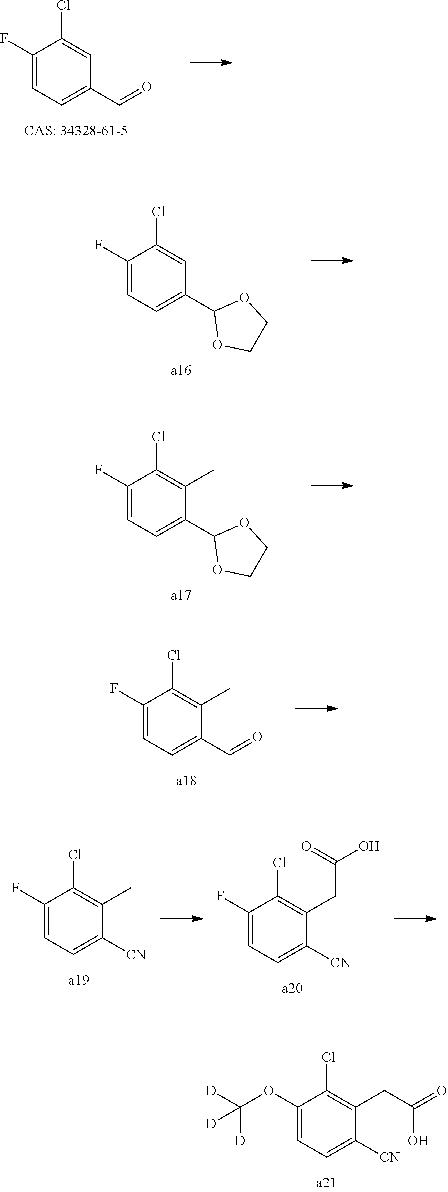

### A.4.1. Synthesis of 2-(3-chloro-4-fluorophenyl)-1,3-dioxolane a16

To a solution of 3-chloro-4-fluoro-benzaldehyde (10.0 g, 63.3 mmol) in toluene (150 mL) was added ethylene glycol (5.88 g, 95.0 mmol) and p-TSA (1.20 g 6.33 mmol). The reaction mixture was heated to reflux for 18 h, simultaneously H₂O was removed with the help of a dean stark apparatus. Progress of the reaction was monitored by TLC. After completion, the reaction mixture was concentrated under vacuum. The crude residue diluted with H₂O (150 mL) and extracted with EtOAc (2×150 mL). The organic layer was washed with H₂O (50 mL), brine (50 mL), dried over anhydrous Na₂SO₄ and concentrated under vacuum. The crude residue obtained was purified by normal phase column chromatography (elution: 5% EtOAc in hexanes) to afford 9.00 g of 2-(3-chloro-4-fluorophenyl)-1,3-dioxolane a16 as a colorless liquid.

Yield: 70%.

¹H NMR (400 MHz, DMSO-d₆): δ 7.59 (dd, J=7.21, 1.59 Hz, 1H), 7.40-7.43 (m, 2H), 5.72 (s, 1H), 4.01-4.04 (m, 2H), 3.91-3.95 (m, 2H).

### A.4.2. Synthesis of 2-(3-chloro-4-fluoro-2-methylphenyl)-1,3-dioxolane a17

To a solution of 2-(3-chloro-4-fluorophenyl)-1,3-dioxolane a16 (7.00 g, 34.6 mmol) in THF (140 mL) was added nBuLi (3.32 g, 51.9 mmol) dropwise at −78° C. and reaction mixture was stirred at same temperature for 1 h. Mel (24.6 g, 173 mmol) was added at −78° C. and reaction mixture was stirred at same temperature for 1 h and then at rt for 30 min. Progress of the reaction was monitored by TLC and LCMS. After completion, the reaction mixture was quenched with a saturated aqueous solution of NH₄Cl (70 mL) solution at −78° C. The reaction mixture was extracted with Et₂O (2×100 mL). The organic layer was washed with H₂O (50 mL), brine (50 mL), dried over anhydrous Na₂SO₄ and concentrated under vacuum. The crude residue obtained was purified by normal phase column chromatography (elution: 4% EtOAc in hexanes) to afford 7.00 g of a 7:3 mixture of 2-(3-chloro-4-fluoro-2-methylphenyl)-1,3-dioxolane a17 and its regioisomer 2-(3-chloro-4-fluoro-5-methylphenyl)-1,3-dioxolane a17b.

Yield: 93%.

Basic LCMS Method 1 (ES⁺): 217/219 (M+H)⁺, 89% purity.

¹H NMR (major isomer a17, 400 MHz, DMSO-d₆): δ 7.48 (m, 1H), 7.27 (m, 1H), 5.94 (s, 1H), 4.02-4.07 (m, 2H), 3.96-3.99 (m, 2H), 2.40 (s, 3H).

### A.4.3. Synthesis of 3-chloro-4-fluoro-2-methylbenzaldehyde a18

To a solution of a 7:3 mixture of 2-(3-chloro-4-fluoro-2-methylphenyl)-1,3-dioxolane a17 and its regioisomer 2-(3-chloro-4-fluoro-5-methylphenyl)-1,3-dioxolane a17b (8.80 g, 40.7 mmol) in THF (100 mL) was added a 1N aqueous solution of HCl (100 mL) and reaction mixture was heated to reflux for 4 h. Progress of the reaction was monitored by TLC. After completion, the reaction mixture was basified to pH 8 with a saturated aqueous solution of NaHCO₃ and extracted with Et₂O (3×100 mL). The organic layer was washed with H₂O (150 mL), brine (100 mL), dried over anhydrous Na₂SO₄ and concentrated under vacuum. The crude residue obtained was purified by normal phase column chromatography (elution: 4% EtOAc in hexanes) to afford 5.80 g of a 7:3 mixture of 3-chloro-4-fluoro-2-methylbenzaldehyde a18 and its regioisomer 3-chloro-4-fluoro-5-methylbenzaldehyde a18b.

Yield: 83%.

¹H NMR (major isomer a18, 400 MHz, DMSO-d₆): δ 10.20 (s, 1H), 7.84-7.92 (m, 1H), 7.48 (t, J=8.56 Hz, 1H), 2.68 (s, 3H).

### A.4.4. Synthesis of 3-chloro-4-fluoro-2-methylbenzonitrile a19

To a solution of a 7:3 mixture of 3-chloro-4-fluoro-2-methylbenzaldehyde a18 and its regioisomer 3-chloro-4-fluoro-5-methylbenzaldehyde a18b (6.80 g, 39.5 mmol) in THF (70 mL) and NH₄OH (680 mL) was added I₂ (10.8 g, 39.5 mmol) and stirred at rt for 2 h. Progress of the reaction was monitored by TLC and LCMS. After completion, the reaction mixture was quenched with a saturated aqueous solution of Na₂S₂O₃ (300 mL) solution and extracted with Et₂O (3×250 mL). The organic layer was washed with H₂O (200 mL), brine (250 mL), dried over anhydrous Na₂SO₄ and concentrated under vacuum. The crude residue obtained was purified by normal phase column chromatography (elution: 2% EtOAc in hexanes) to afford 6.00 g of a 7:3 mixture of 3-chloro-4-fluoro-2-methylbenzonitrile a19 and its regioisomer 3-chloro-4-fluoro-5-methylbenzonitrile a19b.

Yield: 89%.

¹H NMR (major isomer a19, 400 MHz, DMSO-d₆): δ 7.90 (dd, J=8.56, 5.14 Hz, 1H), 7.50 (t, J=8.56 Hz, 1H), 2.56 (s, 3H).

### A.4.5. Synthesis of 2-(2-chloro-6-cyano-3-fluorophenyl)acetic acid a20

To a solution of KOtBu (0.36 g, 3.25 mmol) in THF (10 mL) was added LDA (0.35 g, 3.25 mmol) at −78° C. and stirred at same temperature for 10 min. A solution of a 7:3 mixture of 3-chloro-4-fluoro-2-methylbenzonitrile a19 and its regioisomer 3-chloro-4-fluoro-5-methylbenzonitrile a19b (0.50 g, 2.95 mmol) in THF (2 mL) was added at −78° C. The reaction mixture was stirred at −78° C. for 30 min. CO₂ was purged into reaction mixture for 15 min. Progress of reaction was monitored by TLC and LCMS. After completion, the reaction mixture was quenched with H₂O (10 mL) and extracted with Et₂O (2×15 mL). The aqueous layer was acidified to pH 3 with a 3N aqueous solution of HCl and extracted with Et₂O (3×15 mL). The organic layer was dried over anhydrous Na₂SO₄ and concentrated under vacuum. The crude residue obtained was purified by normal phase column chromatography (elution: 40% EtOAc in hexanes) to afford 0.13 g of 2-(2-chloro-6-cyano-3-fluorophenyl)acetic acid a20 as off-white solid.

Yield: 24%.

Basic LCMS Method 1 (ES⁺): 214/216 (M+H)⁺, 95% purity.

¹H NMR (400 MHz, DMSO-d₆): δ 13.05 (brs, 1H), 7.99 (dd, J=8.58, 5.25 Hz, 1H), 7.63 (t, J=8.82 Hz, 1H), 3.99 (s, 2H).

### A.4.6. Synthesis of 2-[2-chloro-6-cyano-3-(trideuteriomethoxy)phenyl]acetic acid a21

Sodium hydride (1.00 g, 25.0 mmol) was slowly added at 0° C. to a solution of 2-(2-chloro-6-cyano-3-fluorophenyl)acetic acid a20 (1.07 g, 5.00 mmol) in CD₃OD (13.3 g, 366 mmol). The reaction mixture was stirred overnight at rt, then concentrated under vacuum. The crude residue was dissolved in MeOH (50 mL), then the mixture was heated at 60° C. for 48 h. The reaction mixture was diluted with EtOAc (150 mL), then successively washed with water (50 mL) and brine (50 mL). The organic layer was dried over MgSO₄, filtered and concentrated under vacuum. The crude was triturated in heptane and filtered off. The solid was dissolved in MeOH (10 mL), then Na (575 mg, 25.0 mmol) was added. The reaction mixture was heated at 60° C. overnight, then diluted with EtOAc (150 mL) and successively washed with a 1N aqueous solution of HCl (50 mL) and brine (50 mL). The organic layer was dried over MgSO₄, filtered and concentrated under vacuum to afford 1.14 g of 2-[2-chloro-6-cyano-3-(trideuteriomethoxy)phenyl]acetic acid a21, which was used in the next steps without further purification.

Yield (crude): quantitative

Basic LCMS Method 2 (ES⁺): 183/185 (M+H)⁺.

¹H NMR (400 MHz, CDCl₃): δ 7.60 (d, J=7.6 Hz, 1H), 6.95 (d, J=7.0 Hz, 1H), 4.10 (s, 2H).

### A.5. Synthesis of 2-[3,5-dichloro-2-(hydroxymethyl)-4-pyridyl]acetic acid a28

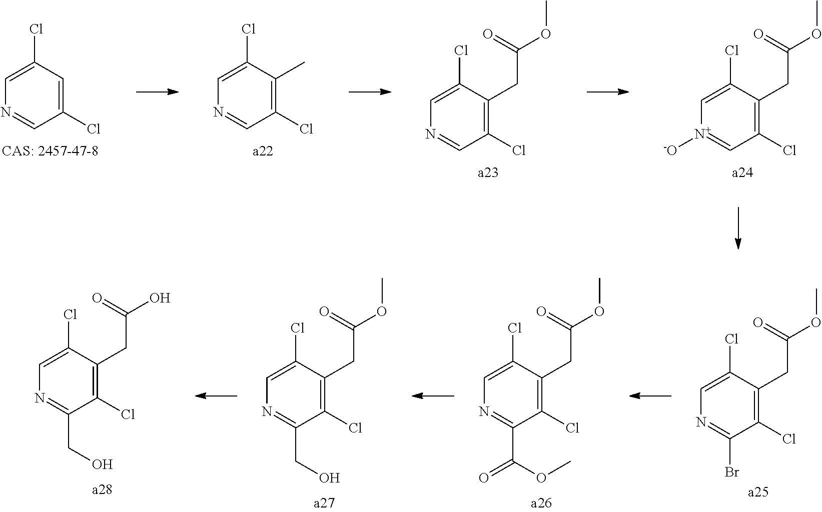

### A.5.1. Synthesis of 3,5-dichloro-4-methyl-pyridine a22

A 2 M solution of LDA in THF (1.86 L, 3.72 mol) and THF (5 L) were charged in a reactor under nitrogen. 3,5-Dichloro-4-methyl-pyridine (commercial, 500 g, 3.38 mol) was added at −20° C. and the mixture was stirred at −10° C. for 30 min. The reaction was cooled down to −70° C. and Mel (815 g, 5.74 mol) was added. The mixture was allowed to warm to rt and was stirred for 4 h. This overall procedure was carried out on 4 batches of the same size in parallel which were worked up together. The mixture was cooled to 0° C. and quenched with water (5 L) and stirred for 10 min. The aqueous layer was extracted with ethyl acetate (2×3 L) and the organic layer was washed twice with brine (10 L), dried over anhydrous Na₂SO₄, filtered and concentrated under vacuum. The crude residue was purified by recrystallization from EtOH (4 L) at −70° C. to afford 1.50 kg of 3,5-dichloro-4-methyl-pyridine a22 as a yellow solid.

Yield: 68%

### A.5.2. Synthesis of methyl 2-(3,5-dichloro-4-pyridyl)acetate a23

3,5-Dichloro-4-methyl-pyridine 22 (375 g, 2.31 mol) and DMF (1.87 L) were charged in a reactor and the mixture was cooled down to 15° C. Potassium tert-butoxide (779 g, 6.94 mol) was added under nitrogen at 10-15° C. and the mixture was stirred at 15° C. for 30 min. Dimethyl carbonate (730 g, 8.10 mol) was added at 10-15° C. and the mixture was stirred for 4 h at 30° C. This overall procedure was carried out on 4 batches of the same size in parallel which were worked up together. The mixture was cooled to 0° C. and the reaction quenched with H₂O (10 L) and stirred for 10 min. The reaction mixture was filtered. The filter cake was washed twice with EtOAc (2 L). The aqueous layer was extracted twice with EtOAc (3 L) and the organic layer was washed twice with brine (5 L), dried over anhydrous Na₂SO₄, filtered and concentrated under vacuum to afford 1.30 kg of methyl 2-(3,5-dichloro-4-pyridyl)acetate a23 as a black brown liquid, which was used in the next step without further purification.

Yield: 64%

### A.5.3. Synthesis of methyl 2-(3,5-dichloro-1-oxido-pyridin-1-ium-4-yl)acetate a24

Methyl 2-(3,5-dichloro-4-pyridyl)acetate a23 (650 g, 2.95 mol) and DCM (3.25 L) were charged in a reactor. m-CPBA (1.27 kg, 5.91 mol, 80% purity) was added at 0° C. under nitrogen and the mixture was stirred at rt for 5 h. This overall procedure was carried out on 4 batches of the same size in parallel which were worked up together. The mixture was cooled to 0° C. and the reaction quenched with water (4 L) and stirred for 10 min. The reaction mixture was filtered. The filter cake was washed twice with DCM (3 L). The aqueous layer was extracted twice with DCM (2 L) and the organic layer was washed thrice with a saturated aqueous solution of Na₂S₂O₃ (15 L) and twice with brine (10 L) then dried over anhydrous Na₂SO₄, filtered and concentrated under vacuum. The crude residue was purified by normal phase column chromatography (elution: from 5 to 50% EtOAc in petroleum ether) to afford 900 g of methyl 2-(3,5-dichloro-1-oxido-pyridin-1-ium-4-yl)acetate a24 as a yellow solid.

Yield: 64%

### A.5.4. Synthesis of methyl 2-(2-bromo-3,5-dichloro-4-pyridyl)acetate a25

Methyl 2-(3,5-dichloro-1-oxido-pyridin-1-ium-4-yl)acetate a24 (900 g, 3.81 mol) and ACN (8 L) were charged in a reactor at rt. Phosphorus oxybromide (1.09 kg, 3.81 mol) was added at 0° C. under nitrogen and the mixture was stirred at rt for 12 h. This overall procedure was carried out on another batch (1.64 mol scale) in parallel and the two batches were worked up together. The mixture was cooled to 0° C. and the reaction quenched with H₂O (3 L) and stirred for 10 min. The aqueous layer was extracted twice with EtOAc (2 L). The organic layer was washed twice with brine (5 L), dried over anhydrous Na₂SO₄, filtered and concentrated under vacuum. The crude residue was purified by normal phase column chromatography (elution: from 2 to 50% EtOAc in petroleum ether) to afford 503 g of methyl 2-(2-bromo-3,5-dichloro-4-pyridyl)acetate a25 as an off-white solid.

Yield: 43%

¹H NMR (400 MHz, CDCl₃): δ 8.32 (s, 1H), 4.07 (s, 2H), 3.75 (s, 3H)

### A.5.5. Synthesis of methyl 3,5-dichloro-4-(2-methoxy-2-oxo-ethyl)pyridine-2-carboxylate a26

To a solution of methyl 2-(2-bromo-3,5-dichloro-4-pyridyl)acetate a25 (3.00 g, 103 mmol) in MeOH (60 mL) was added DIPEA (2.42 mL, 14.6 mmol) and 1,4-bis(diphenylphosphino)butane-palladium (II) chloride (91.0 mg, 0.15 mmol). The reactor was flushed three times with nitrogen, then pressurized (3 flushes) with 5 bars of CO and the mixture was heated at 80° C. for 3 h. The reaction mixture was filtered at rt through a pad of Celite® and the solvent was removed under reduced pressure. The crude residue was purified by normal phase column chromatography (elution: 50% EtOAc in hexane). The solvent was removed under vacuum to afford 1.84 g of 3,5-dichloro-4-(2-methoxy-2-oxo-ethyl)pyridine-2-carboxylate a26 as a yellow liquid.

Yield: 66%

Basic LCMS Method 2 (ES⁺): 278/280/282

¹H NMR (400 MHz, DMSO-d₆): δ 8.75 (s, 1H), 4.12 (s, 2H), 3.93 (s, 3H), 3.68 (s, 3H).

### A.5.6. Synthesis of methyl 2-[3,5-dichloro-2-(hydroxymethyl)-4-pyridyl]acetate a27

To a solution of methyl 3,5-dichloro-4-(2-methoxy-2-oxo-ethyl)pyridine-2-carboxylate a26 (305 mg, 1.09 mmol) in THF (10 mL) was added at rt sodium borohydride (124 mg, 3.29 mmol) and the reaction mixture was allowed to stir at rt for 18 h. The reaction mixture was filtered and the solvent was removed under vacuum. The crude residue was purified by normal phase column chromatography (elution: from 0 to 10% MeOH in DCM) to afford 139 mg of methyl 2-[3,5-dichloro-2-(hydroxymethyl)-4-pyridyl]acetate a27 as a solid.

Yield: 50%

Basic LCMS Method 2 (ES⁺): 250/252/254

¹H NMR (400 MHz, CDCl₃): δ 8.51 (s, 1H), 4.78 (s, 2H), 4.04 (s, 2H), 3.74 (s, 3H). OH proton not observed.

### A.5.7. Synthesis of 2-[3,5-dichloro-2-(hydroxymethyl)-4-pyridyl]acetic acid a28

To a solution of methyl 2-[3,5-dichloro-2-(hydroxymethyl)-4-pyridyl]acetate a27 (98.1 g, 392 mmol) in a mixture of THF (1.1 L) and H₂O (110 mL) was added LiOH·H₂O (25.2 g, 589 mmol). The resulting mixture was stirred at rt for 18 h, then concentrated under vacuum. The crude residue was azeotropically co-evaporated with toluene (3×250 mL) to afford 92.6 g of 2-[3,5-dichloro-2-(hydroxymethyl)-4-pyridyl]acetic acid a28 as a free-flowing off-white powder, which was used in the next step without further purification.

Yield (crude): quantitative

¹H NMR (400 MHz, DMSO-d₆): δ 8.54 (s, 1H), 4.62 (s, 2H), 2.46 (s, 2H). Two OH protons were not seen.

### A.6. Synthesis of 2-(3,5-dichloro-1-methyl-indazol-4-yl)acetic acid a33

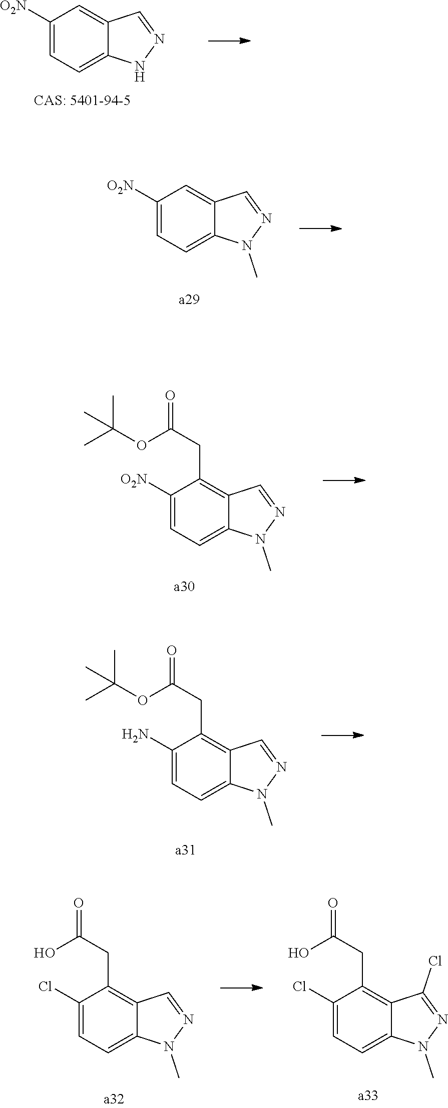 [SMILES: O=[N+]([O-])c1ccc2[nH]ncc2c1 ; Cn1ncc2cc([N+](=O)[O-])ccc21 ; Cn1ncc2c(CC(=O)OC(C)(C)C)c([N+](=O)[O-])ccc21 ; Cn1ncc2c(CC(=O)OC(C)(C)C)c(N)ccc21 ; Cn1ncc2c(CC(=O)O)c(Cl)ccc21 ; Cn1nc(Cl)c2c(CC(=O)O)c(Cl)ccc21]

### A.6.1. Synthesis of 1-methyl-5-nitro-indazole a29

5-Nitro-1H-indazole (commercial, 3.00 kg, 18.4 mol) and DMF (30 L) were charged into a 50 L three-neck round-bottom flask at 15-30° C. KOH (2.06 kg, 36.7 mol) was added in one portion into the reactor at 0-5° C. The mixture was stirred at 0-50° C. for 1 h. Mel (2.87 kg, 20.2 mol) was then added at 0-5° C. and the mixture was stirred for 3 h at 15-30° C. The reaction mixture was added into H₂O (30 L) at 0-10° C. and the mixture was stirred for 10 min, then filtered. The filter cake was washed with H₂O (5 L) and dried. This overall procedure was carried out on 4 batches of the same size in parallel. The solids obtained from the 4 batches were combined to afford 10.0 kg of 1-methyl-5-nitro-indazole a29 as a brown solid, which was used in the next step without further purification.

Yield: 57% (75% purity)

¹H NMR (400 MHz, CDCl₃): δ 8.65 (s, 1H), 8.21 (d, J=9.17 Hz, 1H), 8.13 (s, 1H), 7.39 (d,

J=9.17 Hz, 1H), 4.08 (s, 3H).

### A.6.2. Synthesis of tert-butyl 2-(1-methyl-5-nitro-indazol-4-yl)acetate a30

tBuOK (4.43 kg, 39.5 mol) and THF (30 L) were charged into a 50 L three-neck round-bottom flask and the mixture was cooled to −45/−35° C. under nitrogen and stirring. 1-Methyl-5-nitro-indazole a29 (3.50 kg, 19.7 mol) was then added in portions at −45/−35° C. tButyl 2-chloroacetate (3.57 kg, 23.7 mol) was added dropwise at the same temperature and the mixture was stirred at 1 h. The mixture was warmed up to 15-30° C. and stirred for 5 h. The reaction was quenched by the addition of a saturated aqueous solution of NH₄Cl (9 L) and H₂O (2 L) was added. The aqueous layer was extracted with EtOAc (2×5 L). The organic layer was combined, washed with brine (2 L), dried over Na₂SO₄, filtered and concentrated under vacuum. The crude residue was purified by recrystallization with EtOAc (5 L). This overall procedure was carried out on 2 batches of the same size in parallel. The solids obtained from the two batches were combined and dried together to afford 5.30 kg of tert-butyl 2-(1-methyl-5-nitro-indazol-4-yl)acetate as a yellow solid a30.

Yield: 45%

¹H NMR (400 MHz, CDCl₃): δ 8.18-8.20 (m, 2H), 7.37 (d, J=9.21 Hz, 1H), 4.27 (s, 2H), 4.14 (s, 3H), 1.44 (s, 9H).

### A.6.3. Synthesis of tert-butyl 2-(5-amino-1-methyl-indazol-4-yl)acetate a31

Tert-butyl 2-(1-methyl-5-nitro-indazol-4-yl)acetate a30 (7.30 kg, 25.0 mol) and MeOH (76.0 L) were charged into a reactor. Argon was purged and Pd/C (50%, 760 g, 7.00 mmol) was added. H₂ was added three times and the mixture was stirred at 50° C. under H₂ atmosphere (50 psi) for 3 h. The reaction mixture was filtered and the solid was washed with MeOH (5 L). The mixture was concentrated to afford 6.50 kg of tert-butyl 2-(5-amino-1-methyl-indazol-4-yl)acetate a31 as a brown oil, which was used in the next step without further purification.

Yield: 95%

¹H NMR (400 MHz, CDCl₃): δ 7.72 (s, 1H), 7.27 (d, J=8.80 Hz, 1H), 6.91 (d, J=8.80 Hz, 1H), 4.60 (s, 2H), 3.93 (s, 3H), 3.68 (s, 2H), 1.38 (s, 9H).

### A.6.4. Synthesis of 2-(5-chloro-1-methyl-indazol-4-yl)acetic acid a32

Tert-butyl 2-(5-amino-1-methyl-indazol-4-yl)acetate a31 (2.00 kg, 7.65 mol) and a 12N aqueous concentrated solution of HCl (10 L, 120 mol) were charged into a 50 L three-neck round bottom flask and the mixture was cooled to −10/−5° C. and stirred. A solution sodium nitrite (686 g, 9.95 mol) in H₂O (5 L) was added dropwise at −10/−5° C. and stirred for 30 min. CuCl (833 g, 8.42 mol) and a 12N aqueous concentrated solution of HCl (10.0 L, 120 mol) were charged into a 20 L three-neck round bottom flask and the mixture was stirred for 30 min at −10/−5° C., then added into the other reactor. The mixture was stirred at −10/−5° C. for 1 h, then at 10-30° C. for 16 h. The reaction mixture was filtered and the solid washed with H₂O. This overall procedure was carried out on 3 batches of the same size in parallel. The solids obtained from the 3 batches were combined and dried to afford 4.00 kg of 2-(5-chloro-1-methyl-indazol-4-yl)acetic acid a32 as a yellow solid, which was used in the next step without further purification.

Yield (crude): 71% (92% purity)

### A.6.5. Synthesis of 2-(3,5-dichloro-1-methyl-indazol-4-yl)acetic acid a33

2-(5-Chloro-1-methyl-indazol-4-yl)acetic acid a32 (1.30 kg, 5.79 mol) and DMF (6.50 L) were charged into a 50 L three-neck round bottom flask at rt. NCS (772 g, 5.79 mol) was added portionwise at rt and the mixture was stirred at rt for 2 h. The reaction mixture was poured into H₂O (25 L) and filtered. The crude residue was triturated with isopropyl ether:EtOAc (3:1) (7 L) at rt for 2 h, then the obtained solid was filtered and dried under vacuum. This overall procedure was carried out on 3 batches of the same size in parallel. The solids obtained from the three batches were combined to afford 2.10 kg of 2-(3,5-dichloro-1-methyl-indazol-4-yl)acetic acid a33.

Yield: 45%

¹H NMR (400 MHz, CDCl₃): δ 12.67 (s, 1H), 7.68 (d, J=9.05 Hz, 1H), 7.53 (d, J=9.05 Hz, 1H), 4.20 (s, 2H), 4.02 (s, 3H).

### A.7. Synthesis of 2-(3,5-dichloro-1-methyl-1H-indol-4-yl)acetic acid a39b

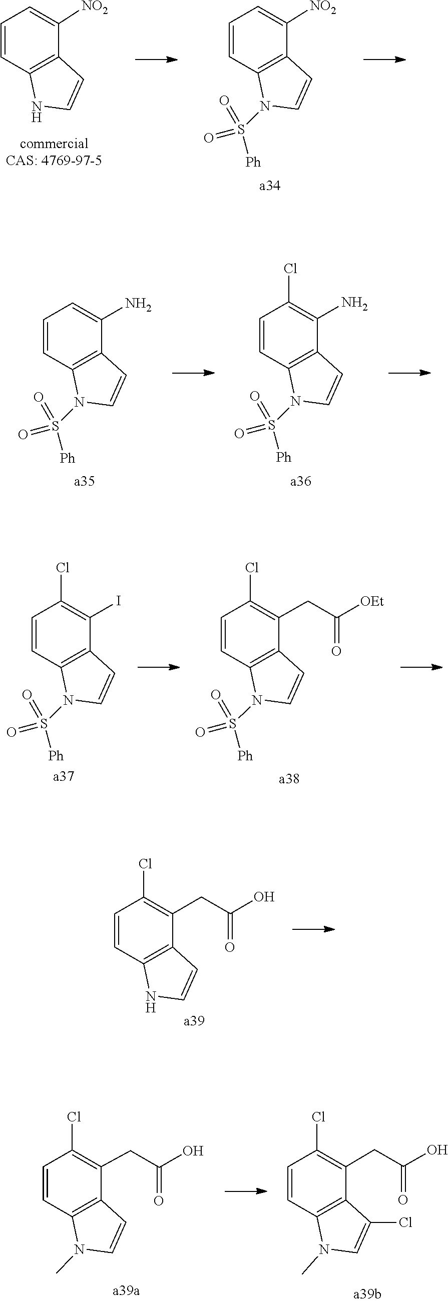

### A.7.1. Synthesis of 1-(benzenesulfonyl)-4-nitro-indole a34

To a solution of commercial 4-nitro-1H-indole (25.0 g, 154 mmol) in ACN (250 mL), DIPEA (29.5 mL, 170 mmol) was added at rt. The reaction was cooled to 0° C. and benzensulfonyl chloride (23.0 mL, 185 mmol) was added. The reaction was heated at 80° C. for 3 h. After completion, the reaction was quenched with a saturated aqueous solution of NaHCO₃ and extracted with EtOAc. The organic layer was washed with H₂O, dried over anhydrous Na₂SO₄, filtered and concentrated under vacuum to afford 34.9 g of 1-(benzenesulfonyl)-4-nitro-indole a34, which was used in the next step without further purification.

Yield (crude): 97%

¹H NMR (400 MHz, DMSO-d₆): δ 8.47-8.39 (m, 1H), 8.26-8.17 (m, 2H), 8.12-8.04 (m, 2H), 7.78-7.68 (m, 1H), 7.67-7.54 (m, 3H), 7.38-7.26 (m, 1H).

### A.7.2. Synthesis of 1-(benzenesulfonyl)indol-4-amine a35

To a stirred solution of 1-(benzenesulfonyl)-4-nitro-indole a34 (25.0 g, 82.8 mmol) in MeOH (250 mL), Fe (69.5 g, 1.24 mol) and NH₄Cl (67.0 g, 1.24 mol) were added and the reaction mixture was heated under reflux for 15 h. After completion, the reaction was filtered through a pad of Celite® and the filtrate was concentrated under reduced pressure. The crude residue was purified by normal phase column chromatography (elution: 10% EtOAc in hexanes) to afford 7.00 g of 1-(benzenesulfonyl)indol-4-amine a35.

Yield: 31%

Basic LCMS Method 1 (ES⁺): 273 (M+H)⁺.

¹H NMR (400 MHz, DMSO-d₆): δ 7.95-7.85 (m, 2H), 7.72-7.49 (m, 4H), 7.14-6.91 (m, 3H), 6.35 (d, J=7.7 Hz, 1H), 5.55 (s, 2H).

### A.7.3. Synthesis of 1-(benzenesulfonyl)-5-chloro-indol-4-amine a36

To a stirred solution of 1-(benzenesulfonyl)indol-4-amine a35 (35.4 g, 130 mmol) in DCM (300 mL) at 0° C., a solution of NCS (17.3 g, 130 mmol) in DCM (100 mL) was added. The mixture was stirred at the same temperature for 1 h, then at rt for 1 h. After completion, the reaction mixture was quenched with a saturated aqueous solution of sodium bicarbonate and extracted with DCM. The organic layer was washed with H₂O, dried over anhydrous Na₂SO₄, filtered and concentrated under vacuum. The crude residue was purified by normal phase column chromatography (elution: 10% EtOAc in hexanes) to afford 14.8 g of 1-(benzenesulfonyl)-5-chloro-indol-4-amine a36.

Yield: 37%

¹H NMR (400 MHz, DMSO-d₆): δ 7.95-7.87 (m, 2H), 7.76-7.54 (m, 4H), 7.09 (dd, J=17.3, 3.3 Hz, 3H), 5.82 (s, 2H).

### A.7.4. Synthesis of 1-(benzenesulfonyl)-5-chloro-4-iodo-indole a37

To a solution of 1-(benzenesulfonyl)-5-chloro-indol-4-amine a36 (13.8 g, 45.1 mmol) in a 12N aqueous solution of HCl (414 mL) at 0° C., a solution of NaNO₂ (7.77 g, 113 mmol) in H₂O (70 mL) was added dropwise. The mixture was stirred for 30 min at the same temperature. A solution of KI (74.84 g, 450.9 mmol) in H₂O (137 mL) was then added dropwise at 0° C. and the mixture was stirred at the same temperature for 3 h. After completion, the reaction was extracted with EtOAc. The organic layer was washed with H₂O, dried over Na₂SO₄, filtered and concentrated under vacuum. The crude residue was purified by normal phase column chromatography (elution: 10% EtOAc in hexanes) to afford 17.2 g of 1-(benzenesulfonyl)-5-chloro-4-iodo-indole a37.

Yield: 92%

¹H NMR (400 MHz, DMSO-d₆): δ 8.05-7.85 (m, 4H), 7.77-7.67 (m, 1H), 7.62 (t, J=7.8 Hz, 2H), 7.51 (d, J=8.8 Hz, 1H), 6.70 (d, J=3.7 Hz, 1H).

### A.7.5. Synthesis of ethyl 2-[1-(benzenesulfonyl)-5-chloro-indol-4-yl]acetate a38

To a stirred solution of activated Zn (12.2 g, 188 mmol) in dry THF (75 mL), chlorotrimethylsilane (2.39 mL, 18.8 mmol) was added. The mixture was stirred at rt for 15 min followed by dropwise addition of ethyl bromo acetate (8.30 mL, 75.4 mmol) at rt. 1-(Benzenesulfonyl)-5-chloro-4-iodo-indole a37 (5.00 g, 12.0 mmol) was dissolved in THF (50 mL) and purged with argon for 15 min. Pd(t-Bu₃P)₂ (608 mg, 1.19 mmol) was added, followed by addition of the above Reformatsky reagent. The reaction was heated at 65° C. for 16 h. After completion, the reaction mixture was quenched with a saturated aqueous solution of ammonium chloride and extracted with EtOAc. The organic layer was washed with H₂O, dried over Na₂SO₄, filtered and concentrated under vacuum. The crude residue was purified by normal phase column chromatography (elution: 10% EtOAc in hexanes) to afford 3.34 g of ethyl 2-[1-(benzenesulfonyl)-5-chloro-indol-4-yl]acetate a38.

Yield: 74%

Basic LCMS Method 1 (ES⁺): 378 (M+H)⁺.

¹H NMR (400 MHz, DMSO-d₆): δ 8.00 (dd, J=7.8, 1.6 Hz, 2H), 7.94-7.85 (m, 2H), 7.71 (t, J=7.4 Hz, 1H), 7.60 (t, J=7.8 Hz, 2H), 7.41 (d, J=8.8 Hz, 1H), 7.02 (d, J=3.8 Hz, 1H), 4.12 (q, J=7.1 Hz, 2H), 4.02 (s, 2H), 1.14 (t, J=7.1 Hz, 3H).

### A.7.6. Synthesis of 2-(5-chloro-1H-indol-4-yl)acetic acid a39

To a stirred solution of ethyl 2-[1-(benzenesulfonyl)-5-chloro-indol-4-yl]acetate a38 (4.55 g, 12.1 mmol) in EtOH (40 mL), a 3N aqueous solution of NaOH (20 mL) was added. The mixture was heated to reflux for 8 h. After completion, the reaction was evaporated under reduced pressure. The crude residue was diluted with H₂O, acidified to pH 2 using a 1N aqueous solution of HCl and extracted with EtOAc. The organic layer was washed with H₂O, dried over anhydrous Na₂SO₄, filtered and concentrated under vacuum to afford 2.50 g of 2-(5-chloro-1H-indol-4-yl)acetic acid a39, which was used in the next step without further purification.

Yield (crude): 99%

¹H NMR (400 MHz, DMSO-d₆): δ 12.31 (s, 1H), 11.27 (s, 1H), 7.38-7.40 (m, 1H), 7.32 (dd, J=8.6, 0.9 Hz, 1H), 7.10 (d, J=8.6 Hz, 1H), 6.50-6.52 (m, 1H), 3.91 (s, 2H).

### A.7.7. Synthesis of 2-(5-chloro-1-methyl-1H-indol-4-yl)acetic acid a39a

To a suspension of NaH (800 mg, 33.3 mmol) in THF (20 mL) was added a solution of 2-(5-chloro-1H-indol-4-yl)acetic acid a39 (1.40 g, 6.69 mmol) in THF (5 mL) at 0° C. and the reaction mixture was stirred at the same temperature for 30 min. Mel (1.42 mL, 22.0 mmol) solution in THF (5 mL) was added dropwise at 0° C. and the reaction mixture was stirred at room temperature for 16 h. Progress of the reaction was monitored by TLC and LCMS. After completion, the reaction mixture was quenched with ice and washed with EtOAc (2×250 mL). The aqueous layer was acidified with a 6N aqueous solution of HCl and extracted with DCM (2×300 mL). The organic layer was dried over anhydrous Na₂SO₄, filtered and concentrated under vacuum to afford 1.35 g of 2-(5-chloro-1-methyl-1H-indol-4-yl)acetic acid a39a as an off-white solid, which was used in the next step without further purification.

Yield (crude): 90%

Basic LCMS Method 1 (ES⁺): 224 (M+H)⁺, 85% purity.

¹H NMR (400 MHz, DMSO-d₆): δ 12.27-12.41 (m, 1H), 7.35-7.43 (m, 2H), 7.17 (d, J=8.80 Hz, 1H), 6.48-6.54 (m, 1H), 3.91 (s, 2H), 3.79 (s, 3H).

### A.7.8. Synthesis of 2-(3,5-dichloro-1-methyl-1H-indol-4-yl)acetic acid a39b

To a solution of 2-(5-chloro-1-methyl-1H-indol-4-yl)acetic acid a39a (1.30 g, 5.82 mmol) in DCM (30 mL) was added NCS (0.78 g, 5.82 mmol) at 0° C. and the reaction mixture was stirred at rt for 3 h. Progress of the reaction was monitored by TLC. After completion, the reaction mixture was diluted with H₂O (150 mL) and extracted with DCM (3×100 mL). The organic layer was dried over anhydrous Na₂SO₄, filtered and concentrated under vacuum. The crude residue was purified by normal phase column chromatography (elution: 2.5% MeOH in DCM) to afford 950 mg of 2-(3,5-dichloro-1-methyl-1H-indol-4-yl)acetic acid a39b as an off-white solid.

Yield: 64%

HPLC Purity: 91%

¹H NMR (400 MHz, DMSO-d₆): δ 12.42 (brs, 1H), 7.55-7.60 (m, 1H), 7.45 (d, J=8.80 Hz, 1H), 7.25 (d, J=8.80 Hz, 1H), 4.21 (s, 2H), 3.65 (s, 3H).

### A.8. Synthesis of 2-(2,6-dichloro-3-(difluoromethoxy)phenyl)acetic acid a45

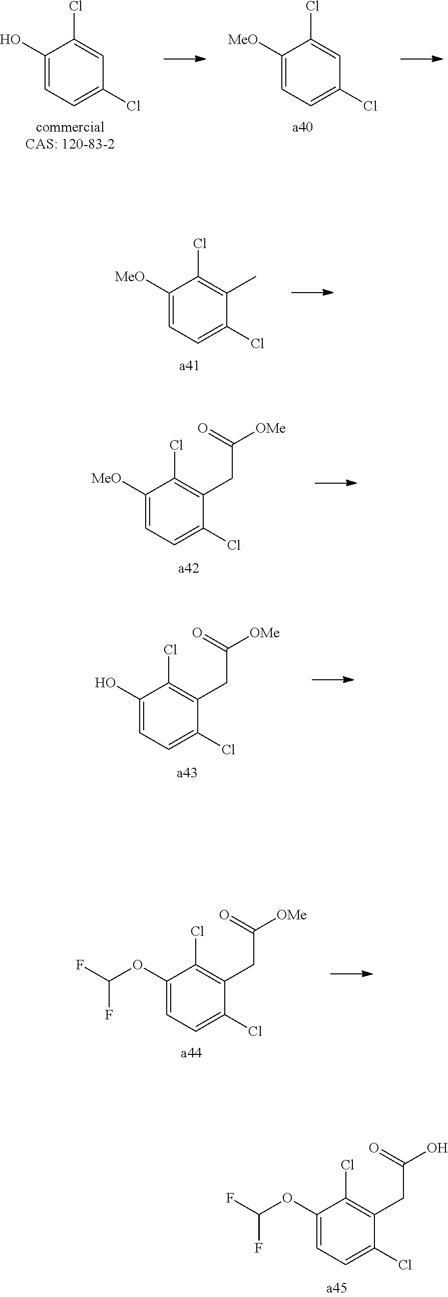

### A.8.1. Synthesis of 2,4-dichloro-1-methoxybenzene a40

To a solution of 2,4-dichlorophenol (commercial, 30.0 g, 184 mmol) in acetone (300 mL) was added K₂CO₃ (31.7 g, 230 mmol) at room temperature followed by addition of Mel (28.6 mL, 460 mmol). The reaction mixture was heated to reflux for 2 h. Progress of the reaction was monitored by TLC and LCMS. After completion, the reaction mixture was concentrated under vacuum. The crude residue was diluted with H₂O (200 mL) and extracted with Et₂O (3×100 mL). The organic layer was dried over anhydrous Na₂SO₄, filtered and concentrated under vacuum to afford 32.0 g of 2,4-dichloro-1-methoxybenzene a40 as a colourless liquid, which was used in the next step without further purification.

Yield (crude): 99%

¹H NMR (400 MHz, DMSO-d₆): δ 7.56 (brs, 1H), 7.38 (d, J=8.31 Hz, 1H), 7.17 (d, J=8.80 Hz, 1H), 3.85 (s, 3H).

### A.8.2. Synthesis of 1,3-dichloro-4-methoxy-2-methylbenzene a41

To a solution of 2,4-dichloro-1-methoxybenzene a40 (21.0 g, 121 mmol) in dry THF (200 mL) was added n-BuLi (74.3 mL, 119 mmol) dropwise at −78° C. and stirred at same temperature for 1 h. Mel (6.61 mL, 131 mmol) was added dropwise at −78° C. and reaction mixture was stirred at same temperature for 1 h. Progress of the reaction was monitored by TLC and LCMS. After completion, the reaction mixture was quenched with a saturated solution of NaHCO₃ (200 mL) at −78° C. and concentrated under vacuum. The crude residue was extracted with EtOAc (3×200 mL). The organic layer was dried over anhydrous Na₂SO₄, filtered and concentrated under vacuum. The crude residue was purified by normal phase column chromatography (elution: from 0 to 3% EtOAc in hexanes) to afford 21.0 g of 1,3-dichloro-4-methoxy-2-methylbenzene a41 as a colourless liquid.

Yield: 92%

¹H NMR (400 MHz, DMSO-d₆): δ 7.40 (d, J=8.80 Hz, 1H), 7.04 (d, J=8.80 Hz, 1H), 3.85 (s, 3H), 2.40 (s, 3H).

### A.8.3. Synthesis of methyl 2-(2,6-dichloro-3-methoxyphenyl)acetate a42

To a solution of 1,3-dichloro-4-methoxy-2-methylbenzene a41 (21.0 g, 109 mmol) in dry THF (200 mL) was added LDA (65.9 mL, 131 mmol) dropwise at −78° C. and stirred at same temperature for 1 h. Dimethylcarbonate (11.0 mL, 131 mmol) was added dropwise at −78° C. and reaction mixture was stirred at same temperature for 1 h. Progress of the reaction was monitored by TLC. After completion, the reaction mixture was quenched with a saturated aqueous solution of NH₄Cl (150 mL) at −78° C. and concentrated under vacuum. The crude residue was extracted with EtOAc (3×100 mL). The organic layer was dried over anhydrous Na₂SO₄, filtered and concentrated under vacuum. The crude residue was purified by normal phase column chromatography (elution: from 0 to 4% EtOAc in hexanes) to afford 15.0 g of 2-(2,6-dichloro-3-methoxyphenyl)acetate a42 as a colourless liquid.

Yield: 55%

¹H NMR (400 MHz, DMSO-d₆): δ 7.46 (d, J=9.29 Hz, 1H), 7.15 (d, J=8.80 Hz, 1H), 3.98 (s, 2H), 3.87 (s, 3H), 3.64 (s, 3H).

### A.8.4. Synthesis of methyl 2-(2,6-dichloro-3-hydroxyphenyl)acetate a43

To a solution of methyl 2-(2,6-dichloro-3-methoxyphenyl)acetate a42 (10.0 g, 40.1 mmol) in DCM (100 mL) was added BBr₃ (9.65 mL, 100 mmol) dropwise at −15° C. and stirred at same temperature for 15 min. The reaction mixture was stirred at 0° C. for 90 min. Progress of the reaction was monitored by TLC and LCMS. After completion, the reaction mixture was poured onto ice-cold MeOH (100 mL), quenched with H₂O (100 mL) and concentrated under vacuum. The crude residue was diluted with H₂O (50 mL) and extracted with EtOAc (3×50 mL). The organic layer was dried over anhydrous Na₂SO₄, filtered and concentrated under vacuum. The crude residue was purified by normal phase column chromatography (elution: 20% EtOAc in hexanes) to afford 9.50 g of methyl 2-(2,6-dichloro-3-hydroxyphenyl)acetate a41 as a white solid.

Yield: 91%

¹H NMR (400 MHz, DMSO-d₆): δ 10.51 (s, 1H), 7.27 (d, J=8.80 Hz, 1H), 6.94 (d, J=8.80 Hz, 1H), 3.94 (s, 2H), 3.63 (s, 3H)

### A.8.5. Synthesis of methyl 2-(2,6-dichloro-3-(difluoromethoxy)phenyl)acetate a44

To a solution of methyl 2-(2,6-dichloro-3-hydroxyphenyl)acetate a43 (5.00 g, 21.2 mmol) in ACN (50 mL) was added KOH (23.8 g, 425 mmol) solution in H₂O (50 mL) dropwise at 0° C. 1-[[Bromo(difluoro)methyl]-ethoxy-phosphoryl]oxyethane (commercial, 7.57 mL, 42.5 mmol) was added dropwise and stirred at 0° C. for 30 min. The reaction mixture was stirred at rt for 2 h. Progress of the reaction was monitored by TLC and LCMS. After completion, the reaction mixture was acidified with a 12N aqueous concentrated solution of HCl (30 mL) and concentrated under vacuum. The crude residue was diluted with H₂O (50 mL) and extracted with EtOAc (3×50 mL). The organic layer was dried over anhydrous Na₂SO₄, filtered and concentrated under vacuum. The crude residue was purified by normal phase column chromatography (elution: 4% EtOAc in hexanes) to afford 4.50 g of 2-(2,6-dichloro-3-(difluoromethoxy)phenyl)acetate a44 as a white solid.

Yield: 74%

¹H NMR (400 MHz, DMSO-d₆): δ 7.58 (d, J=9.29 Hz, 1H), 7.37 (d, J=9.29 Hz, 1H), 7.32 (t, J=74 Hz, 1H), 4.02 (s, 2H), 3.64 (s, 3H)

### A.8.6. Synthesis of 2-(2,6-dichloro-3-(difluoromethoxy)phenyl)acetic acid a45

To a solution of methyl 2-(2,6-dichloro-3-(difluoromethoxy)phenyl)acetate a44 (3.50 g, 12.2 mmol) in MeOH (20 mL) and THF (20 mL) was added To a solution of LiOH (1.41 g, 58.9 mmol) in H₂O (10 mL) dropwise at 0° C. The reaction mixture was stirred at rt for 2 h. Progress of reaction was monitored by TLC and LCMS. After completion, the reaction mixture was quenched with NH₄Cl (3.12 g) and concentrated under vacuum. The crude residue was diluted with H₂O (50 mL), acidified to pH 3 with a 6N aqueous solution of HCl (20 mL) and extracted with EtOAc (3×30 mL). The organic layer was dried over anhydrous Na₂SO₄, filtered and concentrated under vacuum. The crude residue was purified by normal phase column chromatography (elution: from 0 to 20% EtOAc in hexanes) to 3.30 g of afford 2-(2,6-dichloro-3-(difluoromethoxy)phenyl)acetic acid a45 as a white solid.

Yield: 77%

HPLC Purity: 96%

¹H NMR (400 MHz, DMSO-d₆): δ 7.57 (d, J=8.80 Hz, 1H), 7.36 (d, J=8.80 Hz, 1H), 7.32 (t, J=74 Hz, 1H), 3.93 (s, 2H)

### A.9. Synthesis of 2-(3,6-dichloro-[1,2,4]triazolo[4,3-a]pyridin-5-yl)acetic acid a55

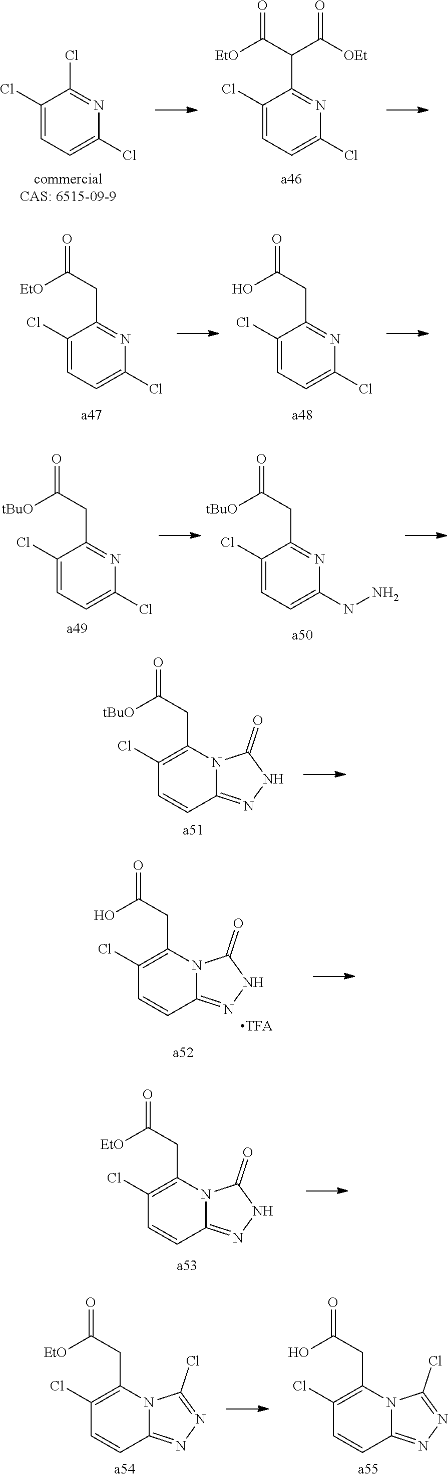

### A.9.1. Synthesis of diethyl 2-(3,6-dichloropyridin-2-yl)malonate a46

To a solution of 2,3,6-trichloropyridine (commercial, 10.0 g, 54.8 mmol) and diethyl malonate (16.7 mL, 110 mmol) in DMF (100 mL) was added Cs₂CO₃ (35.7 g, 110 mmol) and the reaction mixture was heated at 80° C. for 16 h. Progress of reaction was monitored by TLC and LCMS. After completion, the reaction mixture was cooled at rt, diluted with H₂O (500 mL) and extracted with EtOAc (3×200 mL). The organic layer was washed with brine (3×100 mL), dried over anhydrous Na₂SO₄, filtered and concentrated under vacuum. The crude residue was purified by normal phase column chromatography (elution: 4% EtOAc in hexanes) to afford 16.7 g of diethyl 2-(3,6-dichloropyridin-2-yl)malonate a46 as a pale brown liquid.

Yield: quantitative

Basic LCMS Method 1 (ES⁺): 306 (M+H)⁺, 64% purity.

¹H NMR (400 MHz, DMSO-d₆): δ 8.19 (d, J=8.4 Hz, 1H), 7.61 (d, J=8.4 Hz, 1H), 5.29 (s, 1H), 4.14-4.27 (m, 4H), 1.18 (t, J=6.8 Hz, 6H).

### A.9.2. Synthesis of ethyl 2-(3,6-dichloropyridin-2-yl)acetate a47

To a solution of diethyl 2-(3,6-dichloropyridin-2-yl)malonate a46 (15.7 g, 51.3 mmol) in DMSO (50 mL) and H₂O (50 mL) was added LiCl (21.7 g, 513 mmol) and the reaction mixture was heated to 120° C. for 24 h. Progress of reaction was monitored by TLC and LCMS. After completion, the reaction mixture was cooled to rt and concentrated under vacuum. The crude residue was purified by normal phase column chromatography (elution: 4% EtOAc in hexanes) to afford 4.90 g of ethyl 2-(3,6-dichloropyridin-2-yl)acetate a47 as a colorless liquid.

Yield: 41%

Basic LCMS Method 1 (ES⁺): 235 (M+H)⁺, 96% purity.

¹H NMR (400 MHz, DMSO-d₆): δ 8.01 (d, J=8.0 Hz, 1H), 7.51 (d, J=8.4 Hz, 1H), 4.10 (q, J=7.2 Hz, 2H), 3.95 (s, 2H), 1.16 (t, J=7.2 Hz, 3H).

### A.9.3. Synthesis of 2-(3,6-dichloropyridin-2-yl)acetic acid a48

To a solution of ethyl 2-(3,6-dichloropyridin-2-yl)acetate a47 (4.90 g, 20.9 mmol) in MeOH (25 mL), THF (25 mL) and H₂O (10 mL) was added LiOH (0.75 g, 31.4 mmol) at 0° C. and the reaction mixture was stirred at room temperature for 3 h. Progress of reaction was monitored by TLC and LCMS. After completion, the reaction mixture was concentrated under vacuum at 30° C. The crude residue was diluted with H₂O (100 mL) and acidified with a 6N solution of HCl up to pH 2, extracted with EtOAc (3×50 mL). The organic layer was dried over anhydrous Na₂SO₄, filtered and concentrated under vacuum at 30° C. The crude residue was purified by washing with Et₂O (50 mL) and dried to afford 4.31 g of 2-(3,6-dichloropyridin-2-yl)acetic acid a48 as an off-white solid, which was used in the next step without further purification.

Yield (crude): quantitative

Basic LCMS Method 1 (ES⁺): 205.9 (M+H)⁺, 92% purity.

¹H NMR (400 MHz, DMSO-d₆): δ 12.75 (brs, 1H), 8.01 (d, J=8.4 Hz, 1H), 7.51 (d, J=8.4 Hz, 1H), 3.87 (s, 2H).

### A.9.4. Synthesis of tert-butyl 2-(3,6-dichloropyridin-2-yl)acetate a49

To a solution of 2-(3,6-dichloropyridin-2-yl)acetic acid a48 (4.30 g, 20.9 mmol) in t-BuOH (50 mL) was added (Boc)₂O (7.19 mL, 31.3 mmol) followed by addition of DMAP (260 mg, 2.09 mmol) at rt and the reaction mixture was stirred at rt for 16 h. Progress of reaction was monitored by TLC and LCMS. After completion, the reaction mixture was concentrated under vacuum. The crude residue was diluted with H₂O (100 mL) and extracted with EtOAc (3×50 mL). The organic layer was dried over anhydrous Na₂SO₄, filtered and concentrated under vacuum. The crude residue was purified by normal phase column chromatography (elution: 5% EtOAc in hexanes) to afford 4.94 g of tert-butyl 2-(3,6-dichloropyridin-2-yl)acetate a49 as a pale yellow liquid.

Yield: 90%

Basic LCMS Method 1 (ES⁺): 205.9 (M-tBu+H)⁺, 94% purity.

¹H NMR (400 MHz, DMSO-d₆): δ 8.01 (d, J=8.4 Hz, 1H), 7.51 (d, J=8.0 Hz, 1H), 3.86 (s, 2H), 1.40 (s, 9H)

### A.9.5. Synthesis of tert-butyl 2-(3-chloro-6-hydrazineylpyridin-2-yl)acetate a50

To a solution of tert-butyl 2-(3,6-dichloropyridin-2-yl)acetate a49 (4.90 g, 18.7 mmol) in 1,4-dioxane (50 mL) was added hydrazine monohydrate (1.81 mL, 37.4 mmol) and the reaction mixture was heated at 100° C. for 16 h. Progress of reaction was monitored by TLC and LCMS. After completion, the reaction mixture was concentrated under vacuum. The crude residue was diluted with H₂O (100 mL) and extracted with EtOAc (3×50 mL). The organic layer was dried over anhydrous Na₂SO₄, filtered and concentrated under vacuum. The crude residue was purified by normal phase column chromatography (elution: 5% MeOH in DCM) to afford 1.21 g of tert-butyl 2-(3-chloro-6-hydrazineylpyridin-2-yl)acetate a50 as a pale yellow viscous liquid.

Yield: 25%

Basic LCMS Method 1 (ES⁺): 258 (M+H)⁺, 90% purity.

¹H NMR (400 MHz, DMSO-d₆): δ 7.59 (brs, 1H), 7.48 (d, J=8.8 Hz, 1H), 6.66 (d, J=9.2 Hz, 1H), 4.15 (brs, 2H), 3.61 (s, 2H).

### A.9.6. Synthesis of tert-butyl 2-(6-chloro-3-oxo-2,3-dihydro-[1,2,4]triazolo[4,3-a]pyridin-5-yl)acetate a51

To a solution of tert-butyl 2-(3-chloro-6-hydrazineylpyridin-2-yl)acetate a50 (3.94 g, 15.3 mmol) in THF (50 mL) was added CDI (2.97 g, 18.3 mmol) in portions at rt and the reaction mixture was stirred at rt for 16 h. Progress of the reaction was monitored by TLC and LCMS. After completion, the reaction mixture was concentrated under vacuum. The crude residue was purified by washing with Et₂O (50 mL) and the precipitate was dried under vacuum to afford 2.61 g of tert-butyl 2-(6-chloro-3-oxo-2,3-dihydro-[1,2,4]triazolo[4,3-a]pyridin-5-yl)acetate a51 as an off-white solid.

Yield: 60%

Basic LCMS Method 1 (ES⁺): 228 (M-tBu+H)⁺, 99% purity.

¹H NMR (400 MHz, DMSO-d₆): δ 12.58 (brs, 1H), 7.12-7.18 (m, 2H), 4.28 (s, 2H), 1.37 (s, 9H).

### A.9.7. Synthesis of 2-(6-chloro-3-oxo-2,3-dihydro-[1,2,4]triazolo[4,3-a]pyridin-5-yl)acetic acid a52

To a solution of tert-butyl 2-(6-chloro-3-oxo-2,3-dihydro-[1,2,4]triazolo[4,3-a]pyridin-5-yl)acetate a51 (1.20 g, 4.23 mmol) in DCM (12 mL) was added TFA (3.14 mL, 42.3 mmol) at 0° C. and the reaction mixture was stirred at rt for 16 h. Progress of the reaction was monitored by TLC and LCMS. After completion, the reaction mixture was concentrated under vacuum. The crude residue was purified by washing with Et₂O (3×50 mL) and dried under vacuum to afford 0.99 g of 2-(6-chloro-3-oxo-2,3-dihydro-[1,2,4]triazolo[4,3-a]pyridin-5-yl)acetic acid a52 as a TFA salt and as an off-white solid, which was used in the next step without further purification.

Yield: 69%

HPLC Purity: 98%

Basic LCMS Method 1 (ES⁺): 228 (M+H)⁺, 99% purity.

¹H NMR (400 MHz, DMSO-d₆): δ 12.78 (brs, 1H), 12.60 (s, 1H), 7.13-7.18 (m, 2H), 4.31 (s, 2H).

### A.9.8. Synthesis of ethyl 2-(6-chloro-3-oxo-2,3-dihydro-[1,2,4]triazolo[4,3-a]pyridin-5-yl)acetate hydrochloride a53

To a solution of 2-(6-chloro-3-oxo-2,3-dihydro-[1,2,4]triazolo[4,3-a]pyridin-5-yl)acetic acid a52 (2.43 g, 7.11 mmol) in EtOH (50 mL) was added SOCl₂ (1.56 mL, 21.3 mmol) at 0° C. and the reaction mixture was stirred at rt for 16 h. Progress of reaction was monitored by TLC and LCMS. After completion, the reaction mixture was concentrated under vacuum. The crude residue was purified by washing with Et₂O (50 mL) and dried under vacuum to afford 2.00 g of ethyl 2-(6-chloro-3-oxo-2,3-dihydro-[1,2,4]triazolo[4,3-a]pyridin-5-yl)acetate hydrochloride a53 as an off-white solid.

Yield: 96%

HPLC Purity: 98%

Basic LCMS Method 1 (ES⁺): 256 (M+H)⁺, 98% purity.

¹H NMR (400 MHz, DMSO-d₆): δ 12.63 (brs, 1H), 7.14-7.21 (m, 2H), 5.45 (brs, 1H), 4.36 (s, 2H), 4.11 (q, J=6.8 Hz, 2H), 1.17 (t, J=7.2 Hz, 3H).

### A.9.9. Synthesis of ethyl 2-(3,6-dichloro-[1,2,4]triazolo[4,3-a]pyridin-5-yl)acetate a54

To a solution of ethyl 2-(6-chloro-3-oxo-2,3-dihydro-[1,2,4]triazolo[4,3-a]pyridin-5-yl)acetate hydrochloride a53 (1.20 g, 4.11 mmol) in POCl₃ (10 mL, 109 mmol) was added N,N-dimethylaniline (0.10 mL, 0.82 mmol) and the reaction mixture was heated in sealed tube at 100° C. for 36 h. After completion, the reaction mixture was cooled at rt and concentrated under vacuum. The crude residue was diluted with ice H₂O (100 mL) in cold condition, basified with a saturated aqueous solution of NaHCO₃ (20 mL) up to pH 8 and extracted with EtOAc (3×100 mL). The organic layer was dried over anhydrous Na₂SO₄, filtered and concentrated under vacuum. The crude residue was purified by normal phase column chromatography (elution: 5% MeOH in DCM for 30 min, then 2% MeOH in DCM) to afford 0.98 g of ethyl 2-(3,6-dichloro-[1,2,4]triazolo[4,3-a]pyridin-5-yl)acetate a54 as a pale yellow solid.

Yield: 87%

Basic LCMS Method 1 (ES⁺): 274 (M+H)⁺, 93.9% purity.

¹H NMR (400 MHz, DMSO-d₆): δ 7.86 (d, J=10.0 Hz, 1H), 7.55 (d, J=9.6 Hz, 1H), 4.58 (s, 2H), 4.16 (q, J=7.2 Hz, 2H), 1.18 (t, J=7.6 Hz. 3H).

### A.9.10. Synthesis of 2-(3,6-dichloro-[1,2,4]triazolo[4,3-a]pyridin-5-yl)acetic acid a55

To a solution of ethyl 2-(3,6-dichloro-[1,2,4]triazolo[4,3-a]pyridin-5-yl)acetate a54 (0.98 g, 3.58 mmol) in MeOH (5 mL), THF (10 mL) and H₂O (1 mL) was added LiOH (0.13 g, 5.36 mmol) at 0° C. and the reaction mixture was stirred at rt for 3 h. Progress of the reaction was monitored by TLC and LCMS. After completion, the reaction mixture was concentrated under vacuum at 30° C. The crude residue was diluted with H₂O (50 mL), acidified with a 6N aqueous solution of HCl up to pH 2, filtered, washed with Et₂O (100 mL) and dried under vacuum to afford 709 mg of 2-(3,6-dichloro-[1,2,4]triazolo[4,3-a]pyridin-5-yl)acetic acid a55 as an off-white solid, which was used in the next step without further purification.

Yield (crude): 81%

HPLC Purity: 95.6%

Basic LCMS Method 1 (ES⁺): 246 (M+H)⁺, 98% purity.

¹H NMR (400 MHz, DMSO-d₆): δ 13.32 (brs, 1H), 7.85 (d, J=9.6 Hz, 1H), 7.55 (d, J=9.6 Hz, 1H), 4.51 (s, 2H).

### A.10. Synthesis of 2-(3,5-dichloro-7-fluoro-1H-indazol-4-yl)acetic acid a65

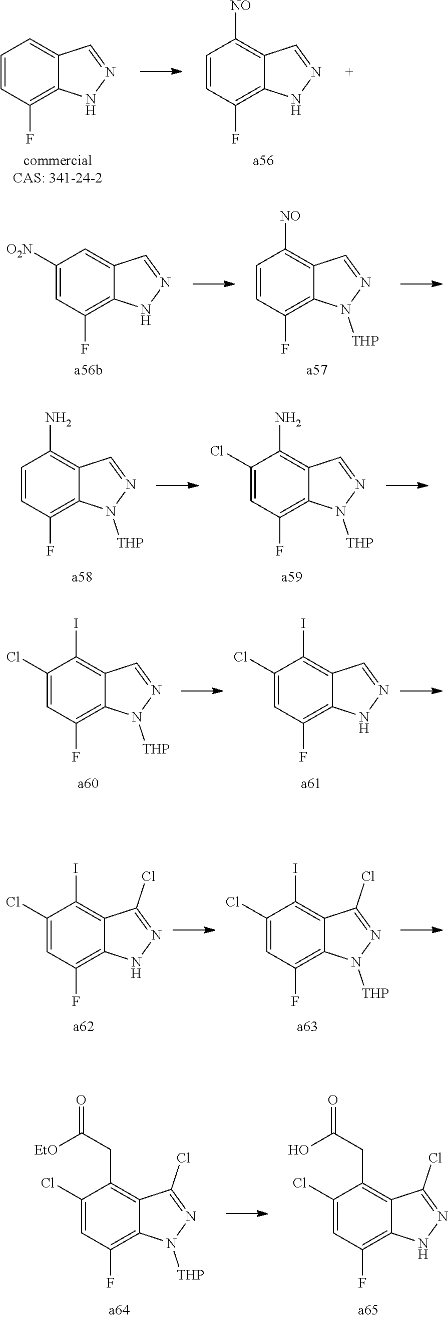

### A.10.1. Synthesis of 7-fluoro-4-nitro-1H-indazole a56

To a solution of 7-fluoro-1H-indazole (commercial, 10.0 g, 73.5 mmol) in concentrated H₂SO₄ (100 mL) was added KNO₃ (7.43 g, 73.5 mmol) at 0° C. and the reaction mixture was stirred at same temperature for 4 h. Progress of the reaction was monitored by TLC and LCMS. After completion, the reaction mixture was poured onto ice H₂O (500 mL), filtered and dried. The crude residue was purified by normal phase column chromatography (elution: 8% EtOAc in hexanes for 30 min, then 10% EtOAc in hexanes for 30 min, and 8% EtOAc in hexanes) to afford 2.60 g of 7-fluoro-4-nitro-1H-indazole a56 as an off-white solid.

Yield: 20%

¹H NMR (400 MHz, DMSO-d₆): δ 14.58 (brs, 1H), 8.64 (brs, 1H), 8.21 (dd, J=4.8, 3.6 Hz, 1H), 7.46 (t, J=9.2 Hz, 1H).

### A.10.2. Synthesis of 7-fluoro-4-nitro-1-(tetrahydro-2H-pyran-2-yl)-1H-indazole a57

To a solution of 7-fluoro-4-nitro-1H-indazole a56 (4.80 g, 26.5 mmol) in DCM (50 mL) was added DHP (4.85 mL, 53.0 mmol) and p-TSA (0.39 g, 2.04 mmol) at rt and the reaction mixture was stirred at rt for 16 h. Progress of the reaction was monitored by TLC and LCMS. After completion, the reaction mixture was quenched with a saturated aqueous solution of NaHCO₃ (200 mL) and extracted with EtOAc (3×50 mL). The organic layer was dried over anhydrous Na₂SO₄, filtered and concentrated under vacuum. The crude residue was purified by normal phase column chromatography (elution: 10% EtOAc in hexanes for 30 min, then 5% EtOAc in hexanes) to afford 6.97 g of 7-fluoro-4-nitro-1-(tetrahydro-2H-pyran-2-yl)-1H-indazole a57 as an off-white solid.

Yield: 99%

¹H NMR (400 MHz, DMSO-d₆): δ 8.64 (s, 1H), 8.24 (dd, J=8.4, 3.6 Hz, 1H), 7.55 (t, J=9.6 Hz, 1H), 5.94-5.96 (m, 1H), 3.87-3.95 (m, 1H), 3.65-3.75 (m, 1H), 2.35-2.48 (m, 1H), 2.05-2.13 (m, 2H), 1.70-1.85 (m, 1H), 1.56-1.60 (m, 2H).

### A.10.3. Synthesis of 7-fluoro-1-(tetrahydro-2H-pyran-2-yl)-1H-indazol-4-amine a58

To a solution of 7-fluoro-4-nitro-1-(tetrahydro-2H-pyran-2-yl)-1H-indazole a57 (6.90 g, 26.0 mmol) in MeOH (200 mL) and EtOAc (200 mL) was added Pd/C (2.00 g, 18.8 mmol) and the reaction mixture was stirred at rt for 16 h under H₂ (balloon pressure). Progress of the reaction was monitored by TLC and LCMS. After completion, the reaction mixture was filtered through a pad of Celite® and the filtrate was concentrated under vacuum. The crude residue was purified by normal phase column chromatography (elution: 20% EtOAc in hexanes for 30 min, then 10% EtOAc in hexanes) to afford 6.10 g of 7-fluoro-1-(tetrahydro-2H-pyran-2-yl)-1H-indazol-4-amine a58 as a brown semi solid.

Yield: 76%

Basic LCMS Method 1 (ES⁺): 151.85 (M+H)⁺, 85% purity.

¹H NMR (400 MHz, DMSO-d₆): δ 8.19 (d, J=1.5 Hz, 1H) 6.89 (dd, J=12.2, 8.3 Hz, 1H), 6.06 (dd, J=8.3, 2.4 Hz, 1H), 5.71 (dd, J=10.3, 1.71 Hz, 1H), 5.68 (s, 2H), 3.88-3.91 (m, 1H), 3.57-3.66 (m, 1H), 2.36-2.45 (m, 1H), 1.97-2.08 (m, 2H), 1.66-1.78 (m, 1H), 1.51-1.55 (m, 2H).

### A.10.4. Synthesis of 5-chloro-7-fluoro-1-(tetrahydro-2H-pyran-2-yl)-1H-indazol-4-amine a59

To a solution of 7-fluoro-1-(tetrahydro-2H-pyran-2-yl)-1H-indazol-4-amine a58 (5.80 g, 24.7 mmol) in DCM (60 mL) was added NCS (3.29 g, 24.7 mmol) at 0° C. and the reaction mixture was stirred at same temperature for 30 min. The reaction mixture was stirred at rt for 2 h. Progress of the reaction was monitored by TLC and LCMS. After completion, the reaction mixture was quenched with a saturated aqueous solution of NaHCO₃ (250 mL) and extracted with DCM (2×100 mL). The organic layer was dried over anhydrous Na₂SO₄, filtered and concentrated under vacuum. The crude residue was purified by normal phase column chromatography (elution: 20% EtOAc in hexanes for 30 min, then 10% EtOAc in hexanes) to afford 2.20 g of 5-chloro-7-fluoro-1-(tetrahydro-2H-pyran-2-yl)-1H-indazol-4-amine a59 as a brown semi solid.

Yield: 33%

Basic LCMS Method 1 (ES⁺): 185.85 (M+H)⁺, 92% purity.

¹H NMR (400 MHz, DMSO-d₆): δ 8.33 (d, J=2.0 Hz, 1H), 7.17 (d, J=11.2 Hz, 1H), 5.95 (s, 2H), 5.70-5.72 (m, 1H), 3.89 (d, J=11.7 Hz, 1H), 3.56-3.68 (m, 1H), 2.32-2.43 (m, 1H), 2.00-2.02 (m, 2H), 1.66-1.78 (m, 1H), 1.52-1.54 (m, 2H).

### A.10.5. Synthesis of 5-chloro-7-fluoro-4-iodo-1-(tetrahydro-2H-pyran-2-yl)-1H-indazole a60

A stirred mixture of Cul (3.11 g, 16.3 mmol) in MeCN (25 mL) was heated at 50° C., followed by dropwise addition of tBuONO (4.85 mL, 40.8 mmol) at 50° C. and the reaction mixture was stirred at same temperature for 30 min. A solution of 5-chloro-7-fluoro-1-(tetrahydro-2H-pyran-2-yl)-1H-indazol-4-amine a59 (2.20 g, 8.16 mmol) in MeCN (5 mL) was added and the reaction mixture was heated at 80° C. for 2 h. Progress of the reaction was monitored by TLC and LCMS. After completion, the reaction mixture was concentrated under vacuum. The crude residue was basified with a saturated aqueous solution of NaHCO₃ (50 mL) and extracted with EtOAc (3×50 mL). The organic layer was dried over anhydrous Na₂SO₄, filtered and concentrated under vacuum. The crude residue was purified by normal phase column chromatography (elution: 10% EtOAc in hexanes for 30 min, then 4% EtOAc in hexanes) to afford 1.47 g of 5-chloro-7-fluoro-4-iodo-1-(tetrahydro-2H-pyran-2-yl)-1H-indazole a60 as a white solid.

Yield: 47%

¹H NMR (400 MHz, DMSO-d₆): δ 8.05 (s, 1H), 7.65 (d, J=11.6 Hz, 1H), 5.79-5.81 (m, 1H), 3.87-3.90 (m, 1H), 3.62-3.68 (m, 1H), 2.30-2.40 (m, 1H), 2.00-2.10 (m, 2H), 1.65-1.80 (m, 1H), 1.50-1.60 (m, 2H).

### A.10.6. Synthesis of 5-chloro-7-fluoro-4-iodo-1H-indazole a61

To a solution of 5-chloro-7-fluoro-4-iodo-1-(tetrahydro-2H-pyran-2-yl)-1H-indazole a60 (200 mg, 0.53 mmol) in DCM (5 mL) was added TFA (500 μL, 6.73 mmol) at 0° C. and the reaction mixture was stirred at rt for 16 h. Progress of the reaction was monitored by TLC and LCMS. After completion, the reaction mixture was concentrated under vacuum. The crude residue was basified with a saturated aqueous solution of NaHCO₃ (50 mL) and extracted with EtOAc (3×50 mL). The organic layer was dried over anhydrous Na₂SO₄, filtered and concentrated under vacuum. The reaction was repeated with 870 mg of a60 and the crude residues from both reactions were combined and purified by normal phase column chromatography (elution: 20% EtOAc in hexanes for 30 min, then 10% EtOAc in hexanes) to afford 640 mg of 5-chloro-7-fluoro-4-iodo-1H-indazole a61 as an off-white solid.

Yield: 77%

¹H NMR (400 MHz, DMSO-d₆): δ 14.18 (brs, 1H), 8.00 (s, 1H), 7.56 (d, J=10.8 Hz, 1H).

### A.10.7. Synthesis of 3,5-dichloro-7-fluoro-4-iodo-1H-indazole a62

To a solution of 5-chloro-7-fluoro-4-iodo-1H-indazole a61 (640 mg, 2.16 mmol) in MeCN (10 mL) was added NCS (580 mg, 4.32 mmol) and the reaction mixture was heated at 70° C. for 16 h. Progress of the reaction was monitored by TLC and LCMS. After completion, the reaction mixture was concentrated under vacuum. The crude residue was diluted with a saturated aqueous solution of NaHCO₃ (50 mL) and extracted with EtOAc (3×50 mL). The organic layer was dried over anhydrous Na₂SO₄, filtered and concentrated under vacuum. The crude residue was purified by normal phase column chromatography (elution: 10% EtOAc in hexanes for 30 min, then 6% EtOAc in hexanes) to afford 360 mg of 3,5-dichloro-7-fluoro-4-iodo-1H-indazole a62 as an off-white solid.

Yield: 46%

Basic LCMS Method 1 (ES⁺): 331.5 (M+H)⁺, 92% purity.

¹H NMR (400 MHz, DMSO-d₆): δ 14.34 (brs, 1H), 7.67 (d, J=10.4 Hz, 1H).

### A.10.8. Synthesis of 3,5-dichloro-7-fluoro-4-iodo-1-(tetrahydro-2H-pyran-2-yl)-1H-indazole a63

To a solution of 3,5-dichloro-7-fluoro-4-iodo-1H-indazole a62 (350 mg, 1.06 mmol) in DCM (10 mL) was added DHP (190 μL, 2.12 mmol) and p-TSA (20.0 mg, 0.11 mmol) at 0° C. and the reaction mixture was stirred at rt for 16 h. Progress of the reaction was monitored by TLC and LCMS. After completion, the reaction mixture was quenched with a saturated aqueous solution of NaHCO₃ (50 mL) and extracted with EtOAc (3×50 mL). The organic layer was dried over anhydrous Na₂SO₄, filtered and concentrated under vacuum. The crude residue was purified by normal phase column chromatography (elution:10% EtOAc in hexanes for 30 min, then 5% EtOAc in hexanes) to afford 440 mg of 3,5-dichloro-7-fluoro-4-iodo-1-(tetrahydro-2H-pyran-2-yl)-1H-indazole a63 as an off-white solid.

Yield: 84%

¹H NMR (400 MHz, DMSO-d₆): δ 7.77 (d, J=11.6 Hz, 1H), 5.79-5.80 (m, 1H), 3.86-3.89 (m, 1H), 3.61-3.67 (m, 1H), 2.10-2.32 (m, 1H), 1.95-2.10 (m, 2H), 1.63-1.80 (m, 1H), 1.44-1.48 (m, 2H).

### A.10.9. Synthesis of ethyl 2-(3,5-dichloro-7-fluoro-1-(tetrahydro-2H-pyran-2-yl)-1H-indazol-4-yl)acetate a64

Synthesis of Reformatsky reagent: To a solution of Zn (3.00 g, 45.9 mmol) in THF (30 mL) was added TMSCI (600 μL, 4.73 mmol) under argon atmosphere and the reaction mixture was stirred at rt for 15 min. Ethyl-2-bromoacetate (3.30 mL, 0.61 mmol) was added dropwise and the reaction mixture was stirred at rt for 15 min.

A mixture of 3,5-dichloro-7-fluoro-4-iodo-1-(tetrahydro-2H-pyran-2-yl)-1H-indazole a63 (960 mg, 2.31 mmol) and Pd(tBu₃P)₂ (120 mg, 0.23 mmol) was purged with argon for 5 min followed by addition of THF (5 mL). The above Reformatsky reagent (0.6 M, 12 mL, 6.93 mmol) was added and the reaction mixture was heated in sealed tube at 65° C. for 16 h. Progress of reaction was monitored by TLC and LCMS. After completion, the reaction mixture was quenched with a saturated aqueous solution of NH₄Cl (50 mL) and extracted with EtOAc (3×50 mL). The organic layer was dried over anhydrous Na₂SO₄, filtered and concentrated under vacuum. The crude residue was purified by normal phase column chromatography (elution: 10% EtOAc in hexanes for 30 min, then 6% EtOAc in hexanes) to afford 680 mg of ethyl 2-(3,5-dichloro-7-fluoro-1-(tetrahydro-2H-pyran-2-yl)-1H-indazol-4-yl)acetate a64 as a pale brown solid.

Yield: 79%

Basic LCMS Method 1 (ES⁺): 291 (M+H)⁺, 93% purity.

¹H NMR (400 MHz, DMSO-d₆): δ 7.66 (d, J=11.6 Hz, 1H), 5.77-5.80 (m, 1H), 4.11 (q, J=7.2 Hz, 2H), 3.86-3.89 (m, 1H), 3.60-3.67 (m, 1H), 2.26-2.30 (m, 1H), 1.99-2.05 (m, 2H), 1.62-1.75 (m, 1H), 1.45-1.55 (m, 2H), 1.17 (t, J=7.2 Hz, 3H).

### A.10.10. Synthesis of 2-(3,5-dichloro-7-fluoro-1H-indazol-4-yl)acetic acid a65

A stirred solution of ethyl 2-(3,5-dichloro-7-fluoro-1-(tetrahydro-2H-pyran-2-yl)-1H-indazol-4-yl)acetate a64 (100 mg, 0.27 mmol) in a 6N aqueous solution of HCl (2.00 mL, 12.0 mmol) was heated at 80° C. for 16 h. Progress of the reaction was monitored by TLC and LCMS. After completion, the reaction mixture was concentrated under vacuum. The crude residue was basified with a saturated aqueous solution of NaHCO₃ (50 mL) and extracted with EtOAc (2×50 mL). The aqueous layer was acidified with a 6N aqueous solution of HCl up to pH 2 and extracted with EtOAc (3×50 mL). The organic layer was dried over anhydrous Na₂SO₄, filtered and concentrated under vacuum. The reaction was repeated on 580 mg of a64 and the crude residues from both reactions were combined, dissolved in EtOAc (10 mL) and concentrated under vacuum. The solid obtained was washed with pentane (10 mL) and dried to afford 305 mg of 2-(3,5-dichloro-7-fluoro-1H-indazol-4-yl)acetic acid a65 as an off-white solid.

Yield: 64%

HPLC Purity: 97%

Basic LCMS Method 1 (ES⁺): 261 (M+H)⁺, 97% purity.

¹H NMR (400 MHz, DMSO-d₆): δ 14.17 (brs, 1H), 12.68 (brs, 1H), 7.54 (d, J=10.4 Hz, 1H), 4.16 (s, 2H).

### A.11. Synthesis of 2-[3,5-dichloro-2-(1-ethoxyvinyl)-4-pyridyl]acetic acid a67

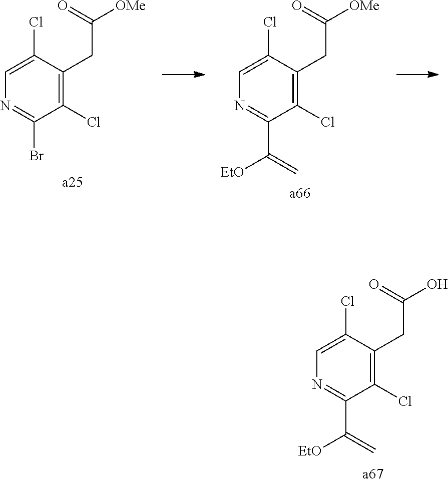 [SMILES: COC(=O)Cc1c(Cl)cnc(Br)c1Cl ; C=C(OCC)c1ncc(Cl)c(CC(=O)OC)c1Cl ; C=C(OCC)c1ncc(Cl)c(CC(=O)O)c1Cl]

### A.11.1. Synthesis of methyl 2-[3,5-dichloro-2-(1-ethoxyvinyl)-4-pyridyl]acetate a66

To a solution of methyl 2-(2-bromo-3,5-dichloro-4-pyridyl)acetate a25 (11.9 g, 40.0 mmol) in toluene (60 mL) was added tributyl(1-ethoxyvinyl)tin (14.6 mL, 42.0 mmol) and tetrakis(triphenylphosphine)palladium(0) (1.86 g, 1.59 mmol) at rt. The reaction mixture was then heated overnight at 120° C. under nitrogen and stirring. The reaction mixture was cooled down to rt. Toluene (250 mL) was added, and the organic layer was washed with a saturated aqueous solution of sodium bicarbonate (200 mL). The organic layer was dried over MgSO₄, filtered and the solvent was removed under vacuum. The crude residue was purified by normal phase column chromatography (elution: 5% EtOAc in hexanes) to afford 8.20 g of methyl 2-[3,5-dichloro-2-(1-ethoxyvinyl)-4-pyridyl]acetate a66.

Yield: 64%

¹H NMR (400 MHz, CDCl₃): δ 8.49 (s, 1H), 4.57-4.46 (m, 2H), 4.05 (s, 2H), 3.96 (q, J=7.0 Hz, 2H), 3.74 (s, 3H), 1.39 (t, J=7.0 Hz, 3H)

### A.11.2. Synthesis of 2-[3,5-dichloro-2-(1-ethoxyvinyl)-4-pyridyl]acetic acid a67

To a solution of methyl 2-[3,5-dichloro-2-(1-ethoxyvinyl)-4-pyridyl]acetate a66 (8.21 g, 28.3 mmol) in THF (80 mL) was added dropwise LiOH·H₂O (1.82 g, 42.5 mmol) dissolved in H₂O (15 mL) and the reaction mixture was allowed to stir overnight at rt. The reaction mixture was evaporated under vacuum to afford 8.10 g of 2-[3,5-dichloro-2-(1-ethoxyvinyl)-4-pyridyl]acetic acid a67 as a white solid, which was used in next steps without further purification.

Yield (crude): quantitative

Basic LCMS Method 2 (ES⁺): 276/278/280 (M+H)⁺.

### B. Synthesis of Intermediates of Formula (III)

### B.1. Synthesis of tert-butyl (1S)-5-bromo-1-methyl-3,4-dihydro-1H-isoquinoline-2-carboxylate b4-(S) and tert-butyl (1R)-5-bromo-1-methyl-3,4-dihydro-1H-isoquinoline-2-carboxylate b4-(R)

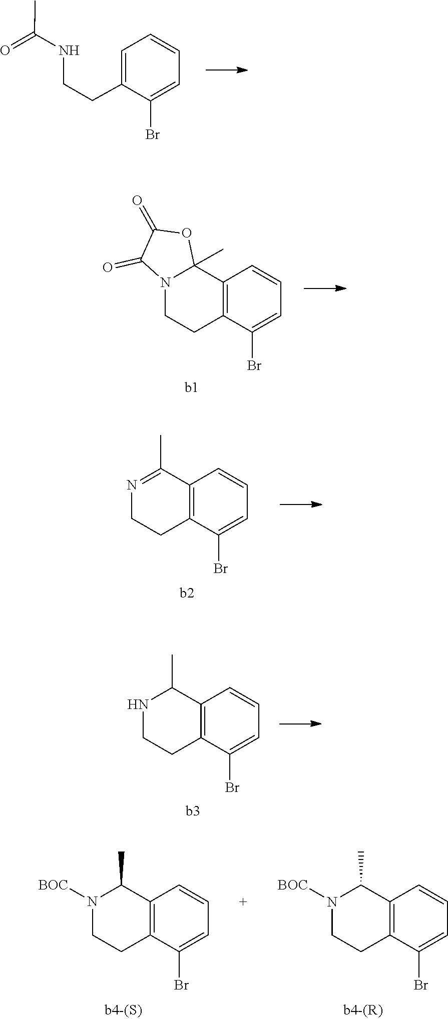 [SMILES: CC(=O)NCCc1ccccc1Br ; CC12OC(=O)C(=O)N1CCc1c(Br)cccc12 ; CC1=NCCc2c(Br)cccc21 ; CC1NCCc2c(Br)cccc21 ; C[C@H]1c2cccc(Br)c2CCN1C(=O)OC(C)(C)C ; C[C@@H]1c2cccc(Br)c2CCN1C(=O)OC(C)(C)C]

### B.1.1. Synthesis of 7-bromo-10b-methyl-5,6-dihydro-[1,3]oxazolo[2,3-a]isoquinoline-2,3-dione b1

To a solution of N-[2-(2-bromophenyl)ethyl]acetamide (commercial, 106 g, 439 mmol) in DCM (1.50 L) was added dropwise at 0° C. oxalyl chloride (72.0 mL, 792 mmol). The mixture was stirred at 0° C. for 2 h, then allowed to warm to rt and stirred for 3 h. The reaction mixture was then cooled to 0° C. and ferric chloride (86.0 g, 530.2 mmol) was added in 2 portions. The reaction mixture was allowed to warm to rt, stirred overnight at rt, diluted with DCM (2.50 L) and then quenched at 0° C. with a 12M concentrated solution of ammonia (200 mL). The organic layer was dried over Na₂SO₄, filtered and concentrated under vacuum to yield 108 g of 7-bromo-10b-methyl-5,6-dihydro-[1,3]oxazolo[2,3-a]isoquinoline-2,3-dione b1 as a brown solid, which was used in next steps without further purification.

Yield (crude): 83%.

Basic LCMS Method 2 (ES⁺): 296/298 (M+H)⁺.

### B.1.2. Synthesis of 5-bromo-1-methyl-3,4-dihydroisoquinoline b2

To a suspension of 7-bromo-10b-methyl-5,6-dihydro-[1,3]oxazolo[2,3-a]isoquinoline-2,3-dione b1 (108 g, 365 mmol) in MeOH (1.50 L) was added dropwise at rt sulfuric acid (75.0 mL). The reaction mixture was stirred overnight at 65° C., then quenched at 0° C. with a 15M concentrated solution of ammonia (300 mL). The mixture was concentrated under vacuum and H₂O (300 mL) was added. The aqueous layer was extracted 6 times with DCM (1.00 L). The organic layer was dried over MgSO₄, filtered and concentrated under vacuum to afford 86.4 g of 5-bromo-1-methyl-3,4-dihydroisoquinoline b2 as a brown solid, which was used in next steps without further purification.

Yield (crude): 95%.

HPLC (Basic Mode): RT 4.75 min, 87% purity.

### B.1.3. Synthesis of 5-bromo-1-methyl-1,2,3,4-tetrahydroisoquinoline b3

To a solution of 5-bromo-1-methyl-3,4-dihydroisoquinoline b2 (86.4 g, 347 mmol) in EtOH (2.00 L) was added at 0° C. sodium borohydride (13.2 g, 349 mmol) by portions (13*1 g). The mixture was stirred at 0° C. for 2 h, then a 5N aqueous solution of HCl (250 mL) was added at 0° C. The reaction mixture was stirred overnight at rt, then EtOH was concentrated under vacuum. DCM (1 L) was added and the mixture was quenched at 0° C. with a 6M concentrated solution of ammonia (400 mL). The organic layer was extracted twice with DCM (500 mL), dried over MgSO₄, filtered and concentrated under vacuum to afford 83.0 g of 5-bromo-1-methyl-1,2,3,4-tetrahydroisoquinoline b3 as a brown solid, which was used in next step without any further purification.

Yield (crude): 85%.

HPLC (Basic Mode): RT 4.53 min, 80% purity.

### B.1.4. Synthesis of tert-butyl (1S)-5-bromo-1-methyl-3,4-dihydro-1H-isoquinoline-2-carboxylate b4-(S) and tert-butyl (1R)-5-bromo-1-methyl-3,4-dihydro-1H-isoquinoline-2-carboxylate b4-(R)

To a solution of 5-bromo-1-methyl-1,2,3,4-tetrahydroisoquinoline b3 (78.0 g, 276 mmol) in DCM (1 L) was added TEA (160 mL, 1.14 mol) at 0° C. A solution of di-tert-butyl dicarbonate (65.0 g, 295 mmol) in DCM (250 mL) was then added dropwise at 0° C. The reaction mixture was stirred overnight at rt and quenched with water (100 mL). The organic layer was dried over MgSO₄, filtered and concentrated under vacuum. The crude residue was triturated twice in a mixture of MeOH and hexanes (1:2, 450 mL) to yield 63.0 g of racemate tert-butyl 5-bromo-1-methyl-3,4-dihydro-1H-isoquinoline-2-carboxylate b4 (Yield: 70%, HPLC (Basic Mode): RT 6.59 min, 98% purity) as a white solid.

Chiral separation (SFC, Whelko 01 (R,R), 50×227 mm, 360 mL/min, 220 nm, 25° C., elution: EtOH 20%—CO₂ 80%) of racemate tert-butyl 5-bromo-1-methyl-3,4-dihydro-1H-isoquinoline-2-carboxylate b4 afforded:

25.1 g of tert-butyl (1 S)-5-bromo-1-methyl-3,4-dihydro-1H-isoquinoline-2-carboxylate b4-(S) as a solid.

Yield: 40%.

HPLC (Basic Mode): RT 6.59 min, 91% purity.

Chiral analysis (LC, Whelko-01 (R,R), 250*4.6 mm, 1 mL/min, 220 nm, 30° C., elution: iPrOH/heptane/DEA 50/50/0.1) RT 4.86 min, 98% ee.

29.3 g of tert-butyl (1R)-5-bromo-1-methyl-3,4-dihydro-1H-isoquinoline-2-carboxylate b4-(R) as a solid.

Yield: 46%.

HPLC (Basic Mode): RT 6.59 min, 98% purity.

Chiral analysis (LC, Whelko-01 (R,R), 250*4.6 mm, 1 mL/min, 220 nm, 30° C., elution: iPrOH/heptane/DEA 50/50/0.1) RT 5.62 min, 92% ee.

### B.2. Synthesis of 2-[2-[(1S,4aR,8aS)-5-formyl-1-methyl-3,4,4a,5,6,7,8,8a-octahydro-1H-isoquinolin-2-yl]-2-oxoethyl]-3-chloro-4-methoxybenzonitrile b11

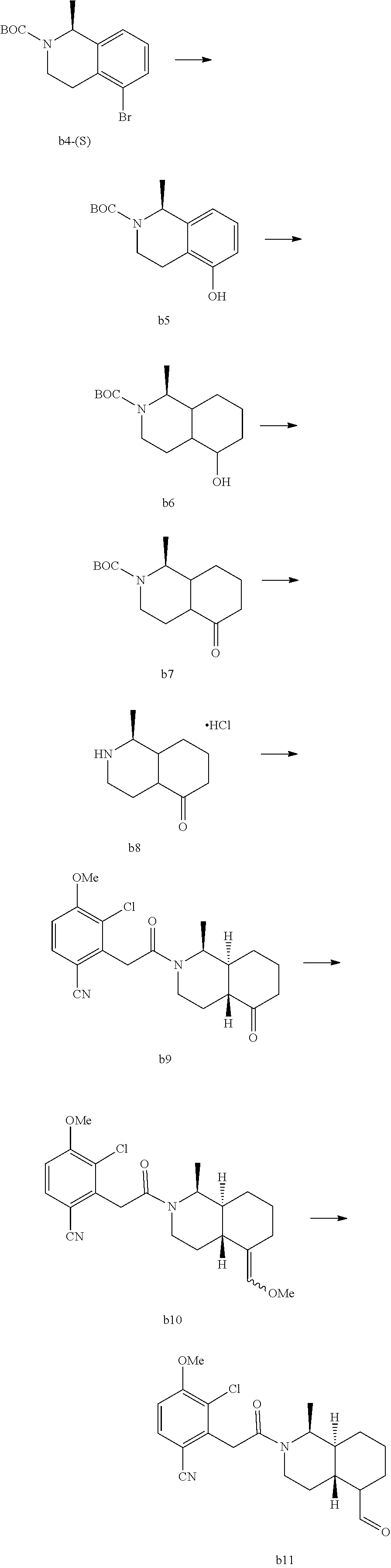

### B.2.1. Synthesis of tert-butyl (1S)-5-hydroxy-1-methyl-3,4-dihydro-1H-isoquinoline-2-carboxylate b5

To a solution of tert-butyl (1S)-5-bromo-1-methyl-3,4-dihydro-1H-isoquinoline-2-carboxylate b4-(S) (24.9 g, 76.5 mmol) in 1,4-dioxane (80 mL) was added tBuXPhos palladacycle (750 mg, 1.09 mmol). Then, a solution of KOH (11.6 g, 176 mmol) in water (20 mL) was added and the reaction mixture was stirred at 85° C. for 2 h. The reaction mixture was quenched at rt with a 1N aqueous solution of HCl (400 mL) and extracted with EtOAc (400 mL). The organic layer was washed twice with H₂O (250 mL). The organic layer was dried over MgSO₄, filtered and concentrated under vacuum. The crude residue was purified by normal phase column chromatography (elution: 20% EtOAc in hexanes) to afford 16.6 g of tert-butyl (1S)-5-hydroxy-1-methyl-3,4-dihydro-1H-isoquinoline-2-carboxylate b5 as a white solid.

Yield: 76%

Basic LCMS Method 2 (ES⁺): 208 (M-tBu+H)⁺, 164 (M-Boc+H)⁺.

### B.2.2. Synthesis of tert-butyl (1S)-5-hydroxy-1-methyl-3,4,4a,5,6,7,8,8a-octahydro-1H-isoquinoline-2-carboxylate b6

To a solution of tert-butyl (1 S)-5-hydroxy-1-methyl-3,4-dihydro-1H-isoquinoline-2-carboxylate b5 (2.00 g, 7.60 mmol) in isopropanol (20 mL) was added rhodium on activated carbon (Johnson-Matthey Type 20C, 234 mg, 0.11 mmol). The reaction mixture was flushed with nitrogen followed by H₂. The reaction mixture was heated at 100° C. under a pressure of 8 bars of H₂ for 72 h. The reaction mixture was cooled down to rt, filtered through a pad of Celite® and the filtrate was concentrated under vacuum to afford 2.37 g of tert-butyl (1S)-5-hydroxy-1-methyl-3,4,4a,5,6,7,8,8a-octahydro-1H-isoquinoline-2-carboxylate b6, which was used in next steps without further purification.

Yield (crude): quantitative

Basic LCMS Method 2 (ES⁺): 214 (M+H)⁺.

### B.2.3. Synthesis of tert-butyl (1S)-1-methyl-5-oxo-1,3,4,4a,6,7,8,8a-octahydroisoquinoline-2-carboxylate b7

To a solution of tert-butyl (1 S)-5-hydroxy-1-methyl-3,4,4a,5,6,7,8,8a-octahydro-1H-isoquinoline-2-carboxylate b6 (68.0 g, 203 mmol) in acetic acid (280 mL, 4.89 mol) was added at 0° C. a 0.87 M aqueous solution of sodium hypochlorite (1.00 L, 870 mmol). The reaction mixture was stirred at 10° C. during the addition, then at rt overnight. The reaction mixture was extracted twice with DCM (250 mL). The organic layer was washed with a saturated aqueous solution of NaHCO₃ (200 mL), dried over MgSO₄, filtered and concentrated under vacuum to afford 69.0 g of tert-butyl (1S)-1-methyl-5-oxo-1,3,4,4a,6,7,8,8a-octahydroisoquinoline-2-carboxylate b7, which was used in next steps without further purification.

Yield (crude): 96%.

Basic LCMS Method 2 (ES⁺): 212 (M+H)⁺.

### B.2.4. Synthesis of (1S)-1-methyl-2,3,4,4a,6,7,8,8a-octahydro-1H-isoquinolin-5-one hydrochloride b8

To a solution of tert-butyl (1 S)-1-methyl-5-oxo-1,3,4,4a,6,7,8,8a-octahydroisoquinoline-2-carboxylate b7 (3.12 g, 11.7 mmol) in iPrOH (6.00 mL) was added dropwise at rt a 5N aqueous solution of HCl in iPrOH (6.00 mL). The reaction mixture was stirred overnight at rt. The solid obtained was filtered off and washed once with the mother liquor phase and twice with fresh iPrOH (6.00 mL). The mother liquor phase was concentrated under vacuum to afford 2.17 g of (1S)-1-methyl-2,3,4,4a,6,7,8,8a-octahydro-1H-isoquinolin-5-one hydrochloride b8 as a brown oil, which was used in next step without any further purification.

Yield (crude): 64%.

Basic LCMS Method 2 (ES⁺): 168 (M+H)⁺.

### B.2.5. Method a (Peptidic Coupling

### Synthesis of 2-[2-[(1S,4aR,8aS)-1-methyl-5-oxo-1,3,4,4a,6,7,8,8a-octahydroisoquinolin-2-yl]-2-oxoethyl]-3-chloro-4-methoxybenzonitrile b9

To a solution of 2-(2-chloro-6-cyano-3-methoxyphenyl)acetic acid a3 (4.43 g, 17.3 mmol) and (1S)-1-methyl-2,3,4,4a,6,7,8,8a-octahydro-1H-isoquinolin-5-one hydrochloride b8 (4.00 g, 19.6 mmol) in DMF (10 mL) were added HBTU (8.19 g, 21.6 mmol), followed by the addition at 0° C. of Et₃N (8.30 mL, 59.5 mmol). The reaction mixture was stirred overnight at rt. The reaction mixture was poured in EtOAc (300 mL), then washed twice with a 1N aqueous solution of HCl (150 mL). The organic layer was dried over MgSO₄, filtered and concentrated under vacuum. The crude residue was triturated with EtOAc (20 mL) and the obtained precipitate was filtered. The mother liquor was sonicated and a second precipitate was filtered. Both precipitates were combined and dried under vacuum to afford 4.21 g of 2-[2-[(1S,4aR,8aS)-1-methyl-5-oxo-1,3,4,4a,6,7,8,8a-octahydroisoquinolin-2-yl]-2-oxoethyl]-3-chloro-4-methoxybenzonitrile b9 as the pure desired isomer.

Yield: 57%

Basic LCMS Method 3 (ES⁺): 375/377 (M+H)⁺, 99% purity.

Acid LCMS Method 2 (ES⁺): 375/377 (M+H)⁺, 99% purity.

### B.2.6. Synthesis of 2-[2-[(1S,4aR,8aS)-5-(methoxymethylidene)-1-methyl-1,3,4,4a,6,7,8,8a-octahydroisoquinolin-2-yl]-2-oxoethyl]-3-chloro-4-methoxybenzonitrile b10

To a solution of (methoxymethyl)triphenylphosphonium chloride (7.68 g, 22.4 mmol) in THF (100 mL) was added dropwise at −78° C. a 2.5 M solution of n-BuLi (8.10 mL, 20.2 mmol) in hexanes. The reaction mixture was allowed to warm slowly to 0° C. and stirred at 0° C. for 15 min. Then, the reaction mixture was cooled down at −78° C. and 2-[2-[(1 S,4aR,8aS)-1-methyl-5-oxo-1,3,4,4a,6,7,8,8a-octahydroisoquinolin-2-yl]-2-oxoethyl]-3-chloro-4-methoxybenzonitrile b9 (4.20 g, 11.2 mmol) was added in portions. The reaction mixture was allowed to warm slowly to rt, stirred at rt for 2 h then diluted with Et₂O (500 mL) The mixture was washed with water (250 mL). The organic layer was dried over MgSO₄, filtered and concentrated under vacuum. The crude residue was purified by normal phase column chromatography (elution: EtOAc/hexanes) to afford 4.10 g of 2-[2-[(1S,4aR,8aS)-5-(methoxymethylidene)-1-methyl-1,3,4,4a,6,7,8,8a-octahydroisoquinolin-2-yl]-2-oxoethyl]-3-chloro-4-methoxybenzonitrile b10.

Yield: 91%

Basic LCMS Method 2 (ES⁺): 403/405 (M+H)⁺.

### B.2.7. Synthesis of 2-[2-[(1S,4aR,8aS)-5-formyl-1-methyl-3,4,4a,5,6,7,8,8a-octahydro-1H-isoquinolin-2-yl]-2-oxoethyl]-3-chloro-4-methoxybenzonitrile b11

To a solution of 2-[2-[(1 S,4aR,8aS)-5-(methoxymethylidene)-1-methyl-1,3,4,4a,6,7,8,8a-octahydroisoquinolin-2-yl]-2-oxoethyl]-3-chloro-4-methoxybenzonitrile b10 (4.00 g, 9.93 mmol) in THF (200 mL) was added dropwise a 1 N aqueous solution of HCl (50 mL) at rt. The reaction mixture was stirred at rt for 48 h. EtOAc (150 mL) was added and the mixture was successively washed with H₂O (50 mL), with a saturated aqueous solution of NaHCO₃ (50 mL) and with brine (50 mL). The organic layer was dried over MgSO₄, filtered and concentrated under vacuum to afford 3.86 g of 2-[2-[(1S,4aR,8aS)-5-formyl-1-methyl-3,4,4a,5,6,7,8,8a-octahydro-1H-isoquinolin-2-yl]-2-oxoethyl]-3-chloro-4-methoxybenzonitrile b11, which was used in the next steps without further purification.

Yield (crude): quantitative

Basic LCMS Method 2 (ES⁺): 389/391 (M+H)⁺.

### B.3. Synthesis of (1S,4aR,8aS)-1-methyl-2,3,4,4a,6,7,8,8a-octahydro-1H-isoquinolin-5-one b8-peak2

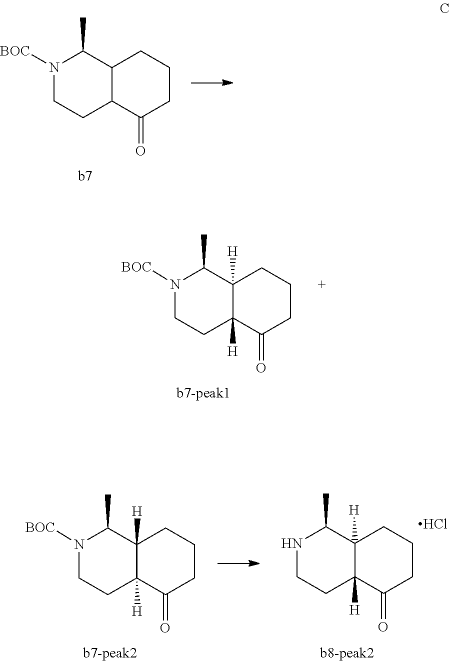

### B.3.1. Synthesis of tert-butyl (1S,4aS,8aR)-1-methyl-5-oxo-1,3,4,4a,6,7,8,8a-octahydroisoquinoline-2-carboxylate b7-peak1 and tert-butyl (1S,4aR,8aS)-1-methyl-5-oxo-1,3,4,4a,6,7,8,8a-octahydroisoquinoline-2-carboxylate b7-peak2

Chiral separation (LC, YMC Chiralart cellulose-sc, 4.6*250 mm, 1 mL/min, 220 nm, 30° C., elution: iPrOH/hexane/NH₃ 10/90/0.1) of 5.50 g of tert-butyl (1S)-1-methyl-5-oxo-1,3,4,4a,6,7,8,8a-octahydroisoquinoline-2-carboxylate b7 afforded:

2.31 g of tert-butyl (1S,4aS,8aR)-1-methyl-5-oxo-1,3,4,4a,6,7,8,8a-octahydroisoquinoline-2-carboxylate b7-peak1 as a mixture of cis and trans undesired isomers.

Yield: 42%.

Basic LCMS Method 1 (ES⁺): 168 (M+H)⁺, 90% purity.

¹H NMR (400 MHz, CDCl₃): δ 4.21-4.31 (m, 1H), 4.08-4.12 (m, 1H), 2.84-2.92 (m, 1H), 2.72-2.78 (m, 1H), 2.42-2.54 (m, 1H), 2.24-2.30 (m, 1H), 2.04-2.12 (m, 1H), 1.79-1.96 (m, 3H), 1.60-1.67 (m, 3H), 1.47 (s, 9H), 1.17 (m, J=6.85 Hz, 3H)

Chiral analysis (LC, IC, 150*4.6 mm, 1.5 mL/min, 220 nm, 30° C., elution: iPrOH/heptane/DEA 10/90/0.1) RT 5.93 min, 72% de+RT 6.33 min, 28% de.

2.16 g of tert-butyl (1S,4aR,8aS)-1-methyl-5-oxo-1,3,4,4a,6,7,8,8a-octahydroisoquinoline-2-carboxylate b7-peak2 as the pure desired isomer.

Yield: 39%

Basic LCMS Method 1 (ES⁺): 168 (M+H)⁺, 100% purity.

¹H NMR (400 MHz, CDCl₃): δ 4.40-4.48 (s, 1H), 4.19-4.29 (m, 1H), 4.03-4.16 (m, 1H), 3.96-4.02 (m, 1H), 2.69-2.90 (m, 1H), 2.40-2.49 (m, 1H), 2.21-2.38 (m, 2H), 2.04-2.12 (m, 1H), 1.65-1.92 (m, 4H), 1.45 (s, 9H), 1.12 (d, J=6.85 Hz, 3H).

Chiral analysis (LC, IC, 150*4.6 mm, 1.5 mL/min, 220 nm, 30° C., elution: iPrOH/heptane/DEA 10/90/0.1) RT 7.80 min, 95% de.

### B.3.2. Synthesis of (1S,4aR,8aS)-1-methyl-2,3,4,4a,6,7,8,8a-octahydro-1H-isoquinolin-5-one b8-peak2

To a solution of tert-butyl (1 S)-1-methyl-5-oxo-1,3,4,4a,6,7,8,8a-octahydroisoquinoline-2-carboxylate b7 (1.00 g, 3.74 mmol) in MeOH (50 mL) was added dropwise at rt a 12 M solution of HCl in MeOH (15.0 mL). The reaction mixture was then allowed to stir overnight at rt for 4 h and concentrated under vacuum to afford 625 mg of (1S)-1-methyl-2,3,4,4a,6,7,8,8a-octahydro-1H-isoquinolin-5-one hydrochloride b8-peak2, which was used in next step without any further purification.

Yield (crude): quantitative.

Basic LCMS Method 2 (ES⁺): 168 (M+H)⁺.

### B.4. Synthesis of (1S)-1-[(1S,4aR,5R,8aS)-1-methyl-1,2,3,4,4a,5,6,7,8,8a-decahydroisoquinolin-5-yl]-2,2-difluoro-ethanol hydrochloride b18-(S)

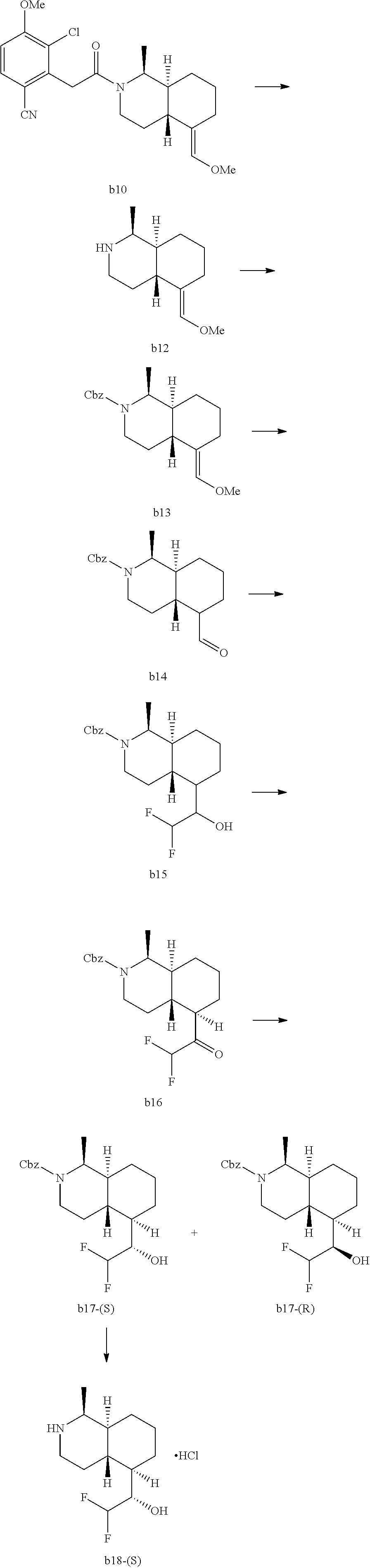

### B.4.1. Synthesis of (1S,4aR,5E,8aS)-5-(methoxymethylene)-1-methyl-2,3,4,4a,6,7,8,8a-octahydro-1H-isoquinoline b12

To a solution of 2-[2-[(1S,4aR,5E,8aS)-5-(methoxymethylene)-1-methyl-1,3,4,4a,6,7,8,8a-octahydroisoquinolin-2-yl]-2-oxo-ethyl]-3-chloro-4-methoxy-benzonitrile b10 (21.5 g, 53.4 mmol) in 1,4-dioxane (200 mL) was added lithium hydroxide monohydrate (32.0 g, 750 mmol) dissolved in water (500 mL) at rt. The reaction mixture was heated 4 days at 130° C. The reaction mixture was cooled down to rt and was extracted with DCM (4×250 mL). The organic layer was dried over MgSO₄, filtered and concentrated under vacuum to afford 11.0 g of (1 S,4aR,5E,8aS)-5-(methoxymethylene)-1-methyl-2,3,4,4a,6,7,8,8a-octahydro-1H-isoquinoline b12 as a solid, which was used in the next step without further purification.

Yield (crude): 100%

Acic LCMS Method 1 (ES⁺): 196 (M+H)⁺.

### B.4.2. Synthesis of benzyl (1S,4aR,5E,8aS)-5-(methoxymethylene)-1-methyl-1,3,4,4a,6,7,8,8a-octahydroisoquinoline-2-carboxylate b13

To a solution of (1 S,4aR,5E,8aS)-5-(methoxymethylene)-1-methyl-2,3,4,4a,6,7,8,8a-octahydro-1H-isoquinoline b12 (11.0 g, 53.5 mmol) in DCM (200 mL) was added N-(benzyloxycarbonyloxy) succinimide (16.4 g, 64.5 mmol) and the reaction mixture was stirred for 5 min. Then, DIPEA (30.0 mL, 180 mmol) was added dropwise and the reaction mixture was stirred at rt for 2 h. The reaction mixture was diluted with DCM (250 mL) and the organic layer was washed with H₂O (2×250 mL). The organic phase was dried over MgSO₄, filtered and concentrated under vacuum. The crude residue was purified by normal phase column chromatography (elution: 10% EtOAc inn-hexanes) to afford 14.0 g of benzyl (1S,4aR,5E,8aS)-5-(methoxymethylene)-1-methyl-1,3,4,4a,6,7,8,8a-octahydroisoquinoline-2-carboxylate b13.

Yield: 79%

Acid LCMS Method 1 (ES⁺): 330 (M+H)⁺.

### B.4.3. Synthesis of benzyl (1S,4aR,8aS)-5-formyl-1-methyl-3,4,4a,5,6,7,8,8a-octahydro-1H-isoquinoline-2-carboxylate b14

To a solution of benzyl (1 S,4aR,5E,8aS)-5-(methoxymethylene)-1-methyl-1,3,4,4a,6,7,8,8a-octahydroisoquinoline-2-carboxylate b13 (14.0 g, 42.5 mmol) in THF (560 mL) was added dropwise at rt a 1N aqueous solution of HCl (85 mL) and the reaction mixture was stirred overnight at rt. EtOAc (200 mL) was added to the reaction mixture and the organic layer was washed with a saturated aqueous solution of sodium bicarbonate (100 mL). The the aqueous layer was extracted with EtOAc (200 mL). The combined organic layers were dried over MgSO₄, filtered and concentrated under high vacuum to afford 13.0 g of benzyl (1S,4aR,8aS)-5-formyl-1-methyl-3,4,4a,5,6,7,8,8a-octahydro-1H-isoquinoline-2-carboxylate b14, which was used in the next step without further purification.

Yield: 96%

Acic LCMS Method 1 (ES⁺): 316 (M+H)⁺.

### B.4.4. Synthesis of benzyl (1S,4aR,8aS)-5-(2,2-difluoro-1-hydroxy-ethyl)-1-methyl-3,4,4a,5,6,7,8,8a-octahydro-1H-isoquinoline-2-carboxylate b15

To a solution of benzyl (1 S,4aR,8aS)-5-formyl-1-methyl-3,4,4a,5,6,7,8,8a-octahydro-1H-isoquinoline-2-carboxylate b14 (3.40 g, 11.0 mmol) in DMF (50 mL) was added cesium fluoride (3.27 g, 21.3 mmol) and the reaction mixture was cooled down to 0° C. Difluoromethyl)trimethylsilane (3.08 mL, 21.3 mmol) was added dropwise and the reaction mixture was stirred for 15 min at 0° C. and warmed to rt for 6 h. To the reaction mixture was added a 37% aqueous solution of HCl (1.80 mL, 22.0 mmol) and the reaction mixture was stirred overnight at rt. EtOAc (250 mL) was added to the reaction. The organic layer was successively washed with a saturated aqueous solution of sodium bicarbonate (100 mL), then with brine (100 mL). The aqueous layer was extracted again with EtOAc (250 mL). The combined organic layers were finally washed with water (250 mL), dried over MgSO₄, filtered and concentrated under high vacuum to afford 4.46 g of benzyl (1S,4aR,8aS)-5-(2,2-difluoro-1-hydroxy-ethyl)-1-methyl-3,4,4a,5,6,7,8,8a-octahydro-1H-isoquinoline-2-carboxylate b15 which was used in next steps without further purification.

Yield (crude): 100%

Acic LCMS Method 1 (ES⁺): 368 (M+H)+

### B.4.5. Synthesis of benzyl (1S,4aR,5R,8aS)-5-(2,2-difluoroacetyl)-1-methyl-3,4,4a,5,6,7,8,8a-octahydro-1H-isoquinoline-2-carboxylate b16

To a solution of benzyl (1S,4aR,8aS)-5-(2,2-difluoro-1-hydroxy-ethyl)-1-methyl-3,4,4a,5,6,7,8,8a-octahydro-1H-isoquinoline-2-carboxylate b15 (4.46 g, 10.9 mmol) in DCM (100 mL) was added portion wise Dess-Martin Periodinane (6.20 g, 14.0 mmol) at 0° C. and the reaction mixture was stirred at rt overnight. DCM (100 mL) was added, followed by a saturated aqueous solution of sodium bicarbonate (100 mL). The aqueous layer was extracted with DCM (100 mL). The organic layer was washed with a saturated aqueous solution of sodium bicarbonate (2×100 mL) and finally with water (100 mL). The organic layer was dried over MgSO₄, filtered and concentrated under vacuum. The crude residue was purified by normal phase column chromatography (elution: from 5% to 60% EtOAc in hexanes) to afford 3.10 g of benzyl (1S,4aR,5R,8aS)-5-(2,2-difluoroacetyl)-1-methyl-3,4,4a,5,6,7,8,8a-octahydro-1H-isoquinoline-2-carboxylate b16.

Yield: 77%

Acic LCMS Method 1 (ES⁺): 366 (M+H)⁺.

### B.4.6. Synthesis of benzyl (1S,4aR,5R,8aS)-5-[(1S)-2,2-difluoro-1-hydroxy-ethyl]-1-methyl-3,4,4a,5,6,7,8,8a-octahydro-1H-isoquinoline-2-carboxylate b17-(S) and benzyl (1S,4aR,5R,8aS)-5-[(1S)-2,2-difluoro-1-hydroxy-ethyl]-1-methyl-3,4,4a,5,6,7,8,8a-octahydro-1H-isoquinoline-2-carboxylate b17-(R)

To a solution of benzyl (1S,4aR,5R,8aS)-5-(2,2-difluoroacetyl)-1-methyl-3,4,4a,5,6,7,8,8a-octahydro-1H-isoquinoline-2-carboxylate b16 (1.55 g, 4.24 mmol) in 2-MeTHF (30.0 mL) was added at 0° C. lithium borohydride (120 mg, 5.23 mmol) and the reaction mixture was stirred overnight. The reaction mixture was quenched with H₂O (5 mL) and stirred for 1 h. Then, a 1 N aqueous solution of HCl (5 mL) was added dropwise and the reaction mixture was stirred for another 2 h. The reaction mixture was diluted with EtOAc (100 mL) and washed once with H₂O. The aqueous layer was extracted with EtOAc (50 mL). The combined organic layers were dried over MgSO₄, filtered and concentrated under vacuum to afford 1.50 g of benzyl (1S,4aR,8aS)-5-(2,2-difluoro-1-hydroxy-ethyl)-1-methyl-3,4,4a,5,6,7,8,8a-octahydro-1H-isoquinoline-2-carboxylate b17.

Yield: 93%

Acic LCMS Method 1 (ES⁺): 368 (M+H)⁺.

Chiral separation (SFC, Chiralpak AD Daicel®, 20 μm, 279×50 mm, 360 mL/min, 220 nm, 30° C., elution: EtOH 20%—CO₂ 80%) of racemate b17 afforded:

910 mg of benzyl (1S,4aR,5R,8aS)-5-[(1S)-2,2-difluoro-1-hydroxy-ethyl]-1-methyl-3,4,4a,5,6,7,8,8a-octahydro-1H-isoquinoline-2-carboxylate b17-(S)

Yield: 63%

Acic LCMS Method 1 (ES⁺): 368 (M+H)⁺, 100% purity.

Chiral analysis (LC, Chiralpak AD Daicel®, 3 μm, 150*4.6 mm, 1.5 mL/min, 220 nm, 30° C., elution: MeOH/DEA 100/0.1): RT 1.72 min, 100% de.

335 mg of benzyl (1S,4aR,5R,8aS)-5-[(1R)-2,2-difluoro-1-hydroxy-ethyl]-1-methyl-3,4,4a,5,6,7,8,8a-octahydro-1H-isoquinoline-2-carboxylate b17-(R)

Yield: 23%

Acic LCMS Method 1 (ES⁺): 368 (M+H)⁺, 100% purity.

Chiral analysis (LC, Chiralpak AD Daicel®, 3 μm, 150×4.6 mm, 1.5 mL/min, 220 nm, 30° C., elution: MeOH/DEA 100/0.1): RT 3.92 min, 100% de.

### B.4.7. Synthesis of (1S)-1-[(1S,4aR,5R,8aS)-1-methyl-1,2,3,4,4a,5,6,7,8,8a-decahydroisoquinolin-5-yl]-2,2-difluoro-ethanol hydrochloride b18-(S)

A solution of benzyl (1S,4aR,5R,8aS)-5-[(1S)-2,2-difluoro-1-hydroxy-ethyl]-1-methyl-3,4,4a,5,6,7,8,8a-octahydro-1H-isoquinoline-2-carboxylate b17-(S) (900 mg, 2.45 mmol) in a 4N solution of HCl in 1,4-dioxane (6 mL) was stirred at 60° C. for 48 h. The reaction mixture was concentrated under vacuum and dried under high vacuum (oven) at 45° C. for 72 h to afford 650 mg of (1S)-1-[(1 S,4aR,5R,8aS)-1-methyl-1,2,3,4,4a,5,6,7,8,8a-decahydroisoquinolin-5-yl]-2,2-difluoro-ethanol hydrochloride b18-(S) as a solid, which was used in the next step without further purification.

Yield (crude): 93%

### B.5. Synthesis of (2S)-2-[(1S,4aR,5R,8aS)-1-methyl-1,2,3,4,4a,5,6,7,8,8a-decahydroisoquinolin-5-yl]-1,1-difluoro-propan-2-ol hydrochloride b20-(S)

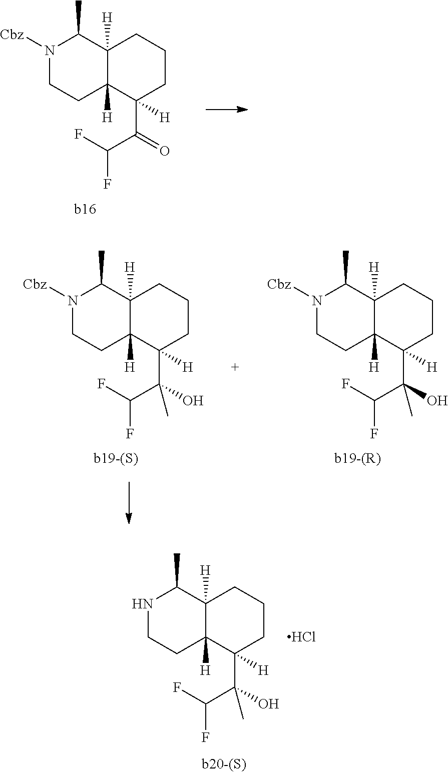 [SMILES: C[C@H]1[C@H]2CCC[C@@H](C(=O)C(F)F)[C@@H]2CCN1C(=O)OCc1ccccc1 ; C[C@H]1[C@H]2CCC[C@@H]([C@](C)(O)C(F)F)[C@@H]2CCN1C(=O)OCc1ccccc1 ; C[C@H]1[C@H]2CCC[C@@H]([C@@](C)(O)C(F)F)[C@@H]2CCN1C(=O)OCc1ccccc1 ; C[C@@H]1NCC[C@@H]2[C@@H]1CCC[C@H]2[C@](C)(O)C(F)F ; Cl]

### B.5.1. Synthesis of benzyl (1S,4aR,5R,8aS)-5-[(1S)-2,2-difluoro-1-hydroxy-1-methyl-ethyl]-1-methyl-3,4,4a,5,6,7,8,8a-octahydro-1H-isoquinoline-2-carboxylate b19-(S) and benzyl (1S,4aR,5R,8aS)-5-[(1R)-2,2-difluoro-1-hydroxy-1-methyl-ethyl]-1-methyl-3,4,4a,5,6,7,8,8a-octahydro-1H-isoquinoline-2-carboxylate b19-(R)

To a solution of benzyl (1S,4aR,5R,8aS)-5-(2,2-difluoroacetyl)-1-methyl-3,4,4a,5,6,7,8,8a-octahydro-1H-isoquinoline-2-carboxylate b16 (1.55 g, 4.24 mmol) in 2-MeTHF (30 mL) was added at 0° C. a 3 M solution of methylmagnesium chloride in THF (1.70 mL) and the reaction mixture was allowed to stir overnight at rt. Then a 3 M solution of methylmagnesium chloride in THF (1.00 mL, 3.00 mmol) was added again at rt and the reaction was stirred for 1 h. The reaction mixture was then quenched with H₂O (5 mL) and stirred for 1 h. Then, a 1 N aqueous solution of HCl (5 mL) was added dropwise and the reaction mixture was stirred for another 2 h. The reaction mixture was diluted with EtOAc (100 mL) and washed with H₂O. The aqueous layer was extracted with EtOAc (50 mL). The organic layer was dried over MgSO₄, filtered and concentrated under vacuum to afford 1.60 g of benzyl (1S,4aR,5R,8aS)-5-(2,2-difluoro-1-hydroxy-1-methyl-ethyl)-1-methyl-3,4,4a,5,6,7,8,8a-octahydro-1H-isoquinoline-2-carboxylate b19 (Yield: 90%, Acic LCMS Method 1 (ES⁺): 382 (M+H)⁺).

Chiral separation (SFC, Chiralpak AD Daicel®, 20 μm, 279×50 mm, 360 mL/min, 220 nm, 30° C., elution: EtOH 20%—CO₂ 80%) of racemate b19 afforded:

640 mg of benzyl (1S,4aR,5R,8aS)-5-[(1 S)-2,2-difluoro-1-hydroxy-1-methyl-ethyl]-1-methyl-3,4,4a,5,6,7,8,8a-octahydro-1H-isoquinoline-2-carboxylate b19-(S)

Yield: 44%

Basic LCMS Method 2 (ES⁺): 382 (M+H)⁺, 97 purity.

Chiral analysis (LC, Chiralpak AD Daicel®, 3 μm, 150×4.6 mm, 1.5 mL/min, 220 nm, 30° C., elution: EtOH/DEA 100/0.1): RT 1.85 min, 100% de.

270 mg of benzyl (1S,4aR,5R,8aS)-5-[(1R)-2,2-difluoro-1-hydroxy-1-methyl-ethyl]-1-methyl-3,4,4a,5,6,7,8,8a-octahydro-1H-isoquinoline-2-carboxylate b19-(R)

Yield: 19%

Basic LCMS Method 2 (ES⁺): 382 (M+H)⁺, 91 purity.

Chiral analysis (LC, Chiralpak AD Daicel®, 3 μm, 150×4.6 mm, 1.5 mL/min, 220 nm, 30° C., elution: EtOH/DEA 100/0.1): RT 2.34 min, 93% de.

### B.5.2. Synthesis of (2S)-2-[(1S,4aR,5R,8aS)-1-methyl-1,2,3,4,4a,5,6,7,8,8a-decahydroisoquinolin-5-yl]-1,1-difluoro-propan-2-ol hydrochloride b20-(S)

A solution of benzyl (1S,4aR,5R,8aS)-5-[(1S)-2,2-difluoro-1-hydroxy-1-methyl-ethyl]-1-methyl-3,4,4a,5,6,7,8,8a-octahydro-1H-isoquinoline-2-carboxylate b19-(S) (630 mg, 1.65 mmol) in a 4N solution of HCl in 1,4-dioxane (4.00 mL) was stirred at 60° C. for 48 h. The reaction mixture was evaporated under vacuum to afford 450 mg of (2S)-2-[(1S,4aR,5R,8aS)-1-methyl-1,2,3,4,4a,5,6,7,8,8a-decahydroisoquinolin-5-yl]-1,1-difluoro-propan-2-ol hydrochloride b20-(S), which was used in the next step without further purification.

Yield (crude): 91%

### B.6. Synthesis of (1S)-1-[(1S,3R,4aS,5S,8aR)-3-(hydroxymethyl)-1-methyl-1,2,3,4,4a,5,6,7,8,8a-decahydroisoquinolin-5-yl]-2,2,2-trifluoro-ethanol b38

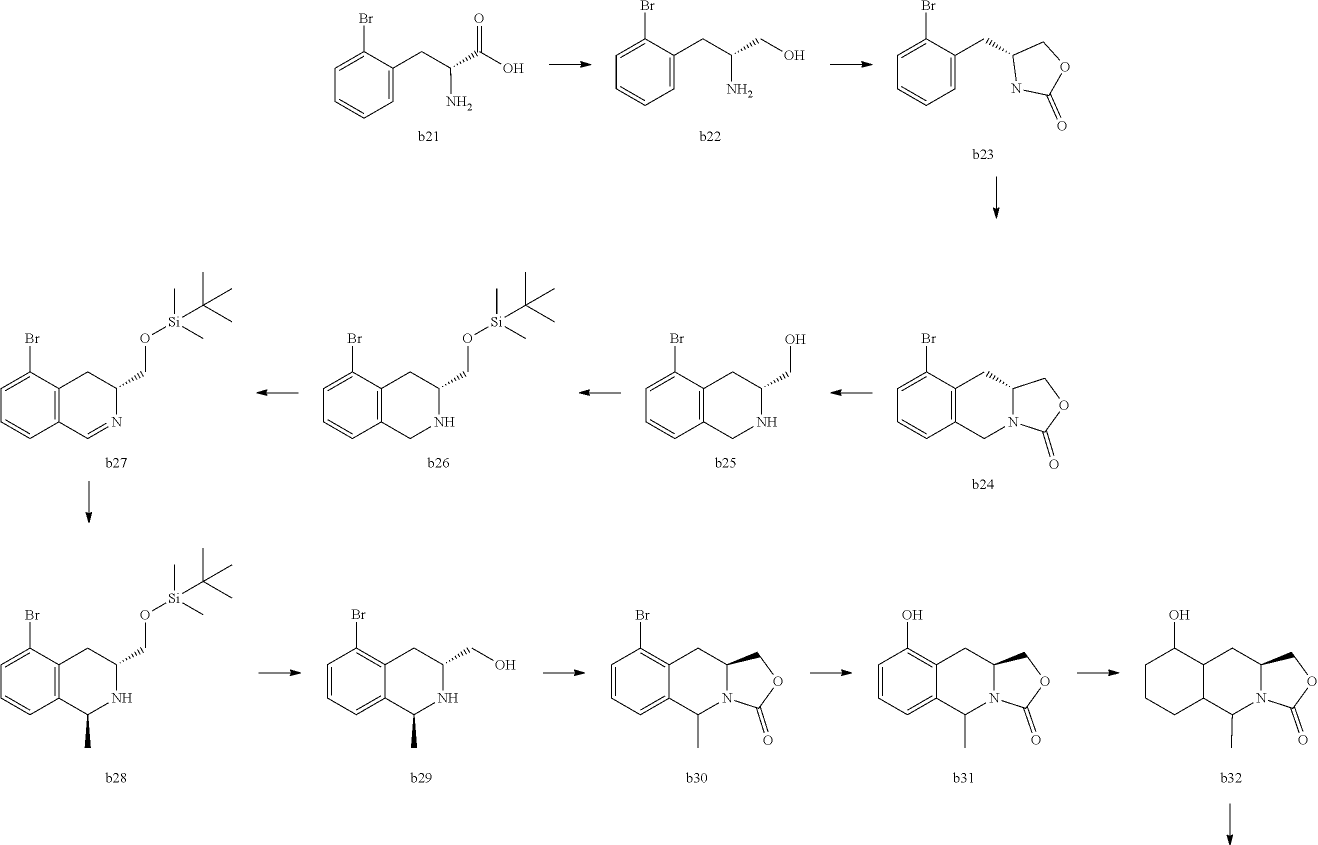

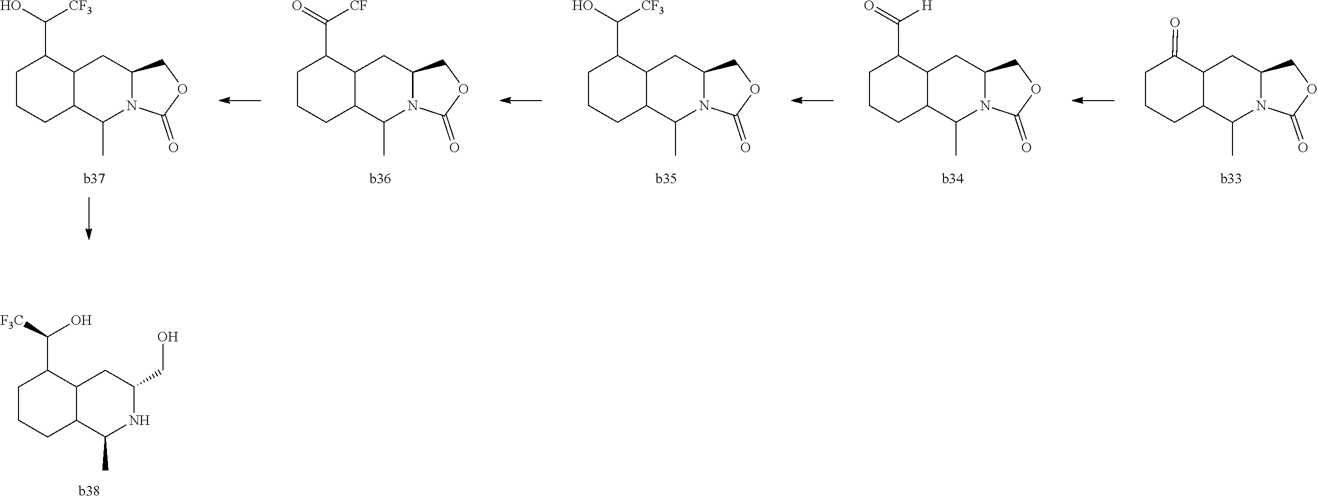

### B.6.1. Synthesis of (2R)-2-amino-3-(2-bromophenyl)propan-1-ol b22

(2R)-2-amino-3-(2-bromophenyl)propanoic acid b21 (34.0 kg, 139 mol) and THF (238 L) were charged into a reactor. Sodium borohydride (15.6 kg, 413 mol) was added slowly at 20-30° C. A solution of I₂ (35.3 kg, 139 mol) in dry THF (20 L) was added slowly at 0-10° C. and the reaction mixture was stirred at 70° C. for 12 h. The reaction was quenched with MeOH (70 L) at 0° C. and heated to 80° C. for 30 min. The mixture was cooled down, concentrated under vacuum. The crude residue was suspended in a 2N aqueous solution of NaOH (30 L), then filtered. The filter cake was dried under vacuum to afford 31.0 kg of (2R)-2-amino-3-(2-bromophenyl)propan-1-ol b22 as a white solid, which was used in the next step without further purification.

Yield (crude): 97%

¹H NMR (400 MHz, CDCl₃): δ 7.57 (d, J=7.7 Hz, 1H), 7.21-7.29 (m, 2H), 7.07-7.15 (m, 1H), 3.66 (dd, J=10.5, 3.6 Hz, 1H), 3.41 (dd, J=10.5, 7.2 Hz, 1H), 3.18-3.29 (m, 1H), 2.95 (dd, J=13.5, 5.5 Hz, 1H), 2.70 (dd, J=13.5, 8.2 Hz, 1H), 1.51-1.91 (m, 3H).

### B.6.2. Synthesis of (4R)-4-[(2-bromophenyl)methyl]oxazolidin-2-one b23

(2R)-2-Amino-3-(2-bromophenyl)propan-1-ol b22 (31.0 kg, 135 mol) and DCM (220 L) were charged into a reactor. Bis(trichloromethyl) carbonate (13.9 kg, 47.1 mol) was added at rt, then DIPEA (39.1 kg, 303 mol) was slowly added at 0-10° C. The reaction mixture was stirred at 0-10° C. for 1 h, then washed with H₂O (50 L) twice, dried over anhydrous Na₂SO₄ and filtered to give (4R)-4-[(2-bromophenyl)methyl]oxazolidin-2-one b23 as a solution in dichloromethane, which was used directly in the next step without further purification.

Synthesis of (10aR)-9-bromo-1,5,10,10a-tetrahydrooxazolo[3,4-b]isoquinolin-3-one b24A solution of (4R)-4-[(2-bromophenyl)methyl]oxazolidin-2-one b23 (135 mol) in DCM (220 L) was charged into a reactor and cooled down to 0-5° C. Trimethylsilyl triflate (35.9 kg, 162 mol) and paraformaldehyde (13.3 kg, 148 mol) were added at 0-5° C., then stirred for 2 h at 15-20° C. H₂O (170 L) was added into the mixture which was then extracted twice with DCM (50 L). The organic layer was dried over anhydrous Na₂SO₄, filtered and concentrated under vacuum. A mixture of petroleum ether:EtOAc (1:1, 45 L) was added. The mixture was stirred at rt for 6 h, then the obtained solid was filtered and dried to afford 29.0 kg of (10aR)-9-bromo-1,5,10,10a-tetrahydrooxazolo[3,4-b]isoquinolin-3-one b24 as an off-white solid.

Yield: 80%

¹H NMR (400 MHz, CDCl₃): δ 7.45-7.52 (m, 1H), 7.08-7.14 (m, 2H), 4.83 (d, J=17.0 Hz, 1H), 4.62 (t, J=8.4 Hz, 1H), 4.36 (d, J=17.0 Hz, 1H), 4.21 (dd, J=8.6, 4.9 Hz, 1H), 3.91-3.99 (m, 1H), 3.25 (dd, J=16.3, 4.2 Hz, 1H), 2.67 (dd, J=16.1, 11.0 Hz, 1H).

### B.6.3. Synthesis of [(3R)-5-bromo-1,2,3,4-tetrahydroisoquinolin-3-yl]methanol b25

EtOH (120 L) and H₂O (60.0 L) were mixed into a reactor. (10aR)-9-bromo-1,5,10,10a-tetrahydrooxazolo[3,4-b]isoquinolin-3-one b24 (29.7 kg, 111 mol) was added, then NaOH (13.3 kg, 332 mol) was slowly added at 15-20° C. The reaction mixture was stirred at 90° C. for 2 h, then cooled down to rt. H₂O (300 L) was added into the mixture which was centrifugated. The centrifugal cake was dried in circulation oven to afford 23.7 kg of [(3R)-5-bromo-1,2,3,4-tetrahydroisoquinolin-3-yl]methanol b25 as a white solid, which was used in the next step without further purification.

Yield (crude): 88%

¹H NMR (400 MHz, CDCl₃): δ 7.37-7.47 (m, 1H), 6.95-7.08 (m, 2H), 4.00-4.10 (m, 2H), 3.85 (dd, J=10.9, 3.7 Hz, 1H), 3.57 (dd, J=10.9, 7.9 Hz, 1H), 3.06 (ddt, J=11.3, 7.6, 4.1, 4.1 Hz, 1H), 2.79 (dd, J=17.1, 4.4 Hz, 1H), 2.40 (dd, J=17.1, 10.9 Hz, 1H), 1.93 (br s, 2H).

### B.6.4. Synthesis of [(3R)-5-bromo-1,2,3,4-tetrahydroisoquinolin-3-yl]methoxy-tert-butyl-dimethyl-silane b26

[(3R)-5-bromo-1,2,3,4-tetrahydroisoquinolin-3-yl]methanol b25 (23.7 kg, 97.8 mol) and DCM (240 L) were charged into a reactor. DMAP (120 g, 978 mmol) and imidazole (13.3 kg, 196 mol) were added. tert-Butyldimethylsilyl chloride (17.7 kg, 117 mol) was slowly added at 15-20° C. and the mixture was stirred for 12 h. A saturated solution of NH₄Cl (100 L) was added into the mixture. The organic phase was washed with H₂O (50 L), dried over anhydrous Na₂SO₄, filtered and concentrated under vacuum to afford 37.6 kg of [(3R)-5-bromo-1,2,3,4-tetrahydroisoquinolin-3-yl]methoxy-tert-butyl-dimethyl-silane b26 as a yellow oil, which is used in the next step without further purification.

Yield (crude): 93%

¹H NMR (400 MHz, CDCl₃): δ 7.36-7.45 (m, 1H), 7.01 (d, J=4.6 Hz, 1H), 4.01-4.13 (m, 2H), 3.84 (dd, J=9.9, 3.7 Hz, 1H), 3.64 (dd, J=9.8, 7.2 Hz, 1H), 2.96-3.08 (m, 1H), 2.75 (dd, J=17.0, 4.2 Hz, 1H), 2.44 (dd, J=17.0, 10.8 Hz, 1H), 1.76-2.20 (m, 2H), 0.89-0.97 (m, 9H), 0.08-0.14 (m, 6H).

### B.6.5. Synthesis of [(3R)-5-bromo-3,4-dihydroisoquinolin-3-yl]methoxy-tert-butyl-dimethyl-silane b27

[(3R)-5-bromo-1,2,3,4-tetrahydroisoquinolin-3-yl]methoxy-tert-butyl-dimethyl-silane b26 (3.42 kg, 8.31 mol) and THF (30 L) were charged into a reactor. NCS (1.17 kg, 8.73 mol) was slowly added at rt. The reaction mixture was stirred at rt for 30 min, then a solution of KOH (1.52 kg, 27.0 mol) in dry MeOH (7 L) was slowly added at rt. The mixture was stirred at rt for 1 h, then quenched with water (10 L) and extracted with a solution of petroleum ether:EtOAc (1:2, 5 L). The organic layer was washed with brine (10 L), dried over anhydrous Na₂SO₄ and filtered. This overall procedure was carried out on 10 batches of the same size in parallel and the 10 reaction filtrates were combined and concentrated under vacuum to afford 28.0 kg of [(3R)-5-bromo-3,4-dihydroisoquinolin-3-yl]methoxy-tert-butyl-dimethyl-silane b27 as a crude brown oil which was used in the next step without further purification.

Yield (crude): 95%

¹H NMR (400 MHz, CDCl₃): δ 8.24 (d, J=2.6 Hz, 1H), 7.58 (dd, J=7.8, 1.2 Hz, 1H), 7.12-7.25 (m, 2H), 4.03 (dd, J=9.5, 4.0 Hz, 1H), 3.67-3.77 (m, 2H), 3.07 (dd, J=17.0, 6.2 Hz, 1H), 2.68 (dd, J=17.1, 10.9 Hz, 1H), 0.88-0.91 (m, 9H), 0.07 (d, J=1.5 Hz, 6H).

### B.6.6. Synthesis of [(1S,3R)-5-bromo-1-methyl-1,2,3,4-tetrahydroisoquinolin-3-yl]methoxy-tert-butyl-dimethyl-silane b28

[(3R)-5-bromo-3,4-dihydroisoquinolin-3-yl]methoxy-tert-butyl-dimethyl-silane b27 (3.10 kg, 8.75 mol) and THF (20 L) were charged into a reactor. The mixture was cooled down to 0° C. and a 3 M solution of methylmagnesium chloride in THF (11.6 L, 34.8 mol) was added. The mixture was stirred at rt for 12 h. The reaction was quenched with a saturated aqueous solution of NH₄Cl. The aqueous layer was extracted twice with petroleum ether:EtOAc (3:1, 5 L). The organic layer was washed with brine (10 L), dried over anhydrous Na₂SO₄ and filtered. This overall procedure was carried out on 9 batches of the same size in parallel and the 9 reaction filtrates were combined and concentrated under vacuum. The crude residue was purified by normal phase column chromatography (elution: 9% EtOAc in petroleum ether) to afford 4.60 kg of [(1S,3R)-5-bromo-1-methyl-1,2,3,4-tetrahydroisoquinolin-3-yl]methoxy-tert-butyl-dimethyl-silane b28 as a brown oil.

Yield: 16%

¹H NMR (400 MHz, DMSO-d₆): δ 7.41 (dd, J=7.7, 0.9 Hz, 1H), 7.12-7.18 (m, 1H), 7.03-7.11 (m, 1H), 4.12 (q, J=6.8 Hz, 1H), 3.62 (d, J=5.7 Hz, 2H), 3.07-3.17 (m, 1H), 2.67-2.76 (m, 1H), 2.26 (dd, J=16.9, 10 Hz, 1H), 2.12 (br s, 1H), 1.32 (d, J=6.8 Hz, 3H), 0.84-0.93 (m, 9H), 0.07 (d, J=0.9 Hz, 6H).

### B.6.7. Synthesis of [(1S,3R)-5-bromo-1-methyl-1,2,3,4-tetrahydroisoquinolin-3-yl]methanol hydrochloride b29

To a solution of [(1S,3R)-5-bromo-1-methyl-1,2,3,4-tetrahydroisoquinolin-3-yl]methoxy-tert-butyl-dimethyl-silane (51.9 g, 140 mmol) b28 in iPrOH (100 mL) was added dropwise a 4N solution of HCl in 1,4-dioxane (200 mL, 800 mmol) at 0° C. and the resulting mixture was allowed to warm to rt overnight. The reaction mixture was concentrated under vacuum to afford 44.3 g of [(1 S,3R)-5-bromo-1-methyl-1,2,3,4-tetrahydroisoquinolin-3-yl]methanol b29 as an hydrochloride salt, which was used in the next step without further purification.

Yield (crude): 97%

Basic LCMS Method 2 (ES⁺): 256/258 (M+H)⁺

### B.6.8. Synthesis of (5S,10aR)-9-bromo-5-methyl-1,5,10,10a-tetrahydrooxazolo[3,4-b]isoquinolin-3-one b30

To a solution of [(1S,3R)-5-bromo-1-methyl-1,2,3,4-tetrahydroisoquinolin-3-yl]methanol hydrochloride b29 (44.0 g, 140 mmol) in DCM (400 mL) and DMF (100 mL), 1,1′-carbonyldiimidazole (44.2 g, 273 mmol) was added at rt. The reaction mixture was stirred for 15 min and DIPEA (115 mL, 660 mmol) was added dropwise. The reaction mixture was stirred overnight at rt. The reaction mixture was diluted with DCM (200 mL). The organic layer was washed with a 1 N aqueous solution of HCl (2×500 mL) and with H₂O (500 mL). The organic layer was dried over MgSO₄, filtered and concentrated under vacuum to afford 41.2 g of (5S,10aR)-9-bromo-5-methyl-1,5,10,10a-tetrahydrooxazolo[3,4-b]isoquinolin-3-one b30, which was used in the next step without further purification.

Yield (crude): quantitative

Acic LCMS Method 1 (ES⁺): 282/284 (M+H)⁺.

### B.6.9. Synthesis of (5S,10aR)-9-hydroxy-5-methyl-1,5,10,10a-tetrahydrooxazolo[3,4-b]isoquinolin-3-one b31

To a solution of (5S,10aR)-9-bromo-5-methyl-1,5,10,10a-tetrahydrooxazolo[3,4-b]isoquinolin-3-one b30 (41.2 g, 136 mmol) in 1,4-dioxane (340 mL) was added KOH (18.5 g, 296 mmol) in solution in H₂O (85.0 mL). The reaction mixture was flushed with nitrogen at 95° C. Then, 2-di-tert-butylphosphino-2′,4′,6′-triisopropylbiphenyl (804 mg, 1.86 mmol) and tris(dibenzylideneacetone)dipalladium(0) (3.17 g, 3.46 mmol) were added and the reaction mixture was stirred at 95° C. for 3 h. The reaction mixture was filtered through a pad of Celite® and concentrated under vacuum. The resulting residue was poured in DCM (500 mL) and washed with a 1 N aqueous solution of HCl (250 mL). The organic layer and the aqueous layer were separated. The suspended solid in the aqueous layer was filtered and dried under vacuum at 45° C. overnight to afford 15.6 g of (5S,10aR)-9-hydroxy-5-methyl-1,5,10,10a-tetrahydrooxazolo[3,4-b]isoquinolin-3-one b31 as an off-white solid, which was used in the next step without further purification

Yield (crude): 52%

Acic LCMS Method 1 (ES⁺): 220 (M+H)⁺

### B.6.10. Synthesis of (5S,10aR)-9-hydroxy-5-methyl-1,5,5a,6,7,8,9,9a,10,10a-decahydrooxazolo[3,4-b]isoquinolin-3-one (mixture of 8 epimers) b32

To a solution of (5S,10aR)-9-hydroxy-5-methyl-1,5,10,10a-tetrahydrooxazolo[3,4-b]isoquinolin-3-one b31 (15.6 g, 71.2 mmol) in iPrOH (150 mL), a 1N aqueous solution of NaOH (14.0 mL, 14.0 mmol) and Rh/C JM Type 20D (2.10 g, 1.00 mmol) were added. The autoclave was pressurized with 50 bars of H₂. The reaction mixture was heated at 100° C. under a vigorous stirring during 3 days. Rh/C JM Type 20D (1.00 g, 0.486 mmol) was added and the reaction mixture was again pressurized with 50 bars of H₂ and heated overnight at 100° C. The reaction mixture was cooled down to rt. The reaction mixture was filtered through a pad of Celite®. Rh/C JM Type 20D (5 g, 2.43 mmol) was added and the reaction mixture was again pressurized with 50 bars of H₂ and heated overnight at 100° C. The reaction mixture was successively filtered through a pad of Celite® and through a SPE Syringe, then concentrated under vacuum. The crude residue was poured into a 0.5N aqueous solution of NaOH (200 mL) and the aqueous layer was extracted with IPAC (3×250 mL). The organic layer was dried over MgSO₄, filtered and concentrated under vacuum to afford 8.80 g of (5S,10aR)-9-hydroxy-5-methyl-1,5,5a,6,7,8,9,9a,10,10a-decahydrooxazolo[3,4-b]isoquinolin-3-one b32 as a mixture of 8 epimers, which was used in the next step without further purification.

Yield (crude): 44%

Acic LCMS Method 1 (ES⁺): 226 (M+H)⁺.

### B.6.11. Synthesis of the isomeric mixture b33: (5S,5aS,9aR,10aR)-5-methyl-5,5a,6,7,8,9a,10,10a-octahydro-1H-oxazolo[3,4-b]isoquinoline-3,9-dione b33-A and (5S,5aR,9aS,10aR)-5-methyl-5,5a,6,7,8,9a,10,10a-octahydro-1H-oxazolo[3,4-b]isoquinoline-3,9-dione b33-B

Dess-Martin periodinane (53.3 mmol, 23.3 g) was added to a solution of (5S,10aR)-9-hydroxy-5-methyl-1,5,5a,6,7,8,9,9a,10,10a-decahydrooxazolo[3,4-b]isoquinolin-3-one b32 (26.6 mmol, 6.00 g) in DCM (250 mL). The reaction mixture was stirred over 48 h at rt. The reaction mixture was diluted with DCM (500 mL), and successively washed with a saturated aqueous solution of sodium carbonate (2×200 mL) and brine (150 mL). The organic layer was dried over MgSO₄, filtered and concentrated under vacuum to afford 5.00 g of crude (5S,5aS,9aR,10aR)-5-methyl-5,5a,6,7,8,9a,10,10a-octahydro-1H-oxazolo[3,4-b]isoquinoline-3,9-dione b33 as a mixture of trans epimers b33-A and b33-B, which was used in the next step without further purification.

Yield (crude): 84%

Basic LCMS Method 1 (ES⁺): 224 (M+H)⁺.

Attribution of the stereochemistry was done according to the literature. Trans isomers are favored. The crude was considered as a mixture of mainly trans isomers: (5S,5aS,9aR,10aR)-5-methyl-5,5a,6,7,8,9a,10,10a-octahydro-1H-oxazolo[3,4-b]isoquinoline-3,9-dione b33-A and (5S,5aR,9aS,10aR)-5-methyl-5,5a,6,7,8,9a,10,10a-octahydro-1H-oxazolo[3,4-b]isoquinoline-3,9-dione b33-B. The cis isomers were present in minor quantity and were considered as marginal. They were discarded during the next steps of the synthesis during the multiple purification processes.

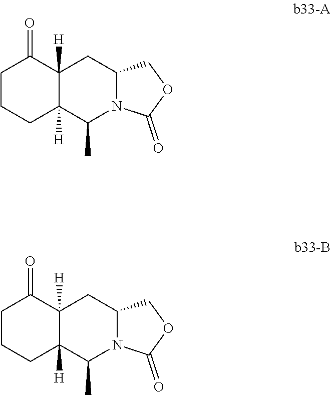 [SMILES: C[C@H]1[C@H]2CCCC(=O)[C@@H]2C[C@@H]2COC(=O)N21 ; C[C@H]1[C@@H]2CCCC(=O)[C@H]2C[C@@H]2COC(=O)N21]

### B.6.12. Synthesis of the isomeric mixture b34: (5S,5aS,9R,9aR,10aR)-5-methyl-3-oxo-1,5,5a,6,7,8,9,9a,10,10a-decahydrooxazolo[3,4-b]isoquinoline-9-carbaldehyde b34-A and (5S,5aR,9S,9aS,10aR)-5-methyl-3-oxo-1,5,5a,6,7,8,9,9a,10,10a-decahydrooxazolo[3,4-b]isoquinoline-9-carbaldehyde b34-B

Sodium tert-butoxide (2.88 g, 29.1 mmol) was added to a solution of (methoxymethyl)triphenylphosphonium chloride (10.7 g, 31.5 mmol) in THF (100 mL) at −78° C. under argon. The reaction mixture was stirred 15 min at 0° C. The reaction mixture was cooled again at −78° C. before adding the mixture of isomers b33 (5.00 g, 22.4 mmol). The reaction mixture was then stirred for 3 days at rt. The reaction mixture was diluted with EtOAc (300 mL) and successively washed with a saturated aqueous solution of sodium carbonate (100 mL) and brine (100 mL). The organic layer was dried over MgSO₄, filtered and concentrated under vacuum. The crude residue was purified by normal phase column chromatography (elution: 10% EtOAc in heptane) to remove the residual triphenylphosphine oxide. The residue was diluted in a mixture of a 1 N aqueous solution of HCl (50 mL) and THF (50 mL), then the mixture was stirred overnight at rt. H₂O (100 mL) was added and the mixture was extracted with DCM (3×200 mL). The organic layer was washed with brine, dried over MgSO₄, filtered and concentrated under vacuum to afford 2.80 g of b34 as a mixture of isomers b34-A and b34-B, which was used in the next step without further purification.

Yield (crude): 53%

Basic LCMS Method 1 (ES⁺): 238 (M+H)⁺

Attribution of the stereochemistry was done according to the literature. Equatorial aldehydes are favored. The crude was considered as a mixture of mainly trans isomers bearing an equatorial aldehyde: (5S,5aS,9R,9aR,10aR)-5-methyl-3-oxo-1,5,5a,6,7,8,9,9a,10,10a-decahydrooxazolo[3,4-b]isoquinoline-9-carbaldehyde b34-A and (5S,5aR,9S,9aS,10aR)-5-methyl-3-oxo-1,5,5a,6,7,8,9,9a,10,10a-decahydrooxazolo[3,4-b]isoquinoline-9-carbaldehyde b34-B

The other minor isomers were considered as marginal and were discarded during the next steps of the synthesis in the multiple purification processes.

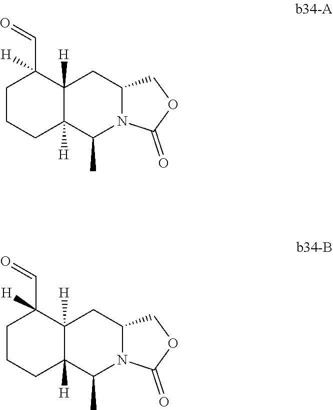 [SMILES: C[C@H]1[C@H]2CCC[C@@H](C=O)[C@@H]2C[C@@H]2COC(=O)N21 ; C[C@H]1[C@@H]2CCC[C@H](C=O)[C@H]2C[C@@H]2COC(=O)N21]

### B.6.13. Synthesis of the isomeric mixture b35: (5S,5aS,9R,9aR,10aR)-5-methyl-9-(2,2,2-trifluoro-1-hydroxy-ethyl)-1,5,5a,6,7,8,9,9a,10,10a-decahydrooxazolo[3,4-b]isoquinolin-3-one b35-A and (5S,5aR,9S,9aS,10aR)-5-methyl-9-(2,2,2-trifluoro-1-hydroxy-ethyl)-1,5,5a,6,7,8,9,9a,10,10a-decahydrooxazolo[3,4-b]isoquinolin-3-one b35-B

Cesium fluoride (272 g, 17.7 mmol) was added to a solution of the isomeric mixture b34 (2.80 g, 11.8 mmol) and (trifluoromethyl)trimethylsilane (2.52 g, 17.7 mmol) in DMF (40 mL) at 0° C. under argon. The reaction mixture was stirred 5 min at 0° C. After quenching with a saturated aqueous solution of NH₄Cl (10 mL), the reaction mixture was extracted with EtOAc (150 mL). The organic layer was dried over MgSO₄, filtered and concentrated under vacuum to afford 2.90 g of b35 as a mixture of isomers (5S,5aS,9R,9aR,10aR)-5-methyl-9-(2,2,2-trifluoro-1-hydroxy-ethyl)-1,5,5a,6,7,8,9,9a,10,10a-decahydrooxazolo[3,4-b]isoquinolin-3-one b35-A and (5S,5aR,9S,9aS,10aR)-5-methyl-9-(2,2,2-trifluoro-1-hydroxy-ethyl)-1,5,5a,6,7,8,9,9a,10,10a-decahydrooxazolo[3,4-b]isoquinolin-3-one b35-B, which was used in the next step without further purification.

Yield (crude): 80%

Basic LCMS Method 1 (ES⁺): 308 (M+H)⁺

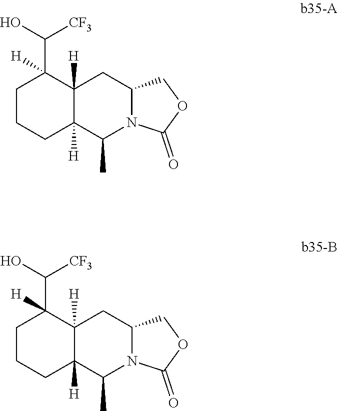 [SMILES: C[C@H]1[C@H]2CCC[C@@H](C(O)C(F)(F)F)[C@@H]2C[C@@H]2COC(=O)N21 ; C[C@H]1[C@@H]2CCC[C@H](C(O)C(F)(F)F)[C@H]2C[C@@H]2COC(=O)N21]

### B.6.14. Synthesis of the isomeric mixture b36: (5S,5aS,9R,9aR,10aR)-5-methyl-9-(2,2,2-trifluoroacetyl)-1,5,5a,6,7,8,9,9a,10,10a-decahydrooxazolo[3,4-b]isoquinolin-3-one b36-A and (5S,5aR,9S,9aS,10aR)-5-methyl-9-(2,2,2-trifluoroacetyl)-1,5,5a,6,7,8,9,9a,10,10a-decahydrooxazolo[3,4-b]isoquinolin-3-one b36-B

Dess-Martin periodinane (7.21 g, 16.5 mmol) was added to a solution of the isomeric mixture b35 (3.38 g, 11.0 mmol) in DCM (50 mL) at 0° C. under argon. The reaction mixture was stirred 2 h at 0° C. The reaction mixture was diluted with DCM (150 mL), then successively washed with a 1 N aqueous solution of HCl (50 mL), a saturated aqueous solution of sodium carbonate (50 mL) and brine (50 mL). The organic layer was dried over MgSO₄, filtered and concentrated under vacuum to afford 2.20 g of b36 as a mixture of (5S,5aS,9R,9aR,10aR)-5-methyl-9-(2,2,2-trifluoroacetyl)-1,5,5a,6,7,8,9,9a,10,10a-decahydrooxazolo[3,4-b]isoquinolin-3-one b36-A and (5S,5aR,9S,9aS,10aR)-5-methyl-9-(2,2,2-trifluoroacetyl)-1,5,5a,6,7,8,9,9a,10,10a-decahydrooxazolo[3,4-b]isoquinolin-3-one b36-B, which was used in the next step without further purification.

Yield (crude): 76%

Basic LCMS Method 1 (ES⁺): 306 (M+H)⁺

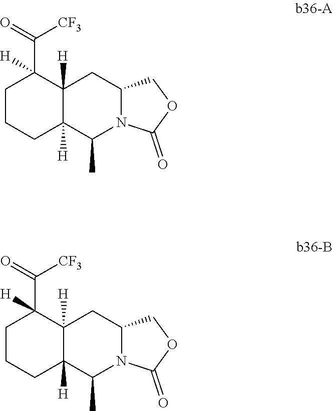 [SMILES: C[C@H]1[C@H]2CCC[C@@H](C(=O)C(F)(F)F)[C@@H]2C[C@@H]2COC(=O)N21 ; C[C@H]1[C@@H]2CCC[C@H](C(=O)C(F)(F)F)[C@H]2C[C@@H]2COC(=O)N21]

### B.6.15. Synthesis of the isomeric mixture b37: (5S,5aR,9S,9aS,10aR)-5-methyl-9-[(1S)-2,2,2-trifluoro-1-hydroxy-ethyl]-1,5,5a,6,7,8,9,9a,10,10a-decahydrooxazolo[3,4-b]isoquinolin-3-one b37-A and (5S,5aS,9R,9aR,10aR)-5-methyl-9-[(1R)-2,2,2-trifluoro-1-hydroxy-ethyl]-1,5,5a,6,7,8,9,9a,10,10a-decahydrooxazolo[3,4-b]isoquinolin-3-one b37-B

Lithium tri-sec-butylborohydride (4.70 g, 5.40 mmol) was added dropwise on a solution of the isomeric mixture b36 (1.10 g, 3.60 mmol) in THF (50 mL) at −78° C. The mixture was stirred overnight while warming up to rt. The reaction mixture was diluted with DCM (150 mL) and successively washed with a 1N aqueous solution of HCl (50 mL), a saturated aqueous solution of sodium carbonate (50 mL) and brine (50 mL). The organic layer was dried over MgSO₄, filtered and concentrated under vacuum to afford 1.10 g of b37 as a mixture of isomers (5S,5aR,9S,9aS,10aR)-5-methyl-9-[(1 S)-2,2,2-trifluoro-1-hydroxy-ethyl]-1,5,5a,6,7,8,9,9a,10,10a-decahydrooxazolo[3,4-b]isoquinolin-3-one b37-A and (5S,5aS,9R,9aR,10aR)-5-methyl-9-[(1R)-2,2,2-trifluoro-1-hydroxy-ethyl]-1,5,5a,6,7,8,9,9a,10,10a-decahydrooxazolo[3,4-b]isoquinolin-3-one b37-B, which was used in the next step without further purification.

Yield (crude): 100%

Basic LCMS Method 1 (ES⁺): 308 (M+H)⁺

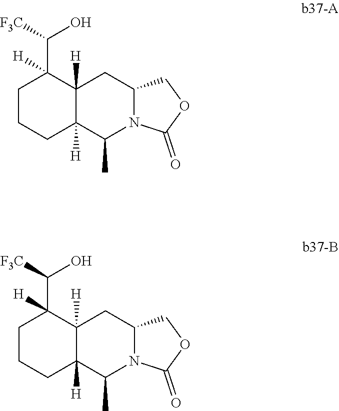 [SMILES: C[C@H]1[C@H]2CCC[C@@H]([C@@H](O)C(F)(F)F)[C@@H]2C[C@@H]2COC(=O)N21 ; C[C@H]1[C@@H]2CCC[C@H]([C@H](O)C(F)(F)F)[C@H]2C[C@@H]2COC(=O)N21]

### B.6.16. Synthesis of the isomeric mixture b38: (1S)-1-[(1S,3R,4aS,5S,8aR)-3-(hydroxymethyl)-1-methyl-1,2,3,4,4a,5,6,7,8,8a-decahydroisoquinolin-5-yl]-2,2,2-trifluoro-ethanol b38-A and (1R)-1-[(1S,3R,4aR,5R,8aS)-3-(hydroxymethyl)-1-methyl-1,2,3,4,4a,5,6,7,8,8a-decahydroisoquinolin-5-yl]-2,2,2-trifluoro-ethanol b38-B

The isomeric mixture b37 (1.10 g, 3.58 mmol) was dissolved in a mixture of a 4N aqueous solution of NaOH (2 mL) and EtOH (6 mL). The reaction mixture was stirred overnight at 80° C. Volatiles were removed under reduced pressure. The reaction mixture was extracted with DCM (3×15 mL). The organic layer was dried over MgSO₄, filtered and concentrated under vacuum. The crude residue was diluted in MeOH (10 mL) and was eluted through an ion-exchange column filled with an acidic polymer (Waters™ PoraPak Rxn CX 60 cc Vac Cartridge, 5 g sorbent per cartridge, 80 μm). The compound was trapped on an acidic polymer. After rinsing the polymer, the compound was extracted with a 2 M solution of ammonia. Volatiles were evaporated to afford 700 mg of b38 as a white solid and as a mixture of isomers (1S)-1-[(1S,3R,4aS,5S,8aR)-3-(hydroxymethyl)-1-methyl-1,2,3,4,4a,5,6,7,8,8a-decahydroisoquinolin-5-yl]-2,2,2-trifluoro-ethanol b38-A and (1R)-1-[(1 S,3R,4aR,5R,8aS)-3-(hydroxymethyl)-1-methyl-1,2,3,4,4a,5,6,7,8,8a-decahydroisoquinolin-5-yl]-2,2,2-trifluoro-ethanol b38-B, which was used in the next step without further purification.

Yield: 69%

Basic LCMS Method 1 (ES⁺): 282 (M+H)⁺

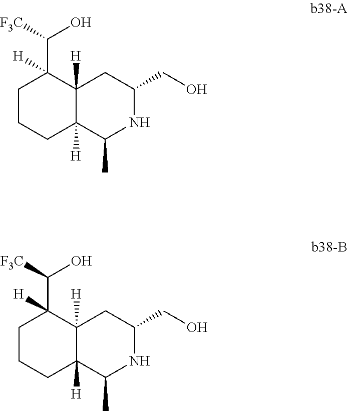 [SMILES: C[C@@H]1N[C@@H](CO)C[C@H]2[C@H]([C@@H](O)C(F)(F)F)CCC[C@@H]21 ; C[C@@H]1N[C@@H](CO)C[C@H]2[C@H]1CCC[C@@H]2[C@H](O)C(F)(F)F]

### EXAMPLES

### C. Synthesis of Compounds of Formula (I)

### C.1. Synthesis of 2-[2-[(1S,4aR,5R,8aS)-1-methyl-5-[(1S)-2,2,2-trifluoro-1-hydroxy-ethyl]-3,4,4a,5,6,7,8,8a-octahydro-1H-isoquinolin-2-yl]-2-oxo-ethyl]-3-chloro-4-methoxybenzonitrile 1-A and 2-[2-[(1S,4aR,5R,8aS)-1-methyl-5-[(1R)-2,2,2-trifluoro-1-hydroxy-ethyl]-3,4,4a,5,6,7,8,8a-octahydro-1H-isoquinolin-2-yl]-2-oxo-ethyl]-3-chloro-4-methoxybenzonitrile 1-B

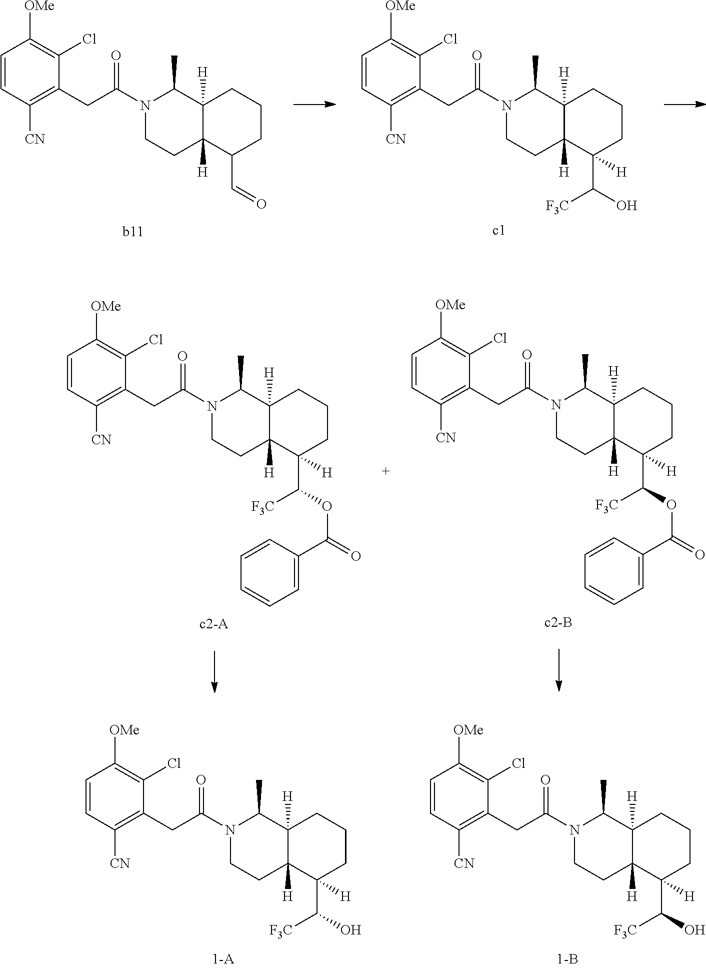 [SMILES: COc1ccc(C#N)c(CC(=O)N2CC[C@H]3C(C=O)CCC[C@@H]3[C@@H]2C)c1Cl ; COc1ccc(C#N)c(CC(=O)N2CC[C@@H]3[C@H](CCC[C@H]3C(O)C(F)(F)F)[C@@H]2C)c1Cl ; COc1ccc(C#N)c(CC(=O)N2CC[C@H]3[C@H]([C@H](OC(=O)c4ccccc4)C(F)(F)F)CCC[C@@H]3[C@@H]2C)c1Cl ; COc1ccc(C#N)c(CC(=O)N2CC[C@H]3[C@H]([C@@H](OC(=O)c4ccccc4)C(F)(F)F)CCC[C@@H]3[C@@H]2C)c1Cl ; COc1ccc(C#N)c(CC(=O)N2CC[C@H]3[C@H]([C@H](O)C(F)(F)F)CCC[C@@H]3[C@@H]2C)c1Cl ; COc1ccc(C#N)c(CC(=O)N2CC[C@H]3[C@H]([C@@H](O)C(F)(F)F)CCC[C@@H]3[C@@H]2C)c1Cl]

### C.1.1. Synthesis of 2-[2-[(1S,4aR,5R,8aS)-1-methyl-5-(2,2,2-trifluoro-1-hydroxyethyl)-3,4,4a,5,6,7,8,8a-octahydro-1H-isoquinolin-2-yl]-2-oxoethyl]-3-chloro-4-methoxybenzonitrile c1

To a solution of 2-[2-[(1 S,4aR,8aS)-5-formyl-1-methyl-3,4,4a,5,6,7,8,8a-octahydro-1H-isoquinolin-2-yl]-2-oxoethyl]-3-chloro-4-methoxybenzonitrile b1l (9.40 g, 24.0 mmol) in DMF (40 mL) was added cesium fluoride (7.40 g, 48.0 mmol) at rt. Then, the reaction mixture was cooled down to 5° C. and (trifluoromethyl)trimethylsilane (0.7 mL, 492 mmol) was added dropwise over a period of 30 min and the reaction mixture was allowed to stir overnight at rt. IPAC (150 mL) was added to the reaction mixture followed by the addition of a 5 N aqueous solution of HCl (200 mL). The reaction mixture was stirred at rt for 72 h and was washed successively with a 1N aqueous solution of HCl (100 mL) and water (100 mL). The organic layer was dried over MgSO₄, filtered and concentrated under vacuum to afford 10.7 g of 2-[2-[(1 S,4aR,5R,8aS)-1-methyl-5-(2,2,2-trifluoro-1-hydroxyethyl)-3,4,4a,5,6,7,8,8a-octahydro-1H-isoquinolin-2-yl]-2-oxoethyl]-3-chloro-4-methoxybenzonitrile c1 as a white foam, which was used in next step without further purification.

Yield (crude): 92%

Basic LCMS Method 2 (ES⁺): 459 (M+H)⁺.

### C.1.2. Synthesis of [(1S)-1-[(1S,4aR,5R,8aS)-2-[2-(2-chloro-6-cyano-3-methoxyphenyl)acetyl]-1-methyl-3,4,4a,5,6,7,8,8a-octahydro-1H-isoquinoin-5-y]-2,2,2-trifluoroethyl] benzoate c2-A and [(1R)-1-[(1S,4aR,5R,8aS)-2-[2-(2-chloro-6-cyano-3-methoxyphenyl)acetyl]-1-methyl-3,4,4a,5,6,7,8,8a-octahydro-1H-isoquinolin-5-yl]-2,2,2-trifluoroethyl] benzoate c2-B

To a solution of 2-[2-[(1 S,4aR,5R,8aS)-1-methyl-5-(2,2,2-trifluoro-1-hydroxyethyl)-3,4,4a,5,6,7,8,8a-octahydro-1H-isoquinolin-2-yl]-2-oxoethyl]-3-chloro-4-methoxybenzonitrile c1 (143 mg, 0.31 mmol) in DCM (1.6 mL) were added pyridine (110 μL, 1.37 mmol), DMAP (8.00 mg, 65.0 μmol) and benzoyl chloride (73.0 μL, 0.62 mmol) at rt. The reaction mixture was stirred overnight at rt, then benzoyl chloride (36.0 μL, 0.31 mmol) was added at rt. The reaction mixture was stirred at rt for 4 h, then diluted with DCM and washed with a saturated aqueous solution of NaHCO₃. The organic layer was dried over MgSO₄, filtered and concentrated under vacuum. The crude residue was purified by preparative TLC using 5% of a 90/10 MeOH/NH₄OH solution in DCM to afford 120 mg of [1-[(1S,4aR,5R,8aS)-2-[2-(2-chloro-6-cyano-3-methoxyphenyl)acetyl]-1-methyl-3,4,4a,5,6,7,8,8a-octahydro-1H-isoquinolin-5-yl]-2,2,2-trifluoroethyl] benzoate c2 a mixture of isomers c2-A and c2-B (Yield: 68%, Basic LCMS Method 2 (ES⁺): 563/565 (M+H)⁺).

Chiral separation (SFC, IA, 50×266 mm, 360 mL/min, 220 nm, 30° C., elution: MeOH 20%—CO₂ 80%) of [1-[(1S,4aR,5R,8aS)-2-[2-(2-chloro-6-cyano-3-methoxyphenyl)acetyl]-1-methyl-3,4,4a,5,6,7,8,8a-octahydro-1H-isoquinolin-5-yl]-2,2,2-trifluoroethyl] benzoate c2 afforded:

44.0 mg of [(1S)-1-[(1 S,4aR,5R,8aS)-2-[2-(2-chloro-6-cyano-3-methoxyphenyl)acetyl]-1-methyl-3,4,4a,5,6,7,8,8a-octahydro-1H-isoquinolin-5-yl]-2,2,2-trifluoroethyl] benzoate c2-A, as a pink solid.

Yield: 25%.

Basic LCMS Method 2 (ES⁺): 563/565 (M+H)⁺, 98% purity.

Chiral analysis (LC, IA, 150*4.6 mm, 1.5 mL/min, 220 nm, 30° C., elution: EtOH/n-heptane/DEA 50/50/0.1): RT 2.10 min, 98% de

54.0 mg of [(1R)-1-[(1 S,4aR,5R,8aS)-2-[2-(2-chloro-6-cyano-3-methoxyphenyl)acetyl]-1-methyl-3,4,4a,5,6,7,8,8a-octahydro-1H-isoquinolin-5-yl]-2,2,2-trifluoroethyl] benzoate c2-B, as a pink solid.

Yield: 31%

Basic LCMS Method 2 (ES⁺): 563/565 (M+H)⁺, 99% purity.

Chiral analysis (LC, AS, 150*4.6 mm, 1.5 mL/min, 220 nm, 30° C., elution: iPrOH/n-heptane/DEA 50/50/0.1): RT 2.59 min, 98% de.

### C.1.3. Synthesis of 2-[2-[(1S,4aR,5R,8aS)-1-methyl-5-[(1S)-2,2,2-trifluoro-1-hydroxy-ethyl]-3,4,4a,5,6,7,8,8a-octahydro-1H-isoquinolin-2-yl]-2-oxo-ethyl]-3-chloro-4-methoxybenzonitrile 1-A and 2-[2-[(1S,4aR,5R,8aS)-1-methyl-5-[(1R)-2,2,2-trifluoro-1-hydroxy-ethyl]-3,4,4a,5,6,7,8,8a-octahydro-1H-isoquinolin-2-yl]-2-oxo-ethyl]-3-chloro-4-methoxybenzonitrile 1-B

To a solution of [(1 S)-1-[(1 S,4aR,5R,8aS)-2-[2-(2-chloro-6-cyano-3-methoxyphenyl)acetyl]-1-methyl-3,4,4a,5,6,7,8,8a-octahydro-1H-isoquinolin-5-yl]-2,2,2-trifluoroethyl] benzoate c2-A (44.0 mg, 78.0 μmol) in EtOH (390 μL) was added a solution of KOH (5.20 mg, 79.0 μmol) in H₂O/EtOH (1:1, 70.0 μL) at rt. The reaction mixture was stirred at rt for 2 h, then concentrated under vacuum. The crude residue was taken up in EtOAc and the solution was washed with H₂O. The organic layer was dried over MgSO₄, filtered and concentrated under vacuum. The crude residue was purified by preparative TLC using 5% of a 90/10 MeOH/NH₄OH solution in DCM to afford 24.0 mg of 2-[2-[(1S,4aR,5R,8aS)-1-methyl-5-[(1S)-2,2,2-trifluoro-1-hydroxy-ethyl]-3,4,4a,5,6,7,8,8a-octahydro-1H-isoquinolin-2-yl]-2-oxo-ethyl]-3-chloro-4-methoxybenzonitrile 1-A, as a white solid.

Yield: 67%.

Acid LCMS Method 2 (ES⁺): 459/461 (M+H)⁺, 100% purity.

Basic LCMS Method 3 (ES⁺): 459/461 (M+H)⁺, 100% purity.

¹H NMR (400 MHz, DMSO-d₆): δ 7.81 (d, J=8.7 Hz, 1H), 7.23 (d, J=8.7 Hz, 1H), 6.05 (dd, J=7.0, 2.7 Hz, 1H), 4.61-4.41 (m, 0.5H), 4.38-4.26 (m, 0.5H), 4.24-4.07 (m, 2H), 4.07-3.87 (m, 5H), 3.26-3.12 (m, 0.5H), 2.66 (m, 0.5H), 2.03-1.82 (m, 1H), 1.75 (t, J=13.8 Hz, 1H), 1.68-1.47 (m, 3H), 1.46-1.23 (m, 4H), 1.22-0.70 (m, 5H).

Chiral analysis (LC, ID, 150×4.6 mm, 1.5 mL/min, 220 nm, 30° C., elution: EtOH/n-heptane/DEA 50/50/0.1): RT 1.70 min, 98% de.

X-Ray diffraction of Example 1-A: A block-like single crystal of Example 1-A was selected and mounted on the inclined MiTeGen MicroLoops E sample holder. Single-crystal X-ray diffraction data were collected using the Oxford Diffraction Gemini R Ultra diffractometer (Mo Kα, graphite monochromator, Ruby CCD area detector). Data collection, unit cells determination and data reduction were carried out using CrysAlis PRO software packagel. Using Olex22 and shelXle3, the structure was solved with the SHELXT 2014/54 structure solution program using Intrinsic Phasing methods and refined by full-matrix least squares on |F|² using SHELXL-2016/65. Non-hydrogen atoms were refined anisotropically. All hydrogen atoms were located from electron density map. Hydrogen atoms of most carbon atoms were placed on calculated positions in riding mode with temperature factors fixed at 1.2 times Ueq of the parent carbon atoms (1.5 for methyl groups).

Crystal Data for C₂₂H₂₆CIF₃N₂O₃ (M=458.90 g/mol): orthorhombic, space group P2₁2₁2₁ (no. 19), a=8.4587 (2) Å, b=10.3992 (4) Å, c=25.2267 (7) Å, V=2219.05 (11) Å³, Z=4, T=295 K, μ(MoKα)=0.223 mm 1, Dcalc=1.374 g/cm³, 11408 reflections measured (4.236°≤2Θ≤55.752°), 5283 unique (Rint=0.0204, Rsigma=0.0300) which were used in all calculations. The final R1 was 0.0421 (I>2σ(I)) and wR² was 0.1055 (all data).

Absolute configuration was established by anomalous-dispersion effects in diffraction measurements on the crystal. Flack x parameter determined using 2219 quotients [(I+)-(I−)]/[(I+)+(I−)] 6 and equal to −0.01 (3) indicated the absolute configuration as displayed in section C.1. above (Example 1-A).

### 2-[2-[(1S,4aR,5R,8aS)-1-methyl-5-[(1R)-2,2,2-trifluoro-1-hydroxy-ethyl]-3,4,4a,5,6,7,8,8a-octahydro-1H-isoquinolin-2-yl]-2-oxo-ethyl]-3-chloro-4-methoxybenzonitrile 1-B

Compound 1-B may be synthesized according to the same method using [(1R)-1-[(1S,4aR,5R,8aS)-2-[2-(2-chloro-6-cyano-3-methoxyphenyl)acetyl]-1-methyl-3,4,4a,5,6,7,8,8a-octahydro-1H-isoquinolin-5-yl]-2,2,2-trifluoroethyl] benzoate c2-B as starting material.

Yield: 68%.

Acid LCMS Method 2 (ES⁺): 459/461 (M+H)⁺, 98% purity.

Basic LCMS Method 3 (ES⁺): 459/461 (M+H)⁺, 98% purity.

Chiral analysis (LC, ID, 150×4.6 mm, 1.5 mL/min, 220 nm, 30° C., elution: EtOH/heptane/DEA 50/50/0.1): RT 2.07 min, 99% de.

### C.2. Synthesis of 2-[2-[(1S,4aR,5R,8aS)-1-methyl-5-[(1S)-2,2,2-trifluoro-1-hydroxyethyl]-3,4,4a,5,6,7,8,8a-octahydro-1H-isoquinolin-2-yl]-2-oxoethyl]-3-chloro-6-methoxybenzonitrile 2

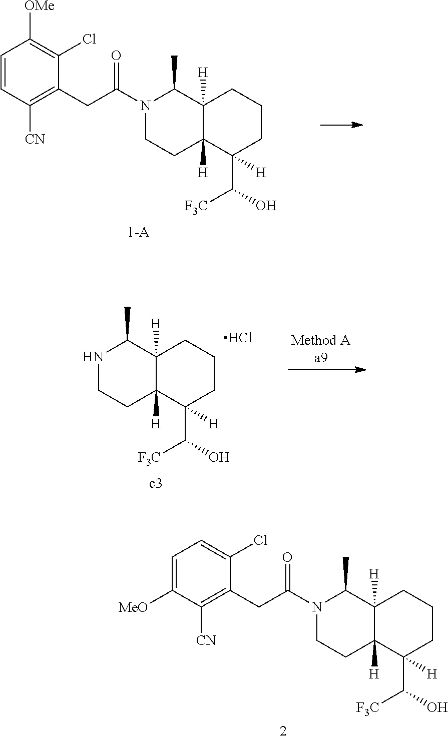 [SMILES: COc1ccc(C#N)c(CC(=O)N2CC[C@H]3[C@H]([C@H](O)C(F)(F)F)CCC[C@@H]3[C@@H]2C)c1Cl ; C[C@@H]1NCC[C@H]2[C@H]([C@H](O)C(F)(F)F)CCC[C@@H]21 ; Cl ; COc1ccc(Cl)c(CC(=O)N2CC[C@H]3[C@H]([C@H](O)C(F)(F)F)CCC[C@@H]3[C@@H]2C)c1C#N]

### C.2.1. Synthesis of (1S)-1-[(1S,4aR,5R,8aS)-1-methyl-1,2,3,4,4a,5,6,7,8,8a-decahydroisoquinolin-5-yl]-2,2,2-trifluoroethanol hydrochloride c3

A suspension of 2-[2-[(1 S,4aR,5R,8aS)-1-methyl-5-[(1S)-2,2,2-trifluoro-1-hydroxy-ethyl]-3,4,4a,5,6,7,8,8a-octahydro-1H-isoquinolin-2-yl]-2-oxo-ethyl]-3-chloro-4-methoxy-benzonitrile 1-A (1.50 g, 3.27 mmol) in a 2N aqueous solution of LiOH (150 mL) was stirred at 130° C. for 3 days. The reaction mixture was extracted with DCM (3×50 mL). The organic layer was washed with a 1 N aqueous solution of HCl (3×50 mL). The acidic aqueous layer was concentrated under vacuum to give 850 mg of (1S)-1-[(1S,4aR,5R,8aS)-1-methyl-1,2,3,4,4a,5,6,7,8,8a-decahydroisoquinolin-5-yl]-2,2,2-trifluoroethanol hydrochloride c3 as white solid, which was used in the next steps without further purification.

Yield (crude): 90%.

Acid LCMS Method 1 (ES⁺): 252 (M+H)⁺.

### C.2.2. Synthesis of 2-[2-[(1S,4aR,5R,8aS)-1-methyl-5-[(1S)-2,2,2-trifluoro-1-hydroxy-ethyl]-3,4,4a,5,6,7,8,8a-octahydro-1H-isoquinolin-2-yl]-2-ox-oethyl]-3-chloro-6-methoxybenzonitrile 2

2-[2-[(1S,4aR,5R,8aS)-1-methyl-5-[(1 S)-2,2,2-trifluoro-1-hydroxy-ethyl]-3,4,4a,5,6,7,8,8a-octahydro-1H-isoquinolin-2-yl]-2-oxo-ethyl]-3-chloro-6-methoxybenzonitrile 2 was prepared according to Method A, by reacting (1 S)-1-[(1S,4aR,5R,8aS)-1-methyl-1,2,3,4,4a,5,6,7,8,8a-decahydroisoquinolin-5-yl]-2,2,2-trifluoroethanol hydrochloride c3 with 2-(6-chloro-2-cyano-3-methoxyphenyl)acetic acid a9 in the presence of HBTU and a base in DMF. Compound 2 was purified by reverse phase column chromatography (acidic LCMS prep) and isolated as a white solid.

Yield: 55%.

Basic LCMS Method 3 (ES⁺): 459/461 (M+H)⁺, 100% purity.

Acid LCMS Method 2 (ES⁺): 459/461 (M+H)⁺, 100% purity.

The following compounds may be synthesized according a method analogous to Method A:

| N° | Structure | Acids | Base | conditions | (%) |
|---|---|---|---|---|---|
| 3 | 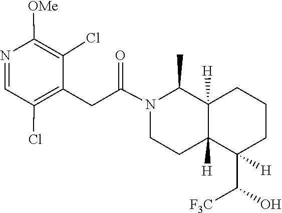 [SMILES: COc1ncc(Cl)c(CC(=O)N2CC[C@H]3[C@H]([C@H](O)C(F)(F)F)CCC[C@@H]3[C@@H]2C)c1Cl] | a15 | 4-methylmorpholine | acidic LCMS prep | 32 |
| 5 | 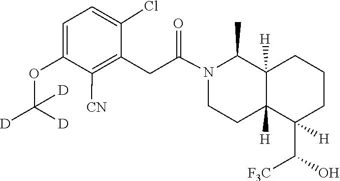 [SMILES: [2H]C([2H])([2H])Oc1ccc(Cl)c(CC(=O)N2CC[C@H]3[C@H]([C@H](O)C(F)(F)F)CCC[C@@H]3[C@@H]2C)c1C#N] | a21 | 4-methylmorpholine | acidic LCMS prep | 50 |
| 11 | 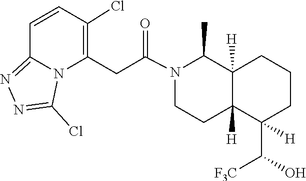 [SMILES: C[C@H]1[C@H]2CCC[C@@H]([C@H](O)C(F)(F)F)[C@@H]2CCN1C(=O)Cc1c(Cl)ccc2nnc(Cl)n12] | A55 | Et₃N (3 equiv) | NP MeOH in DCM (2 to 20%) | 51 |
| 12 | 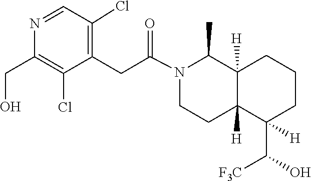 [SMILES: C[C@H]1[C@H]2CCC[C@@H]([C@H](O)C(F)(F)F)[C@@H]2CCN1C(=O)Cc1c(Cl)cnc(CO)c1Cl] | A28 | DIPEA (3 equiv) | NP MeOH in DCM (0 to 2%), then Basic LCMS prep | 37 |
| 14 | 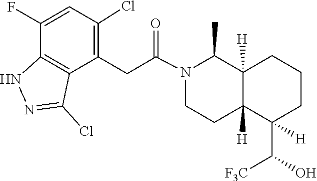 [SMILES: C[C@H]1[C@H]2CCC[C@@H]([C@H](O)C(F)(F)F)[C@@H]2CCN1C(=O)Cc1c(Cl)cc(F)c2[nH]nc(Cl)c12] | A65 | DIPEA (3 equiv) | Basic LCMS prep | 100 |
| 27 | 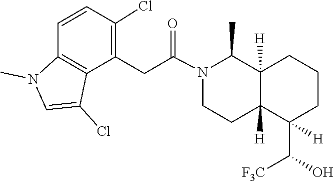 [SMILES: C[C@H]1[C@H]2CCC[C@@H]([C@H](O)C(F)(F)F)[C@@H]2CCN1C(=O)Cc1c(Cl)ccc2c1c(Cl)cn2C] | A39b | DIPEA (3 equiv) | Basic LCMS prep | 100 |
| 19 | 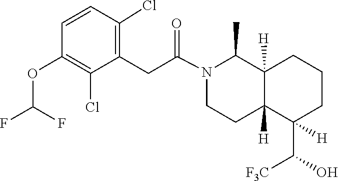 [SMILES: C[C@H]1[C@H]2CCC[C@@H]([C@H](O)C(F)(F)F)[C@@H]2CCN1C(=O)Cc1c(Cl)ccc(OC(F)F)c1Cl] | A45 | DIPEA (3 equiv) | Basic LCMS prep | 99 |
| c4 | 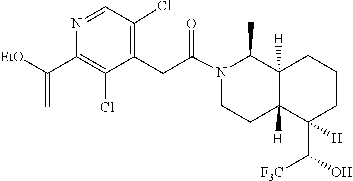 [SMILES: C=C(OCC)c1ncc(Cl)c(CC(=O)N2CC[C@H]3[C@H]([C@H](O)C(F)(F)F)CCC[C@@H]3[C@@H]2C)c1Cl] | A67 | DIPEA (2.5 equiv) | NP Hexane/EtOAc (5 to 50%) | 81 |

### 1-[(1S,4aR,5R,8aS)-1-methyl-5-[(1S)-2,2,2-trifluoro-1-hydroxy-ethyl]-3,4,4a,5,6,7,8,8a-octahydro-1H-isoquinolin-2-yl]-2-(3,5-dichloro-2-methoxypyridin-4-yl)ethanone 3

Basic LCMS Method 3 (ES⁺): 468/470/(M+H)⁺, 100% purity.

Acid LCMS Method 2 (ES⁺): 468/470/(M+H)⁺, 100% purity.

### 2-[2-[(1S,4aR,5R,8aS)-1-methyl-5-[(1S)-2,2,2-trifluoro-1-hydroxy-ethyl]-3,4,4a,5,6,7,8,8a-octahydro-1H-isoquinolin-2-yl]-2-oxo-ethyl]-3-chloro-4-(trideuteriomethoxy)benzonitrile 5

Basic LCMS Method 3 (ES⁺): 462/464 (M+H)⁺, 100% purity.

Acid LCMS Method 2 (ES⁺): 462/464 (M+H)⁺, 100% purity.

### 1-[(1 S,4aR,5R,8aS)-1-methyl-5-[(1 S)-2,2,2-trifluoro-1-hydroxy-ethyl]-3,4,4a,5,6,7,8,8a-octahydro-1H-isoquinolin-2-yl]-2-(3,6-dichloro-[1,2,4]triazolo[4,3-a]pyridin-5-yl)ethanone 11

Basic LCMS Method 3 (ES⁺): 479/481 (M+H)⁺, 97% purity.

Acid LCMS Method 2 (ES⁺): 479/481 (M+H)⁺, 98% purity.

### 1-[(1S,4aR,5R,8aS)-1-methyl-5-[(1 S)-2,2,2-trifluoro-1-hydroxy-ethyl]-3,4,4a,5,6,7,8,8a-octahydro-1H-isoquinolin-2-yl]-2-[3,5-dichloro-2-(hydroxymethyl)-4-pyridyl]ethanone 12

Basic LCMS Method 3 (ES⁺): 469/471/473 (M+H)⁺, 100% purity.

### 1-[(1 S,4aR,5R,8aS)-1-methyl-5-[(1 S)-2,2,2-trifluoro-1-hydroxy-ethyl]-3,4,4a,5,6,7,8,8a-octahydro-1H-isoquinolin-2-yl]-2-(3,5-dichloro-7-fluoro-1H-indazol-4-yl)ethanone 14

Basic LCMS Method 3 (ES⁺): 496/498/500 (M+H)⁺, 100% purity.

### 1-[(1 S,4aR,5R,8aS)-1-methyl-5-[(1 S)-2,2,2-trifluoro-1-hydroxy-ethyl]-3,4,4a,5,6,7,8,8a-octahydro-1H-isoquinolin-2-yl]-2-(3,5-dichloro-1-methyl-indol-4-yl)ethanone 27

Basic LCMS Method 3 (ES⁺): 491/493/495 (M+H)⁺, 96% purity.

### 1-[(1 S,4aR,5R,8aS)-1-methyl-5-[(1 S)-2,2,2-trifluoro-1-hydroxy-ethyl]-3,4,4a,5,6,7,8,8a-octahydro-1H-isoquinolin-2-yl]-2-[2,6-dichloro-3-(difluoromethoxy)phenyl]ethanone 19

Basic LCMS Method 3 (ES⁺): 504/506/508 (M+H)⁺, 99% purity.

### 1-[(1 S,4aR,5R,8aS)-1-methyl-5-[(1 S)-2,2,2-trifluoro-1-hydroxy-ethyl]-3,4,4a,5,6,7,8,8a-octahydro-1H-isoquinolin-2-yl]-2-[3,5-dichloro-2-(1-ethoxyvinyl)-4-pyridyl]ethanone c4

Basic LCMS Method 3 (ES⁺): 509/511/513 (M+H)⁺, 97% purity.

### C.3. Synthesis of 1-[(1S,4aR,5R,8aS)-1-methyl-5-[(1S)-2,2,2-trifluoro-1-hydroxy-ethyl]-3,4,4a,5,6,7,8,8a-octahydro-1H-isoquinolin-2-yl]-2-[3,5-dichloro-2-[1-hydroxyethyl]-4-pyridyl]ethanone Isomer A 9-A and 1-[(1S,4aR,5R,8aS)-1-methyl-5-[(1S)-2,2,2-trifluoro-1-hydroxy-ethyl]-3,4,4a,5,6,7,8,8a-octahydro-1H-isoquinolin-2-yl]-2-[3,5-dichloro-2-[1-hydroxyethyl]-4-pyridyl]ethanone Isomer B 9-B

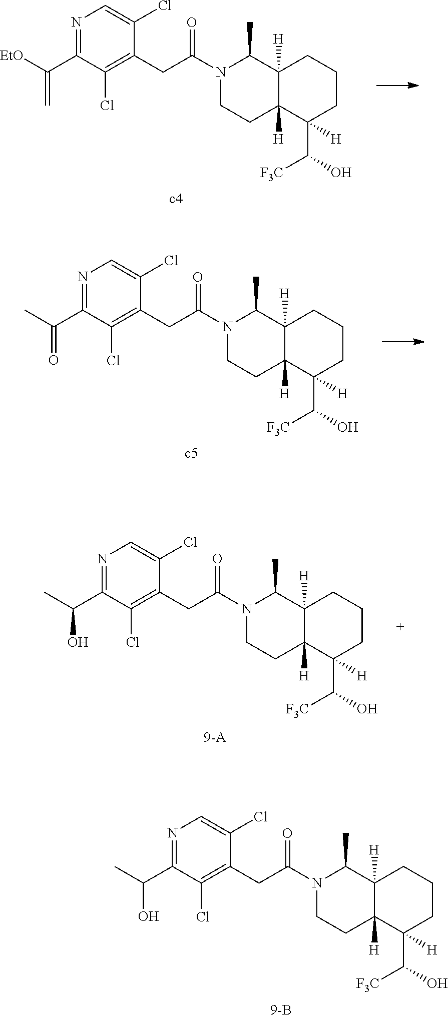 [SMILES: C=C(OCC)c1ncc(Cl)c(CC(=O)N2CC[C@H]3[C@H]([C@H](O)C(F)(F)F)CCC[C@@H]3[C@@H]2C)c1Cl ; CC(=O)c1ncc(Cl)c(CC(=O)N2CC[C@H]3[C@H]([C@H](O)C(F)(F)F)CCC[C@@H]3[C@@H]2C)c1Cl ; C[C@H](O)c1ncc(Cl)c(CC(=O)N2CC[C@H]3[C@H]([C@H](O)C(F)(F)F)CCC[C@@H]3[C@@H]2C)c1Cl ; CC(O)c1ncc(Cl)c(CC(=O)N2CC[C@H]3[C@H]([C@H](O)C(F)(F)F)CCC[C@@H]3[C@@H]2C)c1Cl]

### C.3.1. Synthesis of 2-[3,5-dichloro-2-(1-ethoxyvinyl)-4-pyridyl]acetic acid c5

To a solution of 1-[(1 S,4aR,5R,8aS)-1-methyl-5-[(1S)-2,2,2-trifluoro-1-hydroxy-ethyl]-3,4,4a,5,6,7,8,8a-octahydro-1H-isoquinolin-2-yl]-2-[3,5-dichloro-2-(1-ethoxyvinyl)-4-pyridyl]ethanone c4 (11.7 g, 23.0 mmol) in THF (100 mL) was added dropwise a 1 N aqueous solution of HCl (40 mL) and the reaction mixture was stirred overnight at rt for 3 days. To the reaction mixture was added EtOAc (300 mL) and the organic layer was washed with a saturated aqueous solution of sodium bicarbonate (150 mL). The organic layer was then dried over MgSO₄, filtered and concentrated under vacuum to afford 11.0 g of 1-[(1S,4aR,5R,8aS)-1-methyl-5-[(1 S)-2,2,2-trifluoro-1-hydroxy-ethyl]-3,4,4a,5,6,7,8,8a-octahydro-1H-isoquinolin-2-yl]-2-(2-acetyl-3,5-dichloro-4-pyridyl)ethanone c5, which was used in the next step without further purification.

Yield: 100%

Acid LCMS Method 1 (ES⁺): 481/483/485 (M+H)⁺.

### C.3.2. Synthesis of 1-[(1S,4aR,5R,8aS)-1-methyl-5-[(1S)-2,2,2-trifluoro-1-hydroxy-ethyl]-3,4,4a,5,6,7,8,8a-octahydro-1H-isoquinolin-2-yl]-2-[3,5-dichloro-2-(1-hydroxyethyl)-4-pyridyl]ethanone Isomer A 9-A and 1-[(1S,4aR,5R,8aS)-1-methyl-5-[(1S)-2,2,2-trifluoro-1-hydroxy-ethyl]-3,4,4a,5,6,7,8,8a-octahydro-1H-isoquinolin-2-yl]-2-[3,5-dichloro-2-(hydroxyethyl)-4-pyridyl]ethanone Isomer B 9-B

To a suspension of 1-[(1 S,4aR,5R,8aS)-1-methyl-5-[(1S)-2,2,2-trifluoro-1-hydroxy-ethyl]-3,4,4a,5,6,7,8,8a-octahydro-1H-isoquinolin-2-yl]-2-(2-acetyl-3,5-dichloro-4-pyridyl)ethanone c5 (9.53 g, 19.8 mmol) in MeOH (100 mL) was added portion wise at 0° C. sodium borohydride (824 mg, 21.8 mmol) and the reaction mixture was allowed to stir at 0° C. for 30 min. Then, the reaction mixture was stirred overnight at rt, quenched with water (50 mL) and a 1N aqueous solution of HCl (50 mL). The resulting mixture was stirred for 1 h and was extracted with DCM (4×250 mL). The organic layer was dried over MgSO₄, filtered and concentrated under vacuum to afford 9.60 g of 1-[(1S,4aR,5R,8aS)-1-methyl-5-[(1S)-2,2,2-trifluoro-1-hydroxy-ethyl]-3,4,4a,5,6,7,8,8a-octahydro-1H-isoquinolin-2-yl]-2-[3,5-dichloro-2-(1-hydroxyethyl)-4-pyridyl]ethanone 9 as a mixture of isomers 9-A and 9-B and as a white solid (Yield: 96%, Acid LCMS Method 1 (ES⁺): 483/485/487 (M+H)⁺).

Chiral separation (SFC, IG Daicel®, 20 μm, 250×50 mm, 360 mL/min, 220 nm, 30° C., elution: iPrOH 25%—CO₂ 75%) of the above mixture 1-[(1S,4aR,5R,8aS)-1-methyl-5-[(1S)-2,2,2-trifluoro-1-hydroxy-ethyl]-3,4,4a,5,6,7,8,8a-octahydro-1H-isoquinolin-2-yl]-2-[3,5-dichloro-2-(1-hydroxyethyl)-4-pyridyl]ethanone 9 afforded:

3.60 g of 1-[(1 S,4aR,5R,8aS)-1-methyl-5-[(1S)-2,2,2-trifluoro-1-hydroxy-ethyl]-3,4,4a,5,6,7,8,8a-octahydro-1H-isoquinolin-2-yl]-2-[3,5-dichloro-2-(1-hydroxyethyl)-4-pyridyl]ethanone Isomer A 9-A, as a solid.

Yield: 39% (after precipitation in IPrOH)

Basic LCMS Method 3 (ES⁺): 483/485/487 (M+H)⁺, 100% purity.

Acid LCMS Method 2 (ES⁺): 483/485/487 (M+H)⁺, 100% purity.

Chiral analysis (LC, Chiralpak IA Daicel®, 3 μm, 150×4.6 mm, 1.5 mL/min, 220 nm, 30° C., elution: iPrOH/n-heptane/DEA 30/70/0.1): RT 1.91 min, 100% de.

X-Ray diffraction of Example 9-A: A colourless block-like single crystal was selected and mounted on the MiTeGen MicroMounts sample holder. Single-crystal X-ray diffraction data were collected using the Oxford Diffraction Gemini R Ultra diffractometer (Cu Kα, multilayer mirror, Ruby CCD area detector) at 100 (2) K. Data collection, unit cells determination and data reduction were carried out using CrysAlis PRO software package. Using Olex2 and shelXle, the structure was solved with the SHELXT 2015 structure solution program by Intrinsic Phasing methods and refined by full-matrix least squares on IF12 using SHELXL-2018/3. Non-hydrogen atoms were refined anisotropically. The 3,5-dichloro-2-[(1S)-1-hydroxyethyl]pyridin-4-yl}ethan-1-one groups are disordered over two positions in both molecules in the asymmetric unit. The structure contains one molecule of disordered butanone, solvent was taken into account using PLATON SQUEEZE procedure. Hydrogen atoms were placed on calculated positions in riding mode with temperature factors fixed at 1.2 times Ueq of the parent carbon atoms (1.5 for methyl groups).

Crystal Data for C₄₂H₅₄N₄O₆F₆Cl₄ (2 molecules of C₂₁H₂₇Cl₂F₃N₂O₃, M=966.7 g/mol): orthorhombic, space group P2₁2₁2₁ (no. 19), a=8.57039 (10) Å, b=16.19438 (16) Å, c=35.7015 (3) Å, V=4955.08 (9) Å³, Z=4, T=100 (2) K, λ(CuKα)=1.54184, μcalc=2.767 g/cm³, 27552 reflections measured (4.95°≤2Θ≤134.23°), 8714 independent reflections (Rint=0.0253, Rsigma=0.0227) which were used in all calculations. The final R¹ was 0.0395 (I>2σ(1)) and wR² was 0.1090 (all data).

Absolute configuration established by anomalous-dispersion effects in diffraction measurements on the crystal. Flack x parameter determined using 3403 quotients [(I+)−(I−)]/[(I+)+(I−)] and equal to −0.002 (5) indicating the absolute configuration as displayed in section C.3; above (Example 9-A). The asymmetric unit contains two molecules of Example 9-A and one molecule of disordered butanone.

3.50 g of 1-[(1 S,4aR,5R,8aS)-1-methyl-5-[(1S)-2,2,2-trifluoro-1-hydroxy-ethyl]-3,4,4a,5,6,7,8,8a-octahydro-1H-isoquinolin-2-yl]-2-[3,5-dichloro-2-(1-hydroxyethyl)-4-pyridyl]ethanone Isomer B 9-B, as a solid.

Yield: 38% (after precipitation in IPrOH)

Basic LCMS Method 3 (ES⁺): 483/485/487 (M+H)⁺, 100% purity.

Acid LCMS Method 2 (ES⁺): 483/485/487 (M+H)⁺, 100% purity.

Chiral analysis (LC, Chiralpak IA Daicel®, 3 μm, 150×4.6 mm, 1.5 mL/min, 220 nm, 30° C., elution: iPrOH/n-heptane/DEA 30/70/0.1): RT 2.29 min, 94% de.

### C.4 Synthesis of 2-[2-[(1S,4aR,5R,8aS)-5-(2,2-difluoro-1-hydroxy-ethyl)-1-methyl-3,4,4a,5,6,7,8,8a-octahydro-1H-isoquinolin-2-yl]-2-oxo-ethyl]-3-chloro-4-methoxybenzonitrile Isomer A 6-A and 2-[2-[(1S,4aR,5R,8aS)-5-(2,2-difluoro-1-hydroxy-ethyl)-1-methyl-3,4,4a,5,6,7,8,8a-octahydro-1H-isoquinolin-2-yl]-2-oxo-ethyl]-3-chloro-4-methoxybenzonitrile Isomer B 6-B

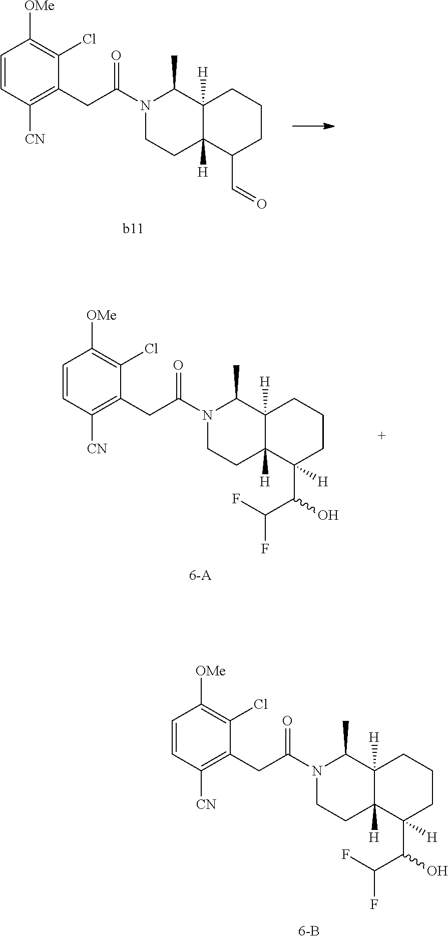 [SMILES: COc1ccc(C#N)c(CC(=O)N2CC[C@H]3C(C=O)CCC[C@@H]3[C@@H]2C)c1Cl ; COc1ccc(C#N)c(CC(=O)N2CC[C@@H]3[C@H](CCC[C@H]3C(O)C(F)F)[C@@H]2C)c1Cl ; COc1ccc(C#N)c(CC(=O)N2CC[C@@H]3[C@H](CCC[C@H]3C(O)C(F)F)[C@@H]2C)c1Cl]

Cesium fluoride (79.0 mg, 0.51 mmol) was added to a solution of difluoromethyltrimethylsilane (65.0 mg, 0.51 mmol) and 2-[2-[(1S,4aR,8aS)-5-formyl-1-methyl-3,4,4a,5,6,7,8,8a-octahydro-1H-isoquinolin-2-yl]-2-oxoethyl]-3-chloro-4-methoxybenzonitrile b11 (100 mg, 0.26 mmol) in DMF (5 mL) was then added. The reaction mixture was stirred overnight at rt. The reaction mixture was diluted with EtOAc (150 mL), washed with a 1 N aqueous solution of HCl (50 mL), a saturated aqueous solution of sodium carbonate (50 mL) and brine (50 mL). The organic layer was dried over MgSO₄, filtered and concentrated under vacuum. The crude residue was successively purified by normal phase column chromatography (elution: 50% EtOAc in heptane), then by reverse phase column chromatography (Basic LCMS prep) to give 40.0 mg of 2-[2-[(1S,4aR,5R,8aS)-5-(2,2-difluoro-1-hydroxyethyl)-1-methyl-3,4,4a,5,6,7,8,8a-octahydro-1H-isoquinolin-2-yl]-2-oxoethyl]-3-chloro-4-methoxybenzonitrile 6 as a mixture of isomers 6-A and 6-B (Yield: 35%,

Basic LCMS Method 2 (ES⁺): 441/443 (M+H)⁺, 94% purity).

Chiral separation (SFC, ID, 50*258 mm, 360 mL/min, 220 nm, 30° C., elution: EtOH 25%—CO₂ 75%) of the above mixture 2-[2-[(1 S,4aR,5R,8aS)-5-(2,2-difluoro-1-hydroxy-ethyl)-1-methyl-3,4,4a,5,6,7,8,8a-octahydro-1H-isoquinolin-2-yl]-2-oxo-ethyl]-3-chloro-4-methoxybenzonitrile 6 afforded:

7.00 mg of 2-[2-[(1S,4aR,5R,8aS)-5-(2,2-difluoro-1-hydroxy-ethyl)-1-methyl-3,4,4a,5,6,7,8,8a-octahydro-1H-isoquinolin-2-yl]-2-oxo-ethyl]-3-chloro-4-methoxybenzonitrile Isomer A 6-A, as a white solid.

Yield: 6%.

Basic LCMS Method 3 (ES⁺): 441/443 (M+H)⁺, 100% purity.

Acid LCMS Method 2 (ES⁺): 441/443 (M+H)⁺, 100% purity.

Chiral analysis (LC, ID, 3 μm, 150×4.6 mm, 1.5 mL/min, 220 nm, 30° C., elution: EtOH/n-heptane/DEA 50/50/0.1): RT 2.27 min, 100% de.

7.00 mg of 2-[2-[(1S,4aR,5R,8aS)-5-(2,2-difluoro-1-hydroxy-ethyl)-1-methyl-3,4,4a,5,6,7,8,8a-octahydro-1H-isoquinolin-2-yl]-2-oxo-ethyl]-3-chloro-4-methoxybenzonitrile Isomer B 6-B, as a white solid.

Yield: 6%.

Basic LCMS Method 3 (ES⁺): 441/443 (M+H)⁺, 100% purity.

Acid LCMS Method 2 (ES⁺): 441/443 (M+H)⁺, 100% purity.

Chiral analysis (LC, ID, 3 μm, 150×4.6 mm, 1.5 mL/min, 220 nm, 30° C., elution: EtOH/n-heptane/DEA 50/50/0.1): RT 2.93 min, 100% de.

### C.5. Synthesis of 2-[2-[(1S,4aR,5R,8aS)-1-methyl-5-[2,2,2-trifluoro-1-hydroxy-1-methyl-ethyl]-3,4,4a,5,6,7,8,8a-octahydro-1H-isoquinolin-2-yl]-2-oxo-ethyl]-3-chloro-4-methoxy-benzonitrile Isomer A 7-A and 2-[2-[(1S,4aR,5R,8aS)-1-methyl-5-[2,2,2-trifluoro-1-hydroxy-1-methyl-ethyl]-3,4,4a,5,6,7,8,8a-octahydro-1H-isoquinolin-2-yl]-2-oxo-ethyl]-3-chloro-4-methoxy-benzonitrile Isomer B 7-B

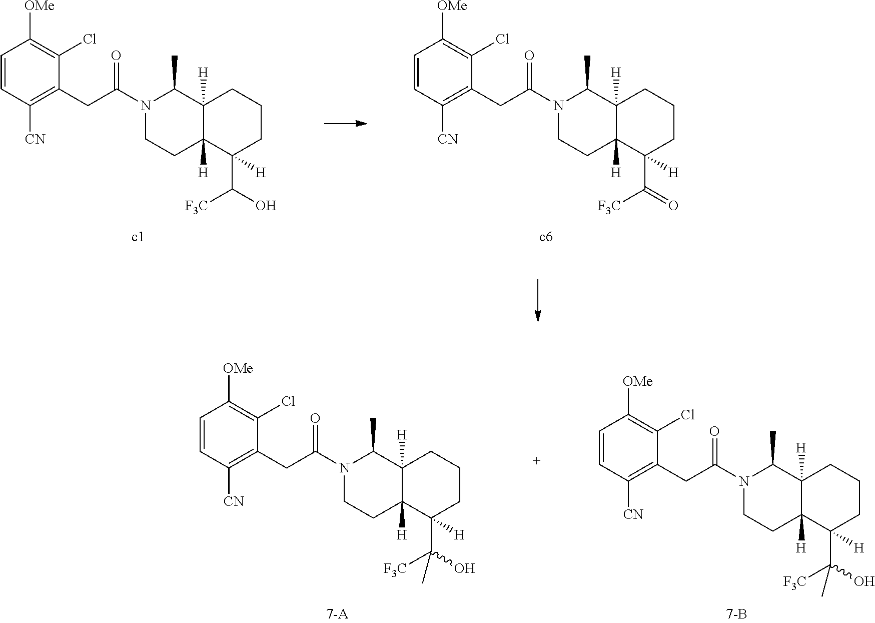 [SMILES: COc1ccc(C#N)c(CC(=O)N2CC[C@@H]3[C@H](CCC[C@H]3C(O)C(F)(F)F)[C@@H]2C)c1Cl ; COc1ccc(C#N)c(CC(=O)N2CC[C@@H]3[C@H](CCC[C@H]3C(=O)C(F)(F)F)[C@@H]2C)c1Cl ; COc1ccc(C#N)c(CC(=O)N2CC[C@@H]3[C@H](CCC[C@H]3C(C)(O)C(F)(F)F)[C@@H]2C)c1Cl ; COc1ccc(C#N)c(CC(=O)N2CC[C@@H]3[C@H](CCC[C@H]3C(C)(O)C(F)(F)F)[C@@H]2C)c1Cl]

### C.5.1. Synthesis of 2-[2-[(1S,4aR,5R,8aS)-1-methyl-5-(2,2,2-trifluoroacetyl)-3,4,4a,5,6,7,8,8a-octahydro-1H-isoquinolin-2-yl]-2-oxoethyl]-3-chloro-4-methoxybenzonitrile c6

To a solution of 2-[2-[(1 S,4aR,5R,8aS)-1-methyl-5-(2,2,2-trifluoro-1-hydroxyethyl)-3,4,4a,5,6,7,8,8a-octahydro-1H-isoquinolin-2-yl]-2-oxoethyl]-3-chloro-4-methoxybenzonitrile c1 (1.49 g, 3.25 mmol) in DCM (15 mL) was added Dess-Martin Periodinane (1.42 g, 3.25 mmol) by portions at 0° C. and the reaction mixture was allowed to warm to rt overnight. Dess-Martin Periodinane (140 mg, 0.32 mmol) was again added at rt and the reaction mixture stirred at rt overnight. The mixture was diluted by DCM (50 mL), followed by the addition of a 1 N aqueous solution of NaOH (50 mL). The reaction mixture was stirred for an additional 30 min at rt, then successively washed with a 1N aqueous solution of NaOH (25 mL) and H₂O (50 mL). The organic layer was dried over MgSO₄, filtered and concentrated under vacuum to afford 1.29 g of 2-[2-[(1S,4aR,5R,8aS)-1-methyl-5-(2,2,2-trifluoroacetyl)-3,4,4a,5,6,7,8,8a-octahydro-1H-isoquinolin-2-yl]-2-oxoethyl]-3-chloro-4-methoxybenzonitrile c6 as a white foam, which was used in next step without further purification.

Yield (crude): 87%.

Basic LCMS Method 2 (ES⁺): 457 (M+H)⁺.

### C.5.2. Synthesis of 2-[2-[(1S,4aR,5R,8aS)-1-methyl-5-[2,2,2-trifluoro-1-hydroxy-1-methyl-ethyl]-3,4,4a,5,6,7,8,8a-octahydro-1H-isoquinolin-2-yl]-2-oxo-ethyl]-3-chloro-4-methoxy-benzonitrile Isomer A 7-A and 2-[2-[(1S,4aR,5R,8aS)-1-methyl-5-[2,2,2-trifluoro-1-hydroxy-1-methyl-ethyl]-3,4,4a,5,6,7,8,8a-octahydro-1H-isoquinolin-2-yl]-2-oxo-ethyl]-3-chloro-4-methoxy-benzonitrile Isomer B7-B

At −78° C., a 3 M solution of methylmagnesium chloride in THF (328 μL, 985 μmol) was added dropwise to a solution of 2-[2-[(1 S,4aR,5R,8aS)-1-methyl-5-(2,2,2-trifluoroacetyl)-3,4,4a,5,6,7,8,8a-octahydro-1H-isoquinolin-2-yl]-2-oxoethyl]-3-chloro-4-methoxybenzonitrile c6 (150 mg, 0.39 mmol) in THF (4.00 mL). The reaction mixture was stirred for 1 h at −78° C., then diluted with EtOAc (150 mL) and successively washed with a 1N aqueous solution of HCl (50 mL), a saturated aqueous solution of sodium carbonate (50 mL) and brine (50 mL). The organic layer was dried over MgSO₄, filtered and concentrated under vacuum. The crude residue was purified by column chromatographies (Basic LCMS prep, then SFC separation (SiO₂, 22×250 mm, 60 mL/min, 220 nm, 40° C., elution: EtOH 5%—CO₂ 95%)) to afford 70.0 mg of 2-[2-[(1 S,4aR,5R,8aS)-1-methyl-5-[2,2,2-trifluoro-1-hydroxy-1-methyl-ethyl]-3,4,4a,5,6,7,8,8a-octahydro-1H-isoquinolin-2-yl]-2-oxo-ethyl]-3-chloro-4-methoxy-benzonitrile 7 as a mixture of isomers 7-A and 7-B (Yield: 45%, Basic LCMS Method 2 (ES⁺): 473/475 (M+H)⁺).

Chiral separation (SFC, IG, 50×250 mm, 360 mL/min, 220 nm, 30° C., elution: MeOH 25%—CO₂ 75%) of the above mixture 2-[2-[(1S,4aR,5R,8aS)-1-methyl-5-[2,2,2-trifluoro-1-hydroxy-1-methyl-ethyl]-3,4,4a,5,6,7,8,8a-octahydro-1H-isoquinolin-2-yl]-2-oxo-ethyl]-3-chloro-4-methoxy-benzonitrile 7 afforded:

2.00 mg of 2-[2-[(1 S,4aR,5R,8aS)-1-methyl-5-[2,2,2-trifluoro-1-hydroxy-1-methyl-ethyl]-3,4,4a,5,6,7,8,8a-octahydro-1H-isoquinolin-2-yl]-2-oxo-ethyl]-3-chloro-4-methoxy-benzonitrile Isomer A 7-A, as a white solid.

Yield: 1%

Basic LCMS Method 3 (ES⁺): 473/475 (M+H)⁺, 97% purity.

Acid LCMS Method 2 (ES⁺): 473/475 (M+H)⁺, 96% purity.

Chiral analysis (LC, IG, 3 μm, 150×4.6 mm, 1.5 mL/min, 220 nm, 30° C., elution: EtOH/n-heptane/DEA 50/50/0.1): RT 2.89 min, 100% de.

5.00 mg of 2-[2-[(1 S,4aR,5R,8aS)-1-methyl-5-[2,2,2-trifluoro-1-hydroxy-1-methyl-ethyl]-3,4,4a,5,6,7,8,8a-octahydro-1H-isoquinolin-2-yl]-2-oxo-ethyl]-3-chloro-4-methoxy-benzonitrile Isomer B 7-B, as a white solid.

Yield: 3%

Basic LCMS Method 3 (ES⁺): 473/475 (M+H)⁺, 99% purity.

Acid LCMS Method 2 (ES⁺): 473/475 (M+H)⁺, 99% purity.

Chiral analysis (LC, IG, 3 μm, 150×4.6 mm, 1.5 mL/min, 220 nm, 30° C., elution: EtOH/n-heptane/DEA 50/50/0.1): RT 3.64 min, 100% de.

### C.6. Synthesis of 1-[(1S,4aR,8aS)-1-methyl-5-[(1R)-2,2,2-trifluoro-1-hydroxy-ethyl]-3,4,4a,5,6,7,8,8a-octahydro-1H-isoquinolin-2-yl]-2-(3,5-dichloro-1-methyl-indazol-4-yl)ethanone Isomer A 8-A and 1-[(1S,4aR,8aS)-1-methyl-5-[(1R)-2,2,2-trifluoro-1-hydroxy-ethyl]-3,4,4a,5,6,7,8,8a-octahydro-1H-isoquinolin-2-yl]-2-(3,5-dichloro-1-methyl-indazol-4-yl)ethanone Isomer B 8-B

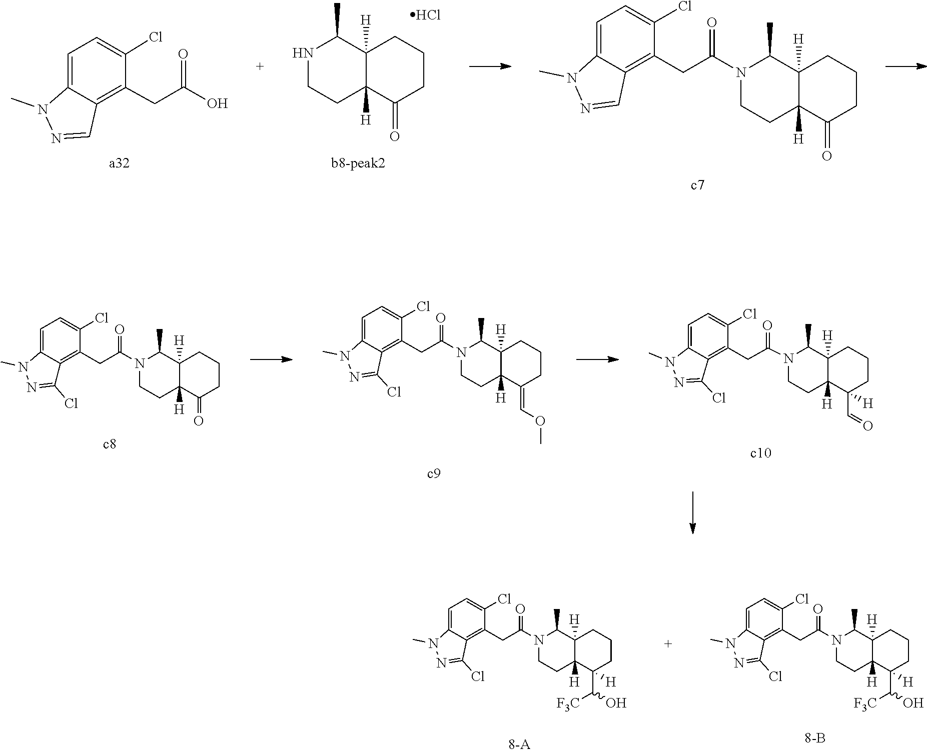

### C.6.1. Synthesis of (1S,4aR,8aS)-2-[2-(5-chloro-1-methyl-indazol-4-yl)acetyl]-1-methyl-1,3,4,4a,6,7,8,8a-octahydroisoquinolin-5-one c7

(1S,4aR,8aS)-2-[2-(5-chloro-1-methyl-indazol-4-yl)acetyl]-1-methyl-1,3,4,4a,6,7,8,8a-octahydroisoquinolin-5-one c7 was prepared according to Method A, by reacting (1S,4aR,8aS)-1-methyl-2,3,4, 4a,6,7,8,8a-octahydro-1H-isoquinolin-5-one b8-peak2 (626 mg, 3.07 mmol) with 2-(5-chloro-1-methyl-indazol-4-yl)acetic acid a32 (759 mg, 3.38 mmol) in the presence of HBTU (10.28 g, 3.38 mmol) and 4-methylmorpholine (933 mg, 9.22 mmol) in DMF (40 mL). c7 was used in the next step without purification.

Yield (crude): 61%

Acid LCMS Method 1 (ES⁺): 374/376 (M+H)⁺.

### C.6.2. Synthesis of (1S,4aR,8aS)-2-[2-(3,5-dichloro-1-methyl-indazol-4-yl)acetyl]-1-methyl-1,3,4,4a,6,7,8,8a-octahydroisoquinolin-5-one c8

To a stirred solution of (1 S,4aR,8aS)-2-[2-(5-chloro-1-methyl-indazol-4-yl)acetyl]-1-methyl-1,3,4,4a,6,7,8,8a-octahydroisoquinolin-5-one c7 (673 mg, 1.80 mmol) in THF (15.0 mL), NCS (294 mg. 2.20 mmol) was added at rt. The reaction mixture was stirred 15 h at rt. then diluted with EtOAc (150 mL) and successively washed with a 1 N aqueous solution of HCl (50 mL), a saturated aqueous solution of sodium carbonate (50 mL) and brine (50 mL). The organic layer was dried over MgSO₄, filtered and concentrated under vacuum to afford 650 mg of (1 S,4aR,8aS)-2-[2-(3,5-dichloro-1-methyl-indazol-4-yl)acetyl]-1-methyl-1,3,4,4a,6,7,8,8a-octahydroisoquinolin-5-one c8, which was used in the next step without further purification.

Yield (crude): 88%.

Acid LCMS Method 1 (ES⁺): 408/410/412 (M+H)⁺

### C.6.3. Synthesis of 1-[(1S,4aR,5E,8aS)-5-(methoxymethylene)-1-methyl-1,3,4,4a,6,7,8,8a-octahydroisoquinolin-2-yl]-2-(3,5-dichloro-1-methyl-indazol-4-yl)ethanone c9

At −78° C. under argon, a 1.6 M solution of nBuLi in hexanes (0.49 mL, 0.78 mmol) was added to a solution of methoxymethyl(triphenyl)phosphonium chloride (250 mg, 0.73 mmol) in THF (5 mL). The reaction mixture was stirred 15 min at 0° C. The reaction mixture was cooled again at −78° C. before adding (1 S,4aR,8aS)-2-[2-(3,5-dichloro-1-methyl-indazol-4-yl)acetyl]-1-methyl-1,3,4,4a,6,7,8,8a-octahydroisoquinolin-5-one c8 (200 mg, 0.49 mmol). The reaction mixture was stirred 2 h at rt. The reaction mixture was diluted with EtOAc (150 mL) and successively washed with a 1 N aqueous solution of HCl (50 mL), a saturated aqueous solution of sodium carbonate (50 mL) and brine (50 mL). The organic layer was dried over MgSO₄, filtered and concentrated under vacuum. The crude residue was purified by normal phase column chromatography (elution: from 0 to 80% EtOAc in heptane) afford 100 mg of a mixture of Z and E isomers of 1-[(1S,4aR,5E,8aS)-5-(methoxymethylene)-1-methyl-1,3,4,4a,6,7,8,8a-octahydroisoquinolin-2-yl]-2-(3,5-dichloro-1-methyl-indazol-4-yl)ethanone c9.

Yield: 47%.

Acid LCMS Method 1 (ES⁺): 436/438/440 (M+H)⁺

### C.6.4. Synthesis of (1S,4aR,8aS)-2-[2-(3,5-dichloro-1-methyl-indazol-4-yl)acetyl]-1-methyl-3,4,4a,5,6,7,8,8a-octahydro-1H-isoquinoline-5-carbaldehyde c10

A 1N aqueous solution of HCl (20.0 mmol, 2.00 mL) was added to a solution of 1-[(1S,4aR,5E,8aS)-5-(methoxymethylene)-1-methyl-1,3,4,4a,6,7,8,8a-octahydroisoquinolin-2-yl]-2-(3,5-dichloro-1-methyl-indazol-4-yl)ethanone c9 (210 mg, 0.48 mmol) in THF (2 mL). The reaction mixture was stirred overnight at rt. The reaction mixture was diluted with EtOAc (50 mL) ans successively washed with a saturated aqueous solution of sodium carbonate (20 mL) and brine (20 mL). The organic layer was dried over MgSO₄, filtered and concentrated under vacuum to afford 95.0 mg of (1 S,4aR,8aS)-2-[2-(3,5-dichloro-1-methyl-indazol-4-yl)acetyl]-1-methyl-3,4,4a,5,6,7,8,8a-octahydro-1H-isoquinoline-5-carbaldehyde c0, which was used in the next step without further purification.

Yield: 47%.

Basic LCMS Method 3 (ES⁺): 422/424/426 (M+H)⁺, 89% purity.

Acid LCMS Method 2 (ES⁺): 422/424/426 (M+H)⁺, 87% purity.

### C.6.5. Synthesis of 1-[(1S,4aR,8aS)-1-methyl-5-[(1R)-2,2,2-trifluoro-1-hydroxy-ethyl]-3,4,4a,5,6,7,8,8a-octahydro-1H-isoquinolin-2-yl]-2-(3,5-dichloro-1-methyl-indazol-4-yl)ethanone Isomer A 8-A and 1-[(1S,4aR,8aS)-1-methyl-5-[(1R)-2,2,2-trifluoro-1-hydroxy-ethyl]-3,4,4a,5,6,7,8,8a-octahydro-1H-isoquinolin-2-yl]-2-(3,5-dichloro-1-methyl-indazol-4-yl)ethanone Isomer B 8-B

Cesium fluoride (130 mg, 0.88 mmol) was added to a solution of trimethyl(trifluoromethyl)silane (125 mg, 0.88 mmol) and 2-[2-[(1 S,4aR,8aS)-5-formyl-1-methyl-3,4,4a,5,6,7,8,8a-octahydro-1H-isoquinolin-2-yl]-2-oxo-ethyl]-3-chloro-4-methoxy-benzonitrile c10 (185 mg, 0.44 mmol) in DMF (5 mL). The reaction mixture was stirred over 48 h at rt. The reaction mixture was diluted with EtOAc (150 mL) and successively washed with a 1 N aqueous solution of HCl (50 mL), a saturated aqueous solution of sodium carbonate (50 mL) and brine (50 mL). The organic layer was dried over MgSO₄, filtered and concentrated under vacuum. The crude residue was purified by reverse phase column chromatography (Basic LCMS prep) to afford 90.0 mg of 1-[(1S,4aR,8aS)-1-methyl-5-[(1R)-2,2,2-trifluoro-1-hydroxy-ethyl]-3,4,4a,5,6,7,8,8a-octahydro-1H-isoquinolin-2-yl]-2-(3,5-dichloro-1-methyl-indazol-4-yl)ethanone 8 as a mixture of isomers 8-A and 8-B (Yield: 42%, Basic LCMS Method 2 (ES⁺): 492/494/496 (M+H)⁺).

Chiral separation (SFC, ID, 50×258 mm, 360 mL/min, 220 nm, 30° C., elution: EtOH 20%—CO₂ 80%) of the above mixture 1-[(1S,4aR,8aS)-1-methyl-5-[(1R)-2,2,2-trifluoro-1-hydroxy-ethyl]-3,4,4a,5,6,7,8,8a-octahydro-1H-isoquinolin-2-yl]-2-(3,5-dichloro-1-methyl-indazol-4-yl)ethanone 8 afforded:

35.0 mg of 1-[(1S,4aR,8aS)-1-methyl-5-[(1R)-2,2,2-trifluoro-1-hydroxy-ethyl]-3,4,4a,5,6,7,8,8a-octahydro-1H-isoquinolin-2-yl]-2-(3,5-dichloro-1-methyl-indazol-4-yl)ethanone Isomer A 8-A

Yield: 39%

Basic LCMS Method 3 (ES⁺): 492/494/496 (M+H)⁺, 100% purity. Acid LCMS Method 2 (ES⁺): 492/494/496 (M+H)⁺, 100% purity.

Chiral analysis (LC, IE3, 150×4.6 mm, 1.5 mL/min, 220 nm, 30° C., elution: iPrOH/n-heptane/DEA 50/50/0.1): RT 2.53 min, 100% de.

35.0 mg of 1-[(1S,4aR,8aS)-1-methyl-5-[(1R)-2,2,2-trifluoro-1-hydroxy-ethyl]-3,4,4a,5,6,7,8,8a-octahydro-1H-isoquinolin-2-yl]-2-(3,5-dichloro-1-methyl-indazol-4-yl)ethanone Isomer B8-B

Yield: 39%

Basic LCMS Method 3 (ES⁺): 492/494/496 (M+H)⁺, 99% purity.

Acid LCMS Method 2 (ES⁺): 492/494/496 (M+H)⁺, 100% purity.

Chiral analysis (LC, IE3, 150×4.6 mm, 1.5 mL/min, 220 nm, 30° C., elution: iPrOH/n-heptane/DEA 50/50/0.1): RT 3.74 min, 100% de.

### C.7. Synthesis of (1S,4aR,5R,8aS)—N-(2,6-dichlorophenyl)-1-methyl-5-[(1S)-2,2,2-trifluoro-1-hydroxy-ethyl]-3,4,4a,5,6,7,8,8a-octahydro-1H-isoquinoline-2-carboxamide 10

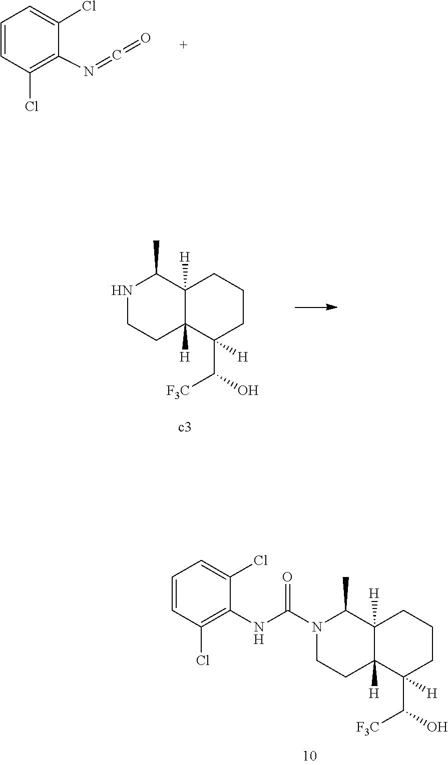 [SMILES: O=C=Nc1c(Cl)cccc1Cl ; C[C@@H]1NCC[C@H]2[C@H]([C@H](O)C(F)(F)F)CCC[C@@H]21 ; C[C@H]1[C@H]2CCC[C@@H]([C@H](O)C(F)(F)F)[C@@H]2CCN1C(=O)Nc1c(Cl)cccc1Cl]

To a stirred solution of (1S)-1-[(1S,4aR,5R,8aS)-1-methyl-1,2,3,4,4a,5,6,7,8,8a-decahydroisoquinolin-5-yl]-2,2,2-trifluoro-ethanol hydrochloride c3 (46.0 mg, 0.16 mmol) in DCM (1 mL), 2,6-dichlorophenyl isocyanate (34.0 mg, 0.18 mmol) and Et₃N (68.0 μL, 0.48 mmol) were successively added at rt. The reaction mixture was stirred 1 h at rt. The reaction mixture was diluted with DCM (50 mL), washed with a 1 N aqueous solution of HCl (20 mL) and brine (20 mL). The organic layer was dried over MgSO₄, filtered and concentrated under vacuum. The crude residue was purified by reverse phase column chromatography (basic LCMS prep) to afford 26.0 mg of (1 S,4aR,5R,8aS)—N-(2,6-dichlorophenyl)-1-methyl-5-[(1 S)-2,2,2-trifluoro-1-hydroxy-ethyl]-3,4,4a,5,6,7,8,8a-octahydro-1H-isoquinoline-2-carboxamide 10 as a white solid.

Yield: 37%.

Acid LCMS Method 2 (ES⁺): 439/441/443 (M+H)⁺, 92% purity.

### C.8. Synthesis of 1-[(1S,4aR,5R,8aS)-1-methyl-5-[(1S)-2,2,2-trifluoro-1-hydroxy-ethyl]-3,4,4a,5,6,7,8,8a-octahydro-1H-isoquinolin-2-yl]-2-[3,5-dichloro-2-(1-hydroxy-1-methyl-ethyl)-4-pyridyl]ethanone 13

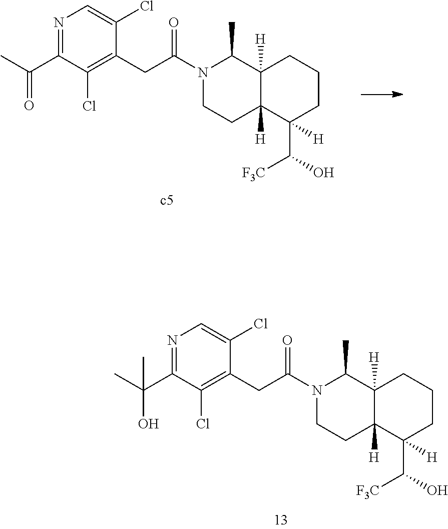 [SMILES: CC(=O)c1ncc(Cl)c(CC(=O)N2CC[C@H]3[C@H]([C@H](O)C(F)(F)F)CCC[C@@H]3[C@@H]2C)c1Cl ; C[C@H]1[C@H]2CCC[C@@H]([C@H](O)C(F)(F)F)[C@@H]2CCN1C(=O)Cc1c(Cl)cnc(C(C)(C)O)c1Cl]

To a stirred solution of 1-[(1 S,4aR,5R,8aS)-1-methyl-5-[(1S)-2,2,2-trifluoro-1-hydroxy-ethyl]-3,4,4a,5,6,7,8,8a-octahydro-1H-isoquinolin-2-yl]-2-(2-acetyl-3,5-dichloro-4-pyridyl)ethanone c5 (100 mg, 0.21 mmol) in THF (4 mL), was added dropwise a 3 M solution of methyllithium in diethoxymethane (0.21 mL, 0.62 mmol) at 0° C. The reaction mixture was stirred 2 h at 0° C. The reaction mixture was diluted with EtOAc (150 mL), washed with a saturated aqueous solution of NaHCO₃ (50 mL) and with brine (3×50 mL). The organic layer was dried over MgSO₄, filtered and concentrated under vacuum. The crude residue was purified by SFC (DIOL 10 μm Kromasil®, 50×250 mm, 360 mL/min, 220 nm, 30° C., elution: EtOH 10%—CO₂ 90%) to afford 27.0 mg of 1-[(1 S,4aR,5R,8aS)-1-methyl-5-[(1S)-2,2,2-trifluoro-1-hydroxy-ethyl]-3,4,4a,5,6,7,8,8a-octahydro-1H-isoquinolin-2-yl]-2-[3,5-dichloro-2-(1-hydroxy-1-methyl-ethyl)-4-pyridyl]ethanone 13, as a gum.

Yield: 26%.

Basic LCMS Method 3 (ES⁺): 497/499/501 (M+H)⁺, 96% purity.

### C.9. Synthesis of 1-[(1S,4aR,5R,8aS)-1-methyl-5-[(1S)-2,2,2-trifluoro-1-hydroxy-ethyl]-3,4,4a,5,6,7,8,8a-octahydro-1H-isoquinolin-2-yl]-2-[3,5-dichloro-2-(2,2-difluoro-1-hydroxy-ethyl)-4-pyridyl]ethanone Isomer A 15-A and 1-[(1S,4aR,5R,8aS)-1-methyl-5-[(1S)-2,2,2-trifluoro-1-hydroxy-ethyl]-3,4,4a,5,6,7,8,8a-octahydro-1H-isoquinolin-2-yl]-2-[3,5-dichloro-2-(2,2-difluoro-1-hydroxy-ethyl)-4-pyridyl]ethanone Isomer B 15-B

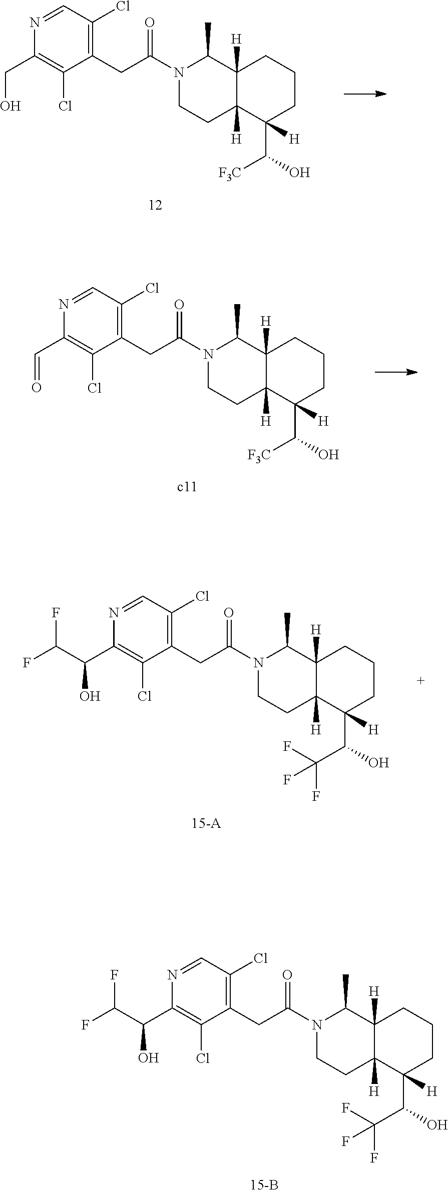 [SMILES: C[C@H]1[C@@H]2CCC[C@H]([C@H](O)C(F)(F)F)[C@@H]2CCN1C(=O)Cc1c(Cl)cnc(CO)c1Cl ; C[C@H]1[C@@H]2CCC[C@H]([C@H](O)C(F)(F)F)[C@@H]2CCN1C(=O)Cc1c(Cl)cnc(C=O)c1Cl ; C[C@H]1[C@@H]2CCC[C@H]([C@H](O)C(F)(F)F)[C@@H]2CCN1C(=O)Cc1c(Cl)cnc([C@@H](O)C(F)F)c1Cl ; C[C@H]1[C@@H]2CCC[C@H]([C@H](O)C(F)(F)F)[C@@H]2CCN1C(=O)Cc1c(Cl)cnc([C@@H](O)C(F)F)c1Cl]

### C.9.1. Synthesis of methyl 4-[2-[(1S,4aR,5R,8aS)-1-methyl-5-[(1S)-2,2,2-trifluoro-1-hydroxy-ethyl]-3,4,4a,5,6,7,8,8a-octahydro-1H-isoquinolin-2-yl]-2-oxo-ethyl]-3,5-dichloro-pyridine-2-carbaldehyde c11

To a solution of 1-[(1 S,4aR,5R,8aS)-1-methyl-5-[(1S)-2,2,2-trifluoro-1-hydroxy-ethyl]-3,4,4a,5,6,7,8,8a-octahydro-1H-isoquinolin-2-yl]-2-[3,5-dichloro-2-(hydroxymethyl)-4-pyridyl]ethanone 12 (254 mg, 0.54 mmol) in 1,4-dioxane (8 mL), manganese dioxide (188 mg, 2.17 mmol) was added and the suspension was stirred overnight at 70° C. The reaction mixture was filtered, and volatiles were removed under vacuum to afford 242 mg of 4-[2-[(1S,4aR,5R,8aS)-1-methyl-5-[(1 S)-2,2,2-trifluoro-1-hydroxy-ethyl]-3,4,4a,5,6,7,8,8a-octahydro-1H-isoquinolin-2-yl]-2-oxo-ethyl]-3,5-dichloro-pyridine-2-carbaldehyde c11.

Yield (crude): 96%.

Basic LCMS Method 2 (ES⁺): 467/469/471 (M+H)⁺.

### C.9.2. Synthesis of 1-[(1S,4aR,5R,8aS)-1-methyl-5-[(1S)-2,2,2-trifluoro-1-hydroxy-ethyl]-3,4,4a,5,6,7,8,8a-octahydro-1H-isoquinolin-2-yl]-2-[3,5-dichloro-2-(2,2-difluoro-1-hydroxy-ethyl)-4-pyridyl]ethanone Isomer A 15-A and 1-[(1S,4aR,5R,8aS)-1-methyl-5-[(1S)-2,2,2-trifluoro-1-hydroxy-ethyl]-3,4,4a,5,6,7,8,8a-octahydro-1H-isoquinolin-2-yl]-2-[3,5-dichloro-2-(2,2-difluoro-1-hydroxy-ethyl)-4-pyridyl]ethanone Isomer B 15-B

To a stirred solution of 4-[2-[(1 S,4aR,5R,8aS)-1-methyl-5-[(1 S)-2,2,2-trifluoro-1-hydroxy-ethyl]-3,4,4a,5,6,7,8,8a-octahydro-1H-isoquinolin-2-yl]-2-oxo-ethyl]-3,5-dichloro-pyridine-2-carbaldehyde c11 (242 mg, 0.52 mmol) and cesium fluoride (318 mg, 2.10 mmol) in DMF (6 mL), difluoromethyl(trimethyl)silane (0.22 mL, 1.6 mmol) was added dropwise. The reaction mixture was stirred 2 h at rt. The reaction mixture was diluted with EtOAc (200 mL) and washed with brine (3×50 mL). The organic layer was dried over MgSO₄, filtered and concentrated under vacuum. The crude residue was purified by normal phase column chromatography (elution: from 20 to 100% of EtOAc in heptane) to afford 82.0 mg of 1-[(1S,4aR,5R,8aS)-1-methyl-5-[(1 S)-2,2,2-trifluoro-1-hydroxy-ethyl]-3,4,4a,5,6,7,8,8a-octahydro-1H-isoquinolin-2-yl]-2-[3,5-dichloro-2-(2,2-difluoro-1-hydroxy-ethyl)-4-pyridyl]ethanone 15 as a mixture of isomers 15-A and 15-B (Yield: 30%, Basic LCMS Method 2 (ES⁺): 519/521/523 (M+H)⁺).

Chiral separation (SFC, DIOL 10 μm Kromasil®, 50×250 mm, 360 mL/min, 220 nm, 30° C., elution: EtOH 10%—CO₂90%) of 72 mg of the above diastereoisomeric mixture 15 afforded:

28.0 mg of 1-[(1 S,4aR,5R,8aS)-1-methyl-5-[(1S)-2,2,2-trifluoro-1-hydroxy-ethyl]-3,4,4a,5,6,7,8,8a-octahydro-1H-isoquinolin-2-yl]-2-[3,5-dichloro-2-(2,2-difluoro-1-hydroxy-ethyl)-4-pyridyl]ethanone Isomer A 15-A.

Yield: 10%

Basic LCMS Method 3 (ES⁺): 519/521/523 (M+H)⁺, 100% purity.

Chiral analysis (LC, Chiralpak AD Daicel®, 3 μm, 4.6×150 mm, 1.5 mL/min, 220 nm, 30° C., elution: iPrOH/n-heptane/DEA 30/70/0.1): RT 1.90 min, 100% de.

6.00 mg of 1-[(1 S,4aR,5R,8aS)-1-methyl-5-[(1S)-2,2,2-trifluoro-1-hydroxy-ethyl]-3,4,4a,5,6,7,8,8a-octahydro-1H-isoquinolin-2-yl]-2-[3,5-dichloro-2-(2,2-difluoro-1-hydroxy-ethyl)-4-pyridyl]ethanone Isomer B 15-B, isolated after additional purifications by normal phase column chromatography (elution: from 20 to 100% of EtOAc in heptane).

Yield: 2%

Basic LCMS Method 3 (ES⁺): 519/521/523 (M+H)⁺, 97% purity. Chiral analysis (LC, Chiralpak AD Daicel®, 3 μm, 4.6×150 mm, 1.5 mL/min, 220 nm, 30° C., elution: iPrOH/n-heptane/DEA 30/70/0.1): RT 2.21 min, 90% de.

### C.10. Synthesis of 1-[(1S,4aR,5R,8aS)-1-methyl-5-[(1R)-2,2,2-trifluoro-1-hydroxy-ethyl]-3,4,4a,5,6,7,8,8a-octahydro-1H-isoquinolin-2-yl]-2-[3,5-dichloro-2-(2,2-difluoro-1-hydroxy-1-methyl-ethyl)-4-pyridyl]ethanone Isomer A 16-A and 1-[(1S,4aR,5R,8aS)-1-methyl-5-[(1R)-2,2,2-trifluoro-1-hydroxy-ethyl]-3,4,4a,5,6,7,8,8a-octahydro-1H-isoquinolin-2-yl]-2-[3,5-dichloro-2-(2,2-difluoro-1-hydroxy-1-methyl-ethyl)-4-pyridyl]ethanone Isomer B 16-B

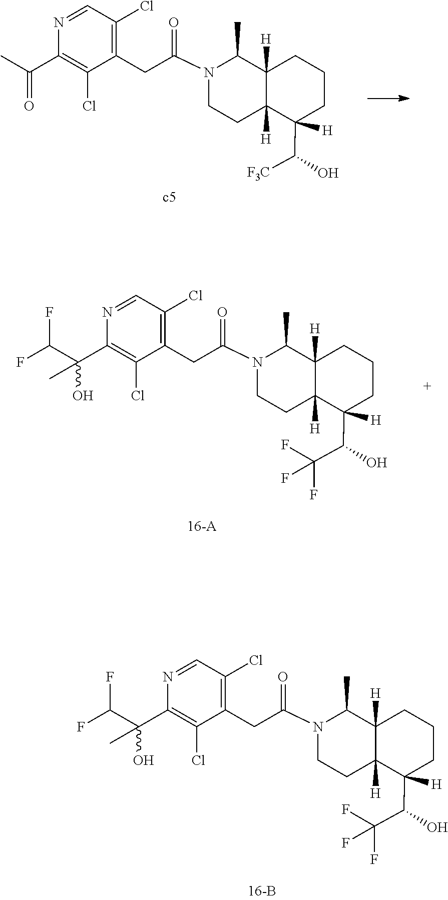 [SMILES: CC(=O)c1ncc(Cl)c(CC(=O)N2CC[C@@H]3[C@@H](CCC[C@@H]3[C@H](O)C(F)(F)F)[C@@H]2C)c1Cl ; C[C@H]1[C@@H]2CCC[C@H]([C@H](O)C(F)(F)F)[C@@H]2CCN1C(=O)Cc1c(Cl)cnc(C(C)(O)C(F)F)c1Cl ; C[C@H]1[C@@H]2CCC[C@H]([C@H](O)C(F)(F)F)[C@@H]2CCN1C(=O)Cc1c(Cl)cnc(C(C)(O)C(F)F)c1Cl]

To a stirred solution of 1-[(1 S,4aR,5R,8aS)-1-methyl-5-[(1S)-2,2,2-trifluoro-1-hydroxy-ethyl]-3,4,4a,5,6,7,8,8a-octahydro-1H-isoquinolin-2-yl]-2-(2-acetyl-3,5-dichloro-4-pyridyl)ethanone c5 (290 mg, 0.60 mmol) and cesium fluoride (370 mg, 2.4 mmol) in DMF (10 mL), difluoromethyl(trimethyl)silane (0.26 mL, 1.81 mmol) was added dropwise at rt. The reaction mixture was stirred 2 h at rt. The reaction mixture was diluted with EtOAc (200 mL) and washed with brine (3×50 mL). The organic layer was dried over MgSO₄, filtered and concentrated under vacuum. The crude residue was purified by normal phase column chromatography (elution: from 0 to 70% EtOAc in heptane) to afford 60.0 mg of 1-[(1S,4aR,5R,8aS)-1-methyl-5-[(1R)-2,2,2-trifluoro-1-hydroxy-ethyl]-3,4,4a,5,6,7,8,8a-octahydro-1H-isoquinolin-2-yl]-2-[3,5-dichloro-2-(2,2-difluoro-1-hydroxy-1-methyl-ethyl)-4-pyridyl]ethanone 16 as a mixture of isomers 16-A and 16-B (Yield: 19%, Basic LCMS Method 2 (ES⁺): 533/535/537 (M+H)⁺).

Chiral separation (LC, AD, 10 μm, 250×10 mm, 4.8 mL/min, 220 nm, 30° C., elution: EtOH/n-heptane 30/70) of 50 mg of the above diastereoisomeric mixture 1-[(1S,4aR,5R,8aS)-1-methyl-5-[(1R)-2,2,2-trifluoro-1-hydroxy-ethyl]-3,4,4a,5,6,7,8,8a-octahydro-1H-isoquinolin-2-yl]-2-[3,5-dichloro-2-(2,2-difluoro-1-hydroxy-1-methyl-ethyl)-4-pyridyl]ethanone 16 afforded:

12.0 mg of 1-[(1S,4aR,5R,8aS)-1-methyl-5-[(1R)-2,2,2-trifluoro-1-hydroxy-ethyl]-3,4,4a,5,6,7,8,8a-octahydro-1H-isoquinolin-2-yl]-2-[3,5-dichloro-2-(2,2-difluoro-1-hydroxy-1-methyl-ethyl)-4-pyridyl]ethanone Isomer A 16-A, after an additional purification by reverse phase column chromatography (Basic LCMS prep), as a solid.

Yield: 4%.

Basic LCMS Method 3 (ES⁺): 533/535/537 (M+H)⁺, 88% purity.

Chiral analysis (LC, Chiralpak AD Daicel®, 3 μm, 4.6×150 mm, 1.5 mL/min, 220 nm, 30° C., elution: IPrOH/n-heptane/DEA 30/70/0.1): RT 1.89 min, 100% de.

12.0 mg of 1-[(1S,4aR,5R,8aS)-1-methyl-5-[(1R)-2,2,2-trifluoro-1-hydroxy-ethyl]-3,4,4a,5,6,7,8,8a-octahydro-1H-isoquinolin-2-yl]-2-[3,5-dichloro-2-(2,2-difluoro-1-hydroxy-1-methyl-ethyl)-4-pyridyl]ethanone Isomer B 16-B after an additional purification by reverse phase column chromatography (Basic LCMS prep), as solid.

Yield: 4%

Basic LCMS Method 3 (ES⁺): 533/535/537 (M+H)⁺, 89% purity.

Chiral analysis (LC, Chiralpak AD Daicel®, 3 μm, 4.6×150 mm, 1.5 mL/min, 220 nm, 30° C., elution: IPrOH/n-heptane/DEA 30/70/0.1): RT 2.15 min, 100% de.

### C.11. Synthesis of 1-[(1S,4aR,5R,8aS)-5-[(1S)-2,2-difluoro-1-hydroxy-ethyl]-1-methyl-3,4,4a,5,6,7,8,8a-octahydro-1H-isoquinolin-2-yl]-2-[3,5-dichloro-2-(1-hydroxyethyl)-4-pyridyl]ethanone Isomer A 17-A and 1-[(1S,4aR,5R,8aS)-5-[(1S)-2,2-difluoro-1-hydroxy-ethyl]-1-methyl-3,4,4a,5,6,7,8,8a-octahydro-1H-isoquinolin-2-yl]-2-[3,5-dichloro-2-(1-hydroxyethyl)-4-pyridyl]ethanone Isomer B 17-B

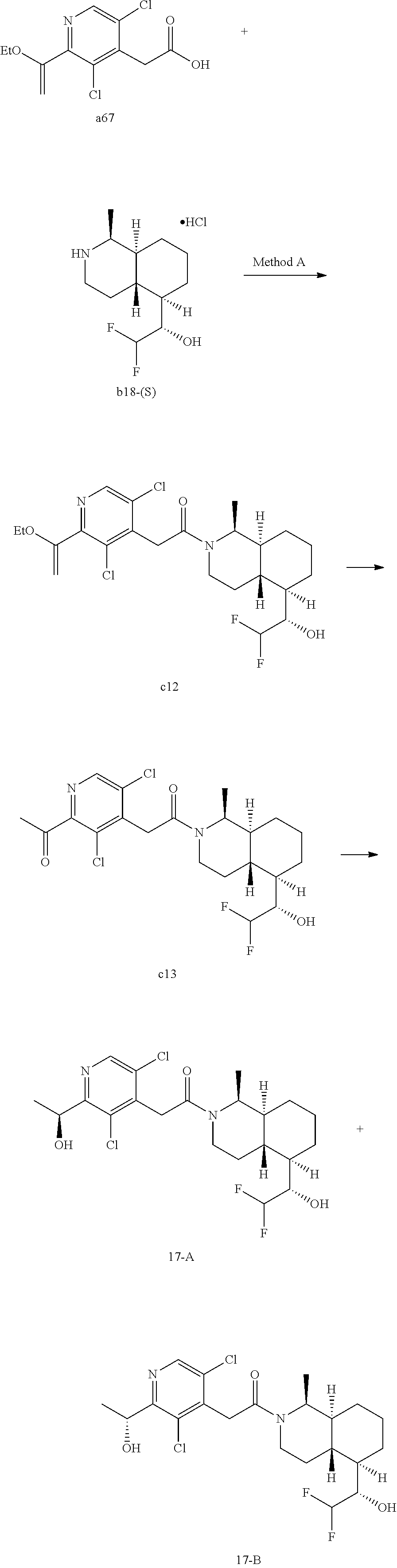

### C.11.1. Synthesis of 1-[(1S,4aR,5R,8aS)-5-[(1S)-2,2-difluoro-1-hydroxy-ethyl]-1-methyl-3,4,4a,5,6,7,8,8a-octahydro-1H-isoquinolin-2-yl]-2-[3,5-dichloro-2-(1-ethoxyvinyl)-4-pyridyl]ethanone c12

1-[(1 S,4aR,5R,8aS)-5-[(1S)-2,2-difluoro-1-hydroxy-ethyl]-1-methyl-3,4,4a,5,6,7,8,8a-octahydro-1H-isoquinolin-2-yl]-2-[3,5-dichloro-2-(1-ethoxyvinyl)-4-pyridyl]ethanone c12 was prepared according to Method A, by reacting (1S)-1-[(1S,4aR,5R,8aS)-1-methyl-1,2,3,4,4a,5,6,7,8,8a-decahydroisoquinolin-5-yl]-2,2-difluoro-ethanol hydrochloride b18-(S) with 2-[3,5-dichloro-2-(1-ethoxyvinyl)-4-pyridyl]acetic acid a67 in the presence of HBTU and DIPEA in DMF. The crude was used in next step without further purification.

Yield (crude): 78%.

Acid LCMS Method 1 (ES⁺): 491/493/495 (M+H)⁺.

### C.11.2. Synthesis of 1-[(1S,4aR,5R,8aS)-5-[(1S)-2,2-difluoro-1-hydroxy-ethyl]-1-methyl-3,4,4a,5,6,7,8,8a-octahydro-1H-isoquinolin-2-yl]-2-(2-acetyl-3,5-dichloro-4-pyridyl)ethanone c13

To a solution of crude 1-[(1S,4aR,5R,8aS)-5-[(1 S)-2,2-difluoro-1-hydroxy-ethyl]-1-methyl-3,4,4a,5,6,7,8,8a-octahydro-1H-isoquinolin-2-yl]-2-[3,5-dichloro-2-(1-ethoxyvinyl)-4-pyridyl]ethanone c12 (235 mg, 0.41 mmol) in THF (5 mL) was added a 1N aqueous solution of HCl (2 mL) and the reaction mixture was stirred overnight at rt. The reaction mixture was quenched with a saturated aqueous solution of sodium bicarbonate (10 mL) and extracted with EtOAc (10 mL). The organic layer was dried over MgSO₄, filtered and concentrated under vacuum to afford 220 mg of 1-[(1 S,4aR,5R,8aS)-5-[(1 S)-2,2-difluoro-1-hydroxy-ethyl]-1-methyl-3,4,4a,5,6,7,8,8a-octahydro-1H-isoquinolin-2-yl]-2-(2-acetyl-3,5-dichloro-4-pyridyl)ethanone c13, which was used in next step without further purification.

Yield (crude): 78%.

Acid LCMS Method 1 (ES⁺): 463/465/467 (M+H)⁺, 87% purity.

### C.11.3. Synthesis of 1-[(1S,4aR,5R,8aS)-5-[(1S)-2,2-difluoro-1-hydroxy-ethyl]-1-methyl-3,4,4a,5,6,7,8,8a-octahydro-1H-isoquinolin-2-yl]-2-[3,5-dichloro-2-(1-hydroxyethyl)-4-pyridyl]ethanone Isomer A 17-A and 1-[(1S,4aR,5R,8aS)-5-[(1S)-2,2-difluoro-1-hydroxy-ethyl]-1-methyl-3,4,4a,5,6,7,8,8a-octahydro-1H-isoquinolin-2-yl]-2-[3,5-dichloro-2-(1-hydroxyethyl)-4-pyridyl]ethanone Isomer B 17-B

To a solution of crude 1-[(1S,4aR,5R,8aS)-5-[(1 S)-2,2-difluoro-1-hydroxy-ethyl]-1-methyl-3,4,4a,5,6,7,8,8a-octahydro-1H-isoquinolin-2-yl]-2-(2-acetyl-3,5-dichloro-4-pyridyl)ethanone c13 (220 mg, 0.32 mmol) in EtOH (6 mL) was added sodium borohydride (14.0 mg, 0.37 mmol) at 0° C. and the reaction mixture was stirred overnight at rt. The reaction mixture was quenched with H₂O (5 mL) and stirred for 1 h. Then, a 1N aqueous solution of HCl (2 mL) was added and the mixture was stirred for another hour. H₂O (25 mL) was added, and the aqueous layer was extracted with DCM (2×50 mL). The organic layer was dried over MgSO₄, filtered and concentrated under vacuum. The crude was purified by reverse phase column chromatography (YMC Triart C18 column, 10 μm, 80×204 mm, elution: from 5 to 95% ACN in H₂O+0.025% NH₄OH) to afford 153 mg of 1-[(1S,4aR,5R,8aS)-5-[(1S)-2,2-difluoro-1-hydroxy-ethyl]-1-methyl-3,4,4a,5,6,7,8,8a-octahydro-1H-isoquinolin-2-yl]-2-[3,5-dichloro-2-(1-hydroxyethyl)-4-pyridyl]ethanone 17 as a mixture of isomers 17-A and 17-B (Yield: 88%, Acid LCMS Method 2 (ES⁺): 465/467/469 (M+H)⁺).

Chiral separation (SFC, Chiralpak AD Daicel®, 20 μm, 279×50 mm, 360 mL/min, 220 nm, 30° C., elution: iPrOH 20%—CO₂ 80%) of the above mixture 17 afforded:

40.0 mg of 1-[(1 S,4aR,5R,8aS)-5-[(1 S)-2,2-difluoro-1-hydroxy-ethyl]-1-methyl-3,4,4a,5,6,7,8,8a-octahydro-1H-isoquinolin-2-yl]-2-[3,5-dichloro-2-[(1S)-(1-hydroxyethyl]-)-4-pyridyl]ethanone Isomer A 17-A, as a solid.

Yield: 30%

Basic LCMS Method 3 (ES⁺): 465/467/469 (M+H)⁺, 99% purity.

Acid LCMS Method 2 (ES⁺): 465/467/469 (M+H)⁺, 99% purity.

Chiral analysis (LC, Chiralpak AD Daicel®, 3 μm, 150×4.6 mm, 1.5 mL/min, 220 nm, 30° C., elution: IPrOH/n-heptane/DEA 50/50/0.1): RT 1.54 min, 100% de.

42.0 mg of 1-[(1 S,4aR,5R,8aS)-5-[(1 S)-2,2-difluoro-1-hydroxy-ethyl]-1-methyl-3,4,4a,5,6,7,8,8a-octahydro-1H-isoquinolin-2-yl]-2-[3,5-dichloro-2-[(1S)-(1-hydroxyethyl]-)-4-pyridyl]ethanone Isomer B 17-B, as a solid.

Yield: 31%

Basic LCMS Method 3 (ES⁺): 465/467/469 (M+H)⁺, 96% purity.

Acid LCMS Method 2 (ES⁺): 465/467/469 (M+H)⁺, 98% purity.

Chiral analysis (LC, Chiralpak AD Daicel®, 3 μm, 150×4.6 mm, 1.5 mL/min, 220 nm, 30° C., elution: iPrOH/n-heptane/DEA 50/50/0.1): RT 1.87 min, 98% de.

### C.12. Synthesis of 1-[(1S,4aR,5R,8aS)-5-[(1S)-2,2-difluoro-1-hydroxy-1-methyl-ethyl]-1-methyl-3,4,4a,5,6,7,8,8a-octahydro-1H-isoquinolin-2-yl]-2-[3,5-dichloro-2-(1-hydroxyethyl)-4-pyridyl]ethanone 18-A and 1-[(1S,4aR,5R,8aS)-5-[(1S)-2,2-difluoro-1-hydroxy-1-methyl-ethyl]-1-methyl-3,4,4a,5,6,7,8,8a-octahydro-1H-isoquinolin-2-yl]-2-[3,5-dichloro-2-(1-hydroxyethyl)-4-pyridyl]ethanone Isomer B 18-B

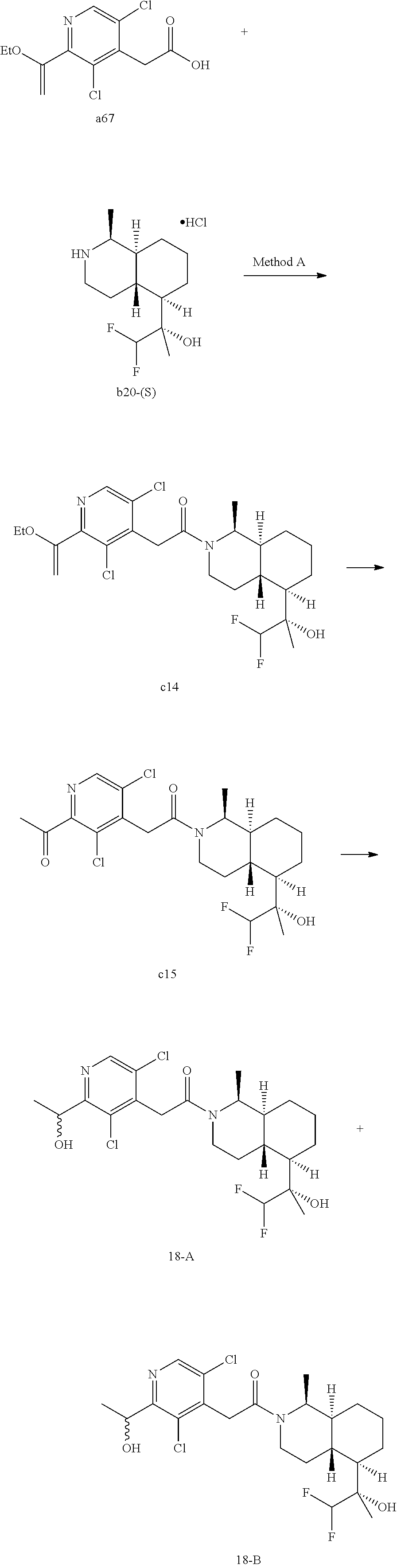

### C.12.1. Synthesis of 1-[(1S,4aR,5R,8aS)-5-[(1S)-2,2-difluoro-1-hydroxy-1-methyl-ethyl]-1-methyl-3,4,4a,5,6,7,8,8a-octahydro-1H-isoquinolin-2-yl]-2-[3,5-dichloro-2-(1-ethoxyvinyl)-4-pyridyl]ethanone c14

1-[(1 S,4aR,5R,8aS)-5-[(1S)-2,2-difluoro-1-hydroxy-1-methyl-ethyl]-1-methyl-3,4,4a,5,6,7,8,8a-octahydro-1H-isoquinolin-2-yl]-2-[3,5-dichloro-2-(1-ethoxyvinyl)-4-pyridyl]ethanone c14 was prepared according to Method A, by reacting (2S)-2-[(1S,4aR,5R,8aS)-1-methyl-1,2,3,4,4a,5,6,7,8,8a-decahydroisoquinolin-5-yl]-1,1-difluoro-propan-2-ol hydrochloride b20-(S) with 2-[3,5-dichloro-2-(1-ethoxyvinyl)-4-pyridyl]acetic acid a67 in the presence of HBTU and DIPEA in DMF. The crude was used in next step without further purification.

Yield (crude): 85%.

Acid LCMS Method 1 (ES⁺): 505/507/509 (M+H)⁺, 93% purity.

### C.12.2. Synthesis of 1-[(1S,4aR,5R,8aS)-5-[(1S)-2,2-difluoro-1-hydroxy-1-methyl-ethyl]-1-methyl-3,4,4a,5,6,7,8,8a-octahydro-1H-isoquinolin-2-yl]-2-(2-acetyl-3,5-dichloro-4-pyridyl)ethanone c15

To a solution of 1-[(1 S,4aR,5R,8aS)-5-[(1S)-2,2-difluoro-1-hydroxy-1-methyl-ethyl]-1-methyl-3,4,4a,5,6,7,8,8a-octahydro-1H-isoquinolin-2-yl]-2-[3,5-dichloro-2-(1-ethoxyvinyl)-4-pyridyl]ethanone c14 (236 mg, 0.43 mmol) in THF (5 mL) was added a 1N aqueous solution of HCl in H₂O (2 mL) and the reaction mixture was stirred overnight at rt. The reaction mixture was quenched with a saturated aqueous solution of sodium bicarbonate (10 mL) and extracted with EtOAc (10 mL). The organic layer was dried over MgSO₄, filtered and concentrated under vacuum to afford 234 mg of 1-[(1S,4aR,5R,8aS)-5-[(1S)-2,2-difluoro-1-hydroxy-1-methyl-ethyl]-1-methyl-3,4,4a,5,6,7,8,8a-octahydro-1H-isoquinolin-2-yl]-2-[3,5-dichloro-2-(1-ethoxyvinyl)-4-pyridyl]ethanone c15, which was used in next step without further purification.

Yield (crude): 88%

Acid LCMS Method 1 (ES⁺): 477/479/481 (M+H)⁺.

### C.12.3. Synthesis of 1-[(1S,4aR,5R,8aS)-5-[(1S)-2,2-difluoro-1-hydroxy-1-methyl-ethyl]-1-methyl-3,4,4a,5,6,7,8,8a-octahydro-1H-isoquinolin-2-yl]-2-[3,5-dichloro-2-(1-hydroxyethyl)-4-pyridyl]ethanone Isomer A 18-A and 1-[(1S,4aR,5R,8aS)-5-[(1S)-2,2-difluoro-1-hydroxy-1-methyl-ethyl]-1-methyl-3,4,4a,5,6,7,8,8a-octahydro-1H-isoquinolin-2-yl]-2-[3,5-dichloro-2-(1-hydroxyethyl)-4-pyridyl]ethenone Isomer B 18-B

To a solution of 1-[(1 S,4aR,5R,8aS)-5-[(1S)-2,2-difluoro-1-hydroxy-1-methyl-ethyl]-1-methyl-3,4,4a,5,6,7,8,8a-octahydro-1H-isoquinolin-2-yl]-2-[3,5-dichloro-2-(1-ethoxyvinyl)-4-pyridyl]ethanone c15 (234 mg, 0.38 mmol) in EtOH (6 mL) was added sodium borohydride (16.0 mg, 0.42 mmol) at 0° C. and the reaction mixture was stirred overnight at rt. The reaction mixture was quenched with H₂O (5 mL) and stirred for 1 h. Then, a 1N aqueous solution of HCl (2 mL) was added and the mixture was stirred for another hour at rt. H₂O (25 mL) was added, and the aqueous layer was extracted with DCM (2×50 mL). The organic layer was dried over MgSO₄, filtered and concentrated under vacuum to give the crude residue which was purified by reverse phase column chromatography (YMC Triart C18 column, 10 μm, 80×204 mm, elution: from 5 to 95% ACN in H₂O+0.025% NH₄OH) to afford 142 mg of 1-[(1S,4aR,5R,8aS)-5-[(1S)-2,2-difluoro-1-hydroxy-1-methyl-ethyl]-1-methyl-3,4,4a,5,6,7,8,8a-octahydro-1H-isoquinolin-2-yl]-2-[3,5-dichloro-2-(1-hydroxyethyl)-4-pyridyl]ethanone 18 as a mixture of isomers 18-A and 18-B (Yield: 68%, Acid LCMS Method 2 (ES⁺): 479/481/483 (M+H)⁺, 88% purity).

Chiral separation (SFC, Chiralpak AD Daicel®, 20 μm, 279×50 mm, 360 mL/min, 220 nm, 30° C., elution: iPrOH 25%—CO₂ 75%) of the above mixture 18 afforded:

44.0 mg of 1-[(1 S,4aR,5R,8aS)-5-[(1 S)-2,2-difluoro-1-hydroxy-1-methyl-ethyl]-1-methyl-3,4,4a,5,6,7,8,8a-octahydro-1H-isoquinolin-2-yl]-2-[3,5-dichloro-2-(1-hydroxyethyl)-4-pyridyl]ethanone Isomer A 18-A, as a solid.

Yield: 34%

Basic LCMS Method 3 (ES⁺): 479/481/483 (M+H)⁺, 98% purity.

Acid LCMS Method 2 (ES⁺): 479/481/483 (M+H)⁺, 98% purity.

Chiral analysis (LC, Chiralpak IG Daicel®, 3 μm, 150×4.6 mm, 1.5 mL/min, 220 nm, 30° C., elution: iPrOH/n-heptane/DEA 50/50/0.1): RT 3.93 min, 100% ee.

39.0 mg of 1-[(1 S,4aR,5R,8aS)-5-[(1 S)-2,2-difluoro-1-hydroxy-1-methyl-ethyl]-1-methyl-3,4,4a,5,6,7,8,8a-octahydro-1H-isoquinolin-2-yl]-2-[3,5-dichloro-2-(1-hydroxyethyl)-4-pyridyl]ethanone Isomer B 18-B, as a solid.

Yield: 30%

Basic LCMS Method 3 (ES⁺): 479/481/483 (M+H)⁺, 97% purity.

Acid LCMS Method 2 (ES⁺): 479/481/483 (M+H)⁺, 99% purity.

Chiral analysis (LC, Chiralpak IG Daicel®, 3 μm, 150×4.6 mm, 1.5 mL/min, 220 nm, 30° C., elution: iPrOH/n-heptane/DEA 50/50/0.1): RT 6.17 min, 97% ee.

### C.13. Synthesis of 2-[2-[(1S,4aS,8aS)-5-(3-hydroxy-3-methyl-but-1-ynyl)-1-methyl-3,4,4a,5,6,7,8,8a-octahydro-1H-isoquinolin-2-yl]-2-oxo-ethyl]-3-chloro-4-methoxy-benzonitrile 20 and 2-[2-[(1S,4aS,5S,8aS)-5-(3-hydroxy-3-methyl-butyl)-1-methyl-3,4,4a,5,6,7,8,8a-octahydro-1H-isoquinolin-2-yl]-2-oxo-ethyl]-3-chloro-4-methoxy-benzonitrile 21

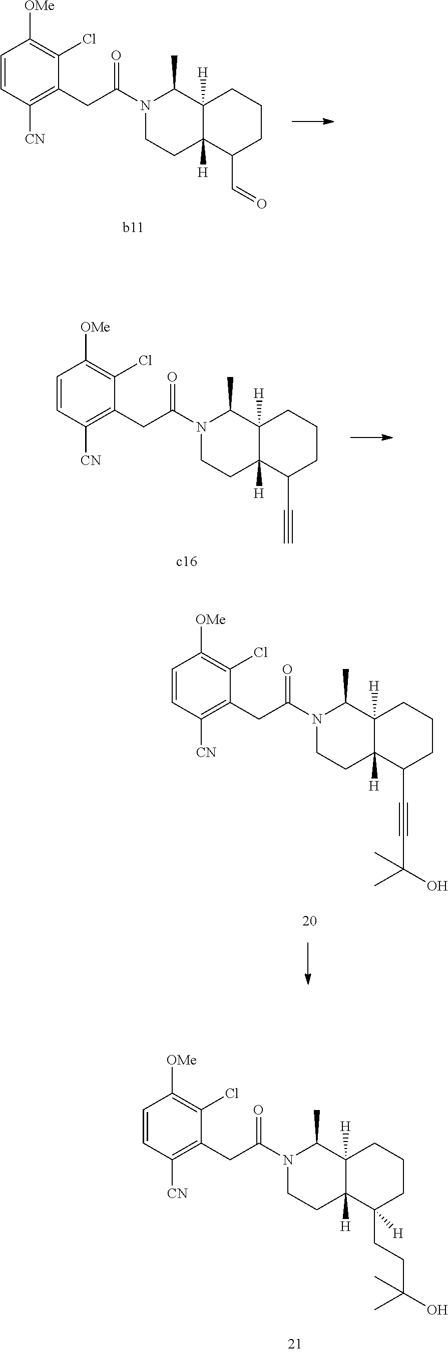 [SMILES: COc1ccc(C#N)c(CC(=O)N2CC[C@H]3C(C=O)CCC[C@@H]3[C@@H]2C)c1Cl ; C#CC1CCC[C@H]2[C@H]1CCN(C(=O)Cc1c(C#N)ccc(OC)c1Cl)[C@H]2C ; COc1ccc(C#N)c(CC(=O)N2CC[C@H]3C(C#CC(C)(C)O)CCC[C@@H]3[C@@H]2C)c1Cl ; COc1ccc(C#N)c(CC(=O)N2CC[C@H]3[C@H](CCC(C)(C)O)CCC[C@@H]3[C@@H]2C)c1Cl]

### C.13.1. Synthesis of 2-[2-[(1S,4aS,5R,8aS)-5-ethynyl-1-methyl-3,4,4a,5,6,7,8,8a-octahydro-1H-isoquinolin-2-yl]-2-oxo-ethyl]-3-chloro-4-methoxy-benzonitrile c16

To a stirred solution of 2-[2-[(1S,4aR,8aS)-5-formyl-1-methyl-3,4,4a,5,6,7,8,8a-octahydro-1H-isoquinolin-2-yl]-2-oxo-ethyl]-3-chloro-4-methoxy-benzonitrile b11 (5.00 g, 13.0 mmol) in MeOH (50 mL) were added 1-diazo-1-dimethoxyphosphoryl-propan-2-one (15.0 mmol, 3.00 g) and K₂CO₃ (3.60 g, 26.0 mmol) at rt. The reaction mixture was stirred overnight at rt, then diluted with EtOAc (150 mL) and successively washed with a 1 N aqueous solution of HCl (50 mL), a saturated aqueous solution of sodium carbonate (50 mL) and brine (50 mL). The organic layer was dried over MgSO₄, filtered and concentrated under vacuum. The crude residue was purified by normal phase column chromatography (elution: From 10 to 90% EtOAc in heptane) to afford 4.70 g of 2-[2-[(1S,4aS,5R,8aS)-5-ethynyl-1-methyl-3,4,4a,5,6,7,8,8a-octahydro-1H-isoquinolin-2-yl]-2-oxo-ethyl]-3-chloro-4-methoxy-benzonitrile c16.

Yield: 95%.

Acid LCMS Method 1 (ES⁺): 385/387/389 (M+H)⁺.

### C.13.2. Synthesis of 2-[2-[(1S,4aS,8aS)-5-(3-hydroxy-3-methyl-but-1-ynyl)-1-methyl-3,4,4a,5,6,7,8,8a-octahydro-1H-isoquinolin-2-yl]-2-oxo-ethyl]-3-chloro-4-methoxy-benzonitrile 20

At −78° C. under argon, a 2.5 M solution of n-BuLi in hexanes (8.70 mL, 21.7 mmol) was added to a solution of 2-[2-[(1 S,4aS,5R,8aS)-5-ethynyl-1-methyl-3,4,4a,5,6,7,8,8a-octahydro-1H-isoquinolin-2-yl]-2-oxo-ethyl]-3-chloro-4-methoxy-benzonitrile c16 (3.70 g, 9.60 mmol) in THF (100 mL). The reaction mixture was stirred 15 min at −78° C. Acetone (2.80 mL, 38.0 mmol) was added. The reaction mixture was stirred 15 min at −78° C. then 2 h at rt. After a quench with a saturated aqueous solution of NH₄Cl (20 mL), the reaction mixture was diluted with EtOAc (150 mL) and successively washed with a 1N aqueous solution of HCl (50 mL), a saturated aqueous solution of sodium carbonate (50 mL) and brine (50 mL). The organic layer was dried over MgSO₄, filtered and concentrated under vacuum. The crude residue was purified by normal phase column chromatography (elution: from 12 to 100% of EtOAc in heptane) to afford 1.80 g of 2-[2-[(1S,4aS,8aS)-5-(3-hydroxy-3-methyl-but-1-ynyl)-1-methyl-3,4,4a,5,6,7,8,8a-octahydro-1H-isoquinolin-2-yl]-2-oxo-ethyl]-3-chloro-4-methoxy-benzonitrile 20, as a solid.

Yield: 42%.

Acid LCMS Method 1 (ES⁺): 443/445/447 (M+H)⁺.

### C.13.3. Synthesis of 2-[2-[(1S,4aS,5S,8aS)-5-(3-hydroxy-3-methyl-butyl)-1-methyl-3,4,4a,5,6,7,8,8a-octahydro-1H-isoquinolin-2-yl]-2-oxo-ethyl]-3-chloro-4-methoxy-benzonitrile 21

2-[2-[(1S,4aS,5R,8aS)-5-(3-hydroxy-3-methyl-but-1-ynyl)-1-methyl-3,4,4a,5,6,7,8,8a-octahydro-1H-isoquinolin-2-yl]-2-oxo-ethyl]-3-chloro-4-methoxy-benzonitrile 20 (650 mg, 1.51 mmol) and Pd/C 20% (Johnson Matthey Type 91 Pearl, 15.6 mg, 0.029 mmol) were mixed in EtOH (10 mL) and 1,4-dioxane (10 mL) in a sealed autoclave. The suspension was subjected to 6 bars of H₂ at rt under a vigorous stirring during 4 h. The reaction mixture was filtered through a pad of Celite® and volatiles were removed under reduced pressure. The crude residue was purified by normal phase column chromatography (elution: from 10 to 90% of EtOAc in heptane) to afford 436 mg of 2-[2-[(1S,4aS,5S,8aS)-5-(3-hydroxy-3-methyl-butyl)-1-methyl-3,4,4a,5,6,7,8,8a-octahydro-1H-isoquinolin-2-yl]-2-oxo-ethyl]-3-chloro-4-methoxy-benzonitrile 21, as a solid.

Yield: 64%.

Basic LCMS Method 3 (ES⁺): 447/449 (M+H)⁺, 90% purity.

Acid LCMS Method 2 (ES⁺): 447/449 (M+H)⁺, 87% purity.

### C.14. Synthesis of 1-[(1S,4aS,5S,8aS)-5-(3-hydroxy-3-methyl-butyl)-1-methyl-3,4,4a,5,6,7,8,8a-octahydro-1H-isoquinolin-2-yl]-2-(3,5-dichloro-1-methyl-indazol-4-yl)ethanone 22

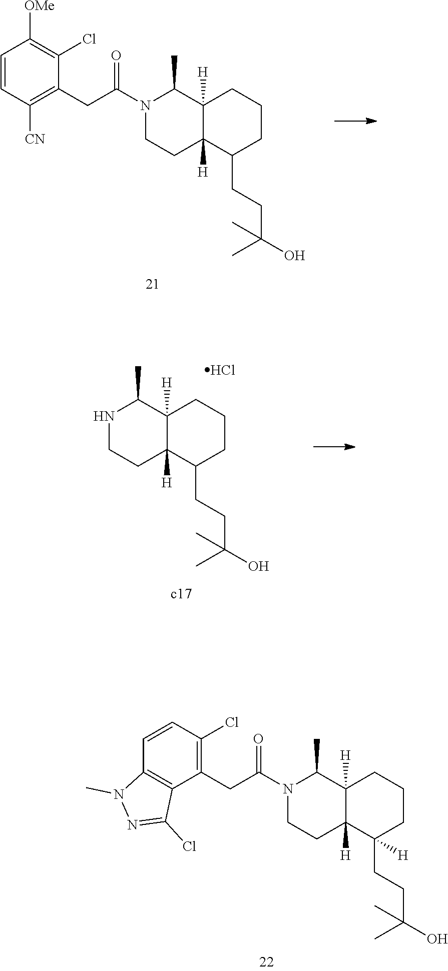 [SMILES: COc1ccc(C#N)c(CC(=O)N2CC[C@H]3C(CCC(C)(C)O)CCC[C@@H]3[C@@H]2C)c1Cl ; C[C@@H]1NCC[C@H]2C(CCC(C)(C)O)CCC[C@H]12 ; Cl ; C[C@H]1[C@H]2CCC[C@@H](CCC(C)(C)O)[C@@H]2CCN1C(=O)Cc1c(Cl)ccc2c1c(Cl)nn2C]

### C.14.1. Synthesis of 4-[(1S,4aS,5S,8aS)-1-methyl-1,2,3,4,4a,5,6,7,8,8a-decahydroisoquinolin-5-yl]-2-methyl-butan-2-ol hydrochloride c17

In a screwed cap vial, 2-[2-[(1S,4aS,5S,8aS)-5-(3-hydroxy-3-methyl-butyl)-1-methyl-3,4,4a,5,6,7,8,8a-octahydro-1H-isoquinolin-2-yl]-2-oxo-ethyl]-3-chloro-4-methoxy-benzonitrile 21 (300 mg, 0.67 mmol) was dissolved in 1,4-dioxane (2 mL) and an 2 M aqueous solution of LiOH (8.00 mL, 16.0 mmol) was added. The reaction mixture was subjected to microwave irradiation for 1 h at 150° C. The mixture was extracted with DCM (5×50 mL). The organic layer was dried over MgSO₄, filtered and concentrated under vacuum. The crude residue was diluted with EtOAc and extracted with 1N aqueous solution of HCl. The aqueous layer was concentrated under vacuum to afford 185 mg of 1-[(1S,4aS,5S,8aS)-1-methyl-1,2,3,4,4a,5,6,7,8,8a-decahydroisoquinolin-5-yl]-2-methyl-butan-2-ol hydrochloride c17 as a white solid.

Yield (crude): 100%.

Acid LCMS Method 1 (ES⁺): 240 (M+H)⁺.

### C.14.2. Synthesis of 1-[(1S,4aS,5S,8aS)-5-(3-hydroxy-3-methyl-butyl)-1-methyl-3,4,4a,5,6,7,8,8a-octahydro-1H-isoquinolin-2-yl]-2-(3,5-dichloro-1-methyl-indazol-4-yl)ethanone 22

1-[(1 S,4aS,5S,8aS)-5-(3-hydroxy-3-methyl-butyl)-1-methyl-3,4,4a,5,6,7,8,8a-octahydro-1H-isoquinolin-2-yl]-2-(3,5-dichloro-1-methyl-indazol-4-yl)ethanone 22 was prepared according to Method A, by reacting 4-[(1S,4aS,5S,8aS)-1-methyl-1,2,3,4,4a,5,6,7,8,8a-decahydroisoquinolin-5-yl]-2-methyl-butan-2-ol hydrochloride c17 with 2-(3,5-dichloro-1-methyl-indazol-4-yl)acetic acid a33 in the presence of HBTU and Et₃N (3 equiv.) in DMF. The crude residue was purified by reverse phase column chromatography (Acid LCMS prep) to afford 1-[(1 S,4aS,5S,8aS)-5-(3-hydroxy-3-methyl-butyl)-1-methyl-3,4,4a,5,6,7,8,8a-octahydro-1H-isoquinolin-2-yl]-2-(3,5-dichloro-1-methyl-indazol-4-yl)ethanone 22, as a white solid.

Yield: 23%

Basic LCMS Method 3 (ES⁺): 480/482/484 (M+H)⁺, 95% purity.

Acid LCMS Method 2 (ES⁺): 480/482/484 (M+H)⁺, 91% purity.

### C.15. Synthesis of 1-[(1S,4aS,5S,8aS)-5-(3-hydroxy-3-methyl-butyl)-1-methyl-3,4,4a,5,6,7,8,8a-octahydro-1H-isoquinolin-2-yl]-2-[3,5-dichloro-2-(hydroxymethyl)-4-pyridyl]ethanone 23

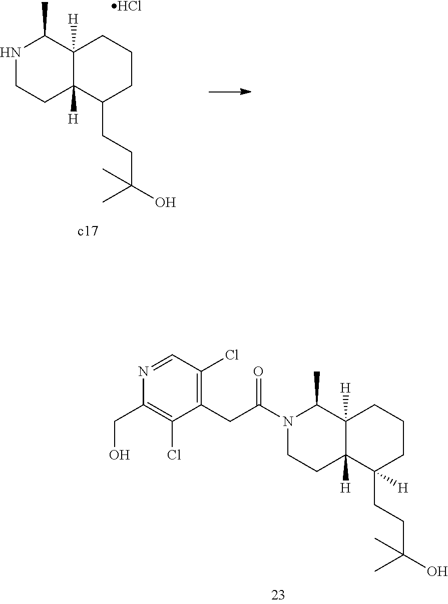 [SMILES: C[C@@H]1NCC[C@H]2C(CCC(C)(C)O)CCC[C@H]12 ; Cl ; C[C@H]1[C@H]2CCC[C@@H](CCC(C)(C)O)[C@@H]2CCN1C(=O)Cc1c(Cl)cnc(CO)c1Cl]

1-[(1 S,4aS,5S,8aS)-5-(3-hydroxy-3-methyl-butyl)-1-methyl-3,4,4a,5,6,7,8,8a-octahydro-1H-isoquinolin-2-yl]-2-[3,5-dichloro-2-(hydroxymethyl)-4-pyridyl]ethanone 23 was prepared according to Method A, by reacting 4-[(1S,4aS,5S,8aS)-1-methyl-1,2,3,4,4a,5,6,7,8,8a-decahydroisoquinolin-5-yl]-2-methyl-butan-2-ol hydrochloride c17 with 2-[3,5-dichloro-2-(hydroxymethyl)-4-pyridyl]acetic acid a28 in the presence of HBTU and Et₃N (3 equiv.) in DMF. The crude residue was purified by reverse phase column chromatography (Conditions: Eternity XT 200 g C18 column, 10 μm, 50×200 mm, 70 mL/min, 215 nm, 35° C., elution: H₂O/ACN+NH₄OH 0.025%) to afford 110 mg of 1-[(1 S,4aS,5S,8aS)-5-(3-hydroxy-3-methyl-butyl)-1-methyl-3,4,4a,5,6,7,8,8a-octahydro-1H-isoquinolin-2-yl]-2-[3,5-dichloro-2-(hydroxymethyl)-4-pyridyl]ethanone 23, as a white solid.

Yield: 17%

Basic LCMS Method 3 (ES⁺): 457/459/461 (M+H)⁺, 97% purity.

Acid LCMS Method 2 (ES⁺): 457/459/461 (M+H)⁺, 94% purity.

### C.16. Synthesis of 1-[(1S,4aS,5S,8aS)-5-(3-hydroxy-3-methyl-butyl)-1-methyl-3,4,4a,5,6,7,8,8a-octahydro-1H-isoquinolin-2-yl]-2-[3,5-dichloro-2-(1-hydroxyethyl)-4-pyridyl]ethanone Isomer A 24-A and 1-[(1S,4aS,5S,8aS)-5-(3-hydroxy-3-methyl-butyl)-1-methyl-3,4,4a,5,6,7,8,8a-octahydro-1H-isoquinolin-2-yl]-2-[3,5-dichloro-2-(1-hydroxyethyl)-4-pyridyl]ethanone Isomer B 24-B

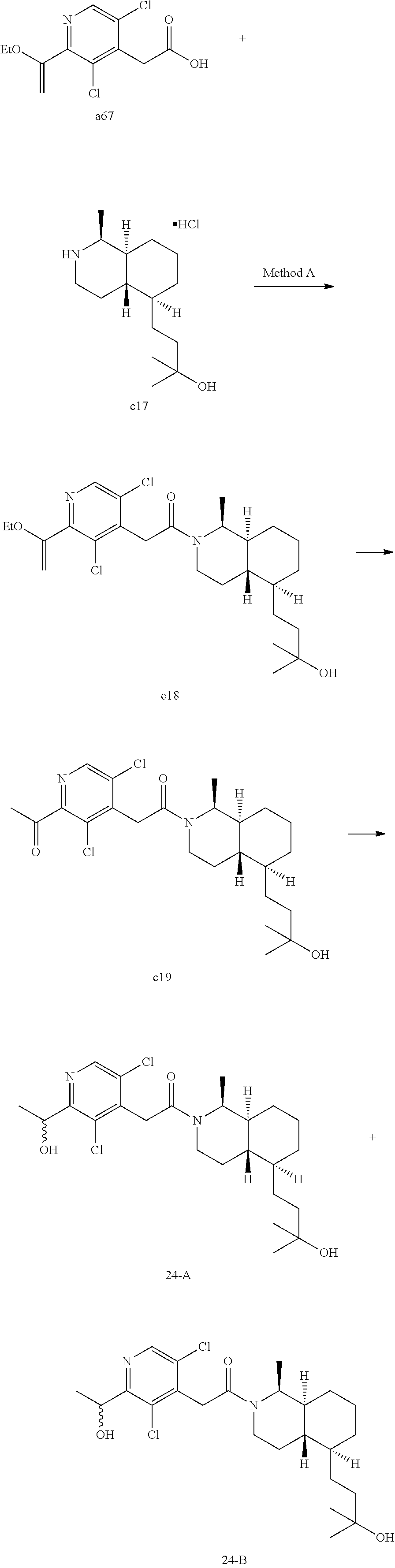

### C.16.1. Synthesis of 1-[(1S,4aS,5S,8aS)-5-(3-hydroxy-3-methyl-butyl)-1-methyl-3,4,4a,5,6,7,8,8a-octahydro-1H-isoquinolin-2-yl]-2-[3,5-dichloro-2-(1-ethoxyvinyl)-4-pyridyl]ethanone c18

1-[(1 S,4aS,5S,8aS)-5-(3-hydroxy-3-methyl-butyl)-1-methyl-3,4,4a,5,6,7,8,8a-octahydro-1H-isoquinolin-2-yl]-2-[3,5-dichloro-2-(1-ethoxyvinyl)-4-pyridyl]ethanone c18 was prepared according to Method A, by reacting 4-[(1S,4aS,5S,8aS)-1-methyl-1,2,3,4,4a,5,6,7,8,8a-decahydroisoquinolin-5-yl]-2-methyl-butan-2-ol hydrochloride c17 with 2-[3,5-dichloro-2-(1-ethoxyvinyl)-4-pyridyl]acetic acid a67 in the presence of HBTU and Et₃N (3 equiv.) in DMF. The crude residue was purified by normal phase column chromatography (elution: from 6 to 100% EtOAc in heptane).

Yield: 53%

Acid LCMS Method 1 (ES⁺): 497/499/501 (M+H)⁺.

### C.16.2. Synthesis of 1-[(1S,4aS,5S,8aS)-5-(3-hydroxy-3-methyl-butyl)-1-methyl-3,4,4a,5,6,7,8,8a-octahydro-1H-isoquinolin-2-yl]-2-(2-acetyl-3,5-dichloro-4-pyridyl)ethanone c19

1-[(1 S,4aS,5S,8aS)-5-(3-hydroxy-3-methyl-butyl)-1-methyl-3,4,4a,5,6,7,8,8a-octahydro-1H-isoquinolin-2-yl]-2-[3,5-dichloro-2-(1-ethoxyvinyl)-4-pyridyl]ethanone c18 (550 mg, 1.10 mmol) was dissolved in acetone (10 mL). A 1N aqueous solution of HCl (2 mL) was added and the reaction mixture was stirred 1 h at rt. The reaction mixture was diluted with EtOAc (150 mL) and washed by a saturated aqueous solution of sodium carbonate (50 mL) and brine (50 mL). The organic layer was dried over MgSO₄, filtered and concentrated under vacuum to afford 519 mg of 1-[(1S,4aS,5S,8aS)-5-(3-hydroxy-3-methyl-butyl)-1-methyl-3,4,4a,5,6,7,8,8a-octahydro-1H-isoquinolin-2-yl]-2-(2-acetyl-3,5-dichloro-4-pyridyl)ethanone c19.

Yield (crude): quantitative

Acid LCMS Method 1 (ES⁺): 469/471/473 (M+H)⁺.

### C.16.3. Synthesis of 1-[(1S,4aS,5S,8aS)-5-(3-hydroxy-3-methyl-butyl)-1-methyl-3,4,4a,5,6,7,8,8a-octahydro-1H-isoquinolin-2-yl]-2-[3,5-dichloro-2-(-1-hydroxyethyl)-4-pyridyl]ethanone Isomer A 24-A and 1-[(1S,4aS,5S,8aS)-5-(3-hydroxy-3-methyl-butyl)-1-methyl-3,4,4a,5,6,7,8,8a-octahydro-1H-isoquinolin-2-yl]-2-[3,5-dichloro-2-(-1-hydroxyethyl)-4-pyridyl]ethanone Isomer B 24-B

Sodium borohydride (76.0 mg, 2.00 mmol) was added to a solution of 1-[(1S,4aS,5S,8aS)-5-(3-hydroxy-3-methyl-butyl)-1-methyl-3,4,4a,5,6,7,8,8a-octahydro-1H-isoquinolin-2-yl]-2-(2-acetyl-3,5-dichloro-4-pyridyl)ethanone c19 (470 mg, 1.00 mmol) in THF (10 mL). The reaction mixture was stirred 15 h at rt. The reaction mixture was diluted with DCM (150 mL) and successively washed with a 1 N aqueous solution of HCl (50 mL), a saturated aqueous solution of sodium carbonate (50 mL) and brine (50 mL). The organic layer was dried over MgSO₄, filtered and concentrated under vacuum. The crude residue was purified by normal phase column chromatography (elution: from 6 to 100% EtOAc in heptane) to afford 472 mg of 1-[(1S,4aS,5S,8aS)-5-(3-hydroxy-3-methyl-butyl)-1-methyl-3,4,4a,5,6,7,8,8a-octahydro-1H-isoquinolin-2-yl]-2-[3,5-dichloro-2-([(1S*)-1-hydroxyethyl])-4-pyridyl]ethanone 24 as a mixture of isomers 24-A and 24-B (Yield: 100%, Acid LCMS Method 1 (ES⁺): 471/473/475 (M+H)⁺).

Chiral separation (LC, LuxCell4, 5 μm, 250×10 mm, 4.8 mL/min, 220 nm, 30° C., elution: EtOH/n-heptane/DEA 30/70/0.1) of the above diastereoisomeric mixture 24 afforded:

150 mg of 1-[(1S,4aS,5S,8aS)-5-(3-hydroxy-3-methyl-butyl)-1-methyl-3,4,4a,5,6,7,8,8a-octahydro-1H-isoquinolin-2-yl]-2-[3,5-dichloro-2-([(1 S*)-1-hydroxyethyl])-4-pyridyl]ethanone Isomer A 24-A, as an off white solid.

Yield: 32%

Basic LCMS Method 3 (ES⁺): 471/473/475 (M+H)⁺, 94% purity.

Acid LCMS Method 2 (ES⁺): 471/473/475 (M+H)⁺, 93% purity.

Chiral analysis (LC, LuxCell4, 3 μm, 150×4.6 mm, 1.5 mL/min, 220 nm, 30° C., elution: EtOH/n-heptane/DEA 30/70/0.1): RT 2.72 min, 100% ee.

150 mg of 1-[(1S,4aS,5S,8aS)-5-(3-hydroxy-3-methyl-butyl)-1-methyl-3,4,4a,5,6,7,8,8a-octahydro-1H-isoquinolin-2-yl]-2-[3,5-dichloro-2-([(1 S*)-1-hydroxyethyl])-4-pyridyl]ethanone Isomer B 24-B, as an off white solid.

Yield: 32%

Basic LCMS (ES⁺) Method 3: 471/473/475 (M+H)⁺, 90% purity.

Acid LCMS (ES⁺) Method 2: 471/473/475 (M+H)⁺, 87% purity.

Chiral analysis (LC, LuxCell4, 3 μm, 150×4.6 mm, 1.5 mL/min, 220 nm, 30° C., elution: EtOH/n-heptane/DEA 30/70/0.1): RT 2.96 min, 97% ee.

### C.17. Synthesis of 2-[2-[(1S,4aR,5R,8aS)-1-methyl-5-(2H-triazol-4-yl)-3,4,4a,5,6,7,8,8a-octahydro-1H-isoquinolin-2-yl]-2-oxoethyl]-3-chloro-4-methoxybenzonitrile 25

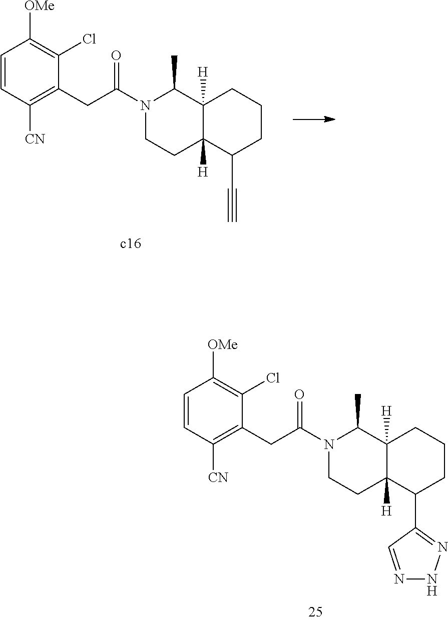 [SMILES: C#CC1CCC[C@H]2[C@H]1CCN(C(=O)Cc1c(C#N)ccc(OC)c1Cl)[C@H]2C ; COc1ccc(C#N)c(CC(=O)N2CC[C@H]3C(c4cn[nH]n4)CCC[C@@H]3[C@@H]2C)c1Cl]

A mixture of 2-[2-[(1S,4aS,5R,8aS)-5-ethynyl-1-methyl-3,4,4a,5,6,7,8,8a-octahydro-1H-isoquinolin-2-yl]-2-oxo-ethyl]-3-chloro-4-methoxy-benzonitrile c16 (222 mg, 0.52 mmol), sodium azide (68.0 mg, 1.04 mmol), sodium ascorbate (10.4 mg, 0.052 mmol), copper(II) sulfate pentahydrate (13.0 mg, 0.052 mmol) and trimethylsilyl azide (189 mg, 1.56 mmol) in 1-butanol (2 mL) and water (2 mL) was stirred at 80° C. for 6 days. The reaction mixture was diluted with EtOAc (150 mL) and successively washed with a 1 N aqueous solution of HCl (50 mL), a saturated aqueous solution of sodium carbonate (50 mL) and brine (50 mL). The organic layer was dried over MgSO₄, filtered and concentrated under vacuum. The crude residue was purified by normal phase column chromatography (elution: from 6 to 100% EtOAc in heptane) to afford 60.0 mg of 2-[2-[(1S,4aR,5R,8aS)-1-methyl-5-(2H-triazol-4-yl)-3,4,4a,5,6,7,8,8a-octahydro-1H-isoquinolin-2-yl]-2-oxoethyl]-3-chloro-4-methoxybenzonitrile 25, as a solid.

Yield: 27%

Basic LCMS Method 3 (ES⁺): 428/430 (M+H)⁺, 93% purity.

Acid LCMS Method 2 (ES⁺): 428/430 (M+H)⁺, 90% purity.

### C.18. Synthesis of 1-[(1S,3R,4aR,5R,8aS)-3-(hydroxymethyl)-1-methyl-5-[(1S)-2,2,2-trifluoro-1-hydroxy-ethyl]-3,4,4a,5,6,7,8,8a-octahydro-1H-isoquinolin-2-yl]-2-(3,5-dichloro-1-methyl-indazol-4-yl)ethanone 26-A and 1-[(1S,3R,4aS,5S,8aR)-3-(hydroxymethyl)-1-methyl-5-[(1S)-2,2,2-trifluoro-1-hydroxy-ethyl]-3,4,4a,5,6,7,8,8a-octahydro-1H-isoquinolin-2-yl]-2-(3,5-dichloro-1-methyl-indazol-4-yl)ethanone 26-B

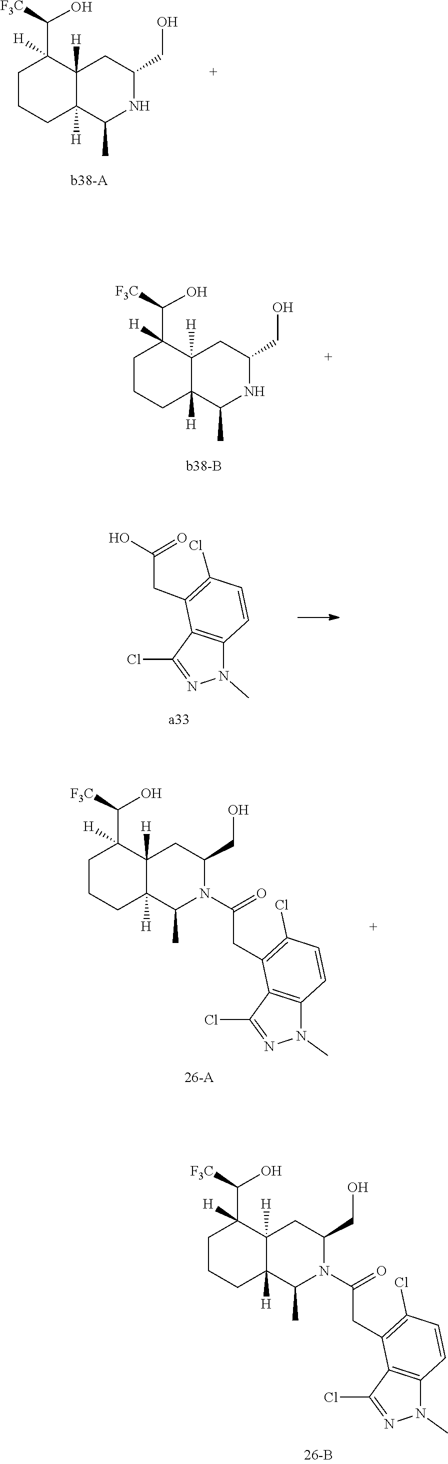 [SMILES: C[C@@H]1N[C@@H](CO)C[C@H]2[C@H]([C@H](O)C(F)(F)F)CCC[C@@H]21 ; C[C@@H]1N[C@@H](CO)C[C@H]2[C@H]1CCC[C@@H]2[C@H](O)C(F)(F)F ; Cn1nc(Cl)c2c(CC(=O)O)c(Cl)ccc21 ; C[C@H]1[C@H]2CCC[C@@H]([C@H](O)C(F)(F)F)[C@@H]2C[C@@H](CO)N1C(=O)Cc1c(Cl)ccc2c1c(Cl)nn2C ; C[C@H]1[C@@H]2CCC[C@H]([C@H](O)C(F)(F)F)[C@H]2C[C@@H](CO)N1C(=O)Cc1c(Cl)ccc2c1c(Cl)nn2C]

To a solution of the isomeric mixture b38 (700 mg, 2.50 mmol) in DMF (8 mL), 2-(3,5-dichloro-1-methyl-indazol-4-yl)acetic acid a33 (970 mg, 3.70 mmol), HBTU (1.10 g, 3.00 mmol) and Et₃N (1.10 mL, 7.50 mmol) were added successively at rt. The reaction mixture was stirred 15 h at rt, then diluted with DCM (150 mL) and successively washed with a 1N aqueous solution of HCl (50 mL), a saturated aqueous solution of sodium carbonate (50 mL) and brine (50 mL). The organic layer was dried over MgSO₄, filtered and concentrated under vacuum. Purification of the crude residue (SFC, P4VP Daicel®, 5 μm, 50×174 mm, 220 nm, 360 mL/min, 30° c., elution: MeOH 10%—CO₂ 90%) afforded 2 fractions:

Fraction 1 was repurified by chiral SFC (Chiralpak AD Daicel®, 20 μm, 50×279 mm 220 nm, 360 mL/min, 35° C., elution: MeOH 15%—CO₂ 85%) to afford 46.0 mg of 1-[(1 S,3R,4aR,5R,8aS)-3-(hydroxymethyl)-1-methyl-5-[(1S)-2,2,2-trifluoro-1-hydroxy-ethyl]-3,4,4a,5,6,7,8,8a-octahydro-1H-isoquinolin-2-yl]-2-(3,5-dichloro-1-methyl-indazol-4-yl)ethanone 26-A, as a white solid.

Yield: 3%

Basic LCMS Method 3 (ES⁺): 522/524/526 (M+H)⁺, 97% purity.

Acid LCMS Method 2 (ES⁺): 522/524/526 (M+H)⁺, 100 purity.

¹H NMR (500 MHz, DMSO-d₆, 75° C.): δ 7.59 (d, J=9.0 Hz, 1H), 7.46 (d, J=8.9 Hz, 1H), 5.91 (d, J=7.0 Hz, 1H), 4.52 (t, J=6.1 Hz, 1H), 4.42 (d, J=16.5 Hz, 1H), 4.16 (d, J=16.5 Hz, 1H), 4.10-4.02 (m, 1H), 4.00 (s, 3H), 3.80-3.69 (m, 2H), 3.65-3.41 (broad peak, 2H), 2.22-2.12 (m, 1H), 1.88 (d, J=12.9 Hz, 1H), 1.78 (dt, J=13.0, 3.3 Hz, 1H), 1.64 (d, J=12.2 Hz, 1H), 1.48-1.19 (m, 9H), 1.02-0.92 (m, 1H).

Chiral analysis (SFC Chiralpak AD, 3 μm, 3×150 mm, 3 mL/min, 30° C., elution: MeOH 20%—CO₂ 80%): RT 0.74 min, 100% de.

Fraction 2 was repurified by chiral SFC (IC, 20 μm, 50×266 mm, 220 nm, 360 mL/min, 35° C., MeOH 25%—CO₂ 75%) to afford 270 mg of 1-[(1S,3R,4aS,5R,8aS)-3-(hydroxymethyl)-1-methyl-5-[(1S)-2,2,2-trifluoro-1-hydroxy-ethyl]-3,4,4a,5,6,7,8,8a-octahydro-1H-isoquinolin-2-yl]-2-(3,5-dichloro-1-methyl-indazol-4-yl)ethenone 26-B, as a white solid.

Yield: 26%

Basic LCMS Method 3 (ES⁺): 522/524/526 (M+H)⁺, 100% purity.

Acid LCMS Method 2 (ES⁺): 522/524/526 (M+H)⁺, 99% purity.

¹H NMR (500 MHz, DMSO-d₆, 75° C.): δ 7.59 (d, J=9.0 Hz, 1H), 7.47 (d, J=9.0 Hz, 1H), 5.86 (d, J=6.7 Hz, 1H), 4.65-4.42 (broad peak, 1H), 4.34 (dd, J=18.5 Hz, 2H), 4.22-4.12 (m, 2H), 4.00 (s, 3H), 3.67 (dd, J=5.8 Hz, 2H), 3.62-3.40 (broad peak, 1H), 1.99 (ddd, J=13.6, 6.0, 3.8 Hz, 1H), 1.80 (dt, J=12.6, 2.9 Hz, 1H), 1.73-1.53 (m, 4H), 1.53-1.28 (m, 4H), 1.20 (d, J=6.7 Hz, 3H), 1.12-1.00 (m, 1H).

Chiral analysis (SFC Chiralpak IC, 3 μm, 3×150 mm, 3 mL/min, 30° C., elution: MeOH 20%—CO₂ 80%): RT 2.26 min, 99% de.

X-Ray diffraction of Example 26-A: a colourless block-like single crystal was selected and mounted on the MiTeGen MicroMounts sample holder. Single-crystal X-ray diffraction data were collected using the Oxford Diffraction Gemini R Ultra diffractometer (Mo Kα, graphite monochromator, Ruby CCD area detector) at 100 (2) K. Data collection, unit cells determination and data reduction were carried out using CrysAlis PRO software package. Using Olex2 and shelXle, the structure was solved with the SHELXT 2015 structure solution program by Intrinsic Phasing methods and refined by full-matrix least squares on |F|² using SHELXL-2018/3. Non-hydrogen atoms were refined anisotropically. Hydrogen atoms were placed on calculated positions in riding mode with temperature factors fixed at 1.2 times Ueq of the parent carbon atoms (1.5 for methyl groups).

The asymmetric unit contains two molecules of Example (26-B) and one molecule of disordered butanone.

Crystal Data for C₂₃H₂₈Cl₂F₃N₃O₃ (M=522.4 g/mol): tetragonal, space group P4₃2₁2 (no. 96), a=b=12.0001 (3) Å, c=35.1592 (11) Å, V=5063.0 (3) Å³, Z=8, T=100 (2) K, λ(MoKα)=0.71073, μcalc=1.371 g/cm³, 26173 reflections measured (4.63°≤2Θ≤52.74°), 5174 independent reflections (Rint=0.0505, Rsigma=0.0325) which were used in all calculations. The final R₁ was 0.0516 (I>2−(I)) and R₂ was 0.1089 (all data).

Absolute configuration established by anomalous-dispersion effects in diffraction measurements on the crystal. Flack x parameter determined using 1843 quotients [(I+)−(I−)]/[(I+)+(I−)]6 and equal to 0.00 (3), indicating the absolute configuration as displayed in section C.18 above (Example 26-B). The asymmetric unit contains one molecule of Example 26-B.

### D. Camp Htrf Assay.

Compounds according to the present invention do not directly activate the dopamine D1 receptor, but potentiate the effect of D1 agonists or the endogenous ligand on D1 receptors, dopamine, through an allosteric mechanism, and are therefore D1 positive allosteric modulators (D1 PAM).

Dopamine and other D1 agonists directly activate the dopamine D1 receptor by themselves.

The present assay allows to measure respectively the effects of compounds of the Examples in the absence of dopamine (“activation assay”) and the effects of compounds of the Examples in the presence of dopamine (“potentiation assay”).

The activation assay measures the stimulation of the production of cyclic adenosinemonophosphate (cAMP) in the HTRF assay, with the maximum increase in cAMP by increasing concentrations of the endogenous agonist, dopamine, defined as 100% activation. When tested compounds of the Examples lack significant direct agonist-like effects in that they produce less than 20% of activation (compared to dopamine maximal response) when present in a concentration of 10 μM.

The potentiation assay measures the ability of compounds to increase the levels of cAMP produced by a low-threshold concentration of dopamine. The concentration of dopamine used ([EC₂₀]) is designed to produce 20% stimulation compared to the maximal response (100%) seen with increasing the concentration of dopamine. To measure this potentiation we incubate increasing concentrations of the compound with the [EC₂₀] of dopamine and measure the potentiation as increases in cAMP production. The pEC₅₀ of a compound is the −log 10 of the concentration of the compound which produces 50% of the potentiation of the cAMP levels and the Erel is the relative efficacy, defined as the maximal % potentiation produced by the compound compared to the maximal response produced by increasing concentrations of dopamine (Erel of 1=dopamine maximum response).

The particular conditions in which the compounds have been tested are described here below.

Methods D1 Cell Culture

Cells were cultured at 37° C. in a humidified atmosphere of 5% CO₂. Cells were grown in DMEM-F12+GlutaMAX™-1 medium (GIBCO®, Invitrogen, Merelbeke, Belgium) containing 10% fetal bovine serum (BioWhittaker®, Lonza, Verviers, Belgium), 400 μg/mL Geneticin (GIBCO®), 100 IU/mL Penicillin and 100 IU/mL Streptomycin (Pen-Strep solution, BioWhittaker®). LMtk (Ltk−) mouse fibroblast cells expressing the dopamine D1 receptor (BioSignal Inc, Montreal, Canada, now Perkin Elmer) were used as they have been shown to couple efficiently and give robust functional responses (Watts et al, 1995).

cAMP Assay

The measurement of changes in intracellular cyclic adenosinemonophopshpate (cAMP) was determined using the HTRF cAMP dynamic assay kit from CisBio (Codolet, France). Using homogenous time-resolved fluoresence technology, the assay is based on competition between native cAMP produced by cells and cAMP labelled with the dye d2. The tracer binding is determined by an anti-cAMP antibody labeled with cryptate. The effects of the compound alone (agonism) was determined by performing the assay in the absence of dopamine, whilst the effect of the compound as a positive allosteric modulator (PAM) was determined in the presence of an EC₂₀ concentration of dopamine. Cells (20,000 per well) are incubated in 384 plates for 1 h at rt in a final volume of 20 μLHBSS (Lonza, with calcium, magnesium and HEPES buffer 20 mM, pH 7.4) containing: isobutyl methylxanthine (Sigma, 0.1 mM final), varying concentrations of test compound (typically 10^{-9.5} M to 10^{-4.5} M) in the presence and absence of dopamine (1.1 nM final). The reaction is then terminated and the cells lysed by adding the d2 detection reagent in lysis buffer (10 μL) and the cryptate reagent in lysis buffer (10 μL) according to manufacturer's instructions. This is then incubated for a further 60 min at rt and changes in HTRF fluorescent emission ratio determined according to manufacturer's instructions using an Envision plate reader (Perkin Elmer, Zaventem, Belgium) with laser excitation. All incubations were performed in duplicate and results were compared to a concentration-effect curve to dopamine. (10⁻¹¹ M to 10⁻⁶ M).

Data Analysis

Data was analyzed using Excel and PRISM (GraphPad Software) to obtain pEC₅₀ and Erel using the 4-parameter logistic equation (DeLean et al, 1978) where Erel is the fitted maximal response of the test compound minus basal expressed as a percentage relative to that obtained with dopamine which was defined as 100%.

When tested in the cAMP HTRF assay, Examples of compounds of formula (I) according to the Examples exhibit values as displayed in Table A below:

TABLE A

| Example # | pEC₅₀ |
|---|---|
| 1-A | 7.5 |
| 1-B | 6.4 |
| 2 | 7.4 |
| 3 | 7.6 |
| 5 | 7.5 |
| 6-A | 7.3 |
| 6-B | 6.7 |
| 7-A | 6.6 |
| 7-B | 6.1 |
| 8-A | 7.1 |
| 8-B | 6.9 |
| 9-A | 7.4 |
| 9-B | 7.6 |
| 10 | 7.9 |
| 11 | 7.2 |
| 12 | 7.8 |
| 13 | 7.3 |
| 14 | 7.5 |
| 15-A | 7.4 |
| 15-B | 7.6 |
| 16-A | 6.8 |
| 16-B | 7.1 |
| 17-A | 6.8 |
| 17-B | 6.9 |
| 18-A | 6.7 |
| 18-B | 6.8 |
| 19 | 6.7 |
| 20 | 6.8 |
| 21 | 7.3 |
| 22 | 6.7 |
| 23 | 7.3 |
| 24-A | 7.1 |
| 24-B | 7.4 |
| 25 | 6.2 |
| 26-A | 5.5 |
| 26-B | 7.3 |
| 27 | 6.6 |

### E. Automated Patch Clamp Studies on the GABA_{A} Receptor Cells

CHO-K1 cells stably expressing human GABA_{A} receptor α1β2 and γ2 subunits were used. The cells were harvested using trypsin and maintained in serum-free medium at room temperature. The cells were washed and re-suspended in extracellular solution before testing.

### Patch Clamp Studies

Experiments on human GABA_{A} (α₁β₂γ2) channels were conducted using an automated patch clamp assay (lonFlux™ HT). The external solution for recording GABA_{A} currents was composed of sodium chloride 137 mM, potassium chloride 4 mM, calcium chloride 1.8 mM, magnesium chloride 1 mM, HEPES 10 mM, and glucose 10 mM. Both external and internal solutions were titrated with NaOH or KOH to obtain a pH of 7.35 and 7.3, respectively. The internal pipette solution contained potassium fluoride 70 mM, potassium chloride 60 mM, sodium chloride 70 mM, HEPES 5 mM, EGTA 5 mM, and Magnesium ATP 4 mM. The final concentration of vehicle used to dilute compounds was 0.33% DMSO in each well. Bicuculline (0.032 to 100 μM) was used as positive control inhibitor. GABA (15 μM) was used as agonist. All recordings were obtained from a holding potential of −60 mV.

The compound addition sequence was the following: one addition of the EC₈₀ concentration of GABA was added to establish baseline response. Each concentration of compound was applied for 30 seconds followed by the addition of 15 μM GABA in the presence of the compound for 2 seconds. The process was repeated with the next ascending concentration of compound. Peak inward currents in response to the GABA additions in the presence of a single concentration of compound were measured. All compound data have been normalized to the baseline peak current induced by addition of 15 μM GABA for 2 seconds.

When tested in the above-mentioned assay, at a concentration of 10 μM, compounds of formula (I) according to the Examples exhibit a percentage of inhibition of the GABA_{A} receptor as displayed in Table B below.

TABLE B

| | % GABA_{A} | | |
|---|---|---|---|
| Example # | inhibition | Example # | pEC₅₀ |
| 1-A | 11.2 | 15-B | 11.6 |
| 1-B | 4.9 | 16-A | 15.8 |
| 2 | 0.0 | 16-B | 16.9 |
| 3 | 13.8 | 17-A | 2.1 |
| 5 | 10.8 | 17-B | −2.3 |
| 6-A | 8.6 | 18-A | −4.0 |
| 6-B | 10.9 | 18-B | 5.8 |
| 7-A | 7.4 | 19 | 17.3 |
| 7-B | 1.4 | 20 | 4.4 |
| 8-A | 2.0 | 21 | −12.3 |
| 8-B | −1.2 | 22 | −10.5 |
| 9-A | −14.1 | 15-A | 14.0 |
| 9-B | −8.4 | 23 | −8.5 |
| 10 | 8.5 | 24-A | 5.4 |
| 11 | 6.0 | 24-B | 4.5 |
| 12 | 9.6 | 25 | 1.4 |
| 13 | −8.0 | 26-B | −1.8 |
| 14 | −8.0 | 27 | −15.3 |

### F. In Vitro Assessment of CYP3A4 Inhibition Potential Using Cryopreserved Human Microsomes

The objective of the human microsome assay is to characterize the inhibition potential of Compound of formula (I) by measuring the CYP3A4 activities after its co-incubation with midazolam, a specific CYP3A4 substrate.

To this aim, cryopreserved human microsomes (pooled donors) are divided on a 48 well collagen coated plate so that the final concentration is 0.25 mg/ml. The UCB compound is then added in the wells at 20 μM concentration in duplicate. After 30 min incubation, midazolam is added at 2.5 μM concentration. After 15 min, an aliquot is removed and placed into an equal volume of methanol containing internal standard. The samples are centrifuged at 2500 rpm at 4° C. for 20 min. An aliquot of supernatant is diluted with deionised water and levels of 1-hydroxymidazolam is quantified using generic LC MS/MS methods.

The concentrations are compared to those obtained after midazolam incubation at the same concentration but without UCB compound pre-incubation. The results are expressed as % of inhibition.

When tested in the above assay, compounds of formula (I) according to the Examples exhibit a percentage of inhibition of CYP3A4 as displayed in the following Table C.

A percentage of inhibition greater than about 70% and lower than about 80% is indicated by +.

A percentage of inhibition greater than about 60% and lower than or equal to about 70% is indicated by ++.

A percentage of inhibition greater than about 40% and lower than or equal to about 60% is indicated by +++.

A percentage of inhibition greater than about 20% and lower than or equal to about 40% is indicated by ++++.

A percentage of inhibition lower than or equal to about 20% is indicated by +++++.

TABLE C

| | % CYP3A4 | | |
|---|---|---|---|
| Example # | inhibition | Example # | pEC₅₀ |
| 1-A | ++ | 15-B | ++++ |
| 1-B | +++ | 16-A | ++++ |
| 2 | ++++ | 16-B | ++++ |
| 3 | ++ | 17-A | +++++ |
| 5 | ++++ | 17-B | +++++ |
| 6-A | ++++ | 18-A | +++++ |
| 6-B | ++++ | 18-B | +++++ |
| 7-A | ++++ | 19 | +++ |
| 8-A | ++++ | 20 | ++++ |
| 8-B | +++ | 21 | +++ |
| 9-A | ++++ | 22 | ++++ |
| 9-B | ++++ | 23 | ++++ |
| 10 | ++++ | 24-A | ++++ |
| 11 | + | 24-B | ++++ |
| 12 | ++++ | 25 | +++ |
| 13 | ++++ | 26-B | ++++ |
| 14 | +++ | 27 | +++++ |
| 15-A | +++++ | | |

1. A compound of formula (I), or a pharmaceutically acceptable salt thereof, 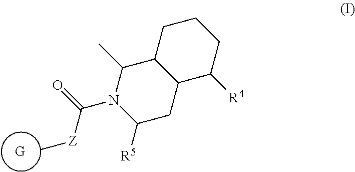
wherein
Z represents CH₂ or NH;
R⁴ represents C₁₋₆ alkyl optionally substituted by one or more substituents selected from hydroxy, halogen and C₁₋₆ alkyl; or C₁₋₆ alkyne optionally substituted by one or more substituents selected from hydroxy and C₁₋₆ alkyl; or C₅₋₈ heteroaryl, optionally substituted by one or more substituents selected from halogen, cyano, C₁₋₆ alkyl and C₁₋₆ alkoxy;
R⁵ represents hydrogen or a C₁₋₆ alkyl optionally substituted by one or more substituents selected from hydroxy and halogen; and
G represents an aromatic group selected from the group consisting of (G^{a}), (G^{b}) and (G^{c}); 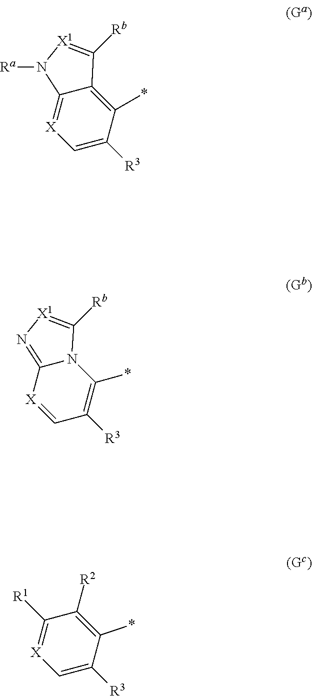
wherein
the asterisk (*) represents the point of attachment of G to the remainder of the molecule;
X represents CH, C—F or N;
R¹ represents hydrogen; or C₁₋₆ alkyl or C₁₋₆ alkoxy optionally substituted by one or more substituents selected from hydroxy and halogen;
R² and R³ represent independently halogen or cyano;
X¹ represents CH or N;
R^{a} represents hydrogen or C₁₋₆ alkyl; and
R^{b} represents C₁₋₆ alkyl or halogen.

2. A compound of formula (I) according to claim 1, represented by formula (IA), or a pharmaceutical acceptable salt thereof 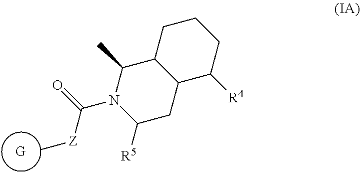
wherein G, R⁴, R⁵, Z and X are as defined in claim 1.

3. A compound of formula (I) according to claim 1, represented by formula (IA-a), or a pharmaceutical acceptable salt thereof 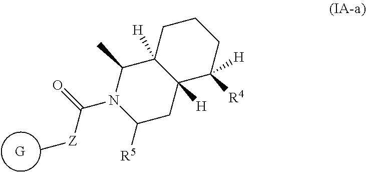
wherein G, R⁴, R⁵, Z and X are as defined in claim 1.

4. A compound of formula (I) according to claim 1 wherein Z represents CH₂.

5. A compound of formula (I) according to claim 1 wherein R⁴ represents C₁₋₆ alkyl substituted by one or more hydroxy and by one or more halogen; C₁₋₆ alkyl substituted by one or more C₁₋₆ alkyl and by one or more hydroxy; C₁₋₆ alkyne substituted by one or more hydroxy and one or more C₁₋₆ alkyl.

6. A compound of formula (I) according to claim 1 wherein G represents (G^{c}).

7. A compound of formula (I) according to claim 1, represented by formula (IB-aa), or a pharmaceutically acceptable salt thereof 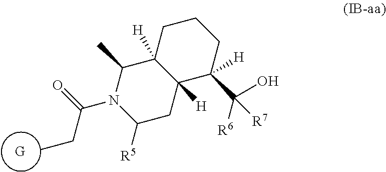
wherein
R⁶ and R⁷ represent independently hydrogen or a C₁₋₆ alkyl, optionally substituted by one or more halogen; and
G, R⁵, and X are as defined in claim 1.

8. A compound of formula (I) according to claim 1 wherein R¹ represents C₁₋₆ alkyl substituted by one or more hydroxy, C₁₋₆ alkyl substituted by one or more hydroxy and by one or more halogen, C₁₋₆ alkoxy, or C₁₋₆ alkoxy substituted by one or more halogen.

9. A compound of formula (I) according claim 1 wherein R⁵ represents hydrogen.

10. A compound of formula (IB-aa) according to claim 7 wherein R⁶ represents hydrogen or C₁₋₆ alkyl and R⁷ represents C₁₋₆ alkyl, which group may be optionally substituted by one or more halogen.

11. A compound of formula (IB-aa) according to claim 7 wherein
G represents (G^{c});
X represents C—H or N;
R¹ represents C₁₋₆ alkyl substituted by one or more hydroxy or C₁₋₆ alkoxy;
R² and R³ represent independently halogen or cyano;
R⁵ represents hydrogen;
R⁶ represents hydrogen or C₁₋₆ alkyl; and
R⁷ represents C₁₋₆ alkyl substituted by one or more halogen.

12. A compound as claimed in claim 1 which is selected from the group consisting of:
2-[2-[(1S,4aR,5R,8aS)-1-methyl-5-[(1S)-2,2,2-trifluoro-1-hydroxy-ethyl]-3,4,4a,5,6,7,8,8a-octahydro-1H-isoquinolin-2-yl]-2-oxo-ethyl]-3-chloro-4-methoxybenzonitrile;
2-[2-[(1S,4aR,5R,8aS)-1-methyl-5-[(1R)-2,2,2-trifluoro-1-hydroxy-ethyl]-3,4,4a,5,6,7,8,8a-octahydro-1H-isoquinolin-2-yl]-2-oxo-ethyl]-3-chloro-4-methoxybenzonitrile;
2-[2-[(1S,4aR,5R,8aS)-1-methyl-5-[(1S)-2,2,2-trifluoro-1-hydroxy-ethyl]-3,4,4a,5,6,7,8,8a-octahydro-1H-isoquinolin-2-yl]-2-oxo-ethyl]-3-chloro-6-methoxybenzonitrile;
1-[(1S,4aR,5R,8aS)-1-methyl-5-[(1S)-2,2,2-trifluoro-1-hydroxy-ethyl]-3,4,4a,5,6,7,8,8a-octahydro-1H-isoquinolin-2-yl]-2-(3,5-dichloro-2-methoxypyridin-4-yl)ethanone;
2-[2-[(1S,4aR,5R,8aS)-1-methyl-5-[(1S)-2,2,2-trifluoro-1-hydroxy-ethyl]-3,4,4a,5,6,7,8,8a-octahydro-1H-isoquinolin-2-yl]-2-oxo-ethyl]-3-chloro-4-(trideuteriomethoxy)benzonitrile;
1-[(1S,4aR,5R,8aS)-1-methyl-5-[(1S)-2,2,2-trifluoro-1-hydroxy-ethyl]-3,4,4a,5,6,7,8,8a-octahydro-1H-isoquinolin-2-yl]-2-(3,6-dichloro-[1,2,4]triazolo[4,3-a]pyridin-5-yl)ethanone;
1-[(1S,4aR,5R,8aS)-1-methyl-5-[(1S)-2,2,2-trifluoro-1-hydroxy-ethyl]-3,4,4a,5,6,7,8,8a-octahydro-1H-isoquinolin-2-yl]-2-[3,5-dichloro-2-(hydroxymethyl)-4-pyridyl]ethanone;
1-[(1S,4aR,5R,8aS)-1-methyl-5-[(1S)-2,2,2-trifluoro-1-hydroxy-ethyl]-3,4,4a,5,6,7,8,8a-octahydro-1H-isoquinolin-2-yl]-2-(3,5-dichloro-7-fluoro-1H-indazol-4-yl)ethanone;
1-[(1S,4aR,5R,8aS)-1-methyl-5-[(1S)-2,2,2-trifluoro-1-hydroxy-ethyl]-3,4,4a,5,6,7,8,8a-octahydro-1H-isoquinolin-2-yl]-2-(3,5-dichloro-1-methyl-indol-4-yl)ethenone;
1-[(1S,4aR,5R,8aS)-1-methyl-5-[(1S)-2,2,2-trifluoro-1-hydroxy-ethyl]-3,4,4a,5,6,7,8,8a-octahydro-1H-isoquinolin-2-yl]-2-[2,6-dichloro-3-(difluoromethoxy)phenyl]ethanone;
1-[(1S,4aR,5R,8aS)-1-methyl-5-[(1S)-2,2,2-trifluoro-1-hydroxy-ethyl]-3,4,4a,5,6,7,8,8a-octahydro-1H-isoquinolin-2-yl]-2-[3,5-dichloro-2-[(1S)-1-hydroxyethyl]-4-pyridyl]ethanone;
1-[(1S,4aR,5R,8aS)-1-methyl-5-[(1S)-2,2,2-trifluoro-1-hydroxy-ethyl]-3,4,4a,5,6,7,8,8a-octahydro-1H-isoquinolin-2-yl]-2-[3,5-dichloro-2-[(1R)-1-hydroxyethyl]-4-pyridyl]ethanone;
2-[2-[(1S,4aR,5R,8aS)-5-[(1R)-2,2-difluoro-1-hydroxy-ethyl]-1-methyl-3,4,4a,5,6,7,8,8a-octahydro-1H-isoquinolin-2-yl]-2-oxo-ethyl]-3-chloro-4-methoxy-benzonitrile;
2-[2-[(1S,4aR,5R,8aS)-5-[(1S)-2,2-difluoro-1-hydroxy-ethyl]-1-methyl-3,4,4a,5,6,7,8,8a-octahydro-1H-isoquinolin-2-yl]-2-oxo-ethyl]-3-chloro-4-methoxy-benzonitrile;
2-[2-[(1S,4aR,5R,8aS)-1-methyl-5-[(1R)-2,2,2-trifluoro-1-hydroxy-1-methyl-ethyl]-3,4,4a,5,6,7,8,8a-octahydro-1H-isoquinolin-2-yl]-2-oxo-ethyl]-3-chloro-4-methoxy-benzonitrile;
2-[2-[(1S,4aR,5R,8aS)-1-methyl-5-[(1S)-2,2,2-trifluoro-1-hydroxy-1-methyl-ethyl]-3,4,4a,5,6,7,8,8a-octahydro-1H-isoquinolin-2-yl]-2-oxo-ethyl]-3-chloro-4-methoxy-benzonitrile;
1-[(1S,4aR,8aS)-1-methyl-5-[(1R)-2,2,2-trifluoro-1-hydroxy-ethyl]-3,4,4a,5,6,7,8,8a-octahydro-1H-isoquinolin-2-yl]-2-(3,5-dichloro-1-methyl-indazol-4-yl)ethanone;
1-[(1S,4aR,8aS)-1-methyl-5-[(1S)-2,2,2-trifluoro-1-hydroxy-ethyl]-3,4,4a,5,6,7,8,8a-octahydro-1H-isoquinolin-2-yl]-2-(3,5-dichloro-1-methyl-indazol-4-yl)ethanone;
(1S,4aR,5R,8aS)—N-(2,6-dichlorophenyl)-1-methyl-5-[(1S)-2,2,2-trifluoro-1-hydroxy-ethyl]-3,4,4a,5,6,7,8,8a-octahydro-1H-isoquinoline-2-carboxamide;
1-[(1S,4aR,5R,8aS)-1-methyl-5-[(1S)-2,2,2-trifluoro-1-hydroxy-ethyl]-3,4,4a,5,6,7,8,8a-octahydro-1H-isoquinolin-2-yl]-2-[3,5-dichloro-2-(1-hydroxy-1-methyl-ethyl)-4-pyridyl]ethanone;
1-[(1S,4aR,5R,8aS)-1-methyl-5-[(1S)-2,2,2-trifluoro-1-hydroxy-ethyl]-3,4,4a,5,6,7,8,8a-octahydro-1H-isoquinolin-2-yl]-2-[3,5-dichloro-2-[(1R)-2,2-difluoro-1-hydroxy-ethyl]-4-pyridyl]ethanone;
1-[(1S,4aR,5R,8aS)-1-methyl-5-[(1S)-2,2,2-trifluoro-1-hydroxy-ethyl]-3,4,4a,5,6,7,8,8a-octahydro-1H-isoquinolin-2-yl]-2-[3,5-dichloro-2-[(1S)-2,2-difluoro-1-hydroxy-ethyl]-4-pyridyl]ethanone;
1-[(1S,4aR,5R,8aS)-1-methyl-5-[(1R)-2,2,2-trifluoro-1-hydroxy-ethyl]-3,4,4a,5,6,7,8,8a-octahydro-1H-isoquinolin-2-yl]-2-[3,5-dichloro-2-[(1R)-2,2-difluoro-1-hydroxy-1-methyl-ethyl]-4-pyridyl]ethanone;
1-[(1S,4aR,5R,8aS)-1-methyl-5-[(1R)-2,2,2-trifluoro-1-hydroxy-ethyl]-3,4,4a,5,6,7,8,8a-octahydro-1H-isoquinolin-2-yl]-2-[3,5-dichloro-2-[(1R)-2,2-difluoro-1-hydroxy-1-methyl-ethyl]-4-pyridyl]ethanone;
1-[(1S,4aR,5R,8aS)-5-[(1S)-2,2-difluoro-1-hydroxy-ethyl]-1-methyl-3,4,4a,5,6,7,8,8a-octahydro-1H-isoquinolin-2-yl]-2-[3,5-dichloro-2-[(1S)-1-hydroxyethyl]-4-pyridyl]ethanone;
1-[(1S,4aR,5R,8aS)-5-[(1S)-2,2-difluoro-1-hydroxy-ethyl]-1-methyl-3,4,4a,5,6,7,8,8a-octahydro-1H-isoquinolin-2-yl]-2-[3,5-dichloro-2-[(1R)-1-hydroxyethyl]-4-pyridyl]ethanone;
1-[(1S,4aR,5R,8aS)-5-[(1S)-2,2-difluoro-1-hydroxy-1-methyl-ethyl]-1-methyl-3,4,4a,5,6,7,8,8a-octahydro-1H-isoquinolin-2-yl]-2-[3,5-dichloro-2-[(1S)-1-hydroxyethyl]-4-pyridyl]ethanone;
1-[(1S,4aR,5R,8aS)-5-[(1S)-2,2-difluoro-1-hydroxy-1-methyl-ethyl]-1-methyl-3,4,4a,5,6,7,8,8a-octahydro-1H-isoquinolin-2-yl]-2-[3,5-dichloro-2-[(1R)-1-hydroxyethyl]-4-pyridyl]ethanone;
2-[2-[(1S,4aS,8aS)-5-(3-hydroxy-3-methyl-but-1-ynyl)-1-methyl-3,4,4a,5,6,7,8,8a-octahydro-1H-isoquinolin-2-yl]-2-oxo-ethyl]-3-chloro-4-methoxy-benzonitrile;
2-[2-[(1S,4aS,5S,8aS)-5-(3-hydroxy-3-methyl-butyl)-1-methyl-3,4,4a,5,6,7,8,8a-octahydro-1H-isoquinolin-2-yl]-2-oxo-ethyl]-3-chloro-4-methoxy-benzonitrile;
1-[(1S,4aS,5S,8aS)-5-(3-hydroxy-3-methyl-butyl)-1-methyl-3,4,4a,5,6,7,8,8a-octahydro-1H-isoquinolin-2-yl]-2-(3,5-dichloro-1-methyl-indazol-4-yl)ethanone;
1-[(1S,4aS,5S,8aS)-5-(3-hydroxy-3-methyl-butyl)-1-methyl-3,4,4a,5,6,7,8,8a-octahydro-1H-isoquinolin-2-yl]-2-[3,5-dichloro-2-(hydroxymethyl)-4-pyridyl]ethanone;
1-[(1S,4aS,5S,8aS)-5-(3-hydroxy-3-methyl-butyl)-1-methyl-3,4,4a,5,6,7,8,8a-octahydro-1H-isoquinolin-2-yl]-2-[3,5-dichloro-2-[(1S)-1-hydroxyethyl]-4-pyridyl]ethanone;
1-[(1S,4aS,5S,8aS)-5-(3-hydroxy-3-methyl-butyl)-1-methyl-3,4,4a,5,6,7,8,8a-octahydro-1H-isoquinolin-2-yl]-2-[3,5-dichloro-2-[(1R)-1-hydroxyethyl]-4-pyridyl]ethanone;
2-[2-[(1S,4aR,5R,8aS)-1-methyl-5-(2H-triazol-4-yl)-3,4,4a,5,6,7,8,8a-octahydro-1H-isoquinolin-2-yl]-2-oxoethyl]-3-chloro-4-methoxybenzonitrile;
1-[(1S,3R,4aR,5R,8aS)-3-(hydroxymethyl)-1-methyl-5-[(1S)-2,2,2-trifluoro-1-hydroxy-ethyl]-3,4,4a,5,6,7,8,8a-octahydro-1H-isoquinolin-2-yl]-2-(3,5-dichloro-1-methyl-indazol-4-yl)ethanone; and
1-[(1S,3R,4aS,5S,8aR)-3-(hydroxymethyl)-1-methyl-5-[(1S)-2,2,2-trifluoro-1-hydroxy-ethyl]-3,4,4a,5,6,7,8,8a-octahydro-1H-isoquinolin-2-yl]-2-(3,5-dichloro-1-methyl-indazol-4-yl)ethanone.

13. (canceled)

14. (canceled)

15. (canceled)

16. (canceled)

17. (canceled)

18. A method for the treatment and/or prevention of disorders for which the administration of D1 positive allosteric modulator is indicated, which comprises administering to a patient in need of such treatment an effective amount of a compound of formula (I) according to claim 1, or a pharmaceutically acceptable salt thereof.

19. A method for the treatment and/or prevention of cognitive and negative symptoms in schizophrenia, cognitive impairment related to neuroleptic therapy, Mild Cognitive Impairment (MCI), impulsivity, Attention-Deficit Hyperactivity Disorder (ADHD), Parkinson's disease and other movement disorders, dystonia, Parkinson's dementia, Huntington's disease, dementia with Lewy Body, Alzheimer's disease, drug addiction, sleep disorders, apathy, traumatic spinal cord injury or neuropathic pain, which comprises administering to a patient in need of such treatment an effective amount of a compound of formula (I) according to claim 1, or a pharmaceutically acceptable salt thereof.

20. A pharmaceutical composition comprising a compound according to claim 1, or a pharmaceutically acceptable salt thereof, in association with a pharmaceutically acceptable carrier.

21. A compound of formula (I) according to claim 3 wherein R¹ represents C₁₋₆ alkyl substituted by one or more hydroxy, C₁₋₆ alkyl substituted by one or more hydroxy and by one or more halogen, C₁₋₆ alkoxy, or C₁₋₆ alkoxy substituted by one or more halogen.

22. A compound of formula (I) according claim 21 wherein R⁵ represents hydrogen.

23. A compound of formula (I) according to claim 7 wherein R¹ represents C₁₋₆ alkyl substituted by one or more hydroxy, C₁₋₆ alkyl substituted by one or more hydroxy and by one or more halogen, C₁₋₆ alkoxy, or C₁₋₆ alkoxy substituted by one or more halogen.

24. A compound of formula (I) according claim 23 wherein R⁵ represents hydrogen.

25. A compound of formula (I) according to claim 4 wherein R¹ represents C₁₋₆ alkyl substituted by one or more hydroxy, C₁₋₆ alkyl substituted by one or more hydroxy and by one or more halogen, C₁₋₆ alkoxy, or C₁₋₆ alkoxy substituted by one or more halogen.
